(19) United States
(12) Patent Application Publication
(10) Pub. No.: US 2026/0275324 A1
(43) Pub. Date: Sep. 17, 2026

(54) UNNATURAL AMINO ACID IN CELLULO SYNTHESIS FOR SITE-SPECIFIC PROTEIN MODIFICATION

(71) Applicant: The Chinese University of Hong Kong, Shatin, New Territories, Hong Kong SAR (CN)

(72) Inventors: Michael Kenneth CHAN, Kowloon, Hong Kong SAR (CN); Marianne LEE, Kowloon, Hong Kong SAR (CN); Jiahui CHENG, Shatin, N.T., Hong Kong SAR (CN)

(21) Appl. No.: 19/470,748

(22) PCT Filed: Mar. 29, 2024
(86) PCT No.: PCT/CN2024/084877

§ 371 Date: Sep. 29, 2025

Related U.S. Application Data
(60) Provisional application No. 63/455,799, filed on Mar. 30, 2023.

(51) Int. Cl. C12N 9/00; C07K 14/47

(52) U.S. Cl. CPC C12N 9/93; C07K 14/4703; C12Y 601/01026

(57) ABSTRACT

Provided are genetically modified host cells containing engineered enzymes that support the synthesis of unnatural amino acids, such as D-Cys-ε-Lys, and the incorporation of such unnatural amino acids into a newly synthesized protein. These cells are therefore capable of in vivo or in cellulo synthesis of a protein containing one or more unnatural amino acids strategically placed at pre-determined locations for site-specific modification of the protein. Also provided are the corresponding methods for in vivo synthesis of modified proteins as well as the modified proteins produced by the method.

### CROSS-REFERENCE TO RELATED APPLICATION

This application claims priority to U.S. Provisional Patent Application No. 63/455,799, filed Mar. 30, 2023, the disclosure of which is herein incorporated by reference in its entirety for all purposes.

### BACKGROUND

Protein therapeutics are important alternatives to small molecule drugs for the treatment of diseases due to their generally higher specificity and lower toxicity. However, one major limitation to protein-based therapeutics is their rapid degradation by proteolytic enzymes in vivo leading to short serum half-lives, which adversely affect their efficacies (Sato, Aaron K et al. Current Opinion in Biotechnology vol. 17, 6 (2006): 638-42). Protein modifications such as cyclization can be a useful approach to overcome this serum stability challenge and cyclized proteins typically exhibit dramatically extended in vivo lifetimes and improved binding affinities due to reduction in the entropic cost of binding (Colgrave, Michelle L, and David J Craik. Biochemistry vol. 43, 20 (2004): 5965-75; Ji, Yanbin et al. Journal of the American Chemical Society vol. 135, 31 (2013): 11623-11633; Ngo, Khac Huy et al. Chemical Communications (Cambridge, England) vol. 56, 7 (2020): 1082-1084; Wilbs, Jonas et al. Nature Communications vol. 11, 1 3890. 4 Aug. 2020; Clardy, Jon, and Christopher Walsh. Nature vol. 432, 7019 (2004): 829-37; Driggers, Edward M et al. Nature Reviews. Drug Discovery vol. 7, 7 (2008): 608-24).

Previously, the use of a cysteine-containing pyrrolysine (Pyl) analog, D-cysteinyl-N^{ε}-L-lysine (D-Cys-ε-Lys, abbreviation: O, FIG. 1B), for cyclizing an arginine-glycine-aspartate (RGD) motif was reported. The RGD motif was appended to an mCherry protein via intein-mediated native chemical ligation (NCL) (Lee, Marianne M et al. Chembiochem: a European Journal of Chemical Biology vol. 15, 12 (2014): 1769-72; Dawson, P E et al. Science (New York, N.Y.) vol. 266, 5186 (1994): 776-9). D-Cys-ε-Lys was genetically encoded by the amber (UAG) codon. Incorporation of D-Cys-ε-Lys into the recombinant protein was enabled by the introduction of a Methanosarcina mazei pyrrolysyl-tRNA synthetase/tRNA^{pyl} (PylRS/tRNA^{Pyl}) pair into Escherichia coli cells (Li, Xin et al. Angewandte Chemie (International ed. in English) vol. 48, 48 (2009): 9184-7). However, while cyclized proteins were obtained at high purity, product yields were low. Factors that impacted production yield included the inefficient incorporation of D-Cys-ε-Lys into a polypeptide and the incomplete and inefficient step of protein cyclization.

Chemical synthesis of noncanonical amino acids (ncAA), such as D-Cys-ε-Lys, followed by its exogenous supplementation to the culture medium is a common strategy used for the production of ncAA-containing proteins. However, this process is both costly and time consuming, and in some cases, the ncAAs may be cell impermeant, rendering them unavailable for translational incorporation. Therefore, there is a need for new ways of synthesizing Pyl analogs and other noncanonical amino acids (ncAAs) that are cost effective and sustainable for large-scale production of proteins containing ncAAs and/or Pyl analogs. As disclosed herein, the inventors have determined compositions and methods that address this need.

### SUMMARY

In a first aspect, the present disclosure provides a recombinant host cell comprising a polynucleotide sequence encoding a mutant PylC capable of synthesizing a noncanonical amino acid (ncAA), wherein the mutant PylC comprises a polypeptide sequence with at least 95% sequence identity with SEQ ID NO: 1 (wild-type M. mazei PylC). In some embodiments, the recombinant host cell of further comprises (a) a polynucleotide sequence encoding a wild-type PylRS or a mutant PylRS capable of using the ncAA as a substrate, and (b) a polynucleotide sequence encoding a tRNA that incorporates the ncAA at an amber codon.

In some embodiments, the mutant PylC comprises a combination of mutations corresponding to residues 177, 179, 233, and 256 of wild-type archaeal PylC. In some embodiments, the mutant PylC combination of mutations is chosen from:
1) S177N, E179P, D233S, and T256V;
2) S177C, E179T, D233S, and T256V;
3) E179C and D233N;
4) S177A, E179A, D233H, and T256M;
5) S177A, E179S, D233H, and T256L;
6) E179C, D233H, and T256V;
7) E179V, D233N, and T256V;
8) E179V, D233H, and T256N;
9) S177T, E179P, D233H, and T256M;
10) S177G, E179V, D233S, and T256L;
11) E179N, D233H, and T256L;
12) S177A, E179V, D233H, and T256I;
13) S177L, E179I, D233H, and T256C;
14) S177A, E179I, D233T, and T256L;
15) S177H, E179L, D233S, and T256V;
16) S177A, E179M, D233S, and T256L;
17) E179I, D233H, and T256I;
18) S177A, E179T, D233H, and T256V;
19) S177A, E179A, D233N, and T256C;
20) S177C, E1179P, D233H, and T256L;
21) S177G, E179I, and D233H;
22) S177M, E179V, D233H, and T256V;
23) S177V, E179V, D233H, and T256V;
24) S177A, E179A, D233S, and T256V;
25) S177G, E179M, D233N, and T256V;
26) S177G, E179I, D233N, and T256V;
27) S177G, E179T, D233S, and T256L;
28) S177A, E179T, D233S, and T256L;
29) S177L, E179T, D233S, and T256I;
30) S177T, E179P, D233H, and T256M;
31) E179L, D233L, and T256V;
32) S177A, E179V, D233Q, and T256C;
33) S177L, E179V, D233S, and T256A;
34) S177A, E179C, D233L, and T256C;
35) S177N, E179C, D233T, and T256C;
36) S177H, E17L9, D233S, and T256S;
37) E179G, D233L, and T256V;
38) S177N, E179P, D233S, and T256V;
39) S177M, E179G, D233H, and T256Y;
40) S177M, E179L, D233S, and T256C;
41) S177A, E179C, AND D233H;
42) E179L, D233L, and T256I;
43) S177L, E179V, D233H, and T256Y;
44) S177A, E179Q, D233N, and T256C; and
45) S177L, E179A, D233H, and T256V.

In some embodiments, the mutant PylC combination of mutations is S177N, E179P, D233S, and T256V.

In some embodiments, the mutant PylC combination of mutations is chosen from:
1) S177G, E179V, D233N, and T256V;
2) S177V, E179L, D233H, and T256L;
3) S177G, E179V, D233H, and T256A;
4) S177V, E179C, D233H, and T256L;
5) S177M, E179V, D233H, and T256Y;
6) S177C, E179P, D233H, and T256L;
7) S177G, E179A, D233S, and T256L;
8) S177V, E179L, D233H, and T256V;
9) S177G E179L, D233H, and T256L;
10) S177I, D233H, and T256V;
11) S177L, E179V, D233T, and T256I;
12) S177L, E179A, D233H, and T256M;
13) S177L, E179V, D233H, and T256A;
14) S177I, E179M, D233H, and T256L;
15) S177A, E179I, D233N, and T256I;
16) S177I, E179C, D233H, and T256L;
17) E179S and D233H;
18) S177V, E179V, D233H, and T256A;
19) S177C, E179V, D233H, and T256Y;
20) S177I, E179A, D233H, and T256H;
21) S177L, E179C, D233H, and T256V;
22) E179V and D233H;
23) S177V, E179V, D233H, and T256L;
24) S177I, E179L, D233H, and T256A;
25) S177L, E179A, D233H, and T256Y;
26) S177G, E179V, D233L, and T256L;
27) E179P, D233N, and T256V;
28) S177G, E179V, D233N, and T256N;
29) S177L, E179C, D233G, and T256H;
30) S177H, E179G, D233S, and T256A;
31) S177A, E179C, D233S, and T256L;
32) S177G, E179V, D233L, and T256Y;
33) S177L, E179T, D233T, and T256M;
34) S177G, E179L, D233W, and T256S;
35) S177V, E179L, D233S, and T256A;
36) E179V, D233L, and T256L;
37) S177L, E179P, D233S, and T256N; and
38) S177V, E179V, D233T, and T256L.

In some embodiments, the mutant PylC combination of mutations is S177G, E179V, D233N, and T256V.

In some embodiments, the mutant PylRS comprises mutations that correspond to (a) G114E, C348V, and S451F of wild-type archaeal PylRS, or (b) L301M, L305I, Y306L, L309A, and C348F of wild-type archaeal PylRS.

In some embodiments, the recombinant host cell genomic sequence encoding Release Factor 1 (RF1) is at least partially deleted.

In some embodiments, the recombinant host cell is a prokaryotic cell. In some embodiments, the recombinant host cell is a bacterial cell. In some embodiments, the recombinant host cell is an E. coli cell.

In a related aspect, the present disclosure provides a composition comprising any recombinant host cell of the present disclosure.

In a related aspect, the present disclosure provides a lysate of any recombinant host cell of the present disclosure.

In a another aspect, the present disclosure provides a method for recombinant synthesis of a protein comprising: (a) introducing a nucleotide sequence encoding the protein into any recombinant host cell of the present disclosure, wherein the nucleotide sequence comprises (i) at least one TAG codon within the nucleotide sequence's coding sequence, and (ii) a TAA codon or a TGA codon at the end of the nucleotide sequence's coding sequence; and (b) incubating the recombinant host cell under conditions permissible for transcription from the nucleotide sequence and for protein synthesis, thereby producing the protein. In some embodiments, the protein comprises one or more ncAA, optionally wherein the one or more ncAA is chosen from D-Cys-ε-Lys, D-Pra-ε-Lys, D-Allyl-ε-Lys, and 2-Chloro-Acetyl-ε-Lys.

In some embodiments, the method further comprises isolating the protein produced in step (b). In some embodiments, the method further comprises placing the isolated protein under conditions permissive for the protein to cyclize by forming a covalent bond between a (i) first moiety on a first ncAA and (ii) a second moiety on a cysteine, C-terminal thioester, or a second moiety on a second ncAA.

In some embodiments, the protein comprises a peptide sequence derived from small ubiquitin-related modifier (SUMO) protein.

In another aspect, the present disclosure provides a method for recombinant synthesis of a protein comprising contacting a nucleotide sequence encoding the protein with a cell lysate of any recombinant host cell of the present disclosure. In some embodiments, the protein is P16.

In a related aspect, the present disclosure provides a nucleic acid comprising a polynucleotide sequence encoding a mutant PylC capable of synthesizing a ncAA. In some embodiments, the mutant PylC comprises a combination of mutations corresponding to residues 177, 179, 233, and 256 of wild-type archaeal PylC. In some embodiments, the mutant PylC combination of mutations is chosen from:
1) S177N, E179P, D233S, and T256V;
2) S177C, E179T, D233S, and T256V;
3) E179C and D233N;
4) S177A, E179A, D233H, and T256M;
5) S177A, E179S, D233H, and T256L;
6) E179C, D233H, and T256V;
7) E179V, D233N, and T256V;
8) E179V, D233H, and T256N;
9) S177T, E179P, D233H, and T256M;
10) S177G, E179V, D233S, and T256L;
11) E179N, D233H, and T256L;
12) S177A, E179V, D233H, and T256I;
13) S177L, E179I, D233H, and T256C;
14) S177A, E179I, D233T, and T256L;
15) S177H, E179L, D233S, and T256V;
16) S177A, E179M, D233S, and T256L;
17) E179I, D233H, and T256I;
18) S177A, E179T, D233H, and T256V;
19) S177A, E179A, D233N, and T256C;
20) S177C, E179P, D233H, and T256L;
21) S177G, E179I, and D233H;
22) S177M, E179V, D233H, and T256V;
23) S177V, E179V, D233H, and T256V;
24) S177A, E179A, D233S, and T256V;
25) S177G, E179M, D233N, and T256V;
26) S177G, E179I, D233N, and T256V;
27) S177G, E179T, D233S, and T256L;
28) S177A, E179T, D233S, and T256L;
29) S177L, E179T, D233S, and T256I;
30) S177T, E179P, D233H, and T256M;
31) E179L, D233L, and T256V;
32) S177A, E179V, D233Q, and T256C;
33) S177L, E179V, D233S, and T256A;
34) S177A, E179C, D233L, and T256C;
35) S177N, E179C, D233T, and T256C;
36) S177H, E17L9, D233S, and T256S;
37) E179G, D233L, and T256V;
38) S177N, E179P, D233S, and T256V;
39) S177M, E179G, D233H, and T256Y;
40) S177M, E179L, D233S, and T256C;
41) S177A, E179C, AND D233H;
42) E179L, D233L, and T256I;
43) S177L, E179V, D233H, and T256Y;
44) S177A, E179Q, D233N, and T256C; and
45) S177L, E179A, D233H, and T256V.

In some embodiments, the mutant PylC combination of mutations is S177N, E179P, D233S, and T256V.

In some embodiments, the mutant PylC combination of mutations is chosen from:
1. S177G, E179V, D233N, and T256V;
2. S177V, E179L, D233H, and T256L;
3. S177G, E179V, D233H, and T256A;
4. S177V, E179C, D233H, and T256L;
5. S177M, E179V, D233H, and T256Y;
6. S177C, E179P, D233H, and T256L;
7. S177G, E179A, D233S, and T256L;
8. S177V, E179L, D233H, and T256V;
9. S177G E179L, D233H, and T256L;
10. S177I, D233H, and T256V;
11. S177L, E179V, D233T, and T256I;
12. S177L, E179A, D233H, and T256M;
13. S177L, E179V, D233H, and T256A;
14. S177I, E179M, D233H, and T256L;
15. S177A, E179I, D233N, and T256I;
16. S177I, E179C, D233H, and T256L;
17. E179S and D233H;
18. S177V, E179V, D233H, and T256A;
19. S177C, E179V, D233H, and T256Y;
20. S177I, E179A, D233H, and T256H;
21. S177L, E179C, D233H, and T256V;
22. E179V and D233H;
23. S177V, E179V, D233H, and T256L;
24. S177I, E179L, D233H, and T256A;
25. S177L, E179A, D233H, and T256Y;
26. S177G, E179V, D233L, and T256L;
27. E179P, D233N, and T256V; and
28. S177G, E179V, D233N, and T256N.

In some embodiments, the mutant PylC combination of mutations is S177G, E179V, D233N, and T256V.

In another aspect, the present disclosure provides a nucleic acid comprising a polynucleotide sequence encoding a mutant PylRS capable of using an ncAA as a substrate. In some embodiments, the mutant PylRS comprises mutations that correspond to (a) G14E, C348V and S451F of wild-type archaeal PylRS, or (b) L301M, L305I, Y306L, L309A, and C348F of wild-type archaeal PylRS.

In a related aspect, the present disclosure provides a composition comprising any nucleic acid of the present disclosure.

In a related aspect, the present disclosure provides an expression cassette or a vector comprising a polynucleotide sequence of any nucleic acid of the present disclosure.

### BRIEF DESCRIPTION OF THE DRAWINGS

FIG. 1A shows incorporation of the noncanonical amino acid (ncAA) D-Cys-ε-Lys into an exemplary protein. In this example, the protein comprises a cyclized therapeutic peptide (e.g., a P16 peptide) on one end and a cyclized targeting peptide (e.g., an arginine-glycine-aspartate (RGD) targeting peptide) on the other end.

FIG. 1B shows schematics of plasmid constructs used in the evolution of PylRS protein. Top portion shows the pPylST-KanR(TAG) construct which has one selection gene (KanR(TAG)) for the first round PylRS screening. Middle portion shows pPylST-KanR(TAG)-mCh(TAG) which has two selection genes (KanR(TAG) and mCh(TAG)) for the second and subsequent rounds of PylRS screening. Bottom portion shows pPylST.tL-mCh(TAG) harboring genes encoding tRNA^{M15} and the evolved PylRS^{EVF}. pPylST.tL-mCh(TAG) was used in the optimized readthrough system for efficient D-Cys-ε-Lys incorporation.

FIGS. 1C-1E show optimization of the D-Cys-ε-Lys readthrough system. FIG. 1C shows the chemical structure of D-Cys-ε-Lys. FIG. 1D shows an mCherry readthrough assay comparing the original and the optimized UAG readthrough system. The lysine codon at position 55 in the mCherry gene was mutated to TAG, and the medium was supplemented with 0, 2 or 5 mM D-Cys-ε-Lys. Data represent mean fluorescence intensity±standard error of the mean (n=3). WT: wild type. M15: tRNA^{M15}. U: Unmodified pPylST. tL: Modified pPylST with the two T7lac promoters replaced by Ptac and PLlacO1 respectively. R2: Rosetta 2 (DE3). C321: C321.ΔA.M9adapted. FIG. 1E shows comparison of the original UAG readthrough system and the optimized system for D-Cys-ε-Lys incorporation into different recombinant proteins. O represents D-Cys-ε-Lys. CaM is short for calmodulin.

FIGS. 2A-2C show engineering PylC for in cellulo synthesis of D-Cys-ε-Lys. FIG. 2A shows schematic representations of the reactions catalyzed by the wild-type PylC and engineered PylC. FIG. 2B shows screening of putative PylC mutants that could recognize D-cysteine and catalyze the production of D-Cys-ε-Lys based on mCherry fluorescence. Site-saturation mutagenesis was performed on four residues of PylC: S177, E179, D233, and T256. Data represent mean fluorescence intensity t standard error of the mean (n=3). FIG. 2C shows comparison of the wild-type PylC (PylC^{WT}) and the evolved PylC mutant (PylC^{NPSV}) in the production of D-Cys-ε-Lys for its incorporation into UAG-containing proteins at different concentrations of D-cysteine. Samples readthrough was benchmarked against the readthrough protein produced by exogenous supplementation of 4 mM D-Cys-ε-Lys.

FIGS. 3A-3E show computational analysis of cyclized P16 peptide. FIG. 3A shows amino acid sequences of cyclic P16 peptide (cycP16p) showing the site of D-Cys-ε-Lys incorporation (denoted by O) and linear P16p. Black bracket denotes D-Cys-ε-Lys mediated cyclization. FIG. 3B shows a model of cycP16p interacting with CDK6. Structure of p16p (ribbon model) and CDK6 (space-filling model) complex was derived from PDB: 1BI7. Residues interacting with CDK6 are labelled. FIG. 3C shows a comparison of molecular dynamic (MD) simulation results between linear P16p (LinP16p) (left) and cycP16p (right). FIG. 3C shows superposition of 10 rounds of LinP16p (left) and CycP16p (right) MD simulations at 10 ns. FIG. 3D shows RMSD and FIG. 3E shows RMSF profiles of LinP16p and CycP16p in 300 ns MD simulation. Results were calculated based on backbone atoms. Error bars present standard deviation (SD) of 3 replicate simulation runs and were plotted as shaded area in RMSD profile.

FIGS. 4A-4C show intramolecular cyclization of GFP-P16p. FIG. 4A shows a schematic of the protein construct GFP-O-P16p-intein-CBD-His7 and mechanism of D-Cys-ε-Lys-based protein cyclization. FIG. 4B shows an SDS-PAGE of reaction samples taken at different time points (hours (h)) during the cyclization of GFP-O-P16p. Shown are gels stained by Coomassie blue (upper) and detected by in-gel GFP fluorescence (lower). FIG. 4C shows a deconvoluted mass spectrum of GFP-cycP16p obtained by ESI-Orbitrap mass spectrometry.

FIG. 5 shows that cyclic P16p (MBP-cycP16p) exhibits higher CDK4-binding affinity than its linear counterpart. Binding curves from MST assays of MBP-P16p and MBP-cycP16p with GST-CDK4 are shown. Error bars represent standard deviation (SD) of 3 replicate measurements.

FIG. 6 shows schematics of protein constructs used in P16p cyclization. Top portion: GFP-cycP16p. Middle portion: MBP-cycP16p. Bottom portion: cycRGD-mCh-cycP16p. “O” represents D-Cys-ε-Lys.

FIGS. 7A-D show the effects of cycRGD-mCh-cycP16p on MCF-7 cells. In FIG. 7A, MCF-7 cells were exposed to different treatments for 24 h followed by cell cycle analysis on a BD flow cytometer. 10 nM actinomycin was included as positive control. FIG. 7B shows MCF-7 cells after 24-h treatment with different peptides. Cell numbers were normalized to PBS control group. Data are presented as the mean SD (n=3). Statistical significances versus cycRGD-mCh-P16p group were shown. FIG. 7C shows percent of arrested MCF-7 cells at G0/G1 phase after exposure to different treatments at different time points. Error bars present standard deviation (SD) of 3 replicate measurements. P values are calculated by one-way ANOVA test and ns represents not significant. *p<0.05, ***p<0.001, ****p<0.0001, ns, not significant. FIG. 7D shows a Western-blot analysis of the phosphorylation status of Rb in MCF-7 cells exposed to different treatments using anti-pRb antibodies.

FIGS. 8A-8B show D-Pra-ε-Lys synthesis with engineered PylC. FIG. 8A shows the use of (R)-2-aminopent-4-yonic acid as a substrate for the synthesis of D-Pra-ε-Lys with endogenous L-lysine. FIG. 8B shows 31 PylC variants that were identified following antibiotic selection. The PylC variants were evaluated using an mCherry fluorescence readthrough assay.

FIGS. 9A-9B show D-Allyl-ε-Lys synthesis with engineered PylC. FIG. 9A shows the reaction of L-Lysine with D-Allyl-OH and conversion to D-Allyl-ε-Lys. FIG. 9B shows mCherry fluorescence intensity from the 35 selected colonies.

FIG. 10 shows in cellulo synthesis of 2-Chloro-Acetyl-ε-Lys (2ClAcK) from 2-chloro-acetic acid and lysine.

FIGS. 11A-11C show the three-dimensional structure and Coomassie-stained gels for an exemplary cyclic SUMO1-derived peptide. FIG. 11A shows the structure of SUMOmini2. Mutations for cyclization are shown in stick figures. The C52S mutation prevents undesirable side reactions. FIG. 11B shows an SDS-PAGE of cyclic His-SUMOmini2-LVPRGS-SUMOCt-MBP before and after thrombin cleavage. FIG. 11C shows an SDS-PAGE of cyclic His-SUMOmini2 purified by RP-HPLC after thrombin cleavage.

FIG. 12 shows incubation of linear, cyclic, and bicyclic His-SUMOmini2 in the presence of 0.1 μg/mL proteinase K at room temperature for up to 1.5 hours.

FIG. 13 shows aggregation kinetics of α-synuclein (aSyn) in the absence or presence of different His-SUMOmini2 constructs in two stoichiometric ratios using a thioflavin T (ThT) assay. The His-SUMOmini2 constructs were linear His-SUMOmini2, monocyclic (“cyclic”) His-SUMOmini2, and bicyclic His-SUMOmini2. At a 1:1 ratio of α-synuclein to His-SUMOmini2 (left panel), all of the SUMOmini2 peptides inhibited α-synuclein aggregation. At a 1:0.2 ratio of α-synuclein to His-SUMOmini2 (right panel), the monocyclic His-SUMOmini2 was worse at inhibiting of α-synuclein aggregation than the linear SUMOmini2, while the bicyclic SUMOmini2 demonstrated enhanced inhibition of α-synuclein aggregation.

### DETAILED DESCRIPTION

### I. Definitions

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

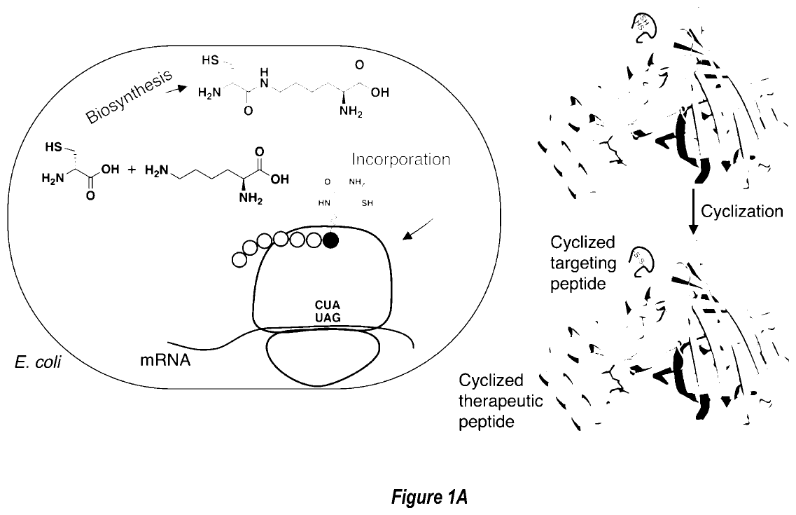

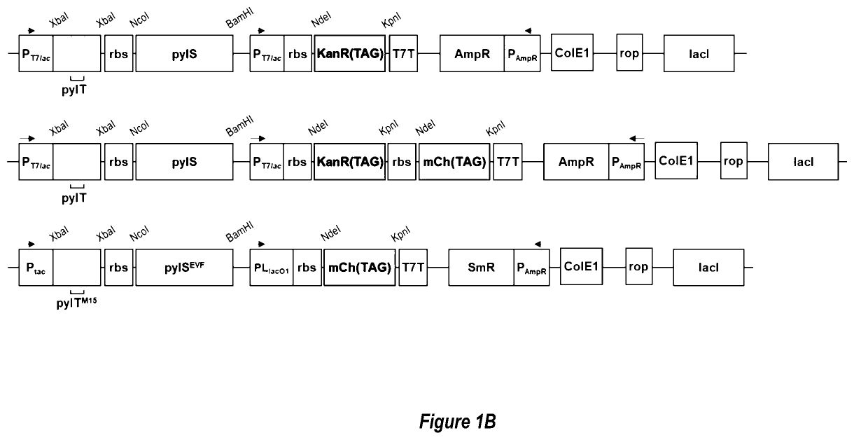

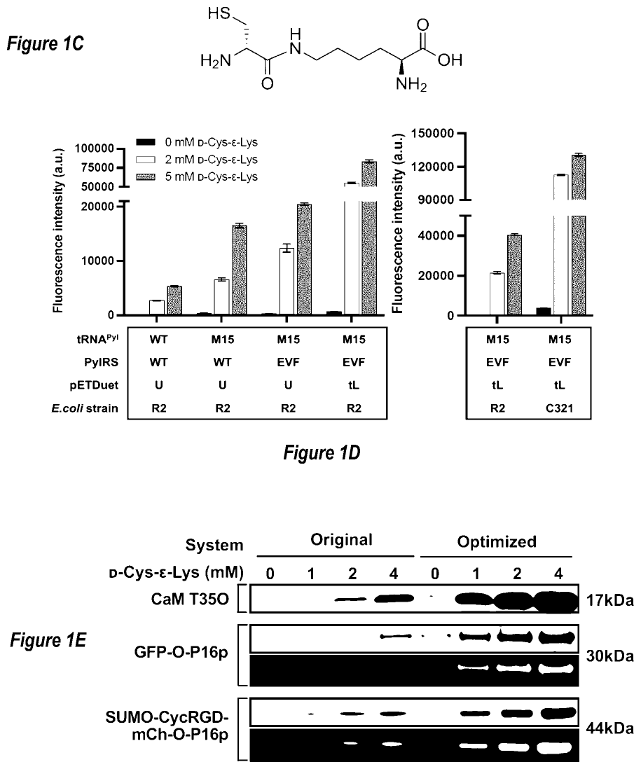

As used herein, the terms “D-Cys-ε-Lys,” “D-Cys-N^{ε}-Lys,” “D-Cys-ε-L-Lys,” “D-Cys-N^{ε}-L-Lys,” “D-Cys-ε-L-Lysine,” “D-Cys-N^{ε}-L-Lysine,” “D-Cysteinyl-ε-Lys,” “D-Cysteinyl-N^{ε}-Lys,” “D-Cysteinyl-ε-L-Lys,” “D-Cysteinyl-N^{ε}-L-Lys,” “D-Cysteinyl-ε-L-Lysine,” and “D-Cysteinyl-N^{ε}-L-Lysine” are synonymous and are used interchangeably to refer to the chemical structure “D-Cys-ε-Lys” as shown in FIG. 1C.

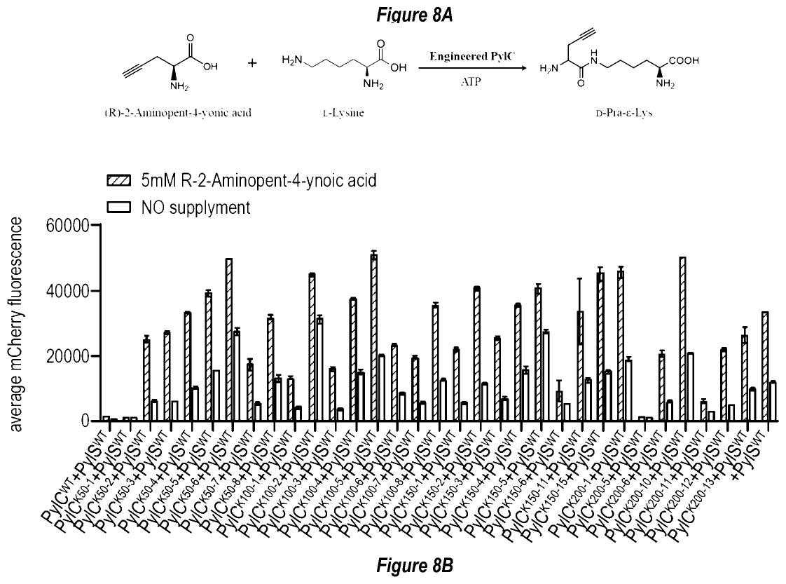

As used herein, the terms “D-Pra-ε-Lys,” “D-Pra-N^{ε}-Lys,” “D-Pra-ε-L-Lysine,” “D-Pra-N^{ε}-L-Lysine,” “D-Pra-ε-L-Lys,” “D-Pra-N^{ε}-L-Lys,” “D-Pra-ε-Lysine,” “D-Pra-N^{ε}-Lysine,” and “D-N6-(2-(R)-Propargylglycyl)lysine” are synonymous and are used interchangeably to refer to the chemical structure “D-Pra-ε-Lys” as shown in FIG. 8A.

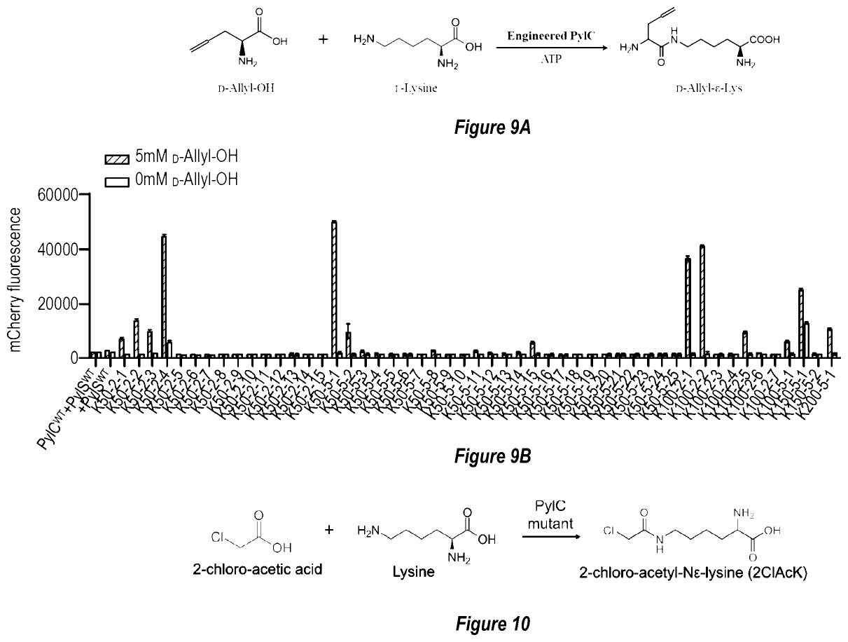

As used herein, the terms “D-Allyl-ε-Lys,” “D-Allyl-N^{ε}-Lys,” “D-Allyl-ε-L-Lys,” “D-Allyl-N^{ε}-L-Lys,” “D-Allyl-ε-Lysine,” “D-Allyl-N^{ε}-Lysine,” “D-Allyl-ε-L-Lysine,” and “D-Allyl-N^{ε}-L-Lysine” are synonymous and are used interchangeably to refer to the chemical structure “D-Allyl-ε-Lys” as shown in FIG. 9A.

As used herein, the terms “2-Chloro-Acetyl-ε-Lys,” “2-Chloro-Acetyl-N^{ε}-Lys,” “2-Chloro-Acetyl-ε-L-Lys,” “2-Chloro-Acetyl-N^{ε}-L-Lys,” “2-Chloro-Acetyl-ε-Lysine,” “2-Chloro-Acetyl-N^{ε}-Lysine,” “2-Chloro-Acetyl-ε-L-Lysine,” “2-Chloro-Acetyl-N^{ε}-L-Lysine,” and “2ClAcK” are synonymous and are used interchangeably to refer to the chemical structure “2-chloro-acetyl-N^{ε}-lysine (2ClAcK)” shown in FIG. 10.

As used herein, the terms “in cellulo” and “in vivo” are synonymous and are used interchangeably to refer to a process that takes place in a living cell. In a non-limiting example, “in cellulo synthesis of D-Cys-ε-Lys” or “in vivo synthesis of D-Cys-ε-Lys” refers to the synthesis of the chemical D-Cys-ε-Lys in a living cell, in contrast to the in vitro chemical synthesis of D-Cys-ε-Lys using chemical substrates in a reaction vessel, such as a test tube. According to the present disclosure, “in cellulo synthesis” or “in vivo synthesis” typically occurs in a recombinant host cell.

As used herein, the term “pyrrolysine,” “Pyl,” or “O” refers to the compound with PubChem CID 5460671, IUPAC name (2S)-2-amino-6-[[(2R,3R)-3-methyl-3,4-dihydro-2H-pyrrole-2-carbonyl]amino]hexanoic acid, and the molecular formula C₁₂H₂₁N₃O₃. Pyl is a lysine derivative, which, under some circumstances, can be incorporated into a polypeptide chain at a location corresponding to an amber (UAG) termination codon.

As used herein, the term “PylC” refers to a protein encoded by a pylC gene that is typically capable of catalyzing the ATP-dependent ligation of lysine and another substrate, thereby producing an ncAA. In some cases, a wild-type PylC catalyzes the ATP-dependent ligation of (3R)-3-methyl-D-ornithine and L-lysine, thereby producing (3R)-3-methyl-D-ornithyl-N6-L-lysine. In some cases, PylC refers to 3-methyl-D-ornithine-L-lysine ligase. While a PylC mutant or variant can be derived from any naturally occurring archaeal or bacterial PylC, in some non-limiting examples, the naturally occurring PylC is the PylC of Methanosarcina mazei (UniProt ID: Q8PWY3; SEQ ID NO: 1). In some cases, a PylC mutant or variant may be used in the synthesis of ncAAs, such as, without limitations, D-Cys-ε-Lys, D-Pra-ε-Lys, D-Allyl-ε-Lys, and/or 2ClAcK.

As used herein, the term “PylRS” refers to a protein that is encoded by a pylS gene and typically capable of catalyzing the attachment of pyrrolysine (Pyl) to a tRNA to produce tRNA^{Pyl}. In some cases, PylRS refers to pyrrolysyl-tRNA synthetase or pyrrolysine-tRNA ligase. While a PylRS mutant or variant can be derived from any naturally occurring archaeal or bacterial PylRS, in some non-limiting examples, the naturally occurring PylRS is the PylRS of Methanosarcina mazei (UniProt ID: Q6WRH6). In some cases, a PylRS mutant or variant may be used to catalyze the attachment of ncAAs, such as, without limitations, D-Cys-ε-Lys, D-Pra-ε-Lys, D-Allyl-ε-Lys, and/or 2ClAcK, onto tRNA. Thus, a PylRS mutant or variant can be used for ribosomal incorporation of ncAAs into nascent polypeptide chains, for example, at amber (TAG/UAG) codons in an mRNA.

As used herein, “UAG,” “TAG”, or “amber;” “UAA,” “TAA,” or “ochre;” and “UGA,” “TGA,” or “opal;” are three termination (stop) codons that interrupt translation. Pyl and certain ncAAs of the present disclosure, e.g., D-Cys-ε-Lys, D-Pra-ε-Lys, D-Allyl-ε-Lys, and/or 2ClAcK, can be encoded by a termination codon. In some cases, an ncAA is encoded by a “TAG,” “UAG” or “amber” codon.

The term “nucleic acid” or “polynucleotide” refers to deoxyribonucleotides or ribonucleotides and polymers thereof in either single- or double-stranded form. Unless specifically limited, the term encompasses nucleic acids containing known analogues of natural nucleotides which have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res., 19:5081 (1991); Ohtsuka et al., J. Biol. Chem., 260:2605-2608 (1985); and Cassol et al., (1992); Rossolini et al., Mol. Cell. Probes, 8:91-98 (1994)). The terms nucleic acid and polynucleotide are used interchangeably with gene, cDNA, and mRNA encoded by a gene.

The terms “polypeptide,” “peptide,” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full length proteins (i.e., antigens), wherein the amino acid residues are linked by covalent peptide bonds.

The term “amino acid” refers to naturally occurring and synthetic amino acids, including amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. As used herein, “canonical amino acids” refer to the 20 common amino acids that are used in ribosomal biosynthesis, i.e., alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, and valine. Thus, as used herein, the terms “noncanonical amino acids,” “ncAAs,” and “unnatural amino acids” refer to amino acids that fall outside the above list of 20 canonical amino acids. Without limitations, exemplary ncAAs include Pyl D-Cys-ε-Lys, D-Pra-ε-Lys, D-Allyl-ε-Lys, and 2-Chloro-Acetyl-ε-Lys. Amino acid analogs refer to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds that have a structure that is different from the general chemical structure of an amino acid, but that functions in a manner similar to a naturally occurring amino acid. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The phrase “percent identical,” “percent identity,” or equivalents used in the context of two nucleic acids or polypeptides, refers to a sequence that has at least a specified level of identity, e.g., at least 50% sequence identity with a reference sequence (e.g., any polypeptide sequence or any nucleotide sequence included herein). Alternatively, percent identity can be any integer from 50% to 100%. Some embodiments include at least: 50%, 55%, 60%, 65%, 70%, 75%, 80%, 85%, 90%, 91%, 92%, 93%, 94%, 95%, 96%, 97%, 98%, or 99%, compared to a reference sequence using the programs described herein, e.g., BLAST using standard parameters, as described below.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window,” as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, WI), or by manual alignment and visual inspection.

Algorithms that are suitable for determining percent sequence identity and sequence similarity are the BLAST and BLAST 2.0 algorithms, which are described in Altschul et al. (1990) J. Mol. Biol. 215: 403-410 and Altschul et al. (1977) Nucleic Acids Res. 25: 3389-3402, respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)).

The BLAST algorithm also performs a statistical analysis of the similarity between two sequences (see, e.g., Karlin & Altschul, Proc. Natl. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.01, more preferably less than about 10-5, and most preferably less than about 10-20.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

An “expression cassette” is a nucleic acid construct, generated recombinantly or synthetically, with a series of specified nucleic acid elements that permit transcription of a particular polynucleotide sequence in a host cell. An expression cassette may be part of a plasmid, viral genome, or nucleic acid fragment. Typically, an expression cassette includes a polynucleotide to be transcribed, operably linked to a promoter. “Operably linked” in this context means two or more genetic elements, such as a polynucleotide coding sequence and a promoter, placed in relative positions that permit the proper biological functioning of the elements, such as the promoter directing transcription of the coding sequence. Other elements that may be present in an expression cassette include those that enhance transcription (e.g., enhancers) and terminate transcription (e.g., terminators), as well as those that confer certain binding affinity or antigenicity to the recombinant protein produced from the expression cassette.

The term “recombinant” or “engineered” when used with reference, e.g., to a cell, nucleic acid, protein, or vector, indicates that the cell, nucleic acid, protein, or vector, has been modified by the introduction of a heterologous nucleic acid or protein or the alteration of a native nucleic acid or protein, or that the cell is derived from a cell so modified. Such modifications are often accomplished by manipulation of isolated segments of nucleic acids and may include, for example, genetic engineering techniques. Thus, for example, recombinant cells or engineered cells express genes that are not found within the native (non-recombinant or non-engineered) form of the cell, or the recombinant cells or engineered cells express native genes that are otherwise abnormally expressed, under expressed or not expressed at all.

The term “inhibiting” or “inhibition,” as used herein, refers to any detectable negative effect on a target biological process, such as protein-protein specific binding or interaction, the biological activity of a target protein, RNA/protein expression of a target gene, cellular signal transduction, cell proliferation, presence/level of an organism especially a micro-organism, any measurable biomarker, bio-parameter, or symptom in a subject, and the like. Typically, an inhibition is reflected in a decrease of at least 10%, 20%, 30%, 40%, 50%, 60%, 70%, 80%, 90% or greater in the target process (e.g., inhibition of α-synuclein aggregation by a protein that was cyclized using an ncAA), or any one of the downstream parameters mentioned above, when compared to a control. “Inhibition” further includes a 100% reduction, i.e., a complete elimination, prevention, or abolition of a target biological process or signal or disease/symptom. The other relative terms such as “suppressing,” “suppression,” “reducing,” and “reduction” are used in a similar fashion in this disclosure to refer to decreases to different levels (e.g., at least 10%, 20%, 30%, 40%, 50%, 60%, 70%, 80%, 90% or greater decrease compared to a control level) up to complete elimination of a target biological process or signal or disease/symptom. On the other hand, terms such as “activate,” “activating,” “activation,” “increase,” “increasing,” “promote,” “promoting,” “enhance,” “enhancing,” or “enhancement” are used in this disclosure to encompass positive changes at different levels (e.g., at least about 5%, 10%, 20%, 30%, 40%, 50%, 60%, 70%, 80%, 90%, 100%, 200%, or greater such as 3, 5, 8, 10, 20-fold increase compared to a control level in a target process, signal, or symptom/disease incidence.

As used in this application, an “increase” or a “decrease” refers to a detectable positive or negative change in quantity from a comparison control, e.g., an established standard control (such as an average level of-synuclein aggregation as measured by the Thioflavin T assay). An increase is a positive change that is typically at least 10%, or at least 20%, or 50%, or 100%, and can be as high as at least 2-fold or at least 5-fold or even 10-fold of the control value. Similarly, a decrease is a negative change that is typically at least 10%, or at least 20%, 30%, or 50%, or even as high as at least 80% or 90% of the control value. Other terms indicating quantitative changes or differences from a comparative basis, such as “more,” “less,” “higher,” and “lower,” as well as terms indicating an action to cause such changes or differences, such as “increase,” “promote,” “enhance,” “decrease,” “inhibit,” and “suppress,” are used in this application in the same fashion as described above. In contrast, the term “substantially the same” or “substantially lack of change” indicates little to no change in quantity from the standard control value, typically within ±10% of the standard control, or within ±5%, 2%, or even less variation from the standard control.

As used in herein, the singular forms “a”, “an” and “the” include plural referents unless the content clearly dictates otherwise. Thus, for example, reference to “an ncAA” optionally includes a combination of two or more such molecules, and the like.

As used herein, the term “about,” when modifying any amount, refers to the variation in that amount typically encountered by one of skill in the art. For example, the term “about” refers to the normal variation encountered in measurements for a given analytical technique, both within and between batches or samples. Thus, the term about can include variation of +/−1-10% of the measured value, such as +/−1%, 2%, 3%, 4%, 5%, 6%, 7%, 8%, 9%, or 10% variation of the measured value. The amounts disclosed herein include equivalents to those amounts, including amounts modified or not modified by the term “about.”

Unless otherwise defined, all technical terms used herein have the same meaning as commonly understood by one of ordinary skill in the art to which this disclosure belongs.

### II. Introduction

Disclosed herein are recombinant cells and related polynucleotides, polypeptides, tRNAs, and methods for the in cellulo synthesis of noncanonical amino acids (ncAAs) and proteins comprising ncAAs. In some embodiments, the ncAA is D-Cys-ε-Lys, D-Pra-ε-Lys, D-Allyl-ε-Lys, and/or 2ClAcK. The in cellulo synthesis of the ncAAs and the proteins comprising ncAAs are achieved by modifying the archaeal pyrrolysine (Pyl; O) biosynthesis pathway.

Archaea are single cell organisms with an appearance similar to bacteria but have distinct characteristics. Archaea are prokaryotes that lack cell nuclei. Archaeal cells possess a series of enzymes, PylB, PylC, and PylD; for the biosynthesis of Pyl, the 22^{nd} proteinogenic amino acid appearing in proteins synthesized in archaeal cells and certain bacterial cells (Meng, Kexin et al. Frontiers in microbiology vol. 13 1007832. 8 Sep. 2022; Wan, Wei et al. Biochimica et Biophysica Acta vol. 1844, 6 (2014): 1059-70). Also present in archaeal cells are pyrrolysine-tRNA synthetase (PylRS) and the pylT tRNA gene, which are responsible for the synthesis of pyrrolysine-tRNA (tRNA^{Pyl}) and thus enable the incorporation of Pyl into nascent polypeptide chains with corresponding amber (UAG) codons (Wan, Wei et al. Biochimica et Biophysica Acta vol. 1844, 6 (2014): 1059-70). In the genome of the Methanosarcinaceae, the pyl genes are present in an uninterrupted cluster as pylTSBCD, with pylT and pylS encoding tRNA^{Pyl} and PylRS, respectively (Borrel, Guillaume et al. Archaea (Vancouver, B.C.) vol. 2014 374146. 27 Jan. 2014).

Amber codon suppression refers to the use of the TAG/UAG (stop) codon as a coding codon in translation. The complementary amber tRNA^{CUA} is aminoacylated by an orthogonal aminoacyl-tRNA synthetase that is specifically designed to accept only ncAAs. This results in a protein with one or more ncAAs incorporated. Developments in amber codon suppression are discussed, e.g., in Brabham, Robin, and Martin A Fascione. Chembiochem: a European Journal of Chemical Biology vol. 18, 20 (2017): 1973-1983; and Wals, Kim, and Huib Ovaa. Frontiers in Chemistry vol. 2 15. 1 Apr. 2014.

The present inventors mutagenized the archaeal enzymes PylC and PylRS by site-saturation mutagenesis and directed evolution, respectively, and acquired PylC variants and PylRS variants capable of synthesizing ncAAs (e.g., without limitations, D-Cys-ε-Lys, D-Pra-ε-Lys, D-Allyl-ε-Lys, and 2ClAcK) and their cognate tRNAs, respectively. As such, this disclosure provides compositions and methods for engineering the enzymes supporting biosynthesis of ncAAs and their corresponding tRNAs, thus enabling the incorporation of these ncAAs into various recombinant proteins. These methods and compositions represent improvements in the field, e.g., as discussed in Lee et al. Chemistry Europe 15(12):1769-1772, 2014); U.S. Pat. No. 8,921,571; and U.S. Patent Application Publication No. 2014/0302553.

In one aspect, the present disclosure provides PylC mutants that are capable of producing an ncAA by joining a first amino acid with a second amino acid—a reactive group on the first amino acid reacts with a lysine on the second amino acid, and a covalent bond is formed at the epsilon-nitrogen (N^{ε}) of the lysine. In some embodiments, the ncAA is D-Cys-ε-Lys, D-Pra-ε-Lys, D-Allyl-ε-Lys, and/or 2ClAcK. In some embodiments, the mutant PylC is derived from a wild-type archaeal PylC and comprises four point mutations at positions 177, 179, 233, and 256 corresponding to a wild-type archaeal PylC amino acid sequence, e.g., the M. mazei PylC (UniProt ID: Q8PWY3; SEQ ID NO: 1).

In another aspect, the present disclosure provides wild-type PylRS or PylRS mutants that are capable of attaching an ncAA (e.g., without limitations, D-Cys-ε-Lys, D-Pra-ε-Lys, D-Allyl-ε-Lys, and 2ClAcK) to a tRNA to produce tRNA^{ncAA}. In some embodiments, the mutant PylRS is derived from a wild-type archaeal PylRS and comprises three point mutations at positions 14, 348, and 451 corresponding to a wild-type archaeal archaeal PylRS amino acid sequence.

In another aspect, the present disclosure provides genetically modified host cells that express PylCs, PylRSs, and tRNAs that support the synthesis of ncAAs (e.g., without limitations, D-Cys-ε-Lys, D-Pra-ε-Lys, D-Allyl-ε-Lys, and 2ClAcK) and the incorporation of the ncAAs into newly synthesized recombinant proteins. These cells are therefore capable of supporting the in cellulo synthesis of a protein containing such ncAAs strategically placed at pre-determined locations so as to allow for protein modifications, e.g., cyclization, oligomerization, and PEGylation. Specifically, the recombinant host cells comprise (1) a polynucleotide sequence encoding a mutant PylC capable of synthesizing an ncAA; (2) a polynucleotide sequence encoding a wild-type or mutant PylRS capable of attaching ncAAs to tRNAs; and (3) a polynucleotide sequence encoding a tRNA that is charged by the PylRS with an ncAA, and the tRNA incorporates the ncAA into nascent polypeptide chains.

In some embodiments, the recombinant host cell has been genetically modified to inactivate its endogenous Release Factor 1 (RF1) in order to enhance the read-through at the UAG codon where ncAA (e.g., without limitations, D-Cys-ε-Lys, D-Pra-ε-Lys, D-Allyl-ε-Lys, and 2ClAcK) is to be incorporated into the protein. For example, the recombinant host cell's genomic sequence encoding RF1 may be deleted, truncated, or mutated, so as to reduce or eliminate RF1 activity. In some embodiments, the host cell is a prokaryotic cell, e.g., a bacterial cell such as E. coli. In some embodiments, the host cell is a eukaryotic cell.

In some embodiments, the recombinant host cell comprises D-Cys-ε-Lys, D-Pra-ε-Lys, D-Allyl-ε-Lys, or 2ClAcK, e.g., as the result of the PylC activity. In some embodiments, the recombinant host cell comprises D-Cys-ε-Lys-tRNA, D-Pra-ε-Lys-tRNA, D-Allyl-ε-Lys-tRNA, or 2ClAcK-tRNA e.g., as the result of PylRS activity.

Also provided herein are methods for in cellulo synthesis of proteins comprising ncAA(s) of the present disclosure, and the methods comprise the use of recombinant host cells disclosed herein. In some embodiments, the ncAAs in the proteins are useful for producing cyclic, oligomeric, and/or PEGylated proteins. In some embodiments, the proteins are modified in cellulo while in other embodiments, the proteins are modified in vitro. In some embodiments, the proteins that are modified, e.g., cyclized, oligomerized, or PEGylated, according to methods disclosed herein demonstrate longer serum half-lives, increased resistance to proteolytic degradation, improved stability in serum, improved binding to a target, and/or improved efficacy.

### III. PylC

PylC is an enzyme capable of catalyzing the ATP-dependent ligation of lysine and a second substrate, thereby producing an ncAA, for example, D-Cysteinyl-ε-L-Lysine (D-Cys-ε-Lys), D-N6-(2-(R)-Propargylglycyl)lysine (D-Pra-ε-Lys), D-Allyl-N^{ε}-L-Lysine (D-Allyl-ε-Lys), or 2-Chloro-Acetyl-N^{ε}-L-Lysine (2ClAcK). Many wild-type archaeal and bacterial PylCs, including proteins are similar to an archaeal or bacterial PylC (e.g., a protein that has at least 50% identity with an archaeal or bacterial PylC) may be used to produce a PylC variant that is capable of producing an ncAA. Non-limiting examples of PylC and proteins similar to PylC include the Methanosarcina acetivorans strain (ATCC 35395/DSM 2834/JCM 12185/C2A) PylC (UniProt ID: Q8TUC0), the Methanosarcina barkeri (strain Fusaro/DSM 804) PylC (UniProt ID: Q46E79), the Methanosarcina mazei (strain ATCC BAA-159/DSM 3647/Goe1/Go1/JCM 11833/OCM 88) (Methanosarcina frisia) PylC (UniProt ID: Q8PWY3), the Methanococcoides methylutens 3-methylornithine--L-lysine ligase (UniProt ID: A0A099T2V5), the Methanosarcina thermophila CHTI-55 pyrrolysine synthetase (UniProt ID: A0A0E3HAD6), the Methanosarcina sp. WH1 pyrrolysine synthetase (UniProt ID: A0A0E3L5I7), the Methanosarcina thermophila (strain ATCC 43570/DSM 1825/OCM 12/VKM B-1830/TM-1) pyrrolysine synthetase (UniProt ID: A0A0E3NDZ8), the Methanosarcina sp. WWM596 pyrrolysine synthetase (UniProt ID: A0A0E3NNH7), the Methanosarcina siciliae T4/M pyrrolysine synthetase (UniProt ID: A0A0E3P0K1), the Methanosarcina sp. MTP4 pyrrolysine synthetase (UniProt ID: A0A0E3P2G1), the Methanosarcina siciliae HI350 pyrrolysine synthetase ((UniProt ID: A0A0E3P9R8), the Methanosarcina siciliae C2J pyrrolysine synthetase (UniProt ID: A0A0E3PJB5), the Methanosarcina mazei WWM610 pyrrolysine synthetase (UniProt ID: A0A0E3Q1K8), the Methanosarcina vacuolata Z-761 pyrrolysine synthetase (UniProt ID: A0A0E3Q9K9), the Methanosarcina sp. Kolksee pyrrolysine synthetase (UniProt ID: A0A0E3QFB4), the Methanosarcina barkeri str. Wiesmoor pyrrolysine synthetase (UniProt ID: A0A0E3QJ27), the Methanosarcina barkeri MS pyrrolysine synthetase (UniProt ID: A0A0E3QRW9), the Methanosarcina barkeri 227 pyrrolysine synthetase (UniProt ID: A0A0E3R2H9), the Methanosarcina mazei SarPi pyrrolysine synthetase (UniProt ID: A0A0E3REK9), the Methanosarcina mazei S-6 pyrrolysine synthetase (UniProt ID: A0A0E3RL10), Methanosarcina mazei LYC pyrrolysine synthetase (UniProt ID: A0A0E3RQK1), the Methanosarcina mazei C16 pyrrolysine synthetase (UniProt ID: A0A0E3S363), the Methanosarcina barkeri 3 pyrrolysine synthetase (UniProt ID: A0A0E3SNN3), the Methanococcoides methylutens MM1 pyrrolysine synthetase (UniProt ID: A0A0E3SP52), the Methanosarcina lacustris Z-7289 pyrrolysine synthetase (UniProt ID: A0A0E3WSH3), the Methanosarcina horonobensis HB-1=JCM 15518 pyrrolysine synthetase (UniProt ID: A0A0E3WWW5), the Methanosarcina barkeri CM1 pyrrolysine biosynthesis protein PylC (UniProt ID: A0A0G3CBZ6), the Methanohalophilus sp. DAL1 3-methylornithine--L-lysine ligase PylC (UniProt ID: A0A1B8WZK4), the Methanohalophilus sp. DAL1 3-methylornithine-L-lysine ligase PylC ((UniProt ID: A0A1B8WZU9), the Methanohalophilus sp. 2-GBenrich ATP-grasp domain-containing protein (UniProt ID: A0A1D2UZX5), the Methanosarcina sp. A14 3-methylornithine--L-lysine ligase PylC (UniProt ID: A0A1D2WWF0), the Methanosarcina sp. Ant1 3-methylornithine--L-lysine ligase PylC (UniProt ID: A0A1E7GEX0), the Methanococcoides vulcani pyrrolysine biosynthesis protein PylC (UniProt ID: A0A1H9Z9J2), the Methanolobus profundi pyrrolysine biosynthesis protein PylC (UniProt ID: A0A1I4T458), the Methanosarcina thermophila pyrrolysine biosynthesis protein PylC (UniProt ID: A0A1I6ZM81), the Methanohalophilus halophilus 3-methylornithine--L-lysine ligase PylC (Pyrrolysine biosynthesis protein PylC) (UniProt ID: A0A1L3Q0L2), the Methanohalophilus portucalensis FDF-1 3-methylornithine--L-lysine ligase PylC (Pyrrolysine biosynthesis protein PylC) (UniProt ID: A0A1L9C5H7), the Methanomethylovorans sp. PtaU1.Bin093 3-methylornithine--L-lysine ligase (UniProt ID: A0A1V4YHV6), the Methanohalophilus euhalobius pyrrolysine biosynthesis protein PylC (UniProt ID: A0A285FYL0), the Methanosarcina spelaei 3-methylornithine--L-lysine ligase PylC (UniProt ID: A0A2A2HRV9), the Methanohalophilus portucalensis 3-methylornithine--L-lysine ligase PylC (UniProt ID: A0A2D3C6Q6), the Methanohalophilus euhalobius 3-methylornithine--L-lysine ligase PylC (Pyrrolysine biosynthesis protein PylC) (UniProt ID: A0A315A1A6), the Methanosarcina thermophila pyrrolysine synthetase (UniProt ID: A0A3G9CRP1), the Methanohalophilus sp. RSK 3-methylornithine--L-lysine ligase PylC (UniProt ID: A0A3M9LRP0), the Methanosarcina sp. MSH10X1 3-methylornithine--L-lysine ligase PylC (UniProt ID: A0A498GWN6), the Methanohalophilus sp. WG1-DM ATP-grasp domain-containing protein (UniProt ID: A0A498H9H1), the Methanolobus halotolerans 3-methylornithine--L-lysine ligase PylC (UniProt ID: A0A4E0Q579), the Methanosarcina mazei (Methanosarcina frisia) 3-methylornithine--L-lysine ligase PylC (UniProt ID: A0A4P8QYJ1), the Methanosarcina flavescens 3-methylornithine--L-lysine ligase PylC (UniProt ID: A0A660HT74), the Methanolobus zinderi 3-methylornithine--L-lysine ligase PylC (UniProt ID: A0A7D513G2), the Methanosarcinaceae archaeon 3-methylornithine--L-lysine ligase PylC (UniProt ID: A0A7J4PI19), the Methanosarcinales archaeon 3-methylornithine--L-lysine ligase PylC (UniProt ID: A0A7L4QV83), the Methanolobus vulcani pyrrolysine biosynthesis protein PylC (UniProt ID: A0A7Z7AZJ6), the Methanolobus vulcani 3-methylornithine--L-lysine ligase PylC (UniProt ID: A0A7Z8P2G7), the Methanosarcina sp 3-methylornithine--L-lysine ligase PylC (UniProt ID: A0A832LB55), the Methanosarcina acetivorans 3-methylornithine--L-lysine ligase PylC (UniProt ID: A0A832SGS6), the Methanosarcina sp 3-methylornithine--L-lysine ligase PylC (UniProt ID: A0A832U1W8), the Methanosarcina sp 3-methylornithine--L-lysine ligase PylC (UniProt ID: A0A847PSL9), the Methanococcoides seepicolus 3-methylornithine--L-lysine ligase PylC (UniProt ID: A0A9E5D7X3), the Methanolobus sp. FTZ2 3-methylornithine--L-lysine ligase PylC (UniProt ID: A0AA51UHC3), the Methanolobus sp. FTZ6 3-methylornithine--L-lysine ligase PylC (UniProt ID: A0AA51UQ13), the Methanohalophilus mahii (strain ATCC 35705/DSM 5219/SLP) ATP-grasp domain-containing protein (UniProt ID: D5E9G2), the Methanohalobium evestigatum (strain ATCC BAA-1072/DSM 3721/NBRC 107634/OCM 161/Z-7303) ATP-grasp domain-containing protein D7E781, the Methanosalsum zhilinae (strain DSM 4017/NBRC 107636/OCM 62/WeN5) (Methanohalophilus zhilinae) ATP-grasp domain-containing protein (UniProt ID: F7XLV6), the Methanomethylovorans hollandica (strain DSM 15978/NBRC 107637/DMS1) pyrrolysine biosynthesis protein PylC (UniProt ID: LOKW95), the Methanosarcina mazei Tuc01 pyrrolysine synthetase (UniProt ID: M1Q3M7), the Methanococcoides burtonii (strain DSM 6242/NBRC 107633/OCM 468/ACE-M) ATP-binding protein with DUF201 domain, pyrrolysine biosynthesis (UniProt ID: Q12UB8), the Methanosarcina barkeri PylC (UniProt ID: Q8NKQ4), and the Methanolobus tindarius DSM 2278 pyrrolysine biosynthesis protein PylC (UniProt ID: W9DXF9).

In some embodiments, site-saturation mutagenesis is the strategy employed to produce PylC variants. In site-saturation mutagenesis, one or more positions along the protein sequence are identified as likely to accommodate beneficial or desired mutations and are then randomized, i.e., the amino acids at these positions are replaced by random ones. Site-saturation mutagenesis is discussed in Nov, Yuval. Applied and environmental microbiology vol. 78, 1 (2012): 258-62; and Gupta, Kritika, and Raghavan Varadarajan. Current opinion in structural biology vol. 50 (2018): 117-125.

In some embodiments, the PylC variant is derived from a wild-type archaeal PylC polypeptide sequence, for example, without limitations, the PylC of Methanosarcina mazei (UniProt ID: Q8PWY3; SEQ ID NO: 1). Non-limiting examples of PylC are discussed in Gaston, Marsha A et al. Current Opinion in Microbiology vol. 14, 3 (2011): 342-9.

In some embodiments, the PylC variant comprises at least 90%, at least 91%, at least 92%, at least 93%, at least 94%, at least 95%, at least 96%, at least 97%, at least 98%, or at least 99% sequence identity with a wild-type archaeal PylC polypeptide sequence, e.g., the M. mazei PylC (UniProt ID: Q8PWY3; SEQ ID NO: 1). In some embodiments, the PylC variant further comprises a polypeptide sequence where the amino acids corresponding to positions S177, E179, D233, and/or T256 of the M. mazei PylC (UniProt ID: Q8PWY3; SEQ ID NO: 1) are substituted with a non-native amino acid. In some embodiments, the amino acid corresponding to S177 of the M. mazei PylC (UniProt ID: Q8PWY3; SEQ ID NO: 1) can be any amino acid other than S. In some embodiments, the amino acid corresponding to E179 of the M. mazei PylC (UniProt ID: Q8PWY3; SEQ ID NO: 1) can be any amino acid other than E. In some embodiments, the amino acid corresponding to D233 of the M. mazei PylC (UniProt ID: Q8PWY3; SEQ ID NO: 1) can be any amino acid other than D. In some embodiments, the amino acid corresponding to T256 of the M. mazei PylC (UniProt ID: Q8PWY3; SEQ ID NO: 1) can be any amino acid other than T.

In some embodiments, the PylC variant is derived from the M. mazei PylC (UniProt ID: Q8PWY3; SEQ ID NO: 1) polypeptide sequence as shown below as SEQ ID NO: 1. In some embodiments, the PylC variant comprises a mutation at one or more of S177, E179, D233, and/or T256 of SEQ ID NO: 1.

a. PylC Variants for Synthesizing D-Cysteinyl-ε-L-Lysine (D-Cys-ε-Lys)

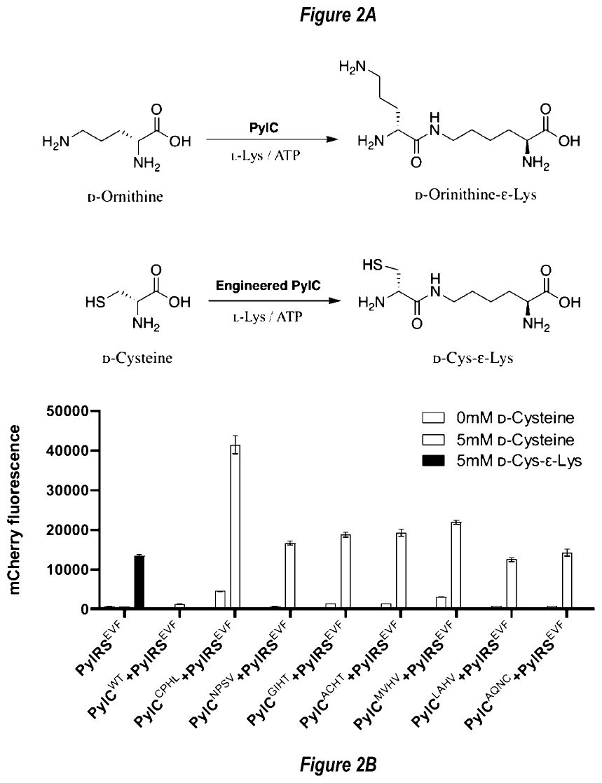

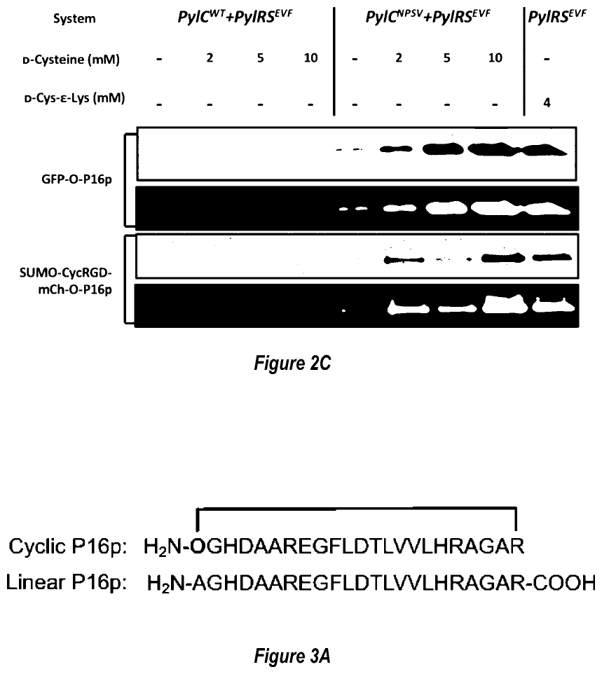

In some embodiments, the PylC variant is capable of producing D-Cys-ε-Lys by using D-cysteine (D-Cys) and L-lysine (L-Lys) as substrates (FIG. 2A). D-Cys-ε-Lys is a di-amino acid that consists of a D-Cys attached to the ε-nitrogen of an L-Lys. D-Cys-ε-Lys may be used for native chemical ligation reactions, i.e., reacting a C-terminal peptide thioester with an N-terminal cysteinyl peptide to produce a native peptide bond between the two fragments. Non-limiting examples of suitable reactions, e.g., protein cyclization and ubiquitination, are discussed in Lee, Marianne M et al., Chembiochem: a European Journal of Chemical Biology vol. 15, 12 (2014): 1769-72; Li, Xin et al., Angewandte Chemie (International ed. In English) vol. 48, 48 (2009): 9184-7; and Tai, Jingxuan et al., Journal of the American Chemical Society vol. 145, 18 (2023): 10249-10258. doi:10.1021/jacs.3c01291.

In some embodiments, the D-Cys-ε-Lys PylC variant comprises a polypeptide sequence with at least 90%, at least 91%, at least 92%, at least 93%, at least 94%, at least 95%, at least 96%, at least 97%, at least 98%, or at least 99% sequence identity with a wild-type archaeal PylC polypeptide sequence, e.g., the M. mazei PylC (UniProt ID: Q8PWY3; SEQ ID NO: 1). In some embodiments, the D-Cys-ε-Lys PylC further comprises mutations at positions that correspond to S177, E179, D233, and/or T256 of the M. mazei PylC (UniProt ID: Q8PWY3; SEQ ID NO: 1), where one or more of those amino acids are substituted with one or more non-native amino acids. Exemplary amino acid mutations for S177, E179, D233, and/or T256 are provided in Table 1 below. In some embodiments, the PylC variant comprises the mutations S177N, E179P, D233S, and T256V (PylC^{NPSV} mutations).

TABLE 1
PylC Variants for D-Cys-ε-Lys Synthesis.

| Variant | S177 | E179 | D233 | T256 |
|---|---|---|---|---|
| K50-4 | C | T | S | V |
| K50-12 | NO MUTATION | C | N | NO MUTATION |
| K50-13 | A | A | H | M |
| K50-15 | A | S | H | L |
| K50-17 | NO MUTATION | C | H | V |
| K50-19 | NO MUTATION | V | N | V |
| K50-45 | NO MUTATION | V | H | N |
| K50-48 | T | P | H | M |
| K50-50 | G | V | S | L |
| K75-1 | NO MUTATION | N | H | L |
| K75-3 | A | V | H | I |
| K75-6 | L | I | H | C |
| K75-13 | A | I | T | L |
| K75-15 | H | L | S | V |
| K75-19 | A | M | S | L |
| K75-22 | NO MUTATION | I | H | I |
| K75-29 | A | T | H | V |
| K75-30 | A | A | N | C |
| K150-1 | C | P | H | L |
| K150-37 | G | I | H | NO MUTATION |
| K200-10 | M | V | H | V |
| K150-21 | V | V | H | V |
| K150-45 | A | A | S | V |
| K150-32 | G | M | N | V |
| K200-46 | G | I | N | V |
| K150-15 | G | T | S | L |
| K150-22 | A | T | S | L |
| K150-35 | L | T | S | I |
| K200-16 | T | P | H | M |
| K200-17 | NO MUTATION | L | L | V |
| K200-12 | A | V | Q | C |
| K200-7 | L | V | S | A |
| K200-6 | A | C | L | C |
| K200-3 | N | C | T | C |
| K200-9 | H | L | S | S |
| K150-23 | NO MUTATION | G | L | V |
| K150-19 | N | P | S | V |
| (PylC^{NPSV}) | | | | |
| K150-60 | M | G | H | Y |
| K200-5 | M | L | S | C |
| K150-54 | A | C | H | NO MUTATION |
| K200-1 | NO MUTATION | L | L | I |
| K150-24 | L | V | H | Y |
| K200-41 | A | Q | N | C |
| K200-40 | L | A | H | V |

In some embodiments, the PylC variant polynucleotide sequence (coding sequence) comprises at least 90%, at least 91%, at least 92%, at least 93%, at least 94%, at least 95%, at least 96%, at least 97%, at least 98%, at least 99%, or 100% sequence identity with any one of the PylC variant polynucleotide sequences as set forth in SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, or 88. In some embodiments, the PylC variant polypeptide sequence comprises at least 90%, at least 91%, at least 92%, at least 93%, at least 94%, at least 95%, at least 96%, at least 97% at least 98%, at least 99%, or 100% sequence identity with any one of the PylC variant polypeptide sequences as set forth in SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, or 89. In some embodiments, the PylC variant polynucleotide sequence comprises one or more mutations at nucleic acids that correspond to S177, E179, D233, and/or T256 of SEQ ID NO: 1. In some embodiments, the PylC variant polynucleotide sequence is the sequence of SEQ ID NO: 74. In some embodiments, the PylC variant polypeptide sequence comprises a mutation at one or more of S177, E179, D233, and/or T256 of SEQ ID NO: 1. In some embodiments, the PylC variant is variant K150-19 (designated as PylC^{NPSV}; SEQ ID NO: 75).

b. PylC Variants for Synthesizing D-N6-(2-(R)-Propargylglycyl)Lysine (D-Pra-ε-Lys)

In some embodiments, the PylC variant is capable of producing D-Pra-ε-Lys by using (R)-2-Aminopent-4-yonic acid and L-Lys as substrates (FIG. 8A). D-Pra-ε-Lys is a di-amino acid that consists of a propargylglycyl amino acid group attached to the ε-nitrogen of an L-Lys. D-Pra-ε-Lys contains an alkyne group that is amendable to click and other reactions (such as reactions involving additions to a cysteine and reactions involving thiols via a thiol-yne reaction) which are discussed in Mons, Elma et al. Journal of the American Chemical Society vol. 143, 17 (2021): 6423-6433; Fairbanks, Benjamin D et al. Macromolecules vol. 42, 1 (2009): 211-217; Lowe, Andrew B. et al. Journal of Materials Chemistry vol. 20 (23): 4745; and Li, Xin et al. Chemistry, an Asian journal vol. 5, 8 (2010): 1765-9. doi: 10.1002/asia.201000205.

In some embodiments, the D-Pra-ε-Lys PylC variant comprises a polypeptide sequence with at least 90%, at least 91%, at least 92%, at least 93%, at least 94%, at least 95%, at least 96%, at least 97%, at least 98%, or at least 99% sequence identity with a wild-type archaeal PylC polypeptide sequence, e.g., the M. mazei PylC (UniProt ID: Q8PWY3; SEQ ID NO: 1). In some embodiments, the D-Pra-ε-Lys PylC further comprises mutations at positions that correspond to S177, E179, D233, and/or T256 of the M. mazei PylC (UniProt ID: Q8PWY3; SEQ ID NO: 1), where one or more of those amino acids are substituted with one or more non-native amino acids. Exemplary amino acid mutations for S177, E179, D233, and/or T256 are provided in Table 2 below. In some embodiments, the PylC variant comprises the mutation D233H. In some embodiments, the PylC variant comprises the mutations S177G, E179V, D233N, and T256V (PylC^{GVNV} mutations).

TABLE 2
PylC Variants For D-Pra-ε-Lys Synthesis.

| Variant | S177 | E179 | D233 | T256 |
|---|---|---|---|---|
| K50-1 | V | L | H | L |
| K50-2 | G | V | H | A |
| K50-3 | V | C | H | L |
| K50-4 | M | V | H | Y |
| K50-5 | C | P | H | L |
| K50-6 | G | A | S | L |
| K50-7 | V | L | H | V |
| K50-8 | G | L | H | L |
| K100-1 | I | NO MUTATION | H | V |
| K100-2 | L | V | T | I |
| K100-3 | L | A | H | M |
| K100-4 | L | V | H | A |
| K100-5 | I | M | H | L |
| K100-6 | A | I | N | I |
| K100-7 | I | C | H | L |
| K100-8 | NO MUTATION | S | H | NO |
| | | | | MUTATION |
| K150-1 | V | V | H | A |
| K150-2 | C | V | H | Y |
| K150-3 | I | A | H | H |
| K150-4 | L | C | G | V |
| K150-5 | NO MUTATION | V | H | NO |
| | | | | MUTATION |
| K150-11 | V | V | H | L |
| K150-15 | I | L | H | A |
| K200-6 | L | A | H | Y |
| K200-10 | G | V | L | L |
| K200-11 | NO MUTATION | P | N | V |
| K200-12 | G | V | N | V |
| (PylC^{GVNV}) | | | | |

In some embodiments, the PylC variant polynucleotide sequence (coding sequence) comprises at least 90%, at least 91%, at least 92%, at least 93%, at least 94%, at least 95%, at least 96%, at least 97%, at least 98%, at least 99%, or 100% sequence identity with any one of the PylC variant polynucleotide sequences as set forth in SEQ ID NO: 90, 92, 94, 96, 98, 100, 102, 104, 106, 108, 110, 112, 114, 116, 118, 120, 122, 124, 126, 130, 132, 134, 136, 138, 140, or 142. In some embodiments, the PylC variant polypeptide sequence comprises at least 90%, at least 91%, at least 92%, at least 93%, at least 94%, at least 95%, at least 96%, at least 97%, at least 98%, at least 99%, or 100% sequence identity with any one of the following PylC variant polypeptide sequences as set forth in SEQ ID NO: 91, 93, 95, 97, 99, 101, 103, 105, 107, 109, 111, 113, 115, 117, 119, 121, 123, 125, 127, 129, 131, 133, 135, 137, 139, 141, or 143. In some embodiments, the PylC variant polynucleotide sequence comprises one or more mutations at nucleic acids that correspond to S177, E179, D233, and/or T256 of SEQ ID NO: 1. In some embodiments, the PylC variant polynucleotide sequence is the sequence of SEQ ID NO: 142. In some embodiments, the PylC variant polypeptide sequence comprises a mutation at one or more of S177, E179, D233, and/or T256 of SEQ ID NO: 1. In some embodiments, the PylC variant is variant K200-12 (designated as PylC_{GVNV}; SEQ ID NO: 143).

c. PylC Variants for Synthesizing D-Allyl-N^{ε}-L-Lysine (D-Allyl-ε-Lys)

In some embodiments, the PylC variant is capable of producing D-Allyl-ε-Lys by using (D)-Allyl-OH and L-Lys as substrates (FIG. 9A). D-Allyl-ε-Lys consists of an allyl group attached to the s-nitrogen of an L-Lys. D-Allyl-ε-Lys comprises a reactive vinyl handle that may be used for thiol-ene reactions (also known as alkene hydrothiolation reactions) involving either a free radical addition reaction or a Michael addition reaction between the alkene of the vinyl handle and a thiol. Thiol-ene chemistry, thiol-Michael addition, and related click chemistry reactions are discussed in detail in Nolan, Mark D, and Eoin M Scanlan, Frontiers in Chemistry vol. 8 583272. 12 Nov. 2020; Hoyle, Charles E, and Christopher N Bowman. Angewandte Chemie (International ed. in English) vol. 49, 9 (2010): 1540-73; and Devatha P. Nair, et al., Chemistry of Materials 2014 26 (1), 724-744.

In some embodiments, the D-Allyl-ε-Lys PylC variant comprises a polypeptide sequence at least 90%, at least 91%, at least 92%, at least 93%, at least 94%, at least 95%, at least 96%, at least 97%, at least 98%, or at least 99% sequence identity with a wild-type archaeal PylC polypeptide sequence, e.g., the M. mazei PylC (UniProt ID: Q8PWY3; SEQ ID NO: 1). In some embodiments, the D-Allyl-ε-Lys PylC further comprises mutations at positions that correspond to S177, E179, D233, and/or T256 of the M. mazei PylC (UniProt ID: Q8PWY3; SEQ ID NO: 1), where one or more of those amino acids are substituted with one or more non-native amino acids. Exemplary amino acid mutations for S177, E179, D233, and/or T256 are provided in Table 3 below. In some embodiments, the PylC variant comprises the mutations S177G, E179V, D233N, and T256V (PylC^{GVNV} mutations).

TABLE 3
PylC Variants for D-Allyl-ε-Lys Synthesis.

| Variant | S177 | E179 | D233 | T256 |
|---|---|---|---|---|
| K200-12 | G | V | N | V |
| (PylC^{GVNV}) | | | | |
| K50-2-2 | L | C | G | H |
| K50-2-3 | H | G | S | A |
| K50-2-4 | A | C | S | L |
| K50-5-1 | G | V | L | Y |
| K50-5-2 | L | T | T | M |
| K100-2-1 | G | L | W | S |
| K100-2-2 | V | L | S | A |
| K100-2-5 | NO MUTATION | V | L | L |
| K150-5-1 | L | P | S | N |
| K200-5-1 | V | V | T | L |

In some embodiments, the PylC variant polynucleotide sequence (coding sequence) comprises at least 90%, at least 91%, at least 92%, at least 93%, at least 94%, at least 95%, at least 96%, at least 97%, at least 98%, at least 99%, or 100% sequence identity with the PylC variant polynucleotide sequence as set forth in SEQ ID NO: 142. In some embodiments, the PylC variant polypeptide sequence comprises at least 90%, at least 91%, at least 92%, at least 93%, at least 94%, at least 95%, at least 96%, at least 97%, at least 98%, at least 99%, or 100% sequence identity with the PylC variant polypeptide sequence as set forth in SEQ ID NO: 143. In some embodiments, the PylC variant polynucleotide sequence comprises one or more mutations at nucleic acids that correspond to S177, E179, D233, and/or T256 of SEQ ID NO: 1. In some embodiments, the PylC variant polynucleotide sequence is the sequence of SEQ ID NO: 142. In some embodiments, the PylC variant polypeptide sequence comprises a mutation at one or more of S177, E179, D233, and/or T256 of SEQ ID NO: 1. In some embodiments, the PylC variant is variant K200-12 (designated as PylC^{GVNV}; SEQ ID NO: 143).

d. PylC Variants for Synthesizing 2-Chloro-Acetyl-ε-Lys (2ClAcK)

In some embodiments, the PylC variant is capable of producing 2ClAcK by using 2-chloro-acetic acid and Lys as substrates (FIG. 10). 2ClAcK consists of a 2-chloro-acetyle group attached to the ε-nitrogen of an L-Lys. 2ClAcK contains a 2-chloro-acetyl group for facilitating protein dimerization, cyclization, and in vivo sumoylation of proteins. In some embodiments, when 2ClAcK is in close proximity with a suitable active group, such as a cysteine, 2ClAcK reacts with the active group to form a crosslink.

In some embodiments, the 2ClAcK PylC variant comprises a polypeptide sequence with at least 90%, at least 91%, at least 92%, at least 93%, at least 94%, at least 95%, at least 96%, at least 97%, at least 98%, or at least 99% sequence identity with a wild-type archaeal PylC polypeptide sequence, e.g., the M. mazei PylC (UniProt ID: Q8PWY3; SEQ ID NO: 1). In some embodiments, the 2ClAcK PylC further comprises mutations at positions that correspond to S177, E179, D233, and/or T256 of the M. mazei PylC (UniProt ID: Q8PWY3; SEQ ID NO: 1), where one or more of those amino acids are substituted with one or more non-native amino acids. Exemplary amino acid mutations for S177, E179, D233, and/or T256 are provided in Tables 1-3 above. In some embodiments, the PylC variant comprises the mutations S177N, E179P, D233S, and T256V (PylC^{NPSV} mutations). In some embodiments, the PylC variant comprises the mutations S177G, E179V, D233N, and T256V (PylC^{GVNV} mutations).

In some embodiments, the PylC variant polynucleotide sequence (coding sequence) comprises at least 90%, at least 91%, at least 92%, at least 93%, at least 94%, at least 95%, at least 96%, at least 97%, at least 98%, at least 99%, or 100% sequence identity with any one of the PylC variant polynucleotide sequences as set forth in SEQ ID NO: 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 130, 132, 134, 136, 138, 140, or 142. In some embodiments, the PylC variant polypeptide sequence comprises at least 90%, at least 91%, at least 92%, at least 93%, at least 94%, at least 95%, at least 96%, at least 97%, at least 98%, at least 99%, or 100% sequence identity with any one of the PylC variant polypeptide sequences as set forth in SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, or 143. In some embodiments, the PylC variant polynucleotide sequence comprises one or more mutations at nucleic acids that correspond to S177, E179, D233, and/or T256 of SEQ ID NO: 1. In some embodiments, the PylC variant polynucleotide sequence is the sequence of SEQ ID NO: 74 or 142. In some embodiments, the PylC variant polypeptide sequence comprises a mutation at one or more of S177, E179, D233, and/or T256 of SEQ ID NO: 1. In some embodiments, the PylC variant is variant K150-19 (designated as PylC^{NPSV}; SEQ ID NO: 75) or variant K200-12 (designated as PylC^{GVNV}; SEQ ID NO: 143).

### IV. PylRS (Pyrrolysyl-tRNA Synthetase)

PylRS is a pyrrolysyl-tRNA synthetase (also known as pyrrolysine-tRNA ligase) that is capable of ligating ncAAs (such as pyrrolysine, D-Cys-ε-Lys, D-Pra-ε-Lys, D-Allyl-ε-Lys, or 2ClAcK) to transfer ribonucleic acids (tRNAs). Cognate PylRSs and amber-suppressing tRNAs function together to introduce ncAAs at amber (TAG/UAG) codons during ribosomal translation of polypeptides (FIG. 1A). Therefore, recombinant host cells of the present disclosure typically comprise genes for both a PylRS and a tRNA, and the PylRS and the tRNA are expressed at the same time. In archaea, PylRS is commonly encoded by a single pylS gene. In some embodiments, PylRS ligates D-Cys-ε-Lys, D-Pra-ε-Lys, D-Allyl-ε-Lys, and/or 2ClAcK to tRNA at the amber (TAG/UAG) codon. Nonlimiting examples of PylRSs and tRNAs are discussed in Wan, Wei et al. Biochimica et Biophysica Scta vol. 1844, 6 (2014): 1059-70; Yuan, Jing et al. FEBS Letters vol. 584, 2 (2010): 342-9; Brabham, Robin, and Martin A Fascione. Chembiochem: a European Journal of Chemical Biology vol. 18, 20 (2017): 1973-1983; and Li, Wen-Tai et al. Journal of Molecular Biology vol. 385, 4 (2009): 1156-64.

PylRS displays high substrate side chain promiscuity, low selectivity towards its substrate α-amine, and low selectivity towards the anticodon of tRNA^{Pyl}. These features of PylRS allow for Pyl incorporation machinery to be engineered for the genetic incorporation of ncAAs or α-hydroxy acids into proteins at the amber UAG codon, and the reassignment of other codons such as ochre (TAA/UAA), opal (TGA/UGA), and four-base AGGA codons to code ncAAs (Wan, Wei et al. Biochimica et Biophysica Acta vol. 1844, 6 (2014): 1059-70). Amber suppression tRNAs are discussed below in detail. In some embodiments, the PylRS attaches Pyl, D-Cys-ε-Lys, D-Pra-ε-Lys, D-Allyl-ε-Lys, or 2ClAcK, or any combination thereof, onto tRNAs.

Many wild-type or modified archaeal and bacterial PylRS may be used in accordance with the present disclosure, for example, without limitations, a wild-type or modified PylRS from Desulfosporosinus orientis, Eubacterium limosum, Methanohalophilus mahii, Methanohalophilus halophilus, Methanosarcina barkeri, Methanosarcina mazei, Methanosarcina thermophila, Methanosarcina acetivorans, Methanosarcina vacuolata, Methanolobus tindarius, Methanococcoides methylutens, Desulfobacter sp., Desulfobacterium vacuolatum, Methanohalobium evestigatum. Acetohalobium arabaticum, Methanococcoides burtonii, Pseudobacteroides cellulosolvens, Bilophila wadsworthia, Desulfacinum infernum, Desulfitobacterium dehalogenans, Methanolobus vulcani, Methanosarcina siciliae, Methanohalophilus portucalensis, Methanosalsum zhilinae, Sporomusa sphaeroides, Desulfitobacterium hafniense, Methanohalophilus euhalobius, Dehalobacterium formicoaceticum, Desulfitobacterium chlororespirans, Acetobacterium fimetarium, Desulfospira joergensenii, Carboxydothermus ferrireducens, Sporomusa silvacetica, Desulfofarcimen acetoxidans, Desulfosporosinus meridiei, Thermacetogenium phaeum, Desulforhopalus singaporensis, Methanimicrococcus blatticola, Methanomethylovorans hollandica, Desulfallas gibsoniae, Sporomusa acidovorans, Sporomusa malonica, Parasporobacterium paucivorans, Desulfitobacterium sp. PCE1, uncultured Eubacterium sp., Methanosarcina lacustris, Desulfitibacter alkalitolerans, Thermincola ferriacetica, uncultured Sporomusa sp., Halarsenatibacter silvermanii, Desulfosporosinus lacus, Dethiosulfatibacter aminovorans, Olavius algarvensis, Delta endosymbiont, Desulfosporosinus youngiae, Methanosarcina horonobensis, Aminipila butyrica, Megamonas funiformis, Methanolobus profindi, Syntrophaceticus schinkii, Methanolobus zinderi, Desulfosporosinus hippei, Alkalibaculum bacchi, Methermicoccus shengliensis, Bilophila sp. 4 1 30, Olavius sp. associated proteobacterium Delta 1, Pectinatus brassicae, Thermincola potens, Desulfitobacterium sp. LBE, Sporomusa sp. KB1, Methanosarcina soligelidi, Methanosarcina spelaei, Thermoplasmatales archaeon BRNA1, Methanomassiliicoccus luminyensis PylRS1, Methanomassiliicoccus luminyensis PylRS2, Methanolobus psychrophilus R15, Sporomusa ovata DSM 2662, Methanococcoides sp. AM1, Methanococcoides sp. NM1, Desulfamplus magnetovallimortis, Eubacterium sp. 68-3-10, Firmicutes bacterium CAG:238, candidatus Methanomethylophilus alvus, Methanococcoides vulcani, candidatus Methanomassiliicoccus intestinalis, Methanosarcina sp. MTP4, Methanosarcina sp. WWM596, Methanogenic archaeon mixed culture IOS4-G1, Methanosarcina sp. 2.H.T.1A.6, Methanosarcina sp. 1.H.A.2.2, Methanosarcina sp. 1.H.T.1A.1, Peptococcaceae bacterium SCADC1 2 3, Desulfosporosinus sp. HMP52, Methanogenic archaeon ISO4-H5, Peptostreptococcaceae bacterium pGA-8, Desulfosporosinus sp. BICA1-9, Sporomusa sp. GT1, Methanomassilicoccaceae archaeon DOK, candidatus Methanoplasma termitum, Desulfosporosinus sp. I2, Desulfosporosinus sp. BG, candidate division MSBL1 archaeon SCGC-AAA382A20, Methanomassiliicoccales archaeon RumEn M1, Methanosarcina flavescens, Desulfitibacter sp. BRH c19, candidatus Methanomethylophilus sp. 1R26, Firmicutes bacterium ML8 F2, Emergencia_timonensis, Methanohalophilus sp. T328-1, Methanolobus sp. T82-4, Spirochaetes bacterium GWB1 66 5, Methanomethylovorans sp. PtaU1.Bin093, Methanomassiliicoccales archaeon PtaU1.Bin030, Methanomassiliicoccales archaeon PtaU1.Bin124, Methanomassiliicoccales archaeon Mx-06, Actinobacteria bacterium ADurb.BinA094, Methanohalophilus sp. DAL1, Desulfobulbaceae bacterium S5133MH15, Methanolobus psychrotolerans, Methanosarcina sp. Ant1, Desulfosporosinus sp. OL, Lachnospiraceae bacterium, Acetobacterium sp. MES1, candidatus Methanohalarchaeum thermophilum PylRS1, candidatus Methanohalarchaeum thermophilum PylRS2, Methanonatronarchaeum thermophilum, Methylomusa anaerophila, Methanosarcinaceae archaeon, Desulfosporosinus sp. FKB, Desulfobacteraceae bacterium 4572 123, Desulfosporosinus fructosivorans, candidatus Bathyarchaeota archaeon, Phycisphaeraceae bacterium, Thaumarchaeota archaeon, Thermoleophilia bacterium, Methanolobus sp. SY-01, Methanohalophilus profundi, Nitrososphaeria archaeon, Thermoplasmatales archaeon, Desulfacinum sp., Emergencia sp. 1XD21-50, Methanohalophilus sp. RSK, Methanohalophilus sp. WG1-DM, Methanosarcina sp. MSH10X1, Aminipila sp. JN-18, Zhaonella formicivorans, Desulfosporosinus sp. Sb-LF, Desulfobacula sp., Selenomonas sp. DSM 106892, Aminipila sp. CBA3637, candidatus Cryptoclostridium obscurum, Methanococcoides sp. SA1, candidatus Methanomethylicus mesodigestum, candidatus Hydrothermarchaeum profundi JdFR-18, candidatus Bathyarchaeota archaeon JdFR-11, candidatus Korarchaeota archaeon, Methanomicrobia archaeon JdFR-19, candidatus Sifarchaeum lw 55 reseq mb2.7, and candidatus Borrarchaeum weybense. For PylRS sequences from these organisms, see Guo, Li-Tao et al. The Journal of Biological Chemistry vol. 298, 11 (2022): 102521.

In some embodiments, the PylRS is a wild-type archaeal PylRS polypeptide sequence, for example, without limitations, the PylRS of Methanosarcina mazei (UniProt ID: Q8PWY1). In some embodiments, the PylRS variant is derived from a wild-type archaeal PylRS polypeptide sequence, for example, without limitations, the PylRS of Methanosarcina mazei (UniProt ID: Q8PWY1; SEQ ID NO: 144), the PylRS of Methanosarcina barkeri (UniProt ID: Q6WRH6, the PylRS of Methanosarcina acetivorans (UniProt ID: Q8TUB8), the PylRS of Desulfitobacterium hafniense, of the PylRS of Methanogenic archaeon ISO4-G1. In some embodiments, directed evolution is the strategy employed to produce PylRS variants (Tai, Jingxuan et al. Journal of the American Chemical Society vol. 145, 18 (2023): 10249-10258).

In some embodiments, the PylRS variant comprises a polypeptide sequence with at least 90%, at least 91%, at least 92%, at least 93%, at least 94%, at least 95%, at least 96%, at least 97%, at least 98%, or at least 99% sequence identity with a wild-type archaeal PylRS polypeptide sequence, e.g., the Methanosarcina mazei PylRS (UniProt ID: Q8PWY1; SEQ ID NO: 144). In some embodiments, the PylRS variant further comprises mutations at positions that correspond to L301, L305, Y306, L309, and/or C348 of M. mazei PylRS (UniProt ID: Q8PWY1; SEQ ID NO: 144), where one or more of those amino acids are substituted with one or more non-native amino acids. In some embodiments, the amino acid corresponding to L301 of the M. mazei PylRS (UniProt ID: Q8PWY1; SEQ ID NO: 144) can be any amino acid other than L. In some embodiments, the amino acid corresponding to L305 of the M. mazei PylRS (UniProt ID: Q8PWY1; SEQ ID NO: 144) can be any amino acid other than L. In some embodiments, the amino acid corresponding to Y306 of the M. mazei PylRS (UniProt ID: Q8PWY1; SEQ ID NO: 144) can be any amino acid other than Y. In some embodiments, the amino acid corresponding to L309 of the M. mazei PylRS (UniProt ID: Q8PWY1; SEQ ID NO: 144) can be any amino acid other than L. In some embodiments, the amino acid corresponding to C348 of the M. mazei PylRS (UniProt ID: Q8PWY1; SEQ ID NO: 144) can be any amino acid other than C. In some embodiments, the PylRS variant comprises the mutations L301M, L305I, Y306L, L309A, and/or C348F. See, e.g., Kobayashi et al. Journal of the American Chemical Society 2016 138 (45), 14832-14835. In some embodiments, the PylRS variant comprises the mutations L301M, L305I, Y306L, L309A, and C348F (PylS* mutations).

In some embodiments, the PylRS variant comprises a polypeptide sequence with at least 90%, at least 91%, at least 92%, at least 93%, at least 94%, at least 95%, at least 96%, at least 97%, at least 98%, or at least 99% sequence identity with a wild-type archaeal PylRS polypeptide sequence, e.g., the M. mazei PylRS (UniProt ID: Q8PWY1; SEQ ID NO: 144). In some embodiments, the PylRS variant further comprises mutations at positions that correspond to G14, S451, and/or C348 of the M. mazei PylRS (UniProt ID: Q8PWY1; SEQ ID NO: 144), where one or more of those amino acids are substituted with one or more non-native amino acids. In some embodiments, the amino acid corresponding to G14 of the M. mazei PylRS (UniProt ID: Q8PWY1; SEQ ID NO: 144) can be any amino acid other than G. In some embodiments, the amino acid corresponding to S451 of the M. mazei PylRS (UniProt ID: Q8PWY1; SEQ ID NO: 144) can be any amino acid other than S. In some embodiments, the amino acid corresponding to C348 of the M. mazei PylRS (UniProt ID: Q8PWY1; SEQ ID NO: 144) can be any amino acid other than C. In some embodiments, the PylRS variant comprises the mutations G14E, S451F, and/or C348V. In some embodiments, the PylRS variant comprises the mutations G14E, S451F, and C348V (PylRS^{EVF} mutations).

In some embodiments, the PylRS variant polynucleotide sequence (coding sequence) comprises at least 90%, at least 91%, at least 92%, at least 93%, at least 94%, at least 95%, at least 96%, at least 97%, at least 98%, at least 99%, or 100% sequence identity with the PylRS variant polynucleotide sequence as set forth in SEQ ID NO: 145. In some embodiments, the PylRS variant polypeptide sequence comprises at least 90%, at least 91%, at least 92%, at least 93%, at least 94%, at least 95%, at least 96%, at least 97%, at least 98%, at least 99%, or 100% sequence identity with the PylRS variant polypeptide sequence as set forth in SEQ ID NO: 146. In some embodiments, the PylRS variant polynucleotide sequence comprises one or more mutations at nucleic acids that correspond to G14, S451, and/or C348 of the M. mazei PylRS (UniProt ID: Q8PWY1; SEQ ID NO: 144). In some embodiments, the PylRS variant polynucleotide sequence is the sequence of SEQ ID NO: 145. In some embodiments, the PylC variant polypeptide sequence comprises a mutation atone or more of G14, S451, and/or C348 of the M. mazei PylRS (UniProt ID: Q8PWY1; SEQ ID NO: 144). In some embodiments, the PylRS polypeptide variant is the PylRS^{EVF} variant (as set forth in SEQ ID NO: 146). In some embodiments, the PylRS variant is the PylS* variant comprising the mutations L301M, L305I, Y306L, L309A, and C348F, as discussed in Kobayashi et al. Journal of the American Chemical Society 2016 138 (45), 14832-14835.

In some embodiments, the PylRS capable of ligating D-Cys-ε-Lys to tRNA at amber (TAG/UAG) codons (and thus able to incorporate D-Cys-ε-Lys into proteins) is a PylRS with the amino acid sequence of SEQ ID NO: 144, or a PylRS with the amino acid sequence of SEQ ID NO: 146 (PylRS^{EVF}).

In some embodiments, the PylRS capable of ligating D-Pra-ε-Lys to tRNA at amber (TAG/UAG) codons (and thus able to incorporate D-Pra-ε-Lys into proteins) is a PylRS with the amino acid sequence of SEQ ID NO: 144, or a PylRS with the amino acid sequence of SEQ ID NO: 146 (PylRS^{EVF}).

In some embodiments, the PylRS capable of ligating D-Allyl-ε-Lys to tRNA at amber (TAG/UAG) codons (and thus able to incorporate D-Allyl-ε-Lys into proteins) is a PylRS with the amino acid sequence of SEQ ID NO: 144, or a PylRS with the amino acid sequence of SEQ ID NO: 146 (PylRS^{EVF}).

In some embodiments, the PylRS capable of ligating 2ClAcK to tRNA at amber (TAG/UAG) codons (and thus able to incorporate 2ClAcK into proteins) is a PylRS with the amino acid sequence of SEQ ID NO: 144, a PylRS with the amino acid sequence of SEQ ID NO: 146 (PylRS^{EVF}), or a PylRS with the amino acid sequence of SEQ ID NO: 144 comprising mutations corresponding to L301M, L305I, Y306L, L309A, and C348F (PylS* mutations) of SEQ ID NO: 144.

### V. Amber Suppression Transfer Ribonucleic Acids (tRNAs)

PylT encodes a pylT mRNA transcript that functions as wild-type tRNA^{Pyl}, which contains a CUA anticodon for the recognition of the amber (TAG/UAG) codon. Wild-type tRNA^{Pyl} is charged by wild-type PylRS, and Pyl is then incorporated into the translation of nascent polypeptides (FIG. 1A). tRNA^{Pyl} has been shown to be orthogonal in commonly used bacterial and eukaryotic cells and functional in the reassignment of the amber (TAG/UAG) codon when coupled with PylRS in these cell strains. Use of orthogonal PylRS and tRNA pairs to incorporate ncAAs at amber (TAG/UAG) stop codons effectively suppress translation termination in the presence of the ncAA. Amber suppression and nonlimiting examples of tRNAs and PylRSs are discussed in Wan, Wei et al. Biochimica et Biophysica Acta vol. 1844, 6 (2014): 1059-70; Yuan, Jing et al. FEBS Letters vol. 584, 2 (2010): 342-9; Brabham, Robin, and Martin A Fascione. Chembiochem: a European Journal of Chemical Biology vol. 18, 20 (2017): 1973-1983; Gaston, Marsha A et al. Current Opinion in Microbiology vol. 14, 3 (2011): 342-9; and Li, Wen-Tai et al. Journal of Molecular Biology vol. 385, 4 (2009): 1156-64.

In some embodiments, the tRNA is derived from wild-type archaeal tRNA^{Pyl}, for example, without limitations, tRNA^{Pyl} of Methanosarcina mazei (SEQ ID NO: 147) or the pylT gene of Methanosarcina mazei (see, e.g., GenBank Locus ID: MW879724.1). In some embodiments, the tRNA variant demonstrates stronger tertiary interactions, e.g., the G19:C56 pair between the D-loop and the T-loop in the tRNA (Serfling, Robert et al. Nucleic Acids Research vol. 46, 1 (2018): 1-10). In some embodiments, the tRNA variant further comprises mutations in its polynucleotide sequence at nucleic acids that correspond to positions A11, U15, U19, U25, U29a, and/or A56 of the M. mazei tRNA^{Pyl} (SEQ ID NO: 147). In some embodiments, the nucleic acid at A11 can be any nucleic acid except A. In some embodiments, the nucleic acid at U15 can be any nucleic acid except U. In some embodiments, the nucleic acid at U19 can be any nucleic acid except U. In some embodiments, the nucleic acid at U25 can be any nucleic acid except U. In some embodiments, the nucleic acid at U29a can be any nucleic acid except U. In some embodiments, the nucleic acid at A56 can be any nucleic acid except A.

In some embodiments, the tRNA variant comprises at least 90%, at least 91%, at least 92%, at least 93%, at least 94%, at least 95%, at least 96%, at least 97%, at least 98%, or at least 99% sequence identity with the tRNA^{Pyl} of Methanosarcina mazei (SEQ ID NO: 147) or the pylT gene of Methanosarcina mazei (see, e.g., GenBank Locus ID: MW879724.1). In some embodiments, the tRNA further comprises one or more nucleic acid substitutions corresponding to positions A11G, U15, U19, U25, U29a, and/or A56 of the tRNA^{Pyl} of Methanosarcina mazei (SEQ ID NO: 147) or the pylT gene of Methanosarcina mazei (see, e.g. GenBank Locus ID: MW879724.1). In some embodiments, the tRNA^{Pyl} comprises the A11G, U15G, U19G, U25C, U29aC, and A56C mutations (the tRNA^{M15} mutations). In some embodiments, the tRNA is the tRNA^{M15} discussed in the Examples below and in Serfling, Robert et al. Nucleic Acids Research vol. 46, 1 (2018): 1-10.

### VI. Polynucleotides

Disclosed herein are polynucleotide sequences that encode for PylCs, PylRSs, and tRNAs for in cellulo synthesis of ncAAs and proteins comprising ncAAs. Polynucleotide sequences for PylCs, PylRSs, and tRNAs are disclosed above in their respective sections.

Also disclosed herein are polynucleotide sequences encoding proteins of interest, where each protein of interest comprises one or more amber (TAG/UAG) codons for incorporation of an ncAA at each of the amber (TAG/UAG) codons in the polynucleotide sequence. In some embodiments, the polynucleotide sequence encoding the protein of interest further comprises a stop codon for translation termination and that stop codon is not an amber (TAG/UAG) codon, but an ochre (TAA/UAA) or an opal (TGA/UGA) codon instead. Many proteins are suitable for incorporation of an ncAA in their polypeptide sequences, e.g., without limitations, therapeutic peptides, antibodies, contractile proteins, enzymes, hormonal proteins, structural proteins, storage proteins, transport proteins, fibrous proteins, globular proteins, membrane proteins, and proteins conjugated to other organic and/or inorganic groups such as metals, lipids, sugars, and/or phosphate. Nonlimiting examples of proteins of interest include P16 peptide, small ubiquitin-related modifier (SUMO) proteins, and variants thereof (including mutants, truncations, and fusions) as discussed in the Examples below. Non-limiting examples of SUMO proteins and methods for SUMOylation are disclosed in U.S. Patent Application Publication No. 2022/0194997.

The polynucleotide sequences disclosed herein may be introduced into host cells, e.g., E. coli, for expression. In some embodiments, one or more polynucleotide sequences are subcloned into one or more expression vectors. Each expression vector may comprise 5′ and 3′ regulatory sequences that are operably linked to the polynucleotide sequence(s). In general, an expression vector comprises a promoter for driving transcription, a transcription/translation terminator, and a ribosome binding site for translation initiation. An expression may also comprise an origin of replication, one or more selection markers, a plurality of restriction sites and/or recombination sites for insertion of the polynucleotides to be under the transcriptional regulation of the regulatory elements. Suitable expression vectors and elements therein for use in host cells are well known in the art and are discussed, e.g., in Sambrook and Russell, Molecular Cloning, A Laboratory Manual (3rd ed. 2001); Kriegler, Gene Transfer and Expression: A Laboratory Manual (1990); Ausubel et al., eds., Current Protocols in Molecular Biology (1994); and Davis et al., eds. (1980) Advanced Bacterial Genetics (Cold Spring Harbor Laboratory Press), Cold Spring Harbor, N.Y.

The polynucleotide sequences disclosed herein may be modified to produce protein variants. Polynucleotide sequences may be modified using a variety of methods that are readily available to one of ordinary skill in the art, e.g., without limitations, site-saturation mutagenesis, directed evolution, and site-directed mutagenesis. See, e.g., Nov, Yuval. Applied and Environmental Microbiology vol. 78, 1(2012): 258-62; Gupta, Kritika, and Raghavan Varadarajan. Current Opinion in Structural Biology vol. 50 (2018): 117-125; Tai, Jingxuan et al. Journal of the American Chemical Society vol. 145, 18 (2023): 10249-10258; and Bachman, Julia. Methods in Enzymology vol. 529 (2013): 241-8.

The polynucleotides disclosed herein may be introduced into a host cell using a variety of methods, for example, without limitations, electroporation, nanoparticle delivery, viral delivery, contact with nanowires or nanotubes, receptor mediated internalization, translocation via cell penetrating peptides, liposome mediated translocation, DEAE dextran, lipofectamine, calcium phosphate or any method now known or identified in the future for introduction of nucleic acids into prokaryotic or eukaryotic cellular hosts. A targeted nuclease system (e.g., an RNA-guided nuclease (e.g., a CRISPR-Cas nuclease), a transcription activator-like effector nuclease (TALEN), a zinc finger nuclease (ZFN), or a megaTAL (MT) can also be used to introduce the polynucleotide into the genome of a host cell. See, e.g., Zhang, Hong-Xia et al. Molecular Therapy: the Journal of the American Society of Gene Therapy vol. 27, 4 (2019): 735-746; and González Castro, Nicolás et al. International Journal of Molecular Sciences vol. 22, 19 10355. 26 Sep. 2021.

### VI. Recombinant Host Cells

Disclosed herein are recombinant host cells capable of expressing the PylCs, PylRSs, and/or tRNAs of the present disclosure. Also disclosed herein are recombinant host cells for in cellulo synthesis of ncAAs and proteins comprising the ncAAs. Nonlimiting examples of ncAAs include D-Cys-ε-Lys, D-Pra-ε-Lys, D-Allyl-ε-Lys, and 2ClAcK.

The recombinant host cell capable of in cellulo synthesis of ncAAs and proteins comprising ncAAs typically comprises a polynucleotide sequence encoding a mutant PylC capable of synthesizing the desired ncAA. In some embodiments, the recombinant host cell also comprises a polynucleotide sequence encoding a wild-type or mutant PylRS that is capable of attaching the desired ncAA to a tRNA. In some embodiments, the recombinant host cell also comprises a polynucleotide sequence encoding a wild-type or mutant tRNA that is charged with an ncAA by the wild-type or mutant PylRS, and the wild-type or mutant tRNA incorporates the ncAA at an amber (TAG/UAG) codon during polypeptide translation. PylCs, PylRSs and tRNAs, and the polynucleotide sequences that encode for them are discussed in detail above. In some embodiments, the recombinant host cell further comprises a polynucleotide sequence encoding a protein of interest with one or more amber (TAG/UAG) codons for incorporation of an ncAA at each of the amber (TAG/UAG) codons in its polypeptide sequence. In some embodiments, the polynucleotide sequence encoding the protein of interest further comprises a stop codon for translation termination and that stop codon is not an amber (TAG/UAG) codon, but an ochre (TAA/UAA) or an opal (TCA/UGA) codon instead.

In some embodiments, the recombinant host cell is capable of synthesizing D-Cys-ε-Lys. In some embodiments, the recombinant host cell comprises PylC_{NPSV}. In some embodiments, the recombinant host cell further comprises wild-type PylRS or PylRS^{EVF}. In some embodiments, the recombinant host cell further comprises tRNA^{Pyl} or tRNA^{M15}. In some embodiments, the recombinant host cell further comprises a polynucleotide sequence encoding a protein of interest with one or more amber (TAG/UAG) codons placed at pre-determined position(s) for incorporation of a D-Cys-ε-Lys at each of the amber (TAG/UAG) codons in its corresponding polypeptide sequence. In some embodiments, the polynucleotide sequence encoding the protein of interest comprises an ochre (TAA/UAA) or an opal (TCA/UGA) stop codon for translation termination.

In some embodiments, the recombinant host cell is capable of synthesizing D-Pra-ε-Lys. In some embodiments, the recombinant host cell comprises PylC^{GVNV}. In some embodiments, the recombinant host cell further comprises PylRS^{EVF}. In some embodiments, the recombinant host cell further comprises tRNA^{Pyl} or tRNA^{M15}. In some embodiments, the recombinant host cell further comprises a polynucleotide sequence encoding a protein of interest with one or more amber (TAG/UAG) codons placed at pre-determined position(s) for incorporation of a D-Pra-ε-Lys at each of the amber (TAG/UAG) codons in its corresponding polypeptide sequence. In some embodiments, the polynucleotide sequence encoding the protein of interest comprises an ochre (TAA/UAA) or an opal (TCA/UGA) stop codon for translation termination.

In some embodiments, the recombinant host cell is capable of synthesizing D-Allyl-ε-Lys. In some embodiments, the recombinant host cell comprises PylC^{GVNV}. In some embodiments, the recombinant host cell further comprises PylRS^{EVF}. In some embodiments, the recombinant host cell further comprises tRNA^{Pyl} or tRNA^{M15}. In some embodiments, the recombinant host cell further comprises a polynucleotide sequence encoding a protein of interest with one or more amber (TAG/UAG) codons at pre-determined position(s) for incorporation of a D-Allyl-ε-Lys at each of the amber (TAG/UAG) codons in its corresponding polypeptide sequence. In some embodiments, the polynucleotide sequence encoding the protein of interest further comprises an ochre (TAA/UAA) or an opal (TCA/UGA) stop codon for translation termination.

In some embodiments, the recombinant host cell is capable of synthesizing 2ClAcK. In some embodiments, the recombinant host cell comprises a PylC variant that is capable of synthesizing 2ClAcK. In some embodiments, the recombinant host cell further comprises PylS* or PylRS^{EVF}. In some embodiments, the recombinant host cell further comprises tRNA^{Pyl} or tRNA^{M15}. In some embodiments, the recombinant host cell further comprises a polynucleotide sequence encoding a protein of interest with one or more one amber (TAG/UAG) codons at pre-determined position(s) for incorporation of a 2ClAcK at each of the amber (TAG/UAG) codons in its corresponding polypeptide sequence. In some embodiments, the polynucleotide sequence encoding the protein of interest further comprises an ochre (TAA/UAA) or an opal (TCA/UGA) stop codon for translation termination.

In some embodiments, the recombinant host cell is a bacterial cell. Methods for culturing bacteria and use of bacteria for protein expression (which includes the in cellulo synthesis of ncAAs and proteins comprising the ncAAs) are well known to those skilled in the art. Bacteria suitable for culture and in cellulo synthesis of ncAAs and proteins comprising the ncAAs include gram-negative bacteria and gram-positive bacteria, for example, Enterobacteriaceae such as Escherichia, Enterobacter, Erwinia, Klebsiella, Proteus, Salmonella, Serratia, Shigella; as well as Bacilli, Pseudomonas, and Streptomyces. A bacteria derived from any strain of these bacteria can be used in the methods of the disclosure. In some embodiments, the bacterial host cell is E. coli. In some embodiments, the E. coli strain is an A (K-12), B, C or D strain. Suitable media for culturing bacteria and for protein overexpression are well-known to one of ordinary skill in the art. Examples of media recipes may be found, for example, in Allikian et al., (2019). Fundamentals of Fermentation Media. In: Berenjian, A. (eds) Essentials in Fermentation Technology. Learning Materials in Biosciences. Springer, Cham.

In some embodiments, the recombinant host cell is a eukaryotic cell. Methods for culturing eukaryotic cells and use of eukaryotic cells for protein and tRNA expression (which includes the in cellulo synthesis of ncAAs and proteins comprising the ncAAs) are well known to those skilled in the art. Non-limiting examples of eukaryotic cells include mammalian cells, yeast, and insect cells, which are well known in the art and are also commercially available. Eukaryotic expression systems are discussed in Geisse, S et al. Protein Expression and Purification vol. 8, 3 (1996): 271-82; and Khan, Kishwar Hayat. Advanced Pharmaceutical Bulletin vol. 3, 2 (2013): 257-63.

Standard transfection methods are used to produce bacterial, mammalian, yeast, or insect cell lines that express the PylCs, PylRSs, and tRNAs of the present disclosure, and for in cellulo synthesis of ncAAs and proteins comprising ncAAs. Transformation of prokaryotic and eukaryotic cells are performed according to standard techniques (see, e.g., Morrison, J. Bact. 132: 349-351 (1977); Clark-Curtiss & Curtiss, Methods in Enzymology 101: 347-362 (Wu et al., eds, 1983)). Any of the well-known procedures for introducing foreign nucleotide sequences into host cells may be used. These include the use of calcium phosphate transfection, polybrene, protoplast fusion, electroporation, liposomes, microinjection, plasma vectors, viral vectors, and any of the other well-known methods for introducing cloned genomic DNA, cDNA, synthetic DNA, or other foreign genetic material into a host cell (see, e.g., Sambrook and Russell, supra). It is only necessary that the particular genetic engineering procedure used be capable of successfully introducing at least one gene into the host cell, thereby enabling expression of the gene product (e.g., PylCs, PylRSs, and tRNAs of the present disclosure), as well as expression of ncAAs and proteins comprising the ncAAs by the recombinant host cell. The proteins of the present disclosure, including proteins comprising the ncAAs, can be purified using standard techniques (see, e.g., Colley et al., Methods in Enzymology vol. 182 (1990): 1-818; and Bonetta, Laura. Nature vol. 439, 7079 (2006): 1018).

a. Genome Modifications

In some embodiments, a recombinant host cell of the present disclosure further comprises a modification in its genomic sequence encoding Release Factor 1 (RF1). RF1 is a protein that allows for the termination of translation by recognizing UAG and UAA stop codons in an mRNA sequence. Disruption of RF1 expression levels or activity can help enhance read-through at the amber (UAG) codon where an ncAA (e.g., without limitations, D-Cys-ε-Lys, D-Pra-ε-Lys, D-Allyl-ε-Lys, and 2ClAcK) is to be incorporated into the protein. For example, the recombinant host cell's genomic sequence encoding RF1 may be deleted, truncated, or mutated, so as to reduce or eliminate RF1 expression or activity. For example, an insertion in the RF1 gene of the recombinant host cell can also reduce or eliminate RF1 expression or activity. Many genome modifying systems and corresponding nucleases may be used to disrupt, reduce, or eliminate RF1 expression or activity, for example, without limitations, to modify or delete the genomic coding sequence for RF1. Non-limiting examples of genome modifying systems and nucleases include a homing nuclease polypeptide; a FokI polypeptide; a transcription activator-like effector nuclease (TALEN) polypeptide; a MegaTAL polypeptide; a meganuclease polypeptide; a zinc finger nuclease (ZFN); an ARCUS nuclease; and the like. The meganuclease can be engineered from an LADLIDADG homing endonuclease (LHE). A megaTAL polypeptide can comprise a TALE DNA binding domain and an engineered meganuclease. See, e.g., International Patent Application Publication No. WO 2004/067736 (homing endonuclease); Urnov et al. (2005) Nature 435:646 (ZFN); Mussolino et al. (2011) Nucleic Acids Research 39:9283 (TALE nuclease); Boissel et al. (2013) Nucleic Acids Research 42:2591 (MegaTAL). In some embodiments, the genome modifying system is a CRISPR-Cas system comprising a CRISPR-Cas nuclease and guide polynucleotides, e.g., guide RNAs (gRNAs). In some embodiments, for example, the CRISPR-Cas nuclease is CRISPR-Cas9. See, e.g., Møller, Henrik Devitt et al. Nucleic Acids Research (2018); Burstein et al. (2017) Nature 542:237; Slaymaker et al. Science, 351(6268):84-8 (2016); and Kleinstiver et al. Nature, 529(7587):490-5 (2016).

In some embodiments, a recombinant host cell of the present disclosure further comprises a modification in its genomic sequence where one, some, or all native TAG/UAG stop codons are mutated. In some embodiments, one, some, or all native TAG/UAG stop codons are mutated to TAA/UAA stop codons, TGA/UGA stop codons, or a combination of both. As discussed above, polynucleotide sequences encoding proteins of interest comprise one or more TAG/UAG codons for incorporation of an ncAA at each TAG/UAG codon in the polynucleotide sequence. Thus, in some embodiments, it can be useful for a recombinant host cell to comprise TAA/UAA stop codons and/or TGA/UGA stop codons (instead of TAG/UAG stop codons) so that ncAAs are not incorporated into any or all polypeptides expressed by the cell in addition to the protein of interest. In some embodiments, recombinant host cells comprise TAA/UAA stop codons and/or TGA/UGA stop codons (instead of TAG/UAG stop codons) to prevent use of the cell's reservoir of ncAAs for the synthesis of proteins other than the proteins of interest. In some embodiments, the recombinant host cell is devoid of any TAG/UAG stop codon.

b. Reagent Cell Lysates

In some embodiments, the recombinant host cells disclosed herein (comprising polynucleotides of the present disclosure as discussed above) can be used to produce a reagent cell lysate for use in cell-free expression systems. The ncAAs D-Cys-ε-Lys, D-Pra-ε-Lys, D-Allyl-ε-Lys, and 2ClAcK, and/or any protein comprising one or more of these ncAAs, can be produced in a cell-free expression system using a reagent cell lysate that comprises the PylCs, PylRSs, and tRNAs of the present disclosure. In some embodiments, the reagent cell lysate comprises at least one PylC variant, one PylRS variant, and one tRNA variant of the present disclosure.

In some embodiments, recombinant host cells of the present disclosure are cultured using appropriate conditions in the presence of appropriate PylC variant substrates to produce one or more ncAAs, e.g., D-Cys-ε-Lys, D-Pra-ε-Lys, D-Allyl-ε-Lys, and/or 2ClAcK. Methods for in cellulo synthesis of ncAAs are discussed in detail below. Typically, the recombinant host cells are harvested from culture and lysed to produce reagent cell lysate.

In some embodiments, polynucleotides comprising a coding sequence for a desired protein is added to the reagent cell lysate for cell-free expression of the desired protein. Typically, the coding sequence for a desired protein comprises (1) one or more amber (TAG/UAG) codons for incorporation of an ncAA at each of the amber (TAG/UAG) codons in the protein sequence, and (2) a stop codon for translation termination, and that stop codon is not an amber (TAG/UAG) codon, but an ochre (TAA/UAA) or an opal (TGA/UGA) codon instead.

In some embodiments, additional reagents are added to the reagent cell lysate for the cell-free expression of a desired protein. In some embodiments, the additional reagent is one or more ncAAs (e.g., D-Cys-ε-Lys, D-Pra-ε-Lys, D-Allyl-ε-Lys, and/or 2ClAcK. In some embodiments, one or more substrates for the PylC variant(s) in the reagent cell lysate are added to the reagent cell lysate for the production of one or more ncAAs (e.g., D-Cys-ε-Lys, D-Pra-ε-Lys, D-Allyl-ε-Lys, and/or 2ClAcK), prior to or in addition to the cell-free expression of a desired protein.

In general, a reagent cell lysate comprises components that are capable of translating mRNA encoding a protein of interest. In some cases, the reagent cell lysate may comprise components that are capable of transcribing DNA encoding a desired protein, for example, without limitations, DNA-directed RNA polymerase (RNA polymerase), transcription activators, tRNAs, aminoacyl-tRNA synthetases, 70S ribosomes, N10-formyltetrahydrofolate, formylmethionine-tRNAfMet synthetase, peptidyl transferase, initiation factors (such as IF-1, IF-2, and IF-3), elongation factors (such as EF-Tu, EF-Ts, and EF-G), release factors (such as RF-2 and RF-3), and the like. Methods of preparing reagent cell lysate may include growing a recombinant host cell of the present disclosure to log phase using suitable growth media and conditions. The recombinant cells are harvested and lysed to produce the reagent cell lysate. Methods of preparing and using reagent cell lysates and cell-free expression systems are described in, e.g., Perez, Jessica G et al. Cold Spring Harbor Perspectives in Biology vol. 8, 12 a023853. 1 Dec. 2016; August et al. Life (Basel, Switzerland) vol. 11, 12 1367. 8 Dec. 2021; Smolskaya, Sviatlana et al. International Journal of Molecular Sciences vol. 21, 3 928. 31 Jan. 2020; Zawada, James F. Methods in Molecular Biology (Clifton, N.J.) vol. 805 (2012): 31-41; Jewett et al., Molecular Systems Biology, 4, 1-10 (2008); and Shin J. and Norieaux V., J. Biol. Eng., 4:8 (2010); Zawada et al., Biotechnol Bioeng, 108:1570-1578 (2011); Yin et al., mAbs, 4(2):217-225 (2012); and Zimmerman et al., Bioconjug Chem, 25(2):351-61 (2014).

In some embodiments, the reagent cell lysate comprises ncAAs for the production of proteins comprising the ncAAs. In some embodiments, reagent ncAAs (e.g., ncAAs from other sources) may be added to the reagent cell lysate for the production of proteins comprising the ncAAs. In some embodiments, substrates for PylC variants (discussed in detail above and in the following section on Methods of Use) are added to the reagent cell lysate for the production of ncAAs and/or for the production of proteins comprising the ncAAs.

### VIII. Methods of Use

a. Noncanonical Amino Acid (ncAA) and Recombinant Protein Synthesis

Also disclosed herein are methods for in cellulo synthesis of ncAAs and for the production of recombinant proteins comprising ncAAs using recombinant host cells of the present disclosure. In some embodiments, the ncAA is D-Cys-ε-Lys, D-Pra-ε-Lys, D-Allyl-ε-Lys, and/or 2ClAcK. In general, recombinant host cells of the present disclosure are cultured in the presence of substrates that are used by a PylC of the present disclosure for ncAA synthesis, and the recombinant host cell is cultured under conditions suitable for ncAA synthesis and protein overexpression. In some embodiments, recombinant host cells for synthesizing D-Cys-ε-Lys are cultured in media with D-Cys and L-Lys. In some embodiments, recombinant host cells for synthesizing D-Pra-ε-Lys are cultured in media with (R)-2-Aminopent-4-yonic acid and L-Lys. In some embodiments, recombinant host cells for synthesizing D-Allyl-ε-Lys are cultured in media with (D)-Allyl-OH and L-Lys. In some embodiments, recombinant host cells for synthesizing 2ClAcK are cultured in media with chloro-acetic acid and Lys. In some embodiments, the recombinant host cells are cultured in culture plates, test tubes, shake flasks, bioreactors, or fermenters. Bacterial, mammalian, and other expression systems and methods for their use are known in the art (see, e.g., Ausubel et al., 1988, Current Protocols in Molecular Biology, Wiley & Sons; Green and Sambrook, 2012, Molecular Cloning—A Laboratory Manual, 4th Ed., Cold Spring Harbor Laboratory Press, New York (2001); and Kaszubska et al., 2000, Protein Expression and Purification 18:213-20).

Also disclosed herein are methods for producing ncAAs (e.g., D-Cys-ε-Lys, D-Pra-ε-Lys, D-Allyl-ε-Lys, or 2ClAcK) and recombinant proteins comprising ncAAs in cell-free reactions using reagent cell lysate prepared from recombinant host cells of the present disclosure. In some embodiments, the reagent cell lysate comprises ncAAs that were previously produced in cellulo. In some embodiments, the reagent cell lysate is used to produce ncAAs. In some embodiments, the reagent cell lysate for synthesizing D-Cys-ε-Lys is supplemented with with D-Cys and L-Lys. In some embodiments, the reagent cell lysate for synthesizing D-Pra-ε-Lys is supplemented with (R)-2-Aminopent-4-yonic acid and L-Lys. In some embodiments, the reagent cell lysate for synthesizing D-Allyl-ε-Lys is supplemented with (D)-Allyl-OH and L-Lys. In some embodiments, the reagent cell lysate for synthesizing 2ClAcK is supplemented with chloro-acetic acid and Lys. Methods of preparing and using reagent cell lysates and cell-free expression systems are described in, e.g., Perez, Jessica G et al. Cold Spring Harbor Perspectives in Biology vol. 8, 12 a023853. 1 Dec. 2016; August et al. Life (Basel, Switzerland) vol. 11, 12 1367. 8 Dec. 2021; Smolskaya, Sviatlana et al. International Journal of Molecular Sciences vol. 21, 3 928. 31 Jan. 2020; Zawada, James F. Methods in Molecular Biology (Clifton, N.J.) vol. 805 (2012): 31-41; Jewett et al., Molecular Systems Biology, 4, 1-10 (2008); and Shin J. and Norieaux V., J. Biol. Eng., 4:8 (2010); Zawada et al., Biotechnol Bioeng, 108:1570-1578 (2011); Yin et al., mAbs, 4(2):217-225 (2012); and Zimmerman et al., Bioconjug Chem, 25(2):351-61 (2014).

The ncAA-containing proteins may be isolated from the recombinant host cells or the cell-free expression systems using a variety of methods that are available to one of ordinary skill in the art. Standard purification methods include electrophoretic, molecular, immunological, and chromatographic techniques, including ion exchange, hydrophobic, affinity, and reverse-phase HPLC chromatography. Ultrafiltration and diafiltration techniques, in conjunction with protein concentration, are also useful. See, e.g., Scopes, 1994, Protein Purification, 3rd edition, Springer-Verlag, New York City, New York.

Methods for determining the yield or purity of a purified proteins are known in the art and include, e.g., Bradford assay, UV spectroscopy, Biuret protein assay, Lowry protein assay, amido black protein assay, high pressure liquid chromatography (HPLC), mass spectrometry (MS), and gel electrophoretic methods (e.g., using a protein stain such as Coomassie Blue or colloidal silver stain).

b. Site-Specific Modification of Proteins

Incorporation of ncAAs (e.g., D-Cys-ε-Lys, D-Pra-ε-Lys, D-Allyl-ε-Lys, or 2ClAcK) into recombinant proteins as disclosed herein are useful for site-specific modification of proteins. Non-limiting examples of protein modifications using ncAAs include protein cyclization, oligomerization, and PEGylation. In some embodiments, proteins that comprise ncAAs and then modified using ncAAs demonstrate longer serum half-lives, increased resistance to proteolytic degradation, improved stability in serum, improved binding to a target, and/or improved efficacy.

An ncAA (e.g., D-Cys-ε-Lys, D-Pra-ε-Lys, D-Allyl-ε-Lys, or 2ClAcK) can be incorporated into a polypeptide sequence where a crosslink (or a staple) is desired between the ncAA and a second amino acid in the same polypeptide sequence. In some embodiments, when the crosslink is formed, either in cellulo or in an in vitro reaction as discussed in the Examples below, a covalent bond is formed. The crosslink is a covalent bond between a first moiety on the ncAA and a second moiety on the second amino acid. In some embodiments, the crosslink forms between two ncAAs, between an ncAA and a thiol group, or between an ncAA and a thioester group, for example a C-terminal thioester group. In some embodiments, the formation of the crosslink causes a polypeptide to cyclize.

In some embodiments, the crosslink is formed in cellulo and the recombinant protein is isolated as a crosslinked protein. In some embodiments where the crosslink formed in cellulo on the recombinant protein causes the recombinant protein to cyclize, the recombinant protein is isolated as a cyclized protein.

In some embodiments, the crosslink is formed in vitro, wherein the recombinant protein is first isolated as uncrosslinked protein, and one or more chemical reactions are later performed to produced crosslinked protein. In some embodiments, the crosslink formed in vitro on the isolated recombinant protein causes the isolated recombinant protein to cyclize.

In some embodiments, ncAAs (e.g., D-Cys-ε-Lys, D-Pra-ε-Lys, D-Allyl-ε-Lys, or 2ClAcK) can be used to form protein oligomers. In some embodiments, a first ncAA is incorporated into a first polypeptide sequence and a second ncAA is incorporated into a second polypeptide sequence. In some embodiments, the method comprises two or more polypeptides and each polypeptide contains one or more ncAAs. In some embodiments, the formation of the crosslink between two or more polypeptides causes the polypeptides to oligomerize. In some embodiments, the one or more ncAAs are different. In some embodiments, the one or more ncAAs are the same. In some embodiments, the two or more polypeptides are different. In some embodiments, the two or more polypeptides are the same. In some embodiments, the crosslinks between ncAAs, between ncAAs and thiol groups, or between ncAAs and thioester groups (for example C-terminal thioesters), are formed in cellulo and the recombinant proteins are isolated as crosslinked, oligomeric proteins. In some embodiments, the crosslinks are formed in vitro, where the recombinant proteins are first isolated as uncrosslinked proteins, and one or more chemical reactions are later performed to produced crosslinked, oligomeric proteins.

In some embodiments, the ncAA is D-Cys-ε-Lys and the crosslink is formed between the D-Cys moiety of D-Cys-ε-Lys and a C-terminal thioester in the same protein. In some embodiments, the protein comprises an intein and the C-terminal thioester is generated by incubating the protein with MESNA (sodium 2-mercaptoethane sulfonate) to catalyze intein cleavage. In these embodiments, the MESNA that becomes linked to the protein is replaced by the thiol of D-Cys-ε-Lys.

In some embodiments, the ncAA is D-Pra-ε-Lys, D-Allyl-ε-Lys, or 2-Chloro-Acetyl-ε-Lys, and the crosslink is formed between the ncAA and a cysteine thiol on another amino acid in the same protein.

In some embodiments, the ncAA is D-Allyl-ε-Lys and the crosslink is formed between two D-Allyl-ε-Lys using Grubbs catalyst.

In some embodiments, the ncAA is 2ClAcK and the crosslink is formed between the 2-chloro-acetyl group on 2ClAcK and a suitable active group, such as a cysteine or a small molecule containing a thiol.

Also disclosed herein are methods for adding polyethylene glycol (PEG) groups to recombinant proteins comprising ncAAs. This process is called site-specific PEGylation. The PEG moiety offers numerous advantages for increasing a protein's stability and circulating half-life. Due to its flexibility, hydrophilicity, variable size, and low toxicity, PEGylation is used for extending protein half-life. PEG has been approved by the Food and Drug Administration (FDA) as “generally recognized as safe” (Gaberc-Porekar, Vladka et al. Current Opinion in Drug Discovery & Development vol. 11, 2 (2008): 242-50). Protein PEGylation is discussed, e.g., in Dozier, Jonathan K, and Mark D Distefano. International Journal of Molecular Sciences vol. 16, 10 25831-64. 28 Oct. 2015; Liu, Yang et al. EJNMMI Research vol. 14, 1 15. 7 Feb. 2024; and Naowarojna, Nathchar et al. Synthetic and Systems Biotechnology vol. 6, 1 32-49. 15 Feb. 2021.

Most therapeutic proteins rely on the non-specific PEGylation of the protein through reactions with the amino groups on the side-chain of lysines and the N-terminus. While this method is convenient for the creation of PEGylated conjugates, it can lead to heterogeneous mixtures of PEGylated material with each PEG conjugate having its own activity and stability profiles. For instance, the PEGylation of INF-α2a creates eight different PEGylated proteins through eight different lysine residues. The activity level of these different PEGylated isomers have a three-fold range between the most active isomer and the least active (Foser, Stefan et al. Protein Expression and Purification vol. 30, 1 (2003): 78-87). Similar results have been observed for PEGylated forms of INF-α2b (Wang, Yu-Sen et al. Advanced Drug Delivery Reviews vol. 54, 4 (2002): 547-70), and a growth factor analogue (Veronese, Francesco M., ed. Springer Science & Business Media, 2009). In contrast, site-specific protein PEGylation using in cellulo synthesis of ncAAs disclosed herein and proteins that incorporate the ncAAs are useful in producing predictable and homogenous populations of protein with desired PEGylation.

In some embodiments, ncAAs (e.g., D-Cys-ε-Lys, D-Pra-ε-Lys, D-Allyl-ε-Lys, or 2ClAcK) can be used to inhibit proteins with thiols at a protein active site or near a protein active site. See, e.g., Mons, Elma et al. Journal of the American Chemical Society vol. 143, 17 (2021): 6423-6433; and Yu, Yongsheng et al. Molecular Pharmaceutics vol. 14, 5 (2017): 1548-1557.

In some embodiments, ncAAs (e.g., D-Cys-ε-Lys, D-Pra-ε-Lys, D-Allyl-ε-Lys, or 2ClAcK) can be used for engineering molecular scaffolds. See, e.g., Lu, Yao et al. Bioconjugate Chemistry vol. 25, 5 (2014): 989-99.

### I. Kits

Also disclosed herein are kits that comprise viable recombinant host cells, polynucleotides, PylCs, PylRSs, tRNAs, and/or reagent cell lysates of the present disclosure. The recombinant host cells may be provided in a variety of formats, e.g., without limitations, as a frozen stock, on an agar plate, in a liquid culture, and/or in a stab culture. The polynucleotides may be provided as one or more expression vectors. The PylCs, PylRSs, and tRNAs may be provided as purified proteins or tRNAs. The reagent cell lysates may be provided as frozen cell lysates prepared from recombinant host cells of the present disclosure. The kits may further comprise growth media, reagents, and/or instructions for use, including instructions for producing a protein of interest that comprises at least one ncAA in its polypeptide sequence.

### EXAMPLES

The Examples below demonstrate a scalable and robust system for the in cellulo synthesis of a noncanonical amino acids (ncAAs) coupled to the Pyl translational machinery for protein modification.

### I. Synthesis and Use of D-Cys-ε-Lys (Overview of Examples 1-7)

The in cellulo synthesis of the ncAA D-Cys-ε-Lys was achieved by hijacking the pyrrolysine (pyl) biosynthesis pathway. D-Cys-ε-Lys was genetically incorporated into proteins using an optimized pyrrolysyl-tRNA synthetase and cognate tRNA pair (PylRS/tRNA^{pyl}) and cell line (FIG. 1A, left panel). This system was then applied to cyclize a 23-mer therapeutic P16 peptide engrafted on a fusion protein, which resulted in near-complete cyclization of the target cyclic subunit in under three hours. The cyclic P16 peptide fusion protein product possessed higher CDK4 binding affinity than its linear counterpart. Further, a bifunctional bicyclic protein with the cyclic P16 peptide was produced and analyzed. The bifunctional bicyclic protein comprised a arginine-glycine-aspartate (RGD) motif for targeting cancer cells on one end, and a cyclic P16 peptide tumor suppressor on the other (FIG. 1A, right panel). This bifunctional bicyclic protein was found to be a potent cell cycle arrestor with improved serum stability.

### Example 1—Materials and Methods

Plasmids construction for directed evolution of PylRS. The plasmid pPylST-KanR(TAG)-mCh(TAG) used for the directed evolution of PylRS was derived from pPylST-mCh(TAG), which is a pETDuetbased plasmid harboring pylS gene encoding PylRS (flanked by NcoI and BamHI sites) and pylT gene encoding tRNA^{pyl} (flanked by XbaI site) from Methanosarcina mazei, as well as an mCherry gene with lysine codon at the 55th position mutated to TAG cloned between NdeI and KpnI sites.⁴⁵⁻⁴⁶ To produce the pPylST-KanR(TAG)-mCh(TAG) plasmid, a kanamycin resistance gene encoding aminoglycoside kinase containing a K42O mutation (i.e., KanR(TAG)) was cloned upstream of the mCh(TAG) gene together with an intergenic spacer region containing an additional ribosome binding site (FIG. 1B, middle portion). A modified plasmid containing tRNA^{M15} was generated by substituting the bases as indicated in the study of Serfling et al.²⁴ in the wild-type pylT gene by performing two rounds of site-directed mutagenesis. The primers used were PylTm15_Fwd (SEQ ID NO: 148) and PylTm15_Rev (SEQ ID NO: 149) for the 1st round of mutagenesis, and PylTstarSecondC_Fwd (SEQ ID NO: 150) and PylTstarSecondC_Rev (SEQ ID NO: 151) for the 2nd round of mutagenesis.

PylRS library construction. To generate the PylRS mutant library, random mutations were introduced into the gene encoding PylRS and its variants by error-prone PCR (epPCR) using either the GeneMorph II Random Mutagenesis Kit according to the manufacturer's protocol, or Taq DNA polymerase with error-prone conditions referencing a previously reported protocol to achieve a mutation frequency of ~2.5 nucleotide mutations per amplicon.⁴⁷ The primers used were PylS Fwd and R4 which flanked the pylS gene. A PylRS mutant containing G14E and S451F mutations was subsequently identified. Since C348 has been reported to be critical for substrate binding, site-saturation mutagenesis (SSM) was performed to randomize the C348 residue of the identified PylRS mutant using primers F2-PylS-Cys348 (with a degenerate NNN codon at C348) and R4. In each round of directed evolution, the gel-purified epPCR or SSM products were subcloned into the pPylSTKanR(TAG)-mCh(TAG) via megaprimer PCR of whole plasmids (MEGAWHOP) with reference to Miyazaki's protocol.⁴⁸ The resultant PCR products were treated with DpnI, followed by transformation into E. coli BL21 (DE3) cells for library screening.

PylRS library screening. E. coli BL21 (DE3) cells were transformed with the mutant library via heat shock or electroporation and allowed to recover by incubating at 37° C. with shaking for 1 h. The transformed cells were spread on selection plates containing LB agar supplemented with 50 μM IPTG, 100 μg/mL ampicillin, 2 mM D-Cys-ε-Lys and 50 to 100 μg/mL kanamycin, and then incubated at 37° C. overnight.

Positive clones from the kanamycin selection were re-streaked onto LB agar containing ampicillin followed by incubation at 37° C. overnight. Fresh clones were then inoculated into wells containing 150 μL of LB medium supplemented with 100 μg/mL ampicillin and grew at 37° C. with shaking at 230 rpm for 5 to 6 h. These cultures were then used in the 2nd screening based on mCherry fluorescence. Towards this end, 5 μL of each culture was added to a well of a 24-well plate containing 495 μL of LB medium or M9 glucose minimal medium (6 g/L Na2HPO4, 3 g/L KH2PO4, 1 g/L NH4Cl, 0.5 g/L NaCl, 3 mg/L CaCl2, 0.4% glucose and 1 mM MgSO4 in MilliQ water) with supplementation of 50 μM IPTG, ampicillin and 2 mM D-Cys-ε-Lys, followed by incubation at 30° C. overnight. 150 μL of each of the induced cultures was transferred to a black 96-well plate with clear bottom for measurements in a microplate reader (Tecan Infinite M1000 PRO). The normalized fluorescence intensity [measured fluorescence (Ex: 587±5 nm; Em: 610±5 nm) divided by the optical density at 700 nm] of each sample was used for selection. Cells that exhibited mCherry fluorescence stronger than the parent PylRS's were selected and cultured in 5 mL LB supplemented with ampicillin overnight for plasmid DNA extraction.

To validate that the selected PylRS mutants were true positives (i.e., do not use any endogenous amino acid as substrate and exhibit higher catalytic efficiency than the parent), the mCherry fluorescence screening was repeated in triplicate using freshly transformed colonies grown in LB medium supplemented with or without D-Cys-ε-Lys. A no ncAA control was included as a negative control.

Construction of pPylST.tL plasmid. To optimize the yield of the ncAA-containing protein, the genetically recoded E. coli strain C321.ΔA.M9adapted that has been engineered to enhance nonstandard amino acid incorporation²⁹ for protein overexpression was used. This E. coli strain, however, is incompatible with the aforementioned pETDuet-derived pPylST vectors for two reasons. Firstly, the strain is ampicillin-resistant, so ampicillin cannot be used as the selection marker for the mutant library transformants, and secondly, it does not produce T7 RNA polymerase that recognizes the T7 promoter in pETDuet-based plasmids, and which is essential for transcription. Thus, in order to utilize the C321.ΔA.M9adapted cells, several modifications were made to the pETDuet-based pPylST-mCh(TAG) vector bearing the tRNA^{M15} and evolved PylRS^{EVF} obtained from the directed evolution studies. In brief, these modifications involved (1) replacing the T7lac promoter located upstream of the 1st multiple cloning site (MCS1) (harboring tRNA^{M15} and PylRS^{EVF} genes) with the Ptac promoter; (2) substituting the T7lac promoter located upstream of the 2nd multiple cloning site (MCS2) (harboring a gene carrying an in-frame TAG codon) with the PLlacO1 promoter; and (3) exchanging the ampicillin resistance gene (amR) with a streptomycin/spectinomycin resistance gene (smR). Promoter sequences are as shown in SEQ ID NO: 152-155. The primers used to amplify the gene fragments for the aforementioned modifications are as shown in SEQ ID NO: 156-182. The amplicons were assembled using Gibson Assembly to build the resultant pPylST.tL-mCh(TAG) plasmid (FIG. 1B, bottom portion).

mCherry readthrough assay. For evaluating the efficiency of different readthrough systems, E. coli competent cells (Rosetta 2 (DE3) or C321.ΔA.M9adapted) cells were transformed with the relevant pPylST-mCh(TAG) or pPylST.tL-mCh(TAG) plasmid. The transformed cells were then inoculated into 50 mL of M9 medium containing appropriate antibiotics (ampicillin and/or spectinomycin) in a 96-well microtiter plate. After incubation at 30° C. with shaking for 5 to 6 h, 250 μL of each culture was added into a 48-well plate. The medium was supplemented with the appropriate antibiotic, 0.5 mM IPTG and different concentration of D-Cys-ε-Lys. The plate was then incubated at 25° C. overnight with shaking. The mCherry fluorescence intensities of the induced cultures were measured as described above. The assay was performed in triplicate.

Generation of PylC mutant library by site-saturation mutagenesis. Site-saturation mutagenesis was performed on the residues S177, E179, D233 and T256 of PylC fused to an N-terminal SUMO tag to facilitate purification using two sets of degenerate primers (primers: PylC-S177E179mut_Fwd, PylC-D233mut_Rev, PylC-D233mut_Fwd and PylC-T256mut_Rev). The SUMO-PylC full-length fragment was extended by overlap extension PCR using another two sets of non-mutated primers (primer: rbs-NdeI-SUMO_Fwd, PylC-S177up_Rev, PylC-T256down_Fwd, and PylC-KpnI-pDuet_Rev). The extended SUMO-PylC mutant insert was cloned into pACYCDuet-1 vector whose promotor was replaced with Plpp to enable constitutive expression of PylC (primer: Plpp-rbs_Fwd). The reaction product yielded a PylC mutant library that could be screened in PylST expressing cells.

Screening of PylC mutant library. For screening of the PylC mutant library, BL21 (DE3) Star (Thermo Fisher) cells were transformed with plasmid pPylST.tL-KanR(TAG)-mCh(TAG). A single colony of the transformed cells was selected for the preparation of pPylST.tLKanR(TAG)-mCh(TAG)-containing competent cells used for the subsequent transformation with the PylC mutant library by electroporation. The mutant library-transformed cells were plated on LB agar plates containing spectinomycin, chloramphenicol, 50 μM IPTG, 5 mM D-cysteine as well as different concentrations of kanamycin (150 and 200 μg/mL). Each transformation yielded approximately 68,300 transformants.

Colonies grew on the positive selection plates were transferred into 96-well microtiter plates containing 150 μL LB medium supplemented with chloramphenicol, spectinomycin, 50 μM IPTG and 5 mM D-cysteine for quantitation of the readthrough efficiency based on mCherry fluorescence as described above. The top 50 mutants that produced mCherry fluorescence exceeding that produced from wild-type PylC were selected for plasmid extraction for subsequent DNA sequencing for the identification of the corresponding mutation(s).

Computational modeling. Modeling of PylRS C348V was based on the crystal structure of MmPylRS C-terminal domain (CTD) bound to adenylated Pyl (PDB: 2ZIM).25 The C348 residue was mutated to valine and the ligand was modified to adenylated D-Cys-ε-Lys in PyMOL.⁴⁹ Then the minimization of protein structure and analysis of surface hydrophobicity were performed using UCSF CHIMERA.²⁸

The MmPylRS and tRNA^{pyl} complex model was manually built in PyMOL from two deposited structures: MmPylRS NTD-tRNA^{pyl} complex (PDB: 5UD5)50 and MmPylRS CTD bound with adenylated Pyl (PDB: 2ZIM), using the D. hafniense PylRS CTD-tRNA^{pyl} complex structure (PDB: 2ZNI)51 as reference for alignment.

To generate the model of MmPylC bound to D-Cys-ε-Lys, model MmPylC bound to D-ornithine-ε-Lys was first build in the SWISSMODEL52 server taking the crystal structure of the M. barkeri PylCWT bound to D-ornithine-ε-Lys (PDB ID: 4FFM)31 as a template. Then the D-ornithine-ε-Lys was replaced with D-Cys-ε-Lys in Pymol and the mutations (S177N, E179P, D233S, T256V) were incorporated into PylC chain. The final structure was refined by simulated annealing/molecular dynamics program from the CNS package.32-33 Binding and surface hydrophobicity analyses were performed using UCSF CHIMERA.

Molecular dynamics (MD) simulation of P16p. MD simulation of P16p was conducted using GROMACS software53 version 2021.4 with opls2001 force field and TIP3P water model. The topology file of D-Cys-ε-Lys was generated using LigParGen server54-56 in GROMACS format. The initial peptide was solvated by a dodecahedral water box with approximately 4000 water molecules and neutralized by adding Cl− ions. The solvated system was minimized by steepest descent method using a tolerance of 1000 KJ/mol·nm and step size of 0.01 nm. The system was gradually heated from 0 to 298 K over 100 ps at the pressure of 1 bar. The production runs were carried out for 300 ns with a step size of 2 fs. The temperature was kept at 298 K by modified Berendsen thermostat with a time constant of 1 ps. The pressure was kept at 1 bar by Parrinello-Rahman scheme with a time constant of 2 ps and an isothermal compressibility of 4.5×10-5 bar-1. Particle mesh Ewald (PME) method was employed to calculate long-range electrostatic interactions and a cut-off distance of 1 nm was used to calculate the short-range electrostatic and van der Waals interactions. The LINCS algorithm was employed to constrain all covalent bonds involving hydrogen atoms. Independent 300 ns simulations of both peptides were run 3 repeats from the same initial structure. Another 10 rounds of 10-ns simulation were run in the same condition for structure comparison. RMSD and RMSF analyses were performed using algorithms in GROMACS with least squares fit calculated based on backbone atoms.

Plasmid construction of proteins targeted for D-Cys-ε-Lys incorporation. The optimized plasmid pPylST.tL was used for subcloning different protein constructs for the subsequent cyclization studies. In brief, gene fragments encoding the protein of interest harboring the UAG codon and intein-CBD-His7 tag were inserted in between KpnI and NdeI sites by Gibson Assembly (NEB). For the construct cycRGDmCh-cycP16p, an N-terminal SUMO (Small ubiquitin-like modifier protein) tag was included upstream to enhance protein expression. The linear counterparts of the D-Cys-ε-Lys incorporated proteins under study were subcloned similarly except the UAG codon in the gene fragment for cyclized proteins was replaced with GCG codon that encodes alanine by mutagenesis using Pfu Turbo DNA polymerase (Agilent Technologies) following manufacturer's instruction and the primers Oto-Ala-mutant_Fwd and O-to-Ala-mutant_Rev.

Protein expression, purification and cyclization of D-Cys-ε-Lys-containing proteins. For the expression of protein using chemically synthesized D-Cys-ε-Lys, the pPylST.tL plasmids harboring the protein constructs for cyclization (FIG. 6) were transformed into E. coli C321 strain. The resultant transformants were inoculated into LB media supplemented with spectinomycin and grew at 30° C./220 rpm until OD600 reached 0.6. The cells were centrifuged to get rid of the culture media and the resultant cell pellets were resuspended in one-seventh of the original culture volume of 2×YT media supplemented with spectinomycin, induced with 0.5 mM IPTG and 4 mM D-Cys-ε-Lys for 16 h at 25° C./220 rpm.

For the expression of protein using in cellulo or in vivo-synthesized D-Cys-ε-Lys, the pACYCDuet-SUMO-PyC^{NPSV} plasmid was co-transformed with the aforementioned pPylST.tL plasmids into E. coli C321 cells and cultured in 200 mL LB medium supplied with spectinomycin, chloramphenicol and 5 mM D-cysteine (pH adjusted to 8.0 with 5 M Tris) at 30° C. When OD600 reached 0.6, the cell culture was centrifuged and resuspended in 50 mL LB medium, induced with 0.5 mM IPTG for 16 h at 25° C./220 rpm.

At the end of the induction period, cells were harvested by centrifugation and resuspended in lysis buffer (20 mM Tris-HCl pH 8.0, 500 mM NaCl, 1 mM PMSF, 1 mM benzamidine). Cells were lysed by sonication on ice for 20 min and purified by Ni2+ affinity chromatography, except for SUMO-cycRGD-O-P16p-intein-CBD-His7, which was purified using chitin resin to facilitate on-column cleavage by SUMO protease to remove the N-terminus SUMO tag and subsequent on-column cyclization as described below. Prior to cyclization, the purity of the purified protein was verified by SDS-PAGE.

Cyclization of the purified protein was achieved by the addition of 100 mM sodium 2-sulfanylethanesulfonate (MESNA) to initiate intein cleavage and 2 mM tris(2-carboxyethyl)phosphine (TCEP, pH adjusted to 8.0) to maintain a reducing environment. The cyclization process was performed in room temperature for 3 h, after which, the reaction mixture was incubated with chitin resin (New England Biolabs) to remove the cleaved intein-CBD-His7, and the cyclized protein was collected from the mobile phase. An additional cyclization step to cyclize the N-terminal RGD of the cycRGD-O-P16p construct was performed in which the protein was subjected to air oxidation at 4° C. with gently shaking for 24 h.

The expression of the linear counterpart of the cyclized proteins was performed in transformed E. coli R2 strain cultured in LB media supplemented with 100 μg/mL ampicillin at 37° C./220 rpm until OD600 reached 0.6, at which point, 0.5 mM IPTG was used to induce expression for 16 h at 25° C./220 rpm. The same purification procedure described above for the corresponding cyclized proteins was used to purify the linear counterparts. To remove the intein-CBD-His7, 50 mM DTT was added to the reaction mixture after which the intein-CBDHis7 was similarly removed by chitin purification as previously described.

Electrospray ionization mass spectrometry. The protein band corresponding to GFP-O-P16p was excised, cut into 1 mm3 pieces and destained by repeated wash steps using 50% MeOH/10 mM NH4HCO3, then dehydrated with ACN followed by vacuum drying. For protein reduction, 25 mM DTT was added and incubated at 56° C. for 1 h, followed by washing steps to remove remaining DTT before dehydration again. Trypsin was added to the dehydrated gel pieces and incubated at 37° C. overnight for digestion. Digested peptide was extracted from gel by sonication and the extracted samples were then separated by HPLC and analyzed on an Orbitrap Fusion Lumos Tribrid Mass Spectrometer (Thermo Fisher Scientific). For mass spectrometric analysis of intact protein, purified GFP-cycP16p was incubated with 25 mM DTT for 24 h, followed by desalting using Bio-Gel P-30 size exclusion resin and denaturation by 0.1% formic acid before subjected to HPLC-MS analysis on an Orbitrap Fusion Lumos Tribrid Mass Spectrometer (Thermo Fisher Scientific). The capillary voltage was set to 3500 V. Spectra were acquired at a resolution of 120000 between 500-2000 m/z. Mass spectra were analyzed and deconvoluted by BioPharma Finder (Thermo Fisher Scientific) using Xtract algorithm.

Analytical size exclusion chromatography. To evaluate whether the cyclized P16p subunit on GFP-cycP16p will bind with CDK4, size analysis was performed on a mixture of GST-CDK4 (0.32 mg/mL, Sino Biological) and GFP-cycP16p (0.13 mg/mL) in 25 μL SEC running buffer (20 mM Tris pH 8.0, 200 mM NaCl) using Superdex 200 increase 5/150 GL analytical size exclusion column (Sigma-Aldrich) at a flow rate at 0.35 mL/min. Individual GST-CDK4 and GFP-cycP16p proteins were analyzed using the same condition as reference.

Binding studies. MicroScale Thermophoresis (MST) analysis was performed to measure the biomolecular interactions between GSTCDK4 (Sino Biological) and linear or cyclic MBP-P16p. The targets MBP-cycP16p or MBP-P16p were dialyzed against labeling buffer (20 mM Tris, 200 mM NaCl, pH 8.0) prior to labeling with Alexa Fluor 647 NHS ester dye (Thermo Fisher Scientific) at a molar ratio 1:10 (protein:dye) at RT for 2 h. Free dyes were removed using the gravity flow column B provided in the Monolith protein labeling Kit (Nanotemper). 16 sets of 1:1 serial dilution of the binding ligand GST-CDK4 was prepared with MST optimized buffer (20 mM Tris, 200 mM NaCl, 0.05% Tween 20, pH 8.0) while the concentration of the labeled targets was kept constant at 70 nM. The highest concentrations of GST-CDK4 used were 1.92 μM and 3.84 μM for MBP-cycP16p and MBP-P16p, respectively. Samples were mixed by pipetting and loaded into Monolith standard capillaries (NanoTemper) and their binding affinities were measured using the Monolith NT.115 with Nano-RED excitation type and the MST power set to medium. The collected data were processed using NanoTemper Affinity analysis software for the determination of the dissociation constant (Kd).

MCF-7 cell lysate pull down. MCF-7 cells were seeded at 4.0×10⁵ cells per well in 6-well plates and incubated in RPMI-1640 medium supplemented with 10% FBS and penicillin/streptomycin (P/S) for 20 h. Cells were washed twice with ice-cold PBS and lysed in NP-40 lysis buffer (50 mM Tris, 150 mM NaCl, 2 mM EDTA, 1% NP-40, 0.1% SDS, pH 7.5) supplemented with protease inhibitor cocktail (Roche), followed by incubation in low temperature with gentle shaking for 20 min. Cell lysate was clarified by centrifugation at 15000 rpm for 15 min at 4° C. The total protein concentration of the supernatant was measured using Pierce™ BCA protein assay kit (Thermo Fisher Scientific) and was diluted to 1.5 mg/mL using PBS. This bait protein solution containing CDK4/6 was then incubated with purified MBPcycP16p immobilized on amylose resin at 4° C. for 3 h with gentle mixing. The reaction mixture was washed 5 times with PBS, after which the resin was analyzed by western blotting using anti-CDK4 (1:500, Biolegend) following standard protocol. The same bait protein solution was incubated with amylose resin only as a negative control.

Cellular uptake assay. MCF-7 cells in RPMI-1640 medium supplemented with 10% FBS and P/S (Cytiva) were seeded at 2.0×10⁵ cells/mL in 35 mm glass bottom confocal dishes (MatTek) and incubated at 37° C./5% CO2 overnight for attachment. Next day the cells were washed with PBS to get rid of non-adherent cells and the adhered cells were starved for 24 h in serum-free RPMI-1640 medium to induce cell cycle synchronization. Cells were then treated with 15 μM cycRGD-mCh-P16p or cycRGD-mCh-cycP16p for 24 h in RPMI-1640 medium supplemented with 10% FBS and P/S. Prior to confocal imaging, the nuclei and plasma membrane of the cells were counterstained with Hoechst 33342 (Thermo Fisher) and wheatgerm agglutinin (Thermo Fisher) respectively. Images were captured using a TCS SP8 Confocal Microscope (Leica) with excitation wavelengths set at 488, 561 and 633 nm.

Cell cycle arrest assay. MCF-7 cells were seeded in 12-well plates (1.5×10⁵ cell per well) and incubated in RPMI-1640 medium supplemented with 10% FBS and P/S at 37° C. After 24-h attachment, cells were starved for another 24 h by replacing the medium with serum-free RMPI-1640 medium. At the end of starvation period, the medium was changed back to RMPI-1640 supplemented with 10% FBS. Cells were then treated with 15 μM R9-P16p, 15 μM cycRGD-mCh-P16p or 15 μM cycRGD-mCh-cycP16p, PBS (negative control), and 10 nM actinomycin (positive control) for different lengths of time (24, 48, 72 h). At the end of treatment period, cells were washed twice with PBS, trypsinized and fixed by 70% ethanol on ice for 45 min. The fixed cells were washed with PBS and stained by PI staining solution (10 μg/mL PI in PBS supplemented with RNase) for 30 min at RT in dark, followed by flow cytometric analysis using FACSVerse Flow Cytometer (BD Biosciences). Flow data was analyzed using ModFit LT 5.0 (BD Biosciences).

Cell proliferation assay. MCF-7 in RPMI-1640 medium supplemented with 10% FBS and P/S were seeded at 1.6×10⁴ cells/well in a 96-well plate to allow for overnight attachment. Cells were then washed with PBS and treated with 5-20 μM cycRGD-mCh-P16p, cycRGD-mCh-cycP16p, or R9-P16p (Pepmic) for 24 h. The number of viable cells was determined using the CellTiter 96 cell proliferation assay kit (Promega) following manufacturer's instruction and scanned on a Tecan Spark 10M Microplate Reader with the absorbance wavelength set at 490 nm. Data were normalized to the value of control cells treated with PBS. Experiment was repeated 3 times with similar results.

Western blot analysis. MCF7 cells were seeded at 4.0×10⁵ cells per well in 6-well plates in RPMI-1640 medium supplemented with 10% FBS and P/S for overnight attachment. The medium was then changed to serum-free RPM-1640 medium and the cells were starved for 24 h. At the end of starvation, serum-free medium was replaced with RPMI-1640 supplemented with 10% FBS and P/S. 15 μM of cycRGD-mCh-P16p or cycRGD-mCh-cycP16p was added to the cells and incubated at 37° C./5% CO2 for 24 h. At the end of the treatment, cells were washed with ice-cold PBS and lysed in NP-40 lysis buffer (50 mM Tris, 150 mM NaCl, 2 mM EDTA, 1% NP-40, 0.10% SDS, pH 7.5) supplemented with protease inhibitor cocktail (Roche). Cell debris were removed by centrifugation at 15000 rpm for 10 minutes at 4° C. Samples were resolved by 10% SDS-PAGE, transferred to nitrocellulose membranes and probed using pRb (Ser780) antibody (1:1000, Cell Signaling), pRb (Ser795) antibody (1:500, Cell Signaling) and β-actin antibody (1:2500, Sigma-Aldrich). Proteins were visualized using the ECL system (Amersham).

Statistical analysis. Data from replicate experiments are presented as mean±standard error (SD) of the mean. Statistical significance is noted in the figure legend where appropriate. For comparison of data in different groups, ordinary one-way ANOVA with Tukey's multiple comparison test was performed using GraphPad Prism 7 software (GraphPad, San Diego, USA). A p-value less than 0.05 is considered statistically significant. *p<0.05, ***p<0.001, ****p<0.0001, ns, not significant.

### Example 2—Optimization of D-Cys-ε-Lys Readthrough System

This Example relates to optimizing the UAG readthrough system for enhancing D-Cys-ε-Lys incorporation into proteins. D-Cys-ε-Lys is not the natural substrate for wild-type pyrrolysyl-tRNA synthetase (PylRS). Improvements in substrate specificity or activity of PylRS for D-Cys-ε-Lys might enhance PylRS catalytic efficiency, thereby leading to more efficient D-Cys-ε-Lys incorporation. To this end, directed evolution was employed to evolve a PylRS mutant that could specifically recognize D-Cys-ε-Lys and display improved efficiency in its genetic incorporation.

To generate the PylRS mutant library, a plasmid tRNA^{M15} was constructed to harbor the genes encoding the wild-type PylRS. tRNA^{M15} was a more stable variant of tRNA^{pyl} with demonstrated improved amber suppression efficiency,²³⁻²⁴ a kanamycin resistance protein aphA-3 coding sequence, and an mCherry protein coding sequence. AphA-3 and mCherry were each engineered with a permissive UAG codon in its coding sequence to facilitate a 2-tier selection and screening protocol for the rapid detection of active PylRS mutants and their corresponding D-Cys-ε-Lys incorporation activity. First, the mutants were subjected to positive selection under kanamycin selection pressure contingent on D-Cys-ε-Lys incorporation into the aphA-3 kanamycin resistance polypeptide. Then, the selected clones were screened for their mCherry fluorescence enabled by the readthrough of the UAG codon in the mCherry transcripts. After iterative mutagenesis and screening, a triple mutant PylRS carrying G14E, C348V and S451F mutations (PylRS^{EVF}) was identified—PylRS exhibited improved readthrough efficiency was identified (FIG. 1D, left panel).

Computational modeling was then performed to ascertain how these three mutations might impact substrate binding. Of the three mutated residues, C348 has been reported to be critical for the binding of substrates.²⁵ Previous studies on the structure of MmPylRS revealed that C348 together with Y306, Y384, V401 and W417 form a deep hydrophobic pocket and is among the key residues involved in the recognition and binding of Pyl and its analogs.²⁶⁻²⁷ Presumably, the substitution of the polar cysteine with the more hydrophobic valine enhances the hydrophobicity of the binding pocket as verified by CHIMERA,²⁸ thereby improving its affinity for D-Cys-ε-Lys. The other two mutated residues, G14 and S451, are located in the N-terminal tRNA binding domain and C-terminal tRNA minimal core binding surface of PylRS, respectively. Subsequent optimization of other parameters, including the replacement of T7 promoter with T7lac promoter resulted in a further yield improvement, with a net ~20-fold increase in mCherry fluorescence compared to protein expression and readthrough using WT PylRS and tRNA^{pyl}.

To further improve the yield of proteins containing D-Cys-ε-Lys, the E. coli strain (C321.ΔA.M9adapted)²⁹ was used. In this strain, the release factor 1 was deleted. Use of this strain brought a further 5.3-fold improvement based on the mCherry fluorescence (FIG. 1E). As shown in FIG. 1E, the improved strain produced higher levels of full-length proteins of different sizes. These results are consistent with enhanced D-Cys-ε-Lys incorporation and UAG readthrough efficiency.

### Example 3—In Cellulo Synthesis of D-Cys-ε-Lys

Most current studies on the use of noncanonical amino acids (ncAAs) to produce ncAA-containing proteins rely on the exogenous supplementation of ncAAs chemically synthesized with the functionalities of interest. However, this approach is cost ineffective and not feasible for large-scale production of ncAAs-containing proteins. A more economical and practical strategy would be to produce the ncAAs of interest from basic carbon sources or amino acids (which can be purchased cheaply) in a workhorse such as E. coli, which are then directly incorporated into proteins in response to the UAG codon.

In this regard, the elucidated Pyl biosynthesis pathway³⁰ offers a blueprint for the establishment of an endogenous biosynthesis machinery of ncAAs, e.g., D-Cys-ε-Lys. Pyl is generated by the PylB, PylC, and PylD enzymes. In the first step, lysine is converted by PylB to (2R,3R)-3-methyl-ornithine. In the second step, (2R,3R)-3-methyl-ornithine is coupled with another lysine by PylC to form L-lysine-Nε-3R-methyl-D-ornithine (Lys-Nε-3MO). Lys-Nε-3MO is a precursor of pyl. In the third step, PylD converts the terminal ornithyl amine to a carbonyl, which then reacts with an amine to form the pyrroline group.

Previous studies have shown that Pyl could be synthesized in the absence of PylB.³⁰ Based on this, PylC was modified to use other amino acids as substrates, such as D-cysteine (D-Cys), to L-lysine to form D-Cys-ε-Lys (FIG. 2A). The complex structure of Methanosarcina barkeri PylC with D-ornithine was previously determined,³¹ and showed that four active site residues, S177, E179, D233, and T256; were important for D-ornithine binding. Thus, to evolve a PylC mutant that could take D-Cys as the substrate, site-saturation mutagenesis was performed on S177, E179, and D233, T256 create a mutant library. PylC variants identified were as shown in Table 1 above. The PylRS^{EVF}/tRNA^{M15} optimized strain from Example 2 above was transformed with this mutant library. A mutant PylC^{NPSV} comprising S177N, E179P, D233S, and T256V mutations was identified by kanamycin selection and mCherry fluorescence screening (FIG. 2B).

Computational models were applied to analyze the basis of switch in substrate specificity of MmPylC^{WT} and MmPylC^{NPSV} bound to D-Cys-ε-Lys were generated using the program CNS.³²⁻³³ Based on these models, it appears that mutations of E179P and T256V convert the original hydrophilic D-ornithine binding site to a hydrophobic pocket that makes binding of the charged amino group of the D-ornithine side chain less favorable. On the other hand, mutations T256V and S177N in PylC^{NPSV} result in Van der Waals interactions with the thiol group of D-Cys-ε-Lys based on analysis using CHIMERA. It is likely that this combination of weaker binding of D-ornithine-ε-Lys and improved binding of the D-Cys-ε-Lys leads to the observed switch in substrate specificity.

To evaluate the readthrough efficiency using in cellulo biosynthesized D-Cys-ε-Lys as opposed to exogenous addition of its chemically synthesized counterpart, different protein constructs were tested. The results indicated that at 5 mM or higher concentrations of D-Cys, PylC^{NPSV} in the optimized PylRS^{EVF}/tRNA^{M15} strain could achieve a readthrough efficiency comparable to that of the exogenous addition of 4 mM chemically synthesized D-Cys-ε-Lys (FIG. 2C). The successful in cellulo biosynthesis of D-Cys-ε-Lys was subsequently confirmed via the identification of the D-Cys-ε-Lys-containing peptide fragment by LC-MS/MS analysis of the corresponding trypsin-digested protein.

### Example 4—Structure-Inspired Cyclization of P16p

This Example relates to developing a method of protein cyclization using D-Cys-ε-Lys. Previously, slow and incomplete cyclization of an RGD motif was observed when the RGD motif placed on the C-terminus of an mCherry fusion protein.¹⁰ At that time, it was speculated that the low yield was due to the multiple degrees of freedom of the reacting groups. Further supporting this observation was the insights from other studies that the relative spatial positions of the N- and C-termini of the peptide to be cyclized greatly affect cyclization rate and yields.³⁴⁻³⁶ Thus, it was hypothesized that utilizing structured fragments where the D-Cys-ε-Lys and C-terminal thioester are better spatially localized for cyclization might lead to enhanced cyclization efficiencies.

The tumor suppressor P16 protein was chosen for cyclizing with D-Cys-ε-Lys because it possesses a pre-formed cyclic motif within its native sequence; the portion of P16 that binds to CDK4/6 forms a contains a helix-turn-helix structure.¹⁹ P16 functions as a CDK4/6 inhibitor by preventing phosphorylation of retinoblastoma (Rb) resulting in G0/G1 cell cycle arrest. The P16 binding sequence to CDK4/6 is a 20-mer peptide encompassing residues 84-103 (P16p).¹⁸ This Example relates to determining whether cyclization of the P16p enhances its binding affinity and cellular stability, thereby increasing its therapeutic potential.

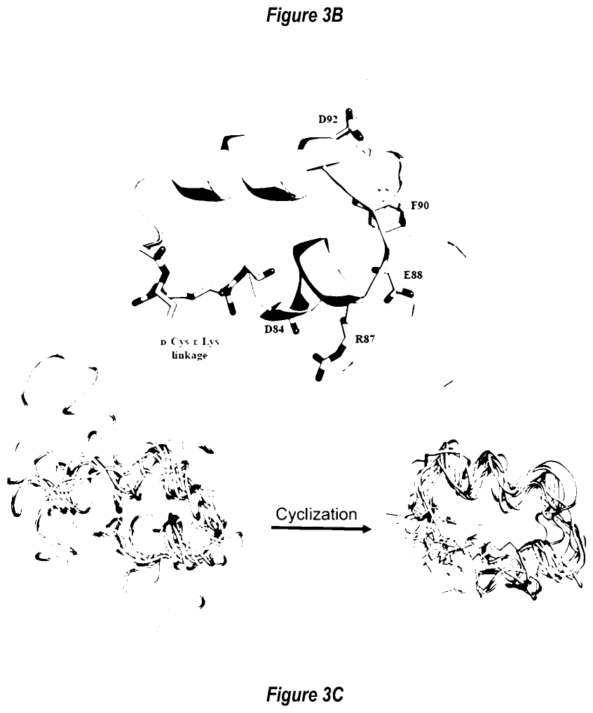

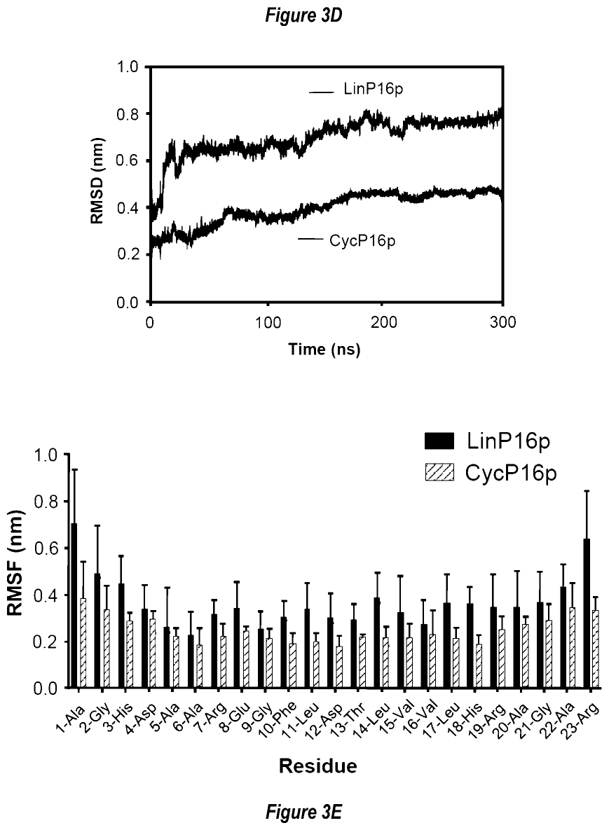

The P16/CDK6 protein complex structure was used to guide the designing of the D-Cys-ε-Lys incorporation site. Molecular simulations were performed to verify the design strategy. Based on structural analysis, the site of incorporation was chosen at the N-terminus of P16p, which was extended by one more residue to include the 83rd residue histidine. In addition, an N-terminal glycine was added to enable D-Cys-ε-Lys to be spatially close to the C-terminal thioester generated during cyclization (FIGS. 3A-3B).

To obtain visual insight of the designed cyclic P16 peptide, the corresponding molecular model was built using Pymol, which showed that the D-Cys-ε-Lys-linkage occurred on the opposite side of the P16-CDK4/6 interface (FIG. 3B). Molecular dynamics simulations were then performed on this peptide as well as its linear counterpart to evaluate their structural stability.

In simulation, linear P16p (LinP16p) displayed significant higher structural flexibility, in particular at the ends of the two helices, while the cyclic P16p (cycP16p) exhibited a much more stable conformation with minimal changes from the reference structure (FIG. 3C). This observation was corroborated by their respective RMSD and RMSF profiles which depicted much larger amplitude fluctuations for the linear P16p compared with those of the cycP16p (FIGS. 3D-3E). These results appeared to support the notion that cyclization would benefit P16p, at least in terms of enhancing its stability given the reduction in configurational entropy.

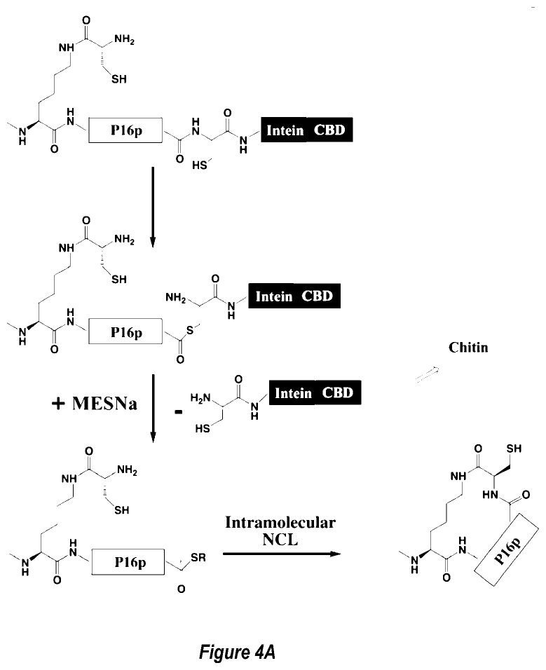

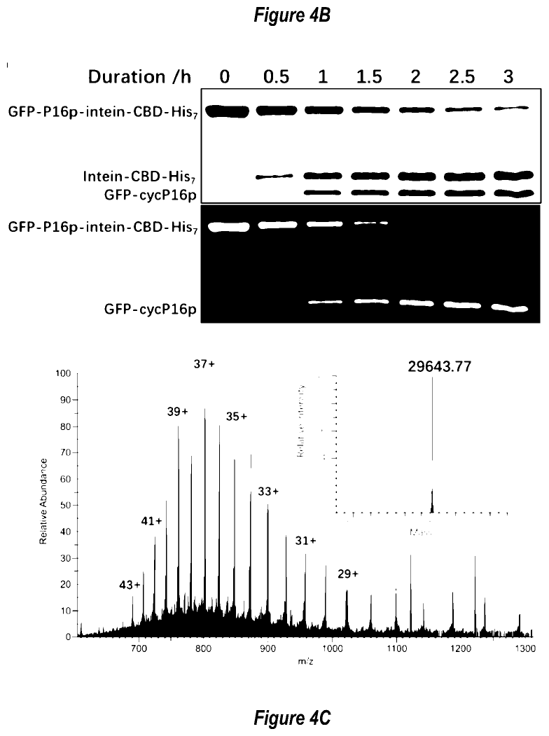

To validate these results experimentally, the aforementioned P16p design was incorporated into a fusion protein comprising a green fluorescent protein (GFP), the P16p, an intein for thioester generation, and a CBD-His7 tag for affinity purification (GFP-O-P16p-intein-CBD-His7) (FIG. 4A). Utilizing the optimized PylR^{SEVF}/tRNA^{M15} pair and the engineered PylC^{NPSV} (i.e., the PylC mutant strain), ~19 mg of purified D-Cys-ε-Lys-containing GFP-O-P16p-intein-CBD-His7 protein was obtained from a 200 mL cell culture grown in medium supplemented with 5 mM D-Cys. This product yield represented a 10-fold improvement over the unoptimized system. Cyclization of the fusion protein was initiated by the addition of MESNA. Cyclization was completed within 3 h (FIG. 4B), as indicated by the GFP signal levelling off after the 2.5-h time point. The resultant cyclization product was subsequently confirmed by mass spectrometric (MS) analysis to be the cyclized form of GFP-P16p (GFP-cycP16p) with a monoisotopic mass of 29,643.77 (theoretical mass is 29,642.72) (FIG. 4C). Moreover, the MS analysis indicated that the GFP-cycP16p was the dominant component in the cyclization reaction mixture. Compared to the pyl-inspired cyclization of RGD in a previous study¹⁰ which took >96 h and reached only 58% completion, the facile cyclization of P16p in under 3 h was nearly quantitative—a drastic enhancement in efficiency and completion. These results thus validated the hypothesis that preformed structural motifs can be used to promote facile cyclizations.

### Example 5—In Vitro Binding Studies of Cyclized P16p to CDK4

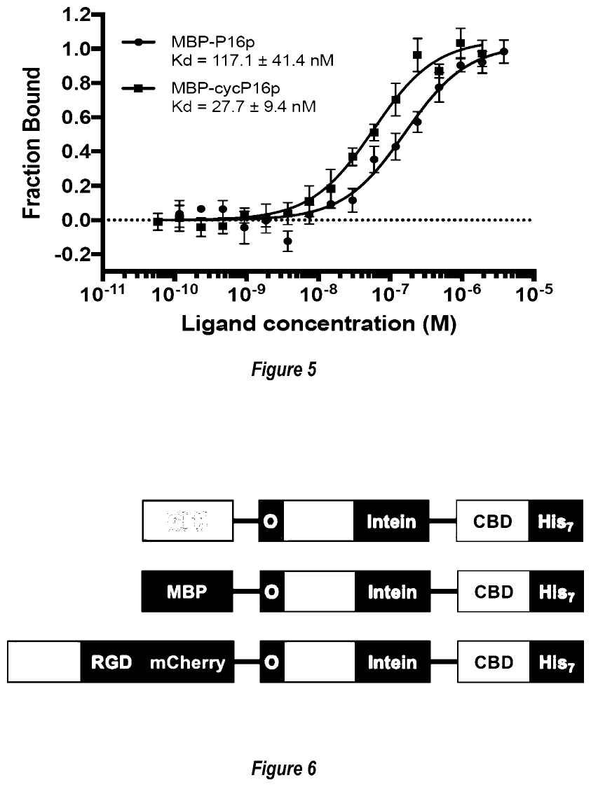

To evaluate the impact of cyclization on the binding affinity of P16p with CDK4, analytical size exclusion chromatography (SEC) and microscale thermophoresis (MST) were performed. SEC analysis showed that GFP-cycP16p interacted with GST-CDK4 to form a complex. In the MST binding assay, the GFP tag of the GFP-cycP16p was replaced with an MBP tag to avoid fluorescence interference. Pull-down assays verified that the resultant MBP-P16p could still capture CDK4 from MCF-7 cell lysate. Subsequent MST binding studies showed that the cyclized MBP-P16p protein (MBP-cycP16p) exhibited 4-fold higher binding affinity with GST-CDK4 (Kd=27.7±9.4 nM; FIG. 5) compared to its linear counterpart MBP-P16p (Kd=117.1±41.4 nM; FIG. 5).

### Example 6—Generation and In Vitro Characterization of Bifunctional Bicyclic CycRGD-mCh-cycP16p

This Example relates to further improving the therapeutic potential of cyclized P16 (cycP16p) by providing it with a cyclic RGD motif for cancer cell targeting. A dumbbell-shaped protein was envisioned where the protein would have (1) cyclic RGD on one end, acting as a tumor-homing device that specifically binds to αvβ3 integrin receptors that often overexpressed on cancer cells, and (2) cyclized P16 on the other end, acting on an intracellular target to elicit an anticancer response.

To this end, the fusion protein cycRGD-mCherry-OP16p-intein-CBD-His7 was designed (FIG. 6). This protein comprised (1) an N-terminal disulfide-based cyclic RGD motif37 (cycRGD) to enhance cellular specificity and uptake via integrin binding, (2) mCherry to facilitate improved confocal imaging,³⁸ (3) P16p as described in the preceding Examples, and (4) the intein-CBD-His7 tag. cycRGD-mCherry-OP16p-intein-CBD-His7 was expressed, purified, and cyclized following similar procedures described for GFP-cycP16p and MBP-cycP16p in the preceding Examples. For cyclization of the N-terminal RGD, the protein was air oxidized for 24 h to produce bicyclic cycRGD-mCh-cycP16p. Cellular uptake and distribution of the cycRGD-mCherry bearing linear uncyclized P16p (cycRGD-mCh-P16p) and the cyclized P16p (cycRGD-mCh-cycP16p) were then evaluated by incubating the individual proteins with MCF-7 breast cancer cells for 20 h. Significant mCherry fluorescence was detected intracellularly in both treated cells as indicated by sectional scanning using confocal laser scanning microscopy. These results indicate that both linear and cyclized cycRGD-mCh-P16p were able to enter cells.

### Example 7—Effect of Cyclization on the Inhibition and Stability of P16 Peptide

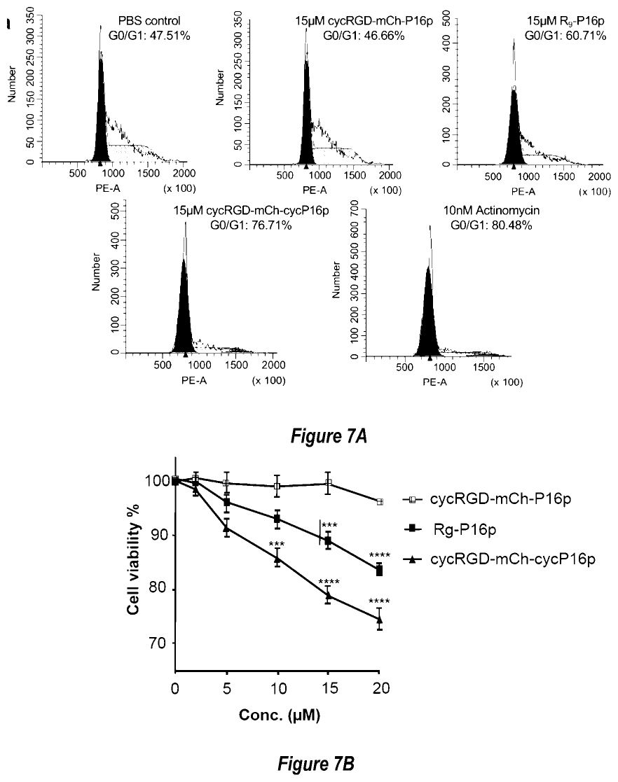

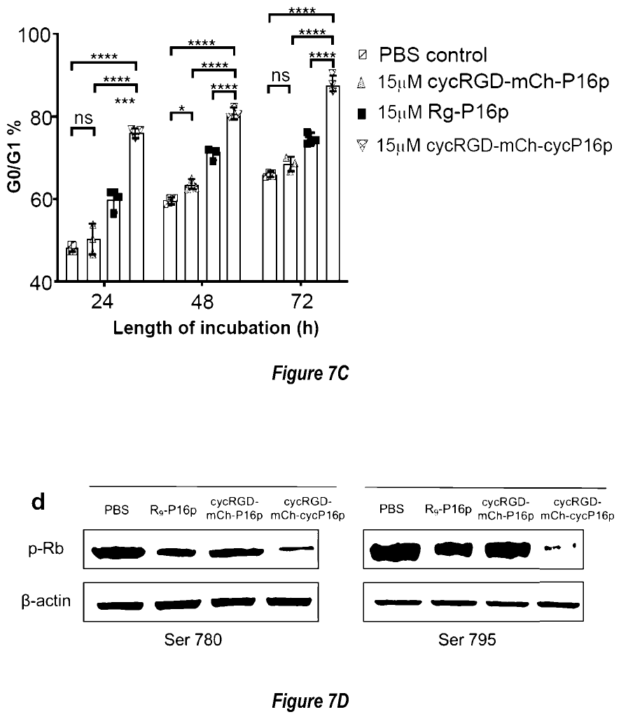

This Example relates to determining whether cyclization of the P16p could indeed endow cycRGD-mCh-cycP16p with the ability to arrest the cell cycle, and enable a more potent inhibitory effect on cancer cell growth. MCF-7 cells were treated with different concentrations of linear or cyclic cycRGD-mCherry-P16p. A linear P16p fused to the well-known poly-arginine cell penetrating sequence (R9-P16p) was also included for comparison. Flow cytometric analysis of the different groups of treated cells revealed that, at 15 μM concentration, cycRGD-mCh-cycP16p was the most potent cell cycle arrestor (76.71%) (FIG. 7A; bottom, left panel), and compared favorably to R9-P16p, which was moderately effective (60.71%) (FIG. 7A; top, right panel), while the linear counterpart cycRGD-mCh-P16p had minimal effect on G0/G1 phase arrest (46.66%) (FIG. 7A; top, middle panel). Similar results were observed in the cell proliferation assay in which cycRGDmCh-cycP16p %) (FIG. 7A; bottom, left panel), exerted the strongest inhibition on MCF-7 cell growth compared with R9-P16p (FIG. 7A; top, right panel) and cycRGD-mCh-P16p (FIG. 7A; top, middle panel) at the concentrations tested. These enhanced performances of cycRGD-mCh-cycP16p could be attributed in part to its higher affinity binding to CDK4 in MCF-7 cells brought upon by the cycP16p subunit as indicated by the binding constant obtained from MST as discussed above in Example 5 and shown in FIG. 5.

The stability of cycRGD-mCh-P16p was evaluated by monitoring its ability to arrest cell cycle for an extended period of up to 72 h. This time course study indicated that the cycRGD-mCh-cycP16p retained its ability to induce G0/G1 cell cycle arrest even after prolonged exposure to serum-supplemented culture medium as shown by the approximately 88% of arrested cells at 72 h (FIG. 7C). By comparison, the linear cycRGDmCh-P16p was not able to induce meaningful cell cycle arrest. These results were further verified by interrogating the phosphorylation status of specific sites on retinoblastoma (Rb) known to be associated with G1-to-S phase transition of cell cycle in the differentially treated MCF-cells. Western blot analysis using phosphor-specific anti-Rb antibodies for serine 780 and serine 795—the two residues whose phosphorylation will activate cell progression and inactive cell cycle arresting function of pRB, respectively—revealed significantly reduced levels of phosphorylation at the indicated sites in MCF-7 cells treated with cyclic cycRGD-mCh-cycP16p compared with those treated by the other two linear P16p's (FIG. 7D). Collectively, the results suggest that cyclization enhances the potency of P16p as a CDK4/6 inhibitor due in part to enhanced serum stability and binding affinity with CDK4. Notably, the use of D-Cys-ε-Lys allowed for the generation of an iso-peptide-linked cyclic P16p subunit that is more resistant to proteolysis and stable in the reducing cytosol—key features critical to the development of peptide therapeutics.

### Summary

The Examples demonstrate that an efficient and robust platform was developed based on the Pyl technology for the production of cyclized proteins. This system is distinct in that the ncAA substrate is intracellularly synthesized from low-cost and commercially available simple amino acids, which greatly reduces the cost of production of the target protein—an essential attribute in the development of therapeutic products. Via the optimization of the readthrough system and the implementation of a structure-inspired macrocyclization strategy, ncAA-bearing proteins can be produced in high yield and cyclized efficiently to near completion. With this optimized system, the P16 peptide subunit of a fusion protein was successfully cyclized under mild and reducing conditions within 3 h. The cyclized P16p subunit exhibited enhanced binding affinity with CDK4 and more potent inhibitory effect on pRb phosphorylation than its linear counterpart, resulting in meaningful G0/G1 arrest in MCF-7 cells. Given the important role of cyclic proteins/peptides in drug development, our incorporation system and cyclization strategy can potentially be applied to cyclize other potential therapeutic drug candidates, thereby providing a distinct and complementary approach in the preparation of cyclic peptide-containing proteins.

### II. Synthesis and Use of D-Pra-ε-Lys and D-Allyl-ε-Lys (Overview of Examples 8-9)

The in cellulo syntheses of the ncAAs D-Pra-ε-Lys and D-Allyl-ε-L-Lys were achieved by hijacking the Pyl biosynthesis pathway. Site-saturation mutagenesis was used to generate PylC libraries to produce suitable PylC variants capable of producing D-Pra-ε-Lys and D-Allyl-ε-L-Lys in the cell. D-Pra-ε-Lys contains a propargyl amino acid and can be used in click chemistry reactions and the like. D-allyl-ε-L-Lys contains a reactive vinyl handle that can be used for Michael additional reactions to thiols.

### Example 8—In Cellulo Synthesis of D-Pra-ε-Lys

This Example relates to synthesis of D-Pra-ε-Lys using PylC variants. The Pyl analog D-Pra-ε-Lys was biosynthesized from the starter chemical (R)-2-aminopent-4-yonic acid with endogenous L-Lysine (FIG. 8A). Site-saturation mutagenesis was performed on Methanosarcina barkeri PylC and variant PylCs capable of producing of D-Pra-ε-Lys were selected as discussed in Examples 1 and 3 above. After selection, 31 colonies survived and each of these was grown in a 250 μL culture and inoculated with ITPG and their mCherry fluorescence intensity were measured as showed in FIG. 8B. The 28 PylC mutants exhibiting the strongest mCherry fluorescence signals were sent for Sanger sequencing. PylC variants identified were as shown in Table 2 above. Analysis of the sequences revealed that the four residues mutated in PylC—S177, E179, D233, and T256—prefer to be hydrophobic with the mutation of D233H being highly conserved.

Next, D-Pra-ε-Lys was incorporated in a protein at UAG codon using the PylC variant PylC^{GVNV}. PylC^{GVNV} was co-expressed with the PylS^{WT}-PylT^{M15}-GFP-UAG-P16p-intein-CBD-His₇ expression vector in the presence of 5 mM propargyl chemical supplement. Recombinant protein expression was induced at 25° C. overnight. The GFP UAG-P16p protein was purified by Ni²⁺ affinity chromatography and the GFP-positive fractions were collected. GFP UAG-P16p protein was treated with DTT and trypsin. According to ESI-MS/MS analysis, D-Pra-ε-Lys was identified in the UAG-embedded GFP protein. The results demonstrate successful in cellulo synthesis of D-Pra-ε-Lys using a PylC variant.

### Example 9—In Cellulo Synthesis of D-Allyl-ε-Lys

This Example relates to synthesis of D-Allyl-ε-Lys using PylC variants. The Pyl analog of D-Allyl-ε-Lys was biosynthesized from the starting compound D-Allyl-OH with endogenous L-Lysine (FIG. 9A). Site-saturation mutagenesis was performed on Methanosarcina barkeri PylC and variant PylCs capable of producing D-Allyl-ε-Lys were identified as discussed in Examples 1, 3, and 8 above, using antibiotic selection and an mCherry fluorescence readthrough assay (FIG. 9B). The top 10 PylC variants with the highest readthrough, and presumably highest levels of D-Allyl-ε-Lys biosynthesis, were sequenced and the site-specific mutations identified as shown in Table 3 above.

Next, D-Allyl-ε-Lys was incorporated in a protein at UAG codon using the PylC variant PylC^{GVNV}. To achieve this, the target cells were transformed with two vectors: a vector constitutively expressing PylC^{GVNV} as well as a PylRS^{WT}-PylT^{M15}-GFP-UAG-P16p-intein-CBD-His₇ expression vector. After an initial growth period in the presence of D-Allyl-OH to allow for PylC^{GVNV}-mediated synthesis of D-Allyl-ε-Lys, GFP UAG-P16p protein expression was induced with isopropyl β-d-l-thiogalactopyranoside (IPTG) and the cells were allowed to grow at 25° C. overnight. The GFP UAG-P16p protein was purified by Ni²⁺ affinity chromatography and the GFP-positive fractions were collected. GFP UAG-P16p protein was treated with DTT and trypsin. According to ESI-MS/MS analysis, D-Allyl-ε-Lys was identified in the UAG-embedded GFP protein. The results demonstrate successful in cellulo synthesis of D-Allyl-ε-Lys using a PylC variant.

### III. Synthesis and Use of 2-Chloro-Acetyl-ε-Lys (Overview of Examples 10-12)

The in cellulo synthesis of the ncAA 2-Chloro-Acetyl-ε-Lys (2ClAcK; FIG. 10) is achieved by hijacking the Pyl biosynthesis pathway. Using directed evolution experiments, a PylRS/tRNA^{pyl} pair is identified that is was suitable for incorporating 2ClAcK into proteins. The unique feature of the 2ClAcK is that the reactivity of its 2-chloro-acetyl group with cysteine is tuned such that the coupling requires extended local proximity of the two moieties for the reaction to take place. This requirement is advantageous since it reduces off target reactions, allowing for selective coupling only following translational incorporation and when 2ClAcK is spatially positioned near a target cysteine within a target protein.

Using a tRNA(UAG) (PylT) and optimized pyrrolysyl tRNA synthetase (PylS*) pair (e.g., a wild-type archaea or bacterial PylT, or the PylT^{M15} mutant as discussed in Serfling, Robert et al. Nucleic Acids Research vol. 46, 1 (2018): 1-10), and the PylS* mutant as discussed in Kobayashi et al. Journal of the American Chemical Society 2016 138 (45), 14832-14835). 2ClAcK was successfully incorporated into a peptide model system derived from SUMO1, His-SUMOmini2, that can be useful as an inhibitor of α-synuclein aggregation and treatment for Parkinson's disease (Liang et al. Cell Chem Biol, 2021, 28, 180).

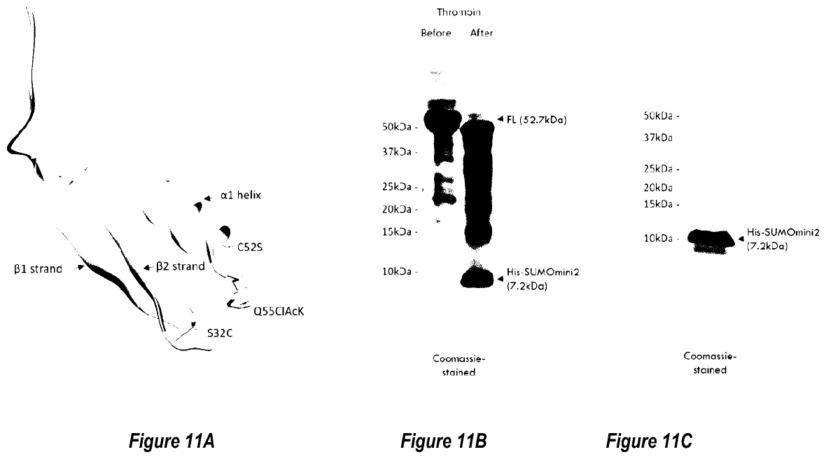

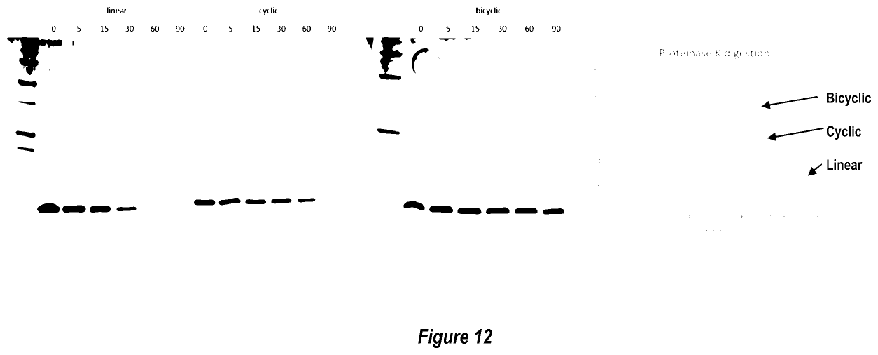

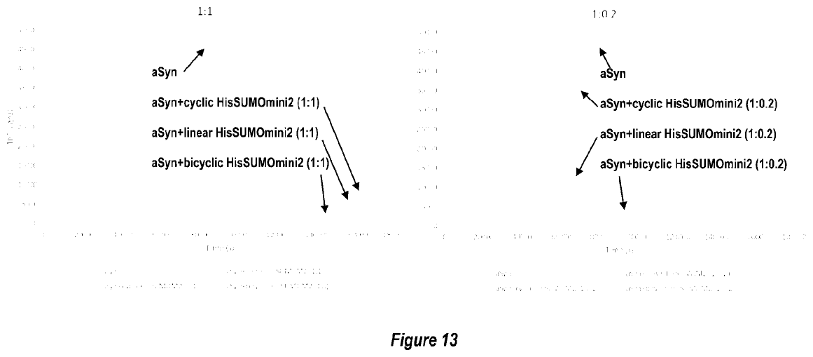

In the SUMO1 peptide case, cyclic His-SUMOmini2 peptide was produced from a His-SUMOmini2-LVPRGSSUMOCt-MBP construct comprised of a core SUMO1 protein domain with an internal thrombin cleavage sequence LVPRGS and a C-terminal maltose binding protein (MBP) purification tag. The core SUMO1 domain served as a scaffold which positioned the 2ClAcK and Cys residues in the proper spatial orientation for chemistry. Cleavage of the construct with thrombin, followed by nickel affinity chromatography allowed for separation of the N-terminal cyclic His-SUMOmini2 peptide from the remaining SUMO1 C-terminal fragment (SUMOCt) and MBP domain (FIGS. 11A-11C). The formation of the 2ClAcK-Cys crosslink in the cyclic His-SUMOmini2 peptide was confirmed by mass spectrometry. Taking advantage of the spatial specificity of the 2ClAcK-Cys coupling reaction, the production of a bicyclic His-SUMOmini2 peptide was also tested. In His-SUMOmini2, two UAG sites were engineered for 2ClAcK incorporation, and two cysteines positioned so that each one was spatially proximal to a 2ClAcK for facile cross-linking. The benefits of bicyclic SUMO1 peptide included enhanced resistance to protease degradation, and improved suppression of α-synuclein aggregation (FIGS. 12 and 13).

### Example 10—In Cellulo Synthesis of 2-Chloro-Acetyl-ε-Lys

This Example relates to synthesis using a PylC variant. The Pyl analog 2-Chloro-Acetyl-ε-Lys (2ClAcK) is biosynthesized from 2-chloro-acetic acid and endogenous lysine (FIG. 10). Site-saturation mutagenesis is performed on Methanosarcina barkeri PylC and variant PylCs capable of producing 2ClAcK are selected as discussed in Examples 1, 3, 8, and 9 above. PylC variants are analyzed by sequencing.

Next, 2ClAcK is incorporated in a protein at UAG codon using an identified PylC variant. The PylC variant is co-expressed with the PylS*T-mCherry(UAG)-kanR(UAG) reporter plasmid which harbors an optimized pyrrolysyl tRNA synthetase (PylS*) that can charge the pyrrolysyl tRNA(UAG) (PylT) with 2ClAcK for translation incorporation. PylC mutants capable of producing 2ClAcK are identified by first screening the co-transformed cells on high concentrations of kanamycin in the presence of IPTG, lysine, and 2-chloro-acetic acid. Since the presence of 2ClAcK is needed for expression of the KanR gene, only cells with a PylC mutant capable of producing 2ClAcK will survive. After the initial round of kanamycin selection, the identified cells are evaluated for their readthrough efficiency by culturing them in small scale (in a 96-well plate) in the presence of IPTG, lysine and 2-chloro-acetic acid and determining the relative level of mCherry fluorescence. After the initial mutants are identified, further rounds of evolution and selection are performed to identify PylC mutants with even better read-through ability for improved 2ClAcK biosynthesis. Identified PylC mutants are overexpressed, purified, and characterized, for example, using protein crystallography to determine their three-dimensional structures to gain insight into how substrate specificity of PylC is altered to catalyze a reaction using lysine and 2-chloroacetic acid as substrates (for 2ClAck) instead of D-ornithine (for Pyl).

### Example 11—Generation of Cyclic SUMO-Derived Peptides

This Example relates to producing cyclic SUMO-derived peptides by incorporation of 2ClAcK. It was previously demonstrated that an earlier linear version of SUMO1 peptide (15-55) (SUMOmini2) can effectively inhibit α-synuclein aggregation in vitro and in vivo (Liang et al. Cell Chem Biol, 2021, 28, 180). To produce monocyclic and bicyclic versions of the His-SUMOmini2 peptide, synthetic 2ClAcK was incorporated into the His-SUMOmini2-LVPRGS-SUMOCt-MBP construct during protein translation. Alternatively, the PylS*T-His-SUMOmini2-LVPRGS-SUMOCt-MBP and a plasmid constitutively expressing the optimized PylC mutant identified in Example 10 above is co-transformed into E. coli. The E. coli is cultured in media supplemented with 2-chloro-acetic acid, thus enabling the in cellulo synthesis of 2ClAcK generated by the mutant PylC, which can then be directly incorporated into a nascent polypeptide of His-SUMOmini2-LVPRGS-SUMOCt-MBP protein as mediated by the optimized tRNA/PylS* pair.

The purified SUMOmini2-LVPRGS-SUMOCt-MBP construct was incubated with thrombin and then purified by nickel affinity chromatography (FIGS. 11A-11C). Notably, previous attempts to produce disulfide-stabilized His-SUMOmini2 peptides using chemical synthesis were unsuccessful. The ability to produce monocyclic and bicyclic His-SUMOmini2 peptide discussed in this Example mitigates this chemical synthesis roadblock. The bicyclic His-SUMOmini2 peptide appears to be the most effective based on its enhanced resistance to proteolytic degradation (FIG. 12) and enhanced ability to suppress α-synuclein aggregation (FIG. 13). The strategy demonstrated here to cyclize SUMO-derived peptides may be useful for suppressing α-synuclein aggregation in Parkinson's disease.

### REFERENCES

The following references relate to Examples 1-7 above.
1. Sato, A. K.; Viswanathan, M.; Kent, R. B.; Wood, C. R., Therapeutic peptides: technological advances driving peptides into development. Curr. Opin. Biotechnol. 2006, 17 (6), 638-642.
2. Colgrave, M. L.; Craik, D. J., Thermal, chemical, and enzymatic stability of the cyclotide kalata B1: the importance of the cyclic cystine knot. Biochemistry 2004, 43 (20), 5965-5975.
3. Ji, Y.; Majumder, S.; Millard, M.; Borra, R.; Bi, T.; Elnagar, A. Y.; Neamati, N.; Shekhtman, A.; Camarero, J. A., In vivo activation of the p53 tumor suppressor pathway by an engineered cyclotide. J. Am. Chem. Soc. 2013, 135 (31), 11623-11633.
4. Ngo, K. H.; Yang, R.; Das, P.; Nguyen, G. K.; Lim, K. W.; Tam, J. P.; Wu, B.; Phan, A. T., Cyclization of a G4-specific peptide enhances its stability and G-quadruplex binding affinity. Chem. Commun. 2020, 56 (7), 1082-1084.
5. Wilbs, J.; Kong, X.-D.; Middendorp, S. J.; Prince, R.; Cooke, A.; Demarest, C. T.; Abdelhafez, M. M.; Roberts, K.; Umei, N.; Gonschorek, P., Cyclic peptide FXII inhibitor provides safe anticoagulation in a thrombosis model and in artificial lungs. Nat. Commun. 2020, 11 (1), 1-13.
6. Clardy, J.; Walsh, C., Lessons from natural molecules. Nature 2004, 432 (7019), 829-837.
7. Driggers, E. M.; Hale, S. P.; Lee, J.; Terrett, N. K., The exploration of macrocycles for drug discovery—an underexploited structural class. Nat. Rev. Drug Discovery 2008, 7 (7), 608-624.
8. Deyle, K.; Kong, X. D.; Heinis, C., Phage selection of cyclic peptides for application in research and drug development. Acc. Chem. Res. 2017, 50 (8), 1866-1874.
9. Tavassoli, A.; Benkovic, S. J., Split-intein mediated circular ligation used in the synthesis of cyclic peptide libraries in E. coli. Nat. Protoc. 2007, 2 (5), 1126-33.
10. Lee, M. M.; Fekner, T.; Lu, J.; Heater, B. S.; Behrman, E. J.; Zhang, L.; Hsu, P. H.; Chan, M. K., Pyrrolysine-inspired protein cyclization. Chembiochem 2014, 15 (12), 1769-1772.
11. Bouclier, C.; Simon, M.; Laconde, G.; Pellerano, M.; Diot, S.; Lantuejoul, S.; Busser, B.; Vanwonterghem, L.; Vollaire, J.; Josserand, V., Stapled peptide targeting the CDK4/cyclin D interface combined with Abemaciclib inhibits KRAS mutant lung cancer growth. Theranostics 2020, 10 (5), 2008.
12. Abdelkader, E. H.; Qianzhu, H.; George, J.; Frkic, R. L.; Jackson, C. J.; Nitsche, C.; Otting, G.; Huber, T., Genetic encoding of cyanopyridylalanine for in-cell protein macrocyclization by the nitrileaminothiol click reaction. Angew. Chem., Int. Ed. 2022, 61 (13), e202114154.
13. Dong, H.; Li, J.; Liu, H.; Lu, S.; Wu, J.; Zhang, Y.; Yin, Y.; Zhao, Y.; Wu, C., Design and ribosomal incorporation of noncanonical disulfide-directing motifs for the development of multicyclic peptide libraries. J. Am. Chem. Soc. 2022, 144 (11), 5116-5125.
14. Dawson, P. E.; Muir, T. W.; Clark-Lewis, I.; Kent, S. B., Synthesis of proteins by native chemical ligation. Science 1994, 266 (5186), 776-779.
15. Li, X.; Fekner, T.; Ottesen, J. J.; Chan, M. K., A pyrrolysine analogue for site-specific protein ubiquitination. Angew. Chem. 2009, 121 (48), 9348-9351.
16. Serrano, M.; Hannon, G. J.; Beach, D., A new regulatory motif in cell-cycle control causing specific inhibition of cyclin D/CDK4. Nature 1993, 366 (6456), 704-707.
17. Finn, R. S.; Martin, M.; Rugo, H. S.; Jones, S.; Im, S. A.; Gelmon, K.; Harbeck, N.; Lipatov, O. N.; Walshe, J. M.; Moulder, S., Palbociclib and letrozole in advanced breast cancer. N. Engl. J. Med. 2016, 375 (20), 1925-1936.
18. Fåhraeus, R.; Paramio, J. M.; Ball, K. L.; Laín, S.; Lane, D. P., Inhibition of pRb phosphorylation and cell-cycle progression by a 20-residue peptide from p16CDKN2/INK4A. Curr. Biol. 1996, 6 (1), 84-91.
19. Russo, A. A.; Tong, L.; Lee, J.-O.; Jeffrey, P. D.; Pavletich, N. P., Structural basis for inhibition of the cyclin-dependent kinase Cdk6 by the tumour suppressor p16INK4a. Nature 1998, 395 (6699), 237-243.
20. Fujimoto, K.; Hosotani, R.; Miyamoto, Y.; Doi, R.; Koshiba, T.; Otaka, A.; Fujii, N.; Beauchamp, R. D.; Imamura, M., Inhibition of pRb phosphorylation and cell cycle progression by an antennapedia p16INK4A fusion peptide in pancreatic cancer cells. Cancer Lett. 2000, 159 (2), 151-158.
21. Hosotani, R.; Miyamoto, Y.; Fujimoto, K.; Doi, R.; Otaka, A.; Fujii, N.; Imamura, M., Trojan p16 peptide suppresses pancreatic cancer growth and prolongs survival in mice. Clin. Cancer Res. 2002, 8 (4), 1271-1276.
22. Camarero, J. A.; Pavel, J.; Muir, T. W., Chemical synthesis of a circular protein domain: evidence for folding-assisted cyclization. Angew. Chem., Int. Ed. 1998, 37 (3), 347-349.
23. Fan, C.; Xiong, H.; Reynolds, N. M.; Soll, D., Rationally evolving tRNA^{pyl} for efficient incorporation of noncanonical amino acids. Nucleic Acids Res. 2015, 43 (22), e156.
24. Serfling, R.; Lorenz, C.; Etzel, M.; Schicht, G.; Böttke, T.; Mörl, M.; Coin, I., Designer tRNAs for efficient incorporation of noncanonical amino acids by the pyrrolysine system in mammalian cells. Nucleic Acids Res. 2018, 46 (1), 1-10.
25. Kavran, J. M.; Gundllapalli, S.; O'Donoghue, P.; Englert, M.; Söll, D.; Steitz, T. A., Structure of pyrrolysyl-tRNA synthetase, an archaeal enzyme for genetic code innovation. Proc. Natl. Acad. Sci. 2007, 104 (27), 11268-11273.
26. Hohl, A.; Karan, R.; Akal, A.; Renn, D.; Liu, X.; Ghorpade, S.; Groll, M.; Rueping, M.; Eppinger, J., Engineering a polyspecific pyrrolysyl-tRNA synthetase by a high throughput FACS screen. Sci. Rep. 2019, 9 (1), 1-9.
27. Nguyen, D. P.; Elliott, T.; Holt, M.; Muir, T. W.; Chin, J. W., Genetically encoded 1, 2-aminothiols facilitate rapid and site-specific protein labeling via a bio-orthogonal cyanobenzothiazole condensation. J. Am. Chem. Soc. 2011, 133 (30), 11418-11421.
28. Pettersen, E. F.; Goddard, T. D.; Huang, C. C.; Couch, G. S.; Greenblatt, D. M.; Meng, E. C.; Ferrin, T. E., UCSF Chimera—a visualization system for exploratory research and analysis. J. Comput. Chem. 2004, 25 (13), 1605-1612.
29. Wannier, T. M.; Kunjapur, A. M.; Rice, D. P.; McDonald, M. J.; Desai, M. M.; Church, G. M., Adaptive evolution of genomically recoded Escherichia coli. Proc. Natl. Acad. Sci. 2018, 115 (12), 3090-3095.
30. Gaston, M. A.; Zhang, L.; Green-Church, K. B.; Krzycki, J. A., The complete biosynthesis of the genetically encoded amino acid pyrrolysine from lysine. Nature 2011, 471 (7340), 647-650.
31. Quitterer, F.; List, A.; Beck, P.; Bacher, A.; Groll, M., Biosynthesis of the 22nd genetically encoded amino acid pyrrolysine: structure and reaction mechanism of PylC at 1.5 Å resolution. J. Mol. Biol. 2012, 424 (5), 270-282.
32. Brünger, A. T.; Adams, P. D.; Clore, G. M.; DeLano, W. L.; Gros, P.; Grosse-Kunstleve, R. W.; Jiang, J.-S.; Kuszewski, J.; Nilges, M.; Pannu, N. S., Crystallography & NMR system: A new software suite for macromolecular structure determination. Acta Crystallogr. D 1998, 54 (5), 905-921.
33. Brunger, A. T., Version 1.2 of the crystallography and NMR system. Nat. Protoc. 2007, 2 (11), 2728-2733.
34. Wadhwani, P.; Afonin, S.; Ieronimo, M.; Buerck, J.; Ulrich, A. S., Optimized protocol for synthesis of cyclic gramicidin S: starting amino acid is key to high yield. J. Org. Chem. 2006, 71 (1), 55-61.
35. Yu, Z.; Yu, X. C.; Chu, Y.-H., MALDI-MS determination of cyclic peptidomimetic sequences on single beads directed toward the generation of libraries. Tetrahedron Lett. 1998, 39 (1-2), 1-4.
36. Perlman, Z. E.; Bock, J. E.; Peterson, J. R.; Lokey, R. S., Geometric diversity through permutation of backbone configuration in cyclic peptide libraries. Bioorg. Med. Chem. Lett. 2005, 15 (23), 5329-5334.
37. Sugahara, K. N.; Teesalu, T.; Karmali, P. P.; Kotamraju, V. R.; Agemy, L.; Girard, O. M.; Hanahan, D.; Mattrey, R. F.; Ruoslahti, E., Tissue-penetrating delivery of compounds and nanoparticles into tumors. Cancer Cell 2009, 16 (6), 510-20.
38. Ettinger, A.; Wittmann, T., Fluorescence live cell imaging. Methods Cell Biol. 2014, 123, 77-94.
39. Ehrlich, M.; Gattner, M. J.; Viverge, B.; Bretzler, J.; Eisen, D.; Stadlmeier, M.; Vrabel, M.; Carell, T., Orchestrating the biosynthesis of an unnatural pyrrolysine amino acid for its direct incorporation into proteins inside living cells. Chem. Eur. J. 2015, 21 (21), 7701-7704.
40. Exner, M. P.; Kuenzl, T.; To, T. M. T.; Ouyang, Z.; Schwagerus, S.; Hoesl, M. G.; Hackenberger, C. P.; Lensen, M. C.; Panke, S.; Budisa, N., Design of S-allylcysteine in situ production and incorporation based on a novel pyrrolysyl-tRNA synthetase variant. Chembiochem 2017, 18 (1), 85-90.
41. Bauer, T. M.; Shaw, A. T.; Johnson, M. L.; Navarro, A.; Gainor, J. F.; Thurm, H.; Pithavala, Y. K.; Abbattista, A.; Peltz, G.; Felip, E., Brain penetration of lorlatinib: cumulative incidences of CNS and non-CNS progression with lorlatinib in patients with previously treated ALK-positive non-small-cell lung cancer. Target. Oncol. 2020, 15 (1), 55-65.
42. Zhang, L.; Tam, J. P., Synthesis and application of unprotected cyclic peptides as building blocks for peptide dendrimers. J. Am. Chem. Soc. 1997, 119 (10), 2363-2370.
43. Le Chevalier Isaad, A.; Papini, A. M.; Chorev, M.; Rovero, P., Side chain-to-side chain cyclization by click reaction. J. Pept. Sci. 2009, 15 (7), 451-454.
44. Jagasia, R.; Holub, J. M.; Bollinger, M.; Kirshenbaum, K.; Finn, M., Peptide cyclization and cyclodimerization by Cul-mediated azidealkyne cycloaddition. J. Org. Chem. 2009, 74 (8), 2964-2974.
45. Li, X.; Fekner, T.; Chan, M. K., N6-(2-(R)-propargylglycyl) lysine as a clickable pyrrolysine mimic. Chem. Asian J. 2010, 5 (8), 1765-9.
46. Fekner, T.; Li, X.; Lee, M. M.; Chan, M. K., A pyrrolysine analogue for protein click chemistry. Angew. Chem. Int. Ed. 2009, 48 (9), 1633-5.
47. Wilson, D. S.; Keefe, A. D., Random mutagenesis by PCR. Curr. Protoc. Mol. Biol. 2000, 51 (1), 8.3.1-8.3.9.
48. Miyazaki, K., MEGAWHOP cloning: a method of creating random mutagenesis libraries via megaprimer PCR of whole plasmids. Meth. Enzymol. 2011, 498, 399-406.
49. DeLano, W. L. The PyMOL molecular graphics system. http://www.pymol.org.
50. Suzuki, T.; Miller, C.; Guo, L.-T.; Ho, J. M.; Bryson, D. I.; Wang, Y.-S.; Liu, D. R.; Söll, D., Crystal structures reveal an elusive functional domain of pyrrolysyl-tRNA synthetase. Nat. Chem. Biol. 2017, 13 (12), 1261-1266.
51. Nozawa, K.; O'Donoghue, P.; Gundllapalli, S.; Araiso, Y.; Ishitani, R.; Umehara, T.; Söll, D.; Nureki, O., Pyrrolysyl-tRNA synthetase-tRNA^{pyl} structure reveals the molecular basis of orthogonality. Nature 2009, 457 (7233), 1163-1167.
52. Waterhouse, A.; Bertoni, M.; Bienert, S.; Studer, G.; Tauriello, G.; Gumienny, R.; Heer, F. T.; de Beer, T. A. P.; Rempfer, C.; Bordoli, L., SWISS-MODEL: homology modelling of protein structures and complexes. Nucleic Acids Res. 2018, 46 (W1), W296-W303.
53. Abraham, M. J.; Murtola, T.; Schulz, R.; Páll, S.; Smith, J. C.; Hess, B.; Lindahl, E., GROMACS: High performance molecular simulations through multi-level parallelism from laptops to supercomputers. SoftwareX 2015, 1, 19-25.
54. Jorgensen, W. L.; Tirado-Rives, J., Potential energy functions for atomic-level simulations of water and organic and biomolecular systems. Proc. Natl. Acad. Sci. 2005, 102 (19), 6665-6670.
55. Dodda, L. S.; Vilseck, J. Z.; Tirado-Rives, J.; Jorgensen, W. L., 1.14*CM1A-LBCC: localized bond-charge corrected CM1A charges for condensed-phase simulations. J. Phys. Chem. B 2017, 121 (15), 3864-3870.
56. Dodda, L. S.; Cabeza de Vaca, I.; Tirado-Rives, J.; Jorgensen, W. L., LigParGen web server: an automatic OPLS-AA parameter generator for organic ligands. Nucleic Acids Res. 2017, 45 (WI), W331-W336.

All publications, issued patents, and patent applications cited in this specification are herein incorporated by reference as if each individual publication or patent application were specifically and individually indicated to be incorporated by reference.

It is to be understood that this disclosure is not limited to the particular methodology, protocols, cell lines, animal species or genera, and reagents described, as such may vary. It is also to be understood that the terminology used herein is for the purpose of describing particular embodiments only and is not intended to limit the scope of the present disclosure, which will be limited only by the appended claims.

INFORMAL SEQUENCE LISTING

| SEQ ID | | |
|---|---|---|
| NO | Description | Sequence |
| 1 | Wild-type M. | MRESWGASLKTICLIGGKLQGFEAAYLSKKAGMKVLVIDK |
| | mazei PylC | NPQALIRNYADEFQCFNITEEPEKLVAISKNVDAILPVNENL |
| | (UniProt ID: | ECIEFLNSIKEKFSCPVLFDFEAYRISRDKRKSKEYFASIGTP |
| | Q8PWY3; amino | TPQDKPSEPPYFVKPPCESSSVGARIIHDRKELKELEPGMLIE |
| | acids that | EYVEGEVVSLEVIGDGNNFAVVKETLVHIDDTYDCHMVTP |
| | correspond to | LPLDPSFRELSYSLAANLPLKGIMDVEAISGPLGLKVIEIDA |
| | S177, E179, D233, | RFPSQTPTAVYYSSGINLIELLFRAFNGGIEEIKTLPEDRYCI |
| | and T256 that may | YEHLMLAENGVLIPVGEQVLSMGNDYGNYYEEPGIEIFLC |
| | be mutated are | KGENPVFTLVFWGRDREEAEARKNKGLSILKSRFGAAA |
| | shown in bold and | |
| | underlined letters | |

Sequences for PylC Variants for Synthesizing D-Cys-ε-Lys

| | | |
|---|---|---|
| 2 | PylC Variant K50- | ATGAGAGAGTCCTGGGGTGCTAGCCTGAAAACAATATG |
| | 4 polynucleotide | CCTTATAGGCGGGAAGCTGCAGGGCTTCGAGGCTGCATA |
| | sequence; | CCTATCTAAGAAAGCCGGAATGAAAGTGCTTGTAATAG |
| | mutations are | ACAAAAACCCGCAGGCGCTTATAAGGAATTATGCGGAT |
| | shown in bold and | GAGTTCCAGTGTTTTAACATAACGGAAGAGCCGGAAAA |
| | underlined letters | ACTCGTCGCGATATCAAAAAATGTTGATGCCATACTGCC |
| | | GGTAAATGAAAACCTTGAATGTATAGAATTTCTGAATTC |
| | | TATAAAAGAAAAATTCTCCTGCCCGGTACTTTTCGATTTT |
| | | GAAGCTTACAGGATCAGCAGGGATAAGAGAAAATCAAA |
| | | AGAATACTTCGCATCCATAGGAACCCCGACCCCTCAGGA |
| | | CAAACCGTCGGAACCACCTTATTTTGTAAAGCCTCCCTG |
| | | CGAAAGCAGCAGTGTGGGAGCGAGAATAATCCATGACA |
| | | GGAAAGAGCTTAAAGAGCTTGAGCCCGGGATGCTCATA |
| | | GAAGAATACGTTGAAGGGGAAGTGGTCTGTCTTACGGT |
| | | CATAGGGGATGGAAATAATTTTGCTGTGGTAAAGGAAA |
| | | CCCTTGTACATATCGATGACACCTATGACTGCCATATGG |
| | | TGACCCCTCTCCCTCTAGACCCTTCCTTCAGGGAACTATC |
| | | CTACTCCCTTGCAGCAAACCTGCCCTTAAAAGGAATTAT |
| | | GTCTGTGGAAGCGATTTCCGGCCCCCTGGGGTTAAAAGT |
| | | TATTGAGATAGATGCCCGTTTCCCGAGCCAGGTTCCGAC |
| | | TGCGGTCTATTATTCTTCCGGGATCAACCTCATAGAACT |
| | | CCTGTTCCGGGCTTTTAATGGAGGCATAGAAGAGATCAA |
| | | AACTCTCCCTGAAGACAGGTACTGCATTTACGAACATCT |
| | | CATGCTTGCAGAAAATGGAGTACTTATCCCTGTGGGAGA |
| | | ACAGGTCCTGTCCATGGGAAATGATTACGGCAATTATTA |
| | | TGAAGAACCTGGAATAGAGATTTTCCTGTGCAAAGGAG |
| | | AGAACCCTGTATTCACCCTGGTTTTCTGGGGCAGAGACA |
| | | GGGAAGAAGCTGAAGCTAGAAAAAACAAAGGGCTTTCG |
| | | ATTCTAAAAAGCCGTTTCGGAGCTGCTGCATAA |
| 3 | PylC Variant K50- | MRESWGASLKTICLIGGKLQGFEAAYLSKKAGMKVLVIDK |
| | 4 Polypeptide | NPQALIRNYADEFQCFNITEEPEKLVAISKNVDAILPVNENL |
| | Sequence; | ECIEFLNSIKEKFSCPVLFDFEAYRISRDKRKSKEYFASIGTP |
| | mutations are | TPQDKPSEPPYFVKPPCESSSVGARIIHDRKELKELEPGMLIE |
| | shown in bold and | EYVEGEVVCLTVIGDGNNFAVVKETLVHIDDTYDCHMVT |
| | underlined letters | PLPLDPSFRELSYSLAANLPLKGIMSVEAISGPLGLKVIEIDA |
| | | RFPSQVPTAVYYSSGINLIELLFRAFNGGIEEIKTLPEDRYCI |
| | | YEHLMLAENGVLIPVGEQVLSMGNDYGNYYEEPGIEIFLC |
| | | KGENPVFTLVFWGRDREEAEARKNKGLSILKSRFGAAA |
| 4 | PylC Variant K50- | ATGAGAGAGTCCTGGGGTGCTAGCCTGAAAACAATATG |
| | 12 Polynucleotide | CCTTATAGGCGGGAAGCTGCAGGGCTTCGAGGCTGCATA |
| | Sequence; | CCTATCTAAGAAAGCCGGAATGAAAGTGCTTGTAATAG |
| | mutations are | ACAAAAACCCGCAGGCGCTTATAAGGAATTATGCGGAT |
| | shown in bold and | GAGTTCCAGTGTTTTAACATAACGGAAGAGCCGGAAAA |
| | underlined letters | ACTCGTCGCGATATCAAAAAATGTTGATGCCATACTGCC |
| | | GGTAAATGAAAACCTTGAATGTATAGAATTTCTGAATTC |
| | | TATAAAAGAAAAATTCTCCTGCCCGGTACTTTTCGATTTT |
| | | GAAGCTTACAGGATCAGCAGGGATAAGAGAAAATCAAA |
| | | AGAATACTTCGCATCCATAGGAACCCCGACCCCTCAGGA |
| | | CAAACCGTCGGAACCACCTTATTTTGTAAAGCCTCCCTG |
| | | CGAAAGCAGCAGTGTGGGAGCGAGAATAATCCATGACA |
| | | GGAAAGAGCTTAAAGAGCTTGAGCCCGGGATGCTCATA |
| | | GAAGAATACGTTGAAGGGGAAGTGGTCTCGCTTTGTGT |
| | | CATAGGGGATGGAAATAATTTTGCTGTGGTAAAGGAAA |
| | | CCCTTGTACATATCGATGACACCTATGACTGCCATATGG |
| | | TGACCCCTCTCCCTCTAGACCCTTCCTTCAGGGAACTATC |
| | | CTACTCCCTTGCAGCAAACCTGCCCTTAAAAGGAATTAT |
| | | GAATGTGGAAGCGATTTCCGGCCCCCTGGGGTTAAAAGT |
| | | TATTGAGATAGATGCCCGTTTCCCGAGCCAGACGCCGAC |
| | | TGCGGTCTATTATTCTTCCGGGATCAACCTCATAGAACT |
| | | CCTGTTCCGGGCTTTTAATGGAGGCATAGAAGAGATCAA |
| | | AACTCTCCCTGAAGACAGGTACTGCATTTACGAACATCT |
| | | CATGCTTGCAGAAAATGGAGTACTTATCCCTGTGGGAGA |
| | | ACAGGTCCTGTCCATGGGAAATGATTACGGCAATTATTA |
| | | TGAAGAACCTGGAATAGAGATTTTCCTGTGCAAAGGAG |
| | | AGAACCCTGTATTCACCCTGGTTTTCTGGGGCAGAGACA |
| | | GGGAAGAAGCTGAAGCTAGAAAAAACAAAGGGCTTTCG |
| | | ATTCTAAAAAGCCGTTTCGGAGCTGCTGCATAA |
| 5 | PylC Variant K50- | MRESWGASLKTICLIGGKLQGFEAAYLSKKAGMKVLVIDK |
| | 12 Polypeptide | NPQALIRNYADEFQCFNITEEPEKLVAISKNVDAILPVNENL |
| | Sequence; | ECIEFLNSIKEKFSCPVLFDFEAYRISRDKRKSKEYFASIGTP |
| | mutations are | TPQDKPSEPPYFVKPPCESSSVGARIIHDRKELKELEPGMLIE |
| | shown in bold and | EYVEGEVVSLCVIGDGNNFAVVKETLVHIDDTYDCHMVTP |
| | underlined letters | LPLDPSFRELSYSLAANLPLKGIMNVEAISGPLGLKVIEIDA |
| | | RFPSQTPTAVYYSSGINLIELLFRAFNGGIEEIKTLPEDRYCI |
| | | YEHLMLAENGVLIPVGEQVLSMGNDYGNYYEEPGIEIFLC |
| | | KGENPVFTLVFWGRDREEAEARKNKGLSILKSRFGAAA |
| 6 | PylC Variant K50- | ATGAGAGAGTCCTGGGGTGCTAGCCTGAAAACAATATG |
| | 13 Polynucleotide | CCTTATAGGCGGGAAGCTGCAGGGCTTCGAGGCTGCATA |
| | Sequence; | CCTATCTAAGAAAGCCGGAATGAAAGTGCTTGTAATAG |
| | mutations are | ACAAAAACCCGCAGGCGCTTATAAGGAATTATGCGGAT |
| | shown in bold and | GAGTTCCAGTGTTTTAACATAACGGAAGAGCCGGAAAA |
| | underlined letters | ACTCGTCGCGATATCAAAAAATGTTGATGCCATACTGCC |
| | | GGTAAATGAAAACCTTGAATGTATAGAATTTCTGAATTC |
| | | TATAAAAGAAAAATTCTCCTGCCCGGTACTTTTCGATTTT |
| | | GAAGCTTACAGGATCAGCAGGGATAAGAGAAAATCAAA |
| | | AGAATACTTCGCATCCATAGGAACCCCGACCCCTCAGGA |
| | | CAAACCGTCGGAACCACCTTATTTTGTAAAGCCTCCCTG |
| | | CGAAAGCAGCAGTGTGGGAGCGAGAATAATCCATGACA |
| | | GGAAAGAGCTTAAAGAGCTTGAGCCCGGGATGCTCATA |
| | | GAAGAATACGTTGAAGGGGAAGTGGTCGCGCTTGCTGT |
| | | CATAGGGGATGGAAATAATTTTGCTGTGGTAAAGGAAA |
| | | CCCTTGTACATATCGATGACACCTATGACTGCCATATGG |
| | | TGACCCCTCTCCCTCTAGACCCTTCCTTCAGGGAACTATC |
| | | CTACTCCCTTGCAGCAAACCTGCCCTTAAAAGGAATTAT |
| | | GCATGTGGAAGCGATTTCCGGCCCCCTGGGGTTAAAAGT |
| | | TATTGAGATAGATGCCCGTTTCCCGAGCCAGATGCCGAC |
| | | TGCGGTCTATTATTCTTCCGGGATCAACCTCATAGAACT |
| | | CCTGTTCCGGGCTTTTAATGGAGGCATAGAAGAGATCAA |
| | | AACTCTCCCTGAAGACAGGTACTGCATTTACGAACATCT |
| | | CATGCTTGCAGAAAATGGAGTACTTATCCCTGTGGGAGA |
| | | ACAGGTCCTGTCCATGGGAAATGATTACGGCAATTATTA |
| | | TGAAGAACCTGGAATAGAGATTTTCCTGTGCAAAGGAG |
| | | AGAACCCTGTATTCACCCTGGTTTTCTGGGGCAGAGACA |
| | | GGGAAGAAGCTGAAGCTAGAAAAAACAAAGGGCTTTCG |
| | | ATTCTAAAAAGCCGTTTCGGAGCTGCTGCATAA |
| 7 | PylC Variant K50- | MRESWGASLKTICLIGGKLQGFEAAYLSKKAGMKVLVIDK |
| | 13 Polypeptide | NPQALIRNYADEFQCFNITEEPEKLVAISKNVDAILPVNENL |
| | Sequence; | ECIEFLNSIKEKFSCPVLFDFEAYRISRDKRKSKEYFASIGTP |
| | mutations are | TPQDKPSEPPYFVKPPCESSSVGARIIHDRKELKELEPGMLIE |
| | shown in bold and | EYVEGEVVALAVIGDGNNFAVVKETLVHIDDTYDCHMVT |
| | underlined letters | PLPLDPSFRELSYSLAANLPLKGIMHVEAISGPLGLKVIEIDA |
| | | RFPSQMPTAVYYSSGINLIELLFRAFNGGIEEIKTLPEDRYCI |
| | | YEHLMLAENGVLIPVGEQVLSMGNDYGNYYEEPGIEIFLC |
| | | KGENPVFTLVFWGRDREEAEARKNKGLSILKSRFGAAA |
| 8 | PylC Variant K50- | ATGAGAGAGTCCTGGGGTGCTAGCCTGAAAACAATATG |
| | 15 Polynucleotide | CCTTATAGGCGGGAAGCTGCAGGGCTTCGAGGCTGCATA |
| | Sequence; | CCTATCTAAGAAAGCCGGAATGAAAGTGCTTGTAATAG |
| | mutations are | ACAAAAACCCGCAGGCGCTTATAAGGAATTATGCGGAT |
| | shown in bold and | GAGTTCCAGTGTTTTAACATAACGGAAGAGCCGGAAAA |
| | underlined letters | ACTCGTCGCGATATCAAAAAATGTTGATGCCATACTGCC |
| | | GGTAAATGAAAACCTTGAATGTATAGAATTTCTGAATTC |
| | | TATAAAAGAAAAATTCTCCTGCCCGGTACTTTTCGATTTT |
| | | GAAGCTTACAGGATCAGCAGGGATAAGAGAAAATCAAA |
| | | AGAATACTTCGCATCCATAGGAACCCCGACCCCTCAGGA |
| | | CAAACCGTCGGAACCACCTTATTTTGTAAAGCCTCCCTG |
| | | CGAAAGCAGCAGTGTGGGAGCGAGAATAATCCATGACA |
| | | GGAAAGAGCTTAAAGAGCTTGAGCCCGGGATGCTCATA |
| | | GAAGAATACGTTGAAGGGGAAGTGGTCGCGCTTTCTGT |
| | | CATAGGGGATGGAAATAATTTTGCTGTGGTAAAGGAAA |
| | | CCCTTGTACATATCGATGACACCTATGACTGCCATATGG |
| | | TGACCCCTCTCCCTCTAGACCCTTCCTTCAGGGAACTATC |
| | | CTACTCCCTTGCAGCAAACCTGCCCTTAAAAGGAATTAT |
| | | GCATGTGGAAGCGATTTCCGGCCCCCTGGGGTTAAAAGT |
| | | TATTGAGATAGATGCCCGTTTCCCGAGCCAGTTGCCGAC |
| | | TGCGGTCTATTATTCTTCCGGGATCAACCTCATAGAACT |
| | | CCTGTTCCGGGCTTTTAATGGAGGCATAGAAGAGATCAA |
| | | AACTCTCCCTGAAGACAGGTACTGCATTTACGAACATCT |
| | | CATGCTTGCAGAAAATGGAGTACTTATCCCTGTGGGAGA |
| | | ACAGGTCCTGTCCATGGGAAATGATTACGGCAATTATTA |
| | | TGAAGAACCTGGAATAGAGATTTTCCTGTGCAAAGGAG |
| | | AGAACCCTGTATTCACCCTGGTTTTCTGGGGCAGAGACA |
| | | GGGAAGAAGCTGAAGCTAGAAAAAACAAAGGGCTTTCG |
| | | ATTCTAAAAAGCCGTTTCGGAGCTGCTGCATAA |
| 9 | PylC Variant K50- | MRESWGASLKTICLIGGKLQGFEAAYLSKKAGMKVLVIDK |
| | 15 Polypeptide | NPQALIRNYADEFQCFNITEEPEKLVAISKNVDAILPVNENL |
| | Sequence; | ECIEFLNSIKEKFSCPVLFDFEAYRISRDKRKSKEYFASIGTP |
| | mutations are | TPQDKPSEPPYFVKPPCESSSVGARIIHDRKELKELEPGMLIE |
| | shown in bold and | EYVEGEVVALSVIGDGNNFAVVKETLVHIDDTYDCHMVTP |
| | underlined letters | LPLDPSFRELSYSLAANLPLKGIMHVEAISGPLGLKVIEIDA |
| | | RFPSQLPTAVYYSSGINLIELLFRAFNGGIEEIKTLPEDRYCI |
| | | YEHLMLAENGVLIPVGEQVLSMGNDYGNYYEEPGIEIFLC |
| | | KGENPVFTLVFWGRDREEAEARKNKGLSILKSRFGAAA |
| 10 | PylC Variant K50- | ATGAGAGAGTCCTGGGGTGCTAGCCTGAAAACAATATG |
| | 17 Polynucleotide | CCTTATAGGCGGGAAGCTGCAGGGCTTCGAGGCTGCATA |
| | Sequence; | CCTATCTAAGAAAGCCGGAATGAAAGTGCTTGTAATAG |
| | mutations are | ACAAAAACCCGCAGGCGCTTATAAGGAATTATGCGGAT |
| | shown in bold and | GAGTTCCAGTGTTTTAACATAACGGAAGAGCCGGAAAA |
| | underlined letters | ACTCGTCGCGATATCAAAAAATGTTGATGCCATACTGCC |
| | | GGTAAATGAAAACCTTGAATGTATAGAATTTCTGAATTC |
| | | TATAAAAGAAAAATTCTCCTGCCCGGTACTTTTCGATTTT |
| | | GAAGCTTACAGGATCAGCAGGGATAAGAGAAAATCAAA |
| | | AGAATACTTCGCATCCATAGGAACCCCGACCCCTCAGGA |
| | | CAAACCGTCGGAACCACCTTATTTTGTAAAGCCTCCCTG |
| | | CGAAAGCAGCAGTGTGGGAGCGAGAATAATCCATGACA |
| | | GGAAAGAGCTTAAAGAGCTTGAGCCCGGGATGCTCATA |
| | | GAAGAATACGTTGAAGGGGAAGTGGTCTCGCTTTGTGT |
| | | CATAGGGGATGGAAATAATTTTGCTGTGGTAAAGGAAA |
| | | CCCTTGTACATATCGATGACACCTATGACTGCCATATGG |
| | | TGACCCCTCTCCCTCTAGACCCTTCCTTCAGGGAACTATC |
| | | CTACTCCCTTGCAGCAAACCTGCCCTTAAAAGGAATTAT |
| | | GCATGTGGAAGCGATTTCCGGCCCCCTGGGGTTAAAAGT |
| | | TATTGAGATAGATGCCCGTTTCCCGAGCCAGGTGCCGAC |
| | | TGCGGTCTATTATTCTTCCGGGATCAACCTCATAGAACT |
| | | CCTGTTCCGGGCTTTTAATGGAGGCATAGAAGAGATCAA |
| | | AACTCTCCCTGAAGACAGGTACTGCATTTACGAACATCT |
| | | CATGCTTGCAGAAAATGGAGTACTTATCCCTGTGGGAGA |
| | | ACAGGTCCTGTCCATGGGAAATGATTACGGCAATTATTA |
| | | TGAAGAACCTGGAATAGAGATTTTCCTGTGCAAAGGAG |
| | | AGAACCCTGTATTCACCCTGGTTTTCTGGGGCAGAGACA |
| | | GGGAAGAAGCTGAAGCTAGAAAAAACAAAGGGCTTTCG |
| | | ATTCTAAAAAGCCGTTTCGGAGCTGCTGCATAA |
| 11 | PylC Variant K50- | MRESWGASLKTICLIGGKLQGFEAAYLSKKAGMKVLVIDK |
| | 17 Polypeptide | NPQALIRNYADEFQCFNITEEPEKLVAISKNVDAILPVNENL |
| | Sequence; | ECIEFLNSIKEKFSCPVLFDFEAYRISRDKRKSKEYFASIGTP |
| | mutations are | TPQDKPSEPPYFVKPPCESSSVGARIIHDRKELKELEPGMLIE |
| | shown in bold and | EYVEGEVVSLCVIGDGNNFAVVKETLVHIDDTYDCHMVTP |
| | underlined letters | LPLDPSFRELSYSLAANLPLKGIMHVEAISGPLGLKVIEIDA |
| | | RFPSQVPTAVYYSSGINLIELLFRAFNGGIEEIKTLPEDRYCI |
| | | YEHLMLAENGVLIPVGEQVLSMGNDYGNYYEEPGIEIFLC |
| | | KGENPVFTLVFWGRDREEAEARKNKGLSILKSRFGAAA |
| 12 | PylC Variant K50- | ATGAGAGAGTCCTGGGGTGCTAGCCTGAAAACAATATG |
| | 19 Polynucleotide | CCTTATAGGCGGGAAGCTGCAGGGCTTCGAGGCTGCATA |
| | Sequence; | CCTATCTAAGAAAGCCGGAATGAAAGTGCTTGTAATAG |
| | mutations are | ACAAAAACCCGCAGGCGCTTATAAGGAATTATGCGGAT |
| | shown in bold and | GAGTTCCAGTGTTTTAACATAACGGAAGAGCCGGAAAA |
| | underlined letters | ACTCGTCGCGATATCAAAAAATGTTGATGCCATACTGCC |
| | | GGTAAATGAAAACCTTGAATGTATAGAATTTCTGAATTC |
| | | TATAAAAGAAAAATTCTCCTGCCCGGTACTTTTCGATTTT |
| | | GAAGCTTACAGGATCAGCAGGGATAAGAGAAAATCAAA |
| | | AGAATACTTCGCATCCATAGGAACCCCGACCCCTCAGGA |
| | | CAAACCGTCGGAACCACCTTATTTTGTAAAGCCTCCCTG |
| | | CGAAAGCAGCAGTGTGGGAGCGAGAATAATCCATGACA |
| | | GGAAAGAGCTTAAAGAGCTTGAGCCCGGGATGCTCATA |
| | | GAAGAATACGTTGAAGGGGAAGTGGTCTCGCTTGTTGT |
| | | CATAGGGGATGGAAATAATTTTGCTGTGGTAAAGGAAA |
| | | CCCTTGTACATATCGATGACACCTATGACTGCCATATGG |
| | | TGACCCCTCTCCCTCTAGACCCTTCCTTCAGGGAACTATC |
| | | CTACTCCCTTGCAGCAAACCTGCCCTTAAAAGGAATTAT |
| | | GAATGTGGAAGCGATTTCCGGCCCCCTGGGGTTAAAAGT |
| | | TATTGAGATAGATGCCCGTTTCCCGAGCCAGGTTCCGAC |
| | | TGCGGTCTATTATTCTTCCGGGATCAACCTCATAGAACT |
| | | CCTGTTCCGGGCTTTTAATGGAGGCATAGAAGAGATCAA |
| | | AACTCTCCCTGAAGACAGGTACTGCATTTACGAACATCT |
| | | CATGCTTGCAGAAAATGGAGTACTTATCCCTGTGGGAGA |
| | | ACAGGTCCTGTCCATGGGAAATGATTACGGCAATTATTA |
| | | TGAAGAACCTGGAATAGAGATTTTCCTGTGCAAAGGAG |
| | | AGAACCCTGTATTCACCCTGGTTTTCTGGGGCAGAGACA |
| | | GGGAAGAAGCTGAAGCTAGAAAAAACAAAGGGCTTTCG |
| | | ATTCTAAAAAGCCGTTTCGGAGCTGCTGCATAA |
| 13 | PylC Variant K50- | MRESWGASLKTICLIGGKLQGFEAAYLSKKAGMKVLVIDK |
| | 19 Polypeptide | NPQALIRNYADEFQCFNITEEPEKLVAISKNVDAILPVNENL |
| | Sequence; | ECIEFLNSIKEKFSCPVLFDFEAYRISRDKRKSKEYFASIGTP |
| | mutations are | TPQDKPSEPPYFVKPPCESSSVGARIIHDRKELKELEPGMLIE |
| | shown in bold and | EYVEGEVVSLVVIGDGNNFAVVKETLVHIDDTYDCHMVTP |
| | underlined letters | LPLDPSFRELSYSLAANLPLKGIMNVEAISGPLGLKVIEIDA |
| | | RFPSQVPTAVYYSSGINLIELLFRAFNGGIEEIKTLPEDRYCI |
| | | YEHLMLAENGVLIPVGEQVLSMGNDYGNYYEEPGIEIFLC |
| | | KGENPVFTLVFWGRDREEAEARKNKGLSILKSRFGAAA |
| 14 | PylC Variant K50- | ATGAGAGAGTCCTGGGGTGCTAGCCTGAAAACAATATG |
| | 45 Polynucleotide | CCTTATAGGCGGGAAGCTGCAGGGCTTCGAGGCTGCATA |
| | Sequence; | CCTATCTAAGAAAGCCGGAATGAAAGTGCTTGTAATAG |
| | mutations are | ACAAAAACCCGCAGGCGCTTATAAGGAATTATGCGGAT |
| | shown in bold and | GAGTTCCAGTGTTTTAACATAACGGAAGAGCCGGAAAA |
| | underlined letters | ACTCGTCGCGATATCAAAAAATGTTGATGCCATACTGCC |
| | | GGTAAATGAAAACCTTGAATGTATAGAATTTCTGAATTC |
| | | TATAAAAGAAAAATTCTCCTGCCCGGTACTTTTCGATTTT |
| | | GAAGCTTACAGGATCAGCAGGGATAAGAGAAAATCAAA |
| | | AGAATACTTCGCATCCATAGGAACCCCGACCCCTCAGGA |
| | | CAAACCGTCGGAACCACCTTATTTTGTAAAGCCTCCCTG |
| | | CGAAAGCAGCAGTGTGGGAGCGAGAATAATCCATGACA |
| | | GGAAAGAGCTTAAAGAGCTTGAGCCCGGGATGCTCATA |
| | | GAAGAATACGTTGAAGGGGAAGTGGTCTCTCTTGTGGT |
| | | CATAGGGGATGGAAATAATTTTGCTGTGGTAAAGGAAA |
| | | CCCTTGTACATATCGATGACACCTATGACTGCCATATGG |
| | | TGACCCCTCTCCCTCTAGACCCTTCCTTCAGGGAACTATC |
| | | CTACTCCCTTGCAGCAAACCTGCCCTTAAAAGGAATTAT |
| | | GCATGTGGAAGCGATTTCCGGCCCCCTGGGGTTAAAAGT |
| | | TATTGAGATAGATGCCCGTTTCCCGAGCCAGAATCCGAC |
| | | TGCGGTCTATTATTCTTCCGGGATCAACCTCATAGAACT |
| | | CCTGTTCCGGGCTTTTAATGGAGGCATAGAAGAGATCAA |
| | | AACTCTCCCTGAAGACAGGTACTGCATTTACGAACATCT |
| | | CATGCTTGCAGAAAATGGAGTACTTATCCCTGTGGGAGA |
| | | ACAGGTCCTGTCCATGGGAAATGATTACGGCAATTATTA |
| | | TGAAGAACCTGGAATAGAGATTTTCCTGTGCAAAGGAG |
| | | AGAACCCTGTATTCACCCTGGTTTTCTGGGGCAGAGACA |
| | | GGGAAGAAGCTGAAGCTAGAAAAAACAAAGGGCTTTCG |
| | | ATTCTAAAAAGCCGTTTCGGAGCTGCTGCATAA |
| 15 | PylC Variant K50- | MRESWGASLKTICLIGGKLQGFEAAYLSKKAGMKVLVIDK |
| | 45 Polypeptide | NPQALIRNYADEFQCFNITEEPEKLVAISKNVDAILPVNENL |
| | Sequence; | ECIEFLNSIKEKFSCPVLFDFEAYRISRDKRKSKEYFASIGTP |
| | mutations are | TPQDKPSEPPYFVKPPCESSSVGARIIHDRKELKELEPGMLIE |
| | shown in bold and | EYVEGEVVSLVVIGDGNNFAVVKETLVHIDDTYDCHMVTP |
| | underlined letters | LPLDPSFRELSYSLAANLPLKGIMHVEAISGPLGLKVIEIDA |
| | | RFPSQNPTAVYYSSGINLIELLFRAFNGGIEEIKTLPEDRYCI |
| | | YEHLMLAENGVLIPVGEQVLSMGNDYGNYYEEPGIEIFLC |
| | | KGENPVFTLVFWGRDREEAEARKNKGLSILKSRFGAAA |
| 16 | PylC Variant K50- | ATGAGAGAGTCCTGGGGTGCTAGCCTGAAAACAATATG |
| | 48 Polynucleotide | CCTTATAGGCGGGAAGCTGCAGGGCTTCGAGGCTGCATA |
| | Sequence; | CCTATCTAAGAAAGCCGGAATGAAAGTGCTTGTAATAG |
| | mutations are | ACAAAAACCCGCAGGCGCTTATAAGGAATTATGCGGAT |
| | shown in bold and | GAGTTCCAGTGTTTTAACATAACGGAAGAGCCGGAAAA |
| | underlined letters | ACTCGTCGCGATATCAAAAAATGTTGATGCCATACTGCC |
| | | GGTAAATGAAAACCTTGAATGTATAGAATTTCTGAATTC |
| | | TATAAAAGAAAAATTCTCCTGCCCGGTACTTTTCGATTTT |
| | | GAAGCTTACAGGATCAGCAGGGATAAGAGAAAATCAAA |
| | | AGAATACTTCGCATCCATAGGAACCCCGACCCCTCAGGA |
| | | CAAACCGTCGGAACCACCTTATTTTGTAAAGCCTCCCTG |
| | | CGAAAGCAGCAGTGTGGGAGCGAGAATAATCCATGACA |
| | | GGAAAGAGCTTAAAGAGCTTGAGCCCGGGATGCTCATA |
| | | GAAGAATACGTTGAAGGGGAAGTGGTCACTCTTCCGGT |
| | | CATAGGGGATGGAAATAATTTTGCTGTGGTAAAGGAAA |
| | | CCCTTGTACATATCGATGACACCTATGACTGCCATATGG |
| | | TGACCCCTCTCCCTCTAGACCCTTCCTTCAGGGAACTATC |
| | | CTACTCCCTTGCAGCAAACCTGCCCTTAAAAGGAATTAT |
| | | GCATGTGGAAGCGATTTCCGGCCCCCTGGGGTTAAAAGT |
| | | TATTGAGATAGATGCCCGTTTCCCGAGCCAGATGCCGAC |
| | | TGCGGTCTATTATTCTTCCGGGATCAACCTCATAGAACT |
| | | CCTGTTCCGGGCTTTTAATGGAGGCATAGAAGAGATCAA |
| | | AACTCTCCCTGAAGACAGGTACTGCATTTACGAACATCT |
| | | CATGCTTGCAGAAAATGGAGTACTTATCCCTGTGGGAGA |
| | | ACAGGTCCTGTCCATGGGAAATGATTACGGCAATTATTA |
| | | TGAAGAACCTGGAATAGAGATTTTCCTGTGCAAAGGAG |
| | | AGAACCCTGTATTCACCCTGGTTTTCTGGGGCAGAGACA |
| | | GGGAAGAAGCTGAAGCTAGAAAAAACAAAGGGCTTTCG |
| | | ATTCTAAAAAGCCGTTTCGGAGCTGCTGCATAA |
| 17 | PylC Variant K50- | MRESWGASLKTICLIGGKLQGFEAAYLSKKAGMKVLVIDK |
| | 48 Polypeptide | NPQALIRNYADEFQCFNITEEPEKLVAISKNVDAILPVNENL |
| | Sequence; | ECIEFLNSIKEKFSCPVLFDFEAYRISRDKRKSKEYFASIGTP |
| | mutations are | TPQDKPSEPPYFVKPPCESSSVGARIIHDRKELKELEPGMLIE |
| | shown in bold and | EYVEGEVVTLPVIGDGNNFAVVKETLVHIDDTYDCHMVTP |
| | underlined letters | LPLDPSFRELSYSLAANLPLKGIMHVEAISGPLGLKVIEIDA |
| | | RFPSQMPTAVYYSSGINLIELLFRAFNGGIEEIKTLPEDRYCI |
| | | YEHLMLAENGVLIPVGEQVLSMGNDYGNYYEEPGIEIFLC |
| | | KGENPVFTLVFWGRDREEAEARKNKGLSILKSRFGAAA |
| 18 | PylC Variant K50- | ATGAGAGAGTCCTGGGGTGCTAGCCTGAAAACAATATG |
| | 50 Polynucleotide | CCTTATAGGCGGGAAGCTGCAGGGCTTCGAGGCTGCATA |
| | Sequence; | CCTATCTAAGAAAGCCGGAATGAAAGTGCTTGTAATAG |
| | mutations are | ACAAAAACCCGCAGGCGCTTATAAGGAATTATGCGGAT |
| | shown in bold and | GAGTTCCAGTGTTTTAACATAACGGAAGAGCCGGAAAA |
| | underlined letters | ACTCGTCGCGATATCAAAAAATGTTGATGCCATACTGCC |
| | | GGTAAATGAAAACCTTGAATGTATAGAATTTCTGAATTC |
| | | TATAAAAGAAAAATTCTCCTGCCCGGTACTTTTCGATTTT |
| | | GAAGCTTACAGGATCAGCAGGGATAAGAGAAAATCAAA |
| | | AGAATACTTCGCATCCATAGGAACCCCGACCCCTCAGGA |
| | | CAAACCGTCGGAACCACCTTATTTTGTAAAGCCTCCCTG |
| | | CGAAAGCAGCAGTGTGGGAGCGAGAATAATCCATGACA |
| | | GGAAAGAGCTTAAAGAGCTTGAGCCCGGGATGCTCATA |
| | | GAAGAATACGTTGAAGGGGAAGTGGTCGGTCTTGTTGT |
| | | CATAGGGGATGGAAATAATTTTGCTGTGGTAAAGGAAA |
| | | CCCTTGTACATATCGATGACACCTATGACTGCCATATGG |
| | | TGACCCCTCTCCCTCTAGACCCTTCCTTCAGGGAACTATC |
| | | CTACTCCCTTGCAGCAAACCTGCCCTTAAAAGGAATTAT |
| | | GTCTGTGGAAGCGATTTCCGGCCCCCTGGGGTTAAAAGT |
| | | TATTGAGATAGATGCCCGTTTCCCGAGCCAGTTGCCGAC |
| | | TGCGGTCTATTATTCTTCCGGGATCAACCTCATAGAACT |
| | | CCTGTTCCGGGCTTTTAATGGAGGCATAGAAGAGATCAA |
| | | AACTCTCCCTGAAGACAGGTACTGCATTTACGAACATCT |
| | | CATGCTTGCAGAAAATGGAGTACTTATCCCTGTGGGAGA |
| | | ACAGGTCCTGTCCATGGGAAATGATTACGGCAATTATTA |
| | | TGAAGAACCTGGAATAGAGATTTTCCTGTGCAAAGGAG |
| | | AGAACCCTGTATTCACCCTGGTTTTCTGGGGCAGAGACA |
| | | GGGAAGAAGCTGAAGCTAGAAAAAACAAAGGGCTTTCG |
| | | ATTCTAAAAAGCCGTTTCGGAGCTGCTGCATAA |
| 19 | PylC Variant K50- | MRESWGASLKTICLIGGKLQGFEAAYLSKKAGMKVLVIDK |
| | 50 Polypeptide | NPQALIRNYADEFQCFNITEEPEKLVAISKNVDAILPVNENL |
| | Sequence; | ECIEFLNSIKEKFSCPVLFDFEAYRISRDKRKSKEYFASIGTP |
| | mutations are | TPQDKPSEPPYFVKPPCESSSVGARIIHDRKELKELEPGMLIE |
| | shown in bold and | EYVEGEVVGLVVIGDGNNFAVVKETLVHIDDTYDCHMVT |
| | underlined letters | PLPLDPSFRELSYSLAANLPLKGIMSVEAISGPLGLKVIEIDA |
| | | RFPSQLPTAVYYSSGINLIELLFRAFNGGIEEIKTLPEDRYCI |
| | | YEHLMLAENGVLIPVGEQVLSMGNDYGNYYEEPGIEIFLC |
| | | KGENPVFTLVFWGRDREEAEARKNKGLSILKSRFGAAA |
| 20 | PylC Variant K75- | ATGAGAGAGTCCTGGGGTGCTAGCCTGAAAACAATATG |
| | 1 Polynucleotide | CCTTATAGGCGGGAAGCTGCAGGGCTTCGAGGCTGCATA |
| | Sequence; | CCTATCTAAGAAAGCCGGAATGAAAGTGCTTGTAATAG |
| | mutations are | ACAAAAACCCGCAGGCGCTTATAAGGAATTATGCGGAT |
| | shown in bold and | GAGTTCCAGTGTTTTAACATAACGGAAGAGCCGGAAAA |
| | underlined letters | ACTCGTCGCGATATCAAAAAATGTTGATGCCATACTGCC |
| | | GGTAAATGAAAACCTTGAATGTATAGAATTTCTGAATTC |
| | | TATAAAAGAAAAATTCTCCTGCCCGGTACTTTTCGATTTT |
| | | GAAGCTTACAGGATCAGCAGGGATAAGAGAAAATCAAA |
| | | AGAATACTTCGCATCCATAGGAACCCCGACCCCTCAGGA |
| | | CAAACCGTCGGAACCACCTTATTTTGTAAAGCCTCCCTG |
| | | CGAAAGCAGCAGTGTGGGAGCGAGAATAATCCATGACA |
| | | GGAAAGAGCTTAAAGAGCTTGAGCCCGGGATGCTCATA |
| | | GAAGAATACGTTGAAGGGGAAGTGGTCTCTCTTAATGT |
| | | CATAGGGGATGGAAATAATTTTGCTGTGGTAAAGGAAA |
| | | CCCTTGTACATATCGATGACACCTATGACTGCCATATGG |
| | | TGACCCCTCTCCCTCTAGACCCTTCCTTCAGGGAACTATC |
| | | CTACTCCCTTGCAGCAAACCTGCCCTTAAAAGGAATTAT |
| | | GCATGTGGAAGCGATTTCCGGCCCCCTGGGGTTAAAAGT |
| | | TATTGAGATAGATGCCCGTTTCCCGAGCCAGCTGCCGAC |
| | | TGCGGTCTATTATTCTTCCGGGATCAACCTCATAGAACT |
| | | CCTGTTCCGGGCTTTTAATGGAGGCATAGAAGAGATCAA |
| | | AACTCTCCCTGAAGACAGGTACTGCATTTACGAACATCT |
| | | CATGCTTGCAGAAAATGGAGTACTTATCCCTGTGGGAGA |
| | | ACAGGTCCTGTCCATGGGAAATGATTACGGCAATTATTA |
| | | TGAAGAACCTGGAATAGAGATTTTCCTGTGCAAAGGAG |
| | | AGAACCCTGTATTCACCCTGGTTTTCTGGGGCAGAGACA |
| | | GGGAAGAAGCTGAAGCTAGAAAAAACAAAGGGCTTTCG |
| | | ATTCTAAAAAGCCGTTTCGGAGCTGCTGCATAA |
| 21 | PylC Variant K75- | MRESWGASLKTICLIGGKLQGFEAAYLSKKAGMKVLVIDK |
| | 1 Polypeptide | NPQALIRNYADEFQCFNITEEPEKLVAISKNVDAILPVNENL |
| | Sequence; | ECIEFLNSIKEKFSCPVLFDFEAYRISRDKRKSKEYFASIGTP |
| | mutations are | TPQDKPSEPPYFVKPPCESSSVGARIIHDRKELKELEPGMLIE |
| | shown in bold and | EYVEGEVVSLNVIGDGNNFAVVKETLVHIDDTYDCHMVTP |
| | underlined letters | LPLDPSFRELSYSLAANLPLKGIMHVEAISGPLGLKVIEIDA |
| | | RFPSQLPTAVYYSSGINLIELLFRAFNGGIEEIKTLPEDRYCI |
| | | YEHLMLAENGVLIPVGEQVLSMGNDYGNYYEEPGIEIFLC |
| | | KGENPVFTLVFWGRDREEAEARKNKGLSILKSRFGAAA |
| 22 | PylC Variant K75- | ATGAGAGAGTCCTGGGGTGCTAGCCTGAAAACAATATG |
| | 3 Polynucleotide | CCTTATAGGCGGGAAGCTGCAGGGCTTCGAGGCTGCATA |
| | Sequence; | CCTATCTAAGAAAGCCGGAATGAAAGTGCTTGTAATAG |
| | mutations are | ACAAAAACCCGCAGGCGCTTATAAGGAATTATGCGGAT |
| | shown in bold and | GAGTTCCAGTGTTTTAACATAACGGAAGAGCCGGAAAA |
| | underlined letters | ACTCGTCGCGATATCAAAAAATGTTGATGCCATACTGCC |
| | | GGTAAATGAAAACCTTGAATGTATAGAATTTCTGAATTC |
| | | TATAAAAGAAAAATTCTCCTGCCCGGTACTTTTCGATTTT |
| | | GAAGCTTACAGGATCAGCAGGGATAAGAGAAAATCAAA |
| | | AGAATACTTCGCATCCATAGGAACCCCGACCCCTCAGGA |
| | | CAAACCGTCGGAACCACCTTATTTTGTAAAGCCTCCCTG |
| | | CGAAAGCAGCAGTGTGGGAGCGAGAATAATCCATGACA |
| | | GGAAAGAGCTTAAAGAGCTTGAGCCCGGGATGCTCATA |
| | | GAAGAATACGTTGAAGGGGAAGTGGTCGCGCTTGTTGT |
| | | CATAGGGGATGGAAATAATTTTGCTGTGGTAAAGGAAA |
| | | CCCTTGTACATATCGATGACACCTATGACTGCCATATGG |
| | | TGACCCCTCTCCCTCTAGACCCTTCCTTCAGGGAACTATC |
| | | CTACTCCCTTGCAGCAAACCTGCCCTTAAAAGGAATTAT |
| | | GCATGTGGAAGCGATTTCCGGCCCCCTGGGGTTAAAAGT |
| | | TATTGAGATAGATGCCCGTTTCCCGAGCCAGATTCCGAC |
| | | TGCGGTCTATTATTCTTCCGGGATCAACCTCATAGAACT |
| | | CCTGTTCCGGGCTTTTAATGGAGGCATAGAAGAGATCAA |
| | | AACTCTCCCTGAAGACAGGTACTGCATTTACGAACATCT |
| | | CATGCTTGCAGAAAATGGAGTACTTATCCCTGTGGGAGA |
| | | ACAGGTCCTGTCCATGGGAAATGATTACGGCAATTATTA |
| | | TGAAGAACCTGGAATAGAGATTTTCCTGTGCAAAGGAG |
| | | AGAACCCTGTATTCACCCTGGTTTTCTGGGGCAGAGACA |
| | | GGGAAGAAGCTGAAGCTAGAAAAAACAAAGGGCTTTCG |
| | | ATTCTAAAAAGCCGTTTCGGAGCTGCTGCATAA |
| 23 | PylC Variant K75- | MRESWGASLKTICLIGGKLQGFEAAYLSKKAGMKVLVIDK |
| | 3 Polypeptide | NPQALIRNYADEFQCFNITEEPEKLVAISKNVDAILPVNENL |
| | Sequence; | ECIEFLNSIKEKFSCPVLFDFEAYRISRDKRKSKEYFASIGTP |
| | mutations are | TPQDKPSEPPYFVKPPCESSSVGARIIHDRKELKELEPGMLIE |
| | shown in bold and | EYVEGEVVALVVIGDGNNFAVVKETLVHIDDTYDCHMVT |
| | underlined letters | PLPLDPSFRELSYSLAANLPLKGIMHVEAISGPLGLKVIEIDA |
| | | RFPSQIPTAVYYSSGINLIELLFRAFNGGIEEIKTLPEDRYCIY |
| | | EHLMLAENGVLIPVGEQVLSMGNDYGNYYEEPGIEIFLCK |
| | | GENPVFTLVFWGRDREEAEARKNKGLSILKSRFGAAA |
| 24 | PylC Variant K75- | ATGAGAGAGTCCTGGGGTGCTAGCCTGAAAACAATATG |
| | 6 Polynucleotide | CCTTATAGGCGGGAAGCTGCAGGGCTTCGAGGCTGCATA |
| | Sequence; | CCTATCTAAGAAAGCCGGAATGAAAGTGCTTGTAATAG |
| | mutations are | ACAAAAACCCGCAGGCGCTTATAAGGAATTATGCGGAT |
| | shown in bold and | GAGTTCCAGTGTTTTAACATAACGGAAGAGCCGGAAAA |
| | underlined letters | ACTCGTCGCGATATCAAAAAATGTTGATGCCATACTGCC |
| | | GGTAAATGAAAACCTTGAATGTATAGAATTTCTGAATTC |
| | | TATAAAAGAAAAATTCTCCTGCCCGGTACTTTTCGATTTT |
| | | GAAGCTTACAGGATCAGCAGGGATAAGAGAAAATCAAA |
| | | AGAATACTTCGCATCCATAGGAACCCCGACCCCTCAGGA |
| | | CAAACCGTCGGAACCACCTTATTTTGTAAAGCCTCCCTG |
| | | CGAAAGCAGCAGTGTGGGAGCGAGAATAATCCATGACA |
| | | GGAAAGAGCTTAAAGAGCTTGAGCCCGGGATGCTCATA |
| | | GAAGAATACGTTGAAGGGGAAGTGGTCCTTCTTATTGT |
| | | CATAGGGGATGGAAATAATTTTGCTGTGGTAAAGGAAA |
| | | CCCTTGTACATATCGATGACACCTATGACTGCCATATGG |
| | | TGACCCCTCTCCCTCTAGACCCTTCCTTCAGGGAACTATC |
| | | CTACTCCCTTGCAGCAAACCTGCCCTTAAAAGGAATTAT |
| | | GCATGTGGAAGCGATTTCCGGCCCCCTGGGGTTAAAAGT |
| | | TATTGAGATAGATGCCCGTTTCCCGAGCCAGTGTCCGAC |
| | | TGCGGTCTATTATTCTTCCGGGATCAACCTCATAGAACT |
| | | CCTGTTCCGGGCTTTTAATGGAGGCATAGAAGAGATCAA |
| | | AACTCTCCCTGAAGACAGGTACTGCATTTACGAACATCT |
| | | CATGCTTGCAGAAAATGGAGTACTTATCCCTGTGGGAGA |
| | | ACAGGTCCTGTCCATGGGAAATGATTACGGCAATTATTA |
| | | TGAAGAACCTGGAATAGAGATTTTCCTGTGCAAAGGAG |
| | | AGAACCCTGTATTCACCCTGGTTTTCTGGGGCAGAGACA |
| | | GGGAAGAAGCTGAAGCTAGAAAAAACAAAGGGCTTTCG |
| | | ATTCTAAAAAGCCGTTTCGGAGCTGCTGCATAA |
| 25 | PylC Variant K75- | MRESWGASLKTICLIGGKLQGFEAAYLSKKAGMKVLVIDK |
| | 6 Polypeptide | NPQALIRNYADEFQCFNITEEPEKLVAISKNVDAILPVNENL |
| | Sequence; | ECIEFLNSIKEKFSCPVLFDFEAYRISRDKRKSKEYFASIGTP |
| | mutations are | TPQDKPSEPPYFVKPPCESSSVGARIIHDRKELKELEPGMLIE |
| | shown in bold and | EYVEGEVVLLIVIGDGNNFAVVKETLVHIDDTYDCHMVTP |
| | underlined letters | LPLDPSFRELSYSLAANLPLKGIMHVEAISGPLGLKVIEIDA |
| | | RFPSQCPTAVYYSSGINLIELLFRAFNGGIEEIKTLPEDRYCI |
| | | YEHLMLAENGVLIPVGEQVLSMGNDYGNYYEEPGIEIFLC |
| | | KGENPVFTLVFWGRDREEAEARKNKGLSILKSRFGAAA |
| 26 | PylC Variant K75- | ATGAGAGAGTCCTGGGGTGCTAGCCTGAAAACAATATG |
| | 13 Polynucleotide | CCTTATAGGCGGGAAGCTGCAGGGCTTCGAGGCTGCATA |
| | Sequence; | CCTATCTAAGAAAGCCGGAATGAAAGTGCTTGTAATAG |
| | mutations are | ACAAAAACCCGCAGGCGCTTATAAGGAATTATGCGGAT |
| | shown in bold and | GAGTTCCAGTGTTTTAACATAACGGAAGAGCCGGAAAA |
| | underlined letters | ACTCGTCGCGATATCAAAAAATGTTGATGCCATACTGCC |
| | | GGTAAATGAAAACCTTGAATGTATAGAATTTCTGAATTC |
| | | TATAAAAGAAAAATTCTCCTGCCCGGTACTTTTCGATTTT |
| | | GAAGCTTACAGGATCAGCAGGGATAAGAGAAAATCAAA |
| | | AGAATACTTCGCATCCATAGGAACCCCGACCCCTCAGGA |
| | | CAAACCGTCGGAACCACCTTATTTTGTAAAGCCTCCCTG |
| | | CGAAAGCAGCAGTGTGGGAGCGAGAATAATCCATGACA |
| | | GGAAAGAGCTTAAAGAGCTTGAGCCCGGGATGCTCATA |
| | | GAAGAATACGTTGAAGGGGAAGTGGTCGCGCTTATTGT |
| | | CATAGGGGATGGAAATAATTTTGCTGTGGTAAAGGAAA |
| | | CCCTTGTACATATCGATGACACCTATGACTGCCATATGG |
| | | TGACCCCTCTCCCTCTAGACCCTTCCTTCAGGGAACTATC |
| | | CTACTCCCTTGCAGCAAACCTGCCCTTAAAAGGAATTAT |
| | | GACTGTGGAAGCGATTTCCGGCCCCCTGGGGTTAAAAGT |
| | | TATTGAGATAGATGCCCGTTTCCCGAGCCAGTTGCCGAC |
| | | TGCGGTCTATTATTCTTCCGGGATCAACCTCATAGAACT |
| | | CCTGTTCCGGGCTTTTAATGGAGGCATAGAAGAGATCAA |
| | | AACTCTCCCTGAAGACAGGTACTGCATTTACGAACATCT |
| | | CATGCTTGCAGAAAATGGAGTACTTATCCCTGTGGGAGA |
| | | ACAGGTCCTGTCCATGGGAAATGATTACGGCAATTATTA |
| | | TGAAGAACCTGGAATAGAGATTTTCCTGTGCAAAGGAG |
| | | AGAACCCTGTATTCACCCTGGTTTTCTGGGGCAGAGACA |
| | | GGGAAGAAGCTGAAGCTAGAAAAAACAAAGGGCTTTCG |
| | | ATTCTAAAAAGCCGTTTCGGAGCTGCTGCATAA |
| 27 | PylC Variant K75- | MRESWGASLKTICLIGGKLQGFEAAYLSKKAGMKVLVIDK |
| | 13 Polypeptide | NPQALIRNYADEFQCFNITEEPEKLVAISKNVDAILPVNENL |
| | Sequence; | ECIEFLNSIKEKFSCPVLFDFEAYRISRDKRKSKEYFASIGTP |
| | mutations are | TPQDKPSEPPYFVKPPCESSSVGARIIHDRKELKELEPGMLIE |
| | shown in bold and | EYVEGEVVALIVIGDGNNFAVVKETLVHIDDTYDCHMVTP |
| | underlined letters | LPLDPSFRELSYSLAANLPLKGIMTVEAISGPLGLKVIEIDAR |
| | | FPSQLPTAVYYSSGINLIELLFRAFNGGIEEIKTLPEDRYCIY |
| | | EHLMLAENGVLIPVGEQVLSMGNDYGNYYEEPGIEIFLCK |
| | | GENPVFTLVFWGRDREEAEARKNKGLSILKSRFGAAA |
| 28 | PylC Variant K75- | ATGAGAGAGTCCTGGGGTGCTAGCCTGAAAACAATATG |
| | 15 Polynucleotide | CCTTATAGGCGGGAAGCTGCAGGGCTTCGAGGCTGCATA |
| | Sequence; | CCTATCTAAGAAAGCCGGAATGAAAGTGCTTGTAATAG |
| | mutations are | ACAAAAACCCGCAGGCGCTTATAAGGAATTATGCGGAT |
| | shown in bold and | GAGTTCCAGTGTTTTAACATAACGGAAGAGCCGGAAAA |
| | underlined letters | ACTCGTCGCGATATCAAAAAATGTTGATGCCATACTGCC |
| | | GGTAAATGAAAACCTTGAATGTATAGAATTTCTGAATTC |
| | | TATAAAAGAAAAATTCTCCTGCCCGGTACTTTTCGATTTT |
| | | GAAGCTTACAGGATCAGCAGGGATAAGAGAAAATCAAA |
| | | AGAATACTTCGCATCCATAGGAACCCCGACCCCTCAGGA |
| | | CAAACCGTCGGAACCACCTTATTTTGTAAAGCCTCCCTG |
| | | CGAAAGCAGCAGTGTGGGAGCGAGAATAATCCATGACA |
| | | GGAAAGAGCTTAAAGAGCTTGAGCCCGGGATGCTCATA |
| | | GAAGAATACGTTGAAGGGGAAGTGGTCCATCTTCTGGT |
| | | CATAGGGGATGGAAATAATTTTGCTGTGGTAAAGGAAA |
| | | CCCTTGTACATATCGATGACACCTATGACTGCCATATGG |
| | | TGACCCCTCTCCCTCTAGACCCTTCCTTCAGGGAACTATC |
| | | CTACTCCCTTGCAGCAAACCTGCCCTTAAAAGGAATTAT |
| | | GTCGGTGGAAGCGATTTCCGGCCCCCTGGGGTTAAAAG |
| | | TTATTGAGATAGATGCCCGTTTCCCGAGCCAGGTGCCGA |
| | | CTGCGGTCTATTATTCTTCCGGGATCAACCTCATAGAAC |
| | | TCCTGTTCCGGGCTTTTAATGGAGGCATAGAAGAGATCA |
| | | AAACTCTCCCTGAAGACAGGTACTGCATTTACGAACATC |
| | | TCATGCTTGCAGAAAATGGAGTACTTATCCCTGTGGGAG |
| | | AACAGGTCCTGTCCATGGGAAATGATTACGGCAATTATT |
| | | ATGAAGAACCTGGAATAGAGATTTTCCTGTGCAAAGGA |
| | | GAGAACCCTGTATTCACCCTGGTTTTCTGGGGCAGAGAC |
| | | AGGGAAGAAGCTGAAGCTAGAAAAAACAAAGGGCTTTC |
| | | GATTCTAAAAAGCCGTTTCGGAGCTGCTGCATAA |
| 29 | PylC Variant K75- | MRESWGASLKTICLIGGKLQGFEAAYLSKKAGMKVLVIDK |
| | 15 Polypeptide | NPQALIRNYADEFQCFNITEEPEKLVAISKNVDAILPVNENL |
| | Sequence; | ECIEFLNSIKEKFSCPVLFDFEAYRISRDKRKSKEYFASIGTP |
| | mutations are | TPQDKPSEPPYFVKPPCESSSVGARIIHDRKELKELEPGMLIE |
| | shown in bold and | EYVEGEVVHLLVIGDGNNFAVVKETLVHIDDTYDCHMVT |
| | underlined letters | PLPLDPSFRELSYSLAANLPLKGIMSVEAISGPLGLKVIEIDA |
| | | RFPSQVPTAVYYSSGINLIELLFRAFNGGIEEIKTLPEDRYCI |
| | | YEHLMLAENGVLIPVGEQVLSMGNDYGNYYEEPGIEIFLC |
| | | KGENPVFTLVFWGRDREEAEARKNKGLSILKSRFGAAA |
| 30 | PylC Variant K75- | ATGAGAGAGTCCTGGGGTGCTAGCCTGAAAACAATATG |
| | 19 Polynucleotide | CCTTATAGGCGGGAAGCTGCAGGGCTTCGAGGCTGCATA |
| | Sequence; | CCTATCTAAGAAAGCCGGAATGAAAGTGCTTGTAATAG |
| | mutations are | ACAAAAACCCGCAGGCGCTTATAAGGAATTATGCGGAT |
| | shown in bold and | GAGTTCCAGTGTTTTAACATAACGGAAGAGCCGGAAAA |
| | underlined letters | ACTCGTCGCGATATCAAAAAATGTTGATGCCATACTGCC |
| | | GGTAAATGAAAACCTTGAATGTATAGAATTTCTGAATTC |
| | | TATAAAAGAAAAATTCTCCTGCCCGGTACTTTTCGATTTT |
| | | GAAGCTTACAGGATCAGCAGGGATAAGAGAAAATCAAA |
| | | AGAATACTTCGCATCCATAGGAACCCCGACCCCTCAGGA |
| | | CAAACCGTCGGAACCACCTTATTTTGTAAAGCCTCCCTG |
| | | CGAAAGCAGCAGTGTGGGAGCGAGAATAATCCATGACA |
| | | GGAAAGAGCTTAAAGAGCTTGAGCCCGGGATGCTCATA |
| | | GAAGAATACGTTGAAGGGGAAGTGGTCGCTCTTATGGT |
| | | CATAGGGGATGGAAATAATTTTGCTGTGGTAAAGGAAA |
| | | CCCTTGTACATATCGATGACACCTATGACTGCCATATGG |
| | | TGACCCCTCTCCCTCTAGACCCTTCCTTCAGGGAACTATC |
| | | CTACTCCCTTGCAGCAAACCTGCCCTTAAAAGGAATTAT |
| | | GAGTGTGGAAGCGATTTCCGGCCCCCTGGGGTTAAAAG |
| | | TTATTGAGATAGATGCCCGTTTCCCGAGCCAGCTTCCGA |
| | | CTGCGGTCTATTATTCTTCCGGGATCAACCTCATAGAAC |
| | | TCCTGTTCCGGGCTTTTAATGGAGGCATAGAAGAGATCA |
| | | AAACTCTCCCTGAAGACAGGTACTGCATTTACGAACATC |
| | | TCATGCTTGCAGAAAATGGAGTACTTATCCCTGTGGGAG |
| | | AACAGGTCCTGTCCATGGGAAATGATTACGGCAATTATT |
| | | ATGAAGAACCTGGAATAGAGATTTTCCTGTGCAAAGGA |
| | | GAGAACCCTGTATTCACCCTGGTTTTCTGGGGCAGAGAC |
| | | AGGGAAGAAGCTGAAGCTAGAAAAAACAAAGGGCTTTC |
| | | GATTCTAAAAAGCCGTTTCGGAGCTGCTGCATAA |
| 31 | PylC Variant K75- | MRESWGASLKTICLIGGKLQGFEAAYLSKKAGMKVLVIDK |
| | 19 Polypeptide | NPQALIRNYADEFQCFNITEEPEKLVAISKNVDAILPVNENL |
| | Sequence; | ECIEFLNSIKEKFSCPVLFDFEAYRISRDKRKSKEYFASIGTP |
| | mutations are | TPQDKPSEPPYFVKPPCESSSVGARIIHDRKELKELEPGMLIE |
| | shown in bold and | EYVEGEVVALMVIGDGNNFAVVKETLVHIDDTYDCHMVT |
| | underlined letters | PLPLDPSFRELSYSLAANLPLKGIMSVEAISGPLGLKVIEIDA |
| | | RFPSQLPTAVYYSSGINLIELLFRAFNGGIEEIKTLPEDRYCI |
| | | YEHLMLAENGVLIPVGEQVLSMGNDYGNYYEEPGIEIFLC |
| | | KGENPVFTLVFWGRDREEAEARKNKGLSILKSRFGAAA |
| 32 | PylC Variant K75- | ATGAGAGAGTCCTGGGGTGCTAGCCTGAAAACAATATG |
| | 22 Polynucleotide | CCTTATAGGCGGGAAGCTGCAGGGCTTCGAGGCTGCATA |
| | Sequence; | CCTATCTAAGAAAGCCGGAATGAAAGTGCTTGTAATAG |
| | mutations are | ACAAAAACCCGCAGGCGCTTATAAGGAATTATGCGGAT |
| | shown in bold and | GAGTTCCAGTGTTTTAACATAACGGAAGAGCCGGAAAA |
| | underlined letters | ACTCGTCGCGATATCAAAAAATGTTGATGCCATACTGCC |
| | | GGTAAATGAAAACCTTGAATGTATAGAATTTCTGAATTC |
| | | TATAAAAGAAAAATTCTCCTGCCCGGTACTTTTCGATTTT |
| | | GAAGCTTACAGGATCAGCAGGGATAAGAGAAAATCAAA |
| | | AGAATACTTCGCATCCATAGGAACCCCGACCCCTCAGGA |
| | | CAAACCGTCGGAACCACCTTATTTTGTAAAGCCTCCCTG |
| | | CGAAAGCAGCAGTGTGGGAGCGAGAATAATCCATGACA |
| | | GGAAAGAGCTTAAAGAGCTTGAGCCCGGGATGCTCATA |
| | | GAAGAATACGTTGAAGGGGAAGTGGTCTCGCTTATTGT |
| | | CATAGGGGATGGAAATAATTTTGCTGTGGTAAAGGAAA |
| | | CCCTTGTACATATCGATGACACCTATGACTGCCATATGG |
| | | TGACCCCTCTCCCTCTAGACCCTTCCTTCAGGGAACTATC |
| | | CTACTCCCTTGCAGCAAACCTGCCCTTAAAAGGAATTAT |
| | | GCATGTGGAAGCGATTTCCGGCCCCCTGGGGTTAAAAGT |
| | | TATTGAGATAGATGCCCGTTTCCCGAGCCAGATTCCGAC |
| | | TGCGGTCTATTATTCTTCCGGGATCAACCTCATAGAACT |
| | | CCTGTTCCGGGCTTTTAATGGAGGCATAGAAGAGATCAA |
| | | AACTCTCCCTGAAGACAGGTACTGCATTTACGAACATCT |
| | | CATGCTTGCAGAAAATGGAGTACTTATCCCTGTGGGAGA |
| | | ACAGGTCCTGTCCATGGGAAATGATTACGGCAATTATTA |
| | | TGAAGAACCTGGAATAGAGATTTTCCTGTGCAAAGGAG |
| | | AGAACCCTGTATTCACCCTGGTTTTCTGGGGCAGAGACA |
| | | GGGAAGAAGCTGAAGCTAGAAAAAACAAAGGGCTTTCG |
| | | ATTCTAAAAAGCCGTTTCGGAGCTGCTGCATAA |
| 33 | PylC Variant K75- | MRESWGASLKTICLIGGKLQGFEAAYLSKKAGMKVLVIDK |
| | 22 Polypeptide | NPQALIRNYADEFQCFNITEEPEKLVAISKNVDAILPVNENL |
| | Sequence; | ECIEFLNSIKEKFSCPVLFDFEAYRISRDKRKSKEYFASIGTP |
| | mutations are | TPQDKPSEPPYFVKPPCESSSVGARIIHDRKELKELEPGMLIE |
| | shown in bold and | EYVEGEVVSLIVIGDGNNFAVVKETLVHIDDTYDCHMVTP |
| | underlined letters | LPLDPSFRELSYSLAANLPLKGIMHVEAISGPLGLKVIEIDA |
| | | RFPSQIPTAVYYSSGINLIELLFRAFNGGIEEIKTLPEDRYCIY |
| | | EHLMLAENGVLIPVGEQVLSMGNDYGNYYEEPGIEIFLCK |
| | | GENPVFTLVFWGRDREEAEARKNKGLSILKSRFGAAA |
| 34 | PylC Variant K75- | ATGAGAGAGTCCTGGGGTGCTAGCCTGAAAACAATATG |
| | 29 Polynucleotide | CCTTATAGGCGGGAAGCTGCAGGGCTTCGAGGCTGCATA |
| | Sequence; | CCTATCTAAGAAAGCCGGAATGAAAGTGCTTGTAATAG |
| | mutations are | ACAAAAACCCGCAGGCGCTTATAAGGAATTATGCGGAT |
| | shown in bold and | GAGTTCCAGTGTTTTAACATAACGGAAGAGCCGGAAAA |
| | underlined letters | ACTCGTCGCGATATCAAAAAATGTTGATGCCATACTGCC |
| | | GGTAAATGAAAACCTTGAATGTATAGAATTTCTGAATTC |
| | | TATAAAAGAAAAATTCTCCTGCCCGGTACTTTTCGATTTT |
| | | GAAGCTTACAGGATCAGCAGGGATAAGAGAAAATCAAA |
| | | AGAATACTTCGCATCCATAGGAACCCCGACCCCTCAGGA |
| | | CAAACCGTCGGAACCACCTTATTTTGTAAAGCCTCCCTG |
| | | CGAAAGCAGCAGTGTGGGAGCGAGAATAATCCATGACA |
| | | GGAAAGAGCTTAAAGAGCTTGAGCCCGGGATGCTCATA |
| | | GAAGAATACGTTGAAGGGGAAGTGGTCGCTCTTACGGT |
| | | CATAGGGGATGGAAATAATTTTGCTGTGGTAAAGGAAA |
| | | CCCTTGTACATATCGATGACACCTATGACTGCCATATGG |
| | | TGACCCCTCTCCCTCTAGACCCTTCCTTCAGGGAACTATC |
| | | CTACTCCCTTGCAGCAAACCTGCCCTTAAAAGGAATTAT |
| | | GCATGTGGAAGCGATTTCCGGCCCCCTGGGGTTAAAAGT |
| | | TATTGAGATAGATGCCCGTTTCCCGAGCCAGGTTCCGAC |
| | | TGCGGTCTATTATTCTTCCGGGATCAACCTCATAGAACT |
| | | CCTGTTCCGGGCTTTTAATGGAGGCATAGAAGAGATCAA |
| | | AACTCTCCCTGAAGACAGGTACTGCATTTACGAACATCT |
| | | CATGCTTGCAGAAAATGGAGTACTTATCCCTGTGGGAGA |
| | | ACAGGTCCTGTCCATGGGAAATGATTACGGCAATTATTA |
| | | TGAAGAACCTGGAATAGAGATTTTCCTGTGCAAAGGAG |
| | | AGAACCCTGTATTCACCCTGGTTTTCTGGGGCAGAGACA |
| | | GGGAAGAAGCTGAAGCTAGAAAAAACAAAGGGCTTTCG |
| | | ATTCTAAAAAGCCGTTTCGGAGCTGCTGCATAA |
| 35 | PylC Variant K75- | MRESWGASLKTICLIGGKLQGFEAAYLSKKAGMKVLVIDK |
| | 29 Polypeptide | NPQALIRNYADEFQCFNITEEPEKLVAISKNVDAILPVNENL |
| | Sequence; | ECIEFLNSIKEKFSCPVLFDFEAYRISRDKRKSKEYFASIGTP |
| | mutations are | TPQDKPSEPPYFVKPPCESSSVGARIIHDRKELKELEPGMLIE |
| | shown in bold and | EYVEGEVVALTVIGDGNNFAVVKETLVHIDDTYDCHMVT |
| | underlined letters | PLPLDPSFRELSYSLAANLPLKGIMHVEAISGPLGLKVIEIDA |
| | | RFPSQVPTAVYYSSGINLIELLFRAFNGGIEEIKTLPEDRYCI |
| | | YEHLMLAENGVLIPVGEQVLSMGNDYGNYYEEPGIEIFLC |
| | | KGENPVFTLVFWGRDREEAEARKNKGLSILKSRFGAAA |
| 36 | PylC Variant K75- | ATGAGAGAGTCCTGGGGTGCTAGCCTGAAAACAATATG |
| | 30 Polynucleotide | CCTTATAGGCGGGAAGCTGCAGGGCTTCGAGGCTGCATA |
| | Sequence; | CCTATCTAAGAAAGCCGGAATGAAAGTGCTTGTAATAG |
| | mutations are | ACAAAAACCCGCAGGCGCTTATAAGGAATTATGCGGAT |
| | shown in bold and | GAGTTCCAGTGTTTTAACATAACGGAAGAGCCGGAAAA |
| | underlined letters | ACTCGTCGCGATATCAAAAAATGTTGATGCCATACTGCC |
| | | GGTAAATGAAAACCTTGAATGTATAGAATTTCTGAATTC |
| | | TATAAAAGAAAAATTCTCCTGCCCGGTACTTTTCGATTTT |
| | | GAAGCTTACAGGATCAGCAGGGATAAGAGAAAATCAAA |
| | | AGAATACTTCGCATCCATAGGAACCCCGACCCCTCAGGA |
| | | CAAACCGTCGGAACCACCTTATTTTGTAAAGCCTCCCTG |
| | | CGAAAGCAGCAGTGTGGGAGCGAGAATAATCCATGACA |
| | | GGAAAGAGCTTAAAGAGCTTGAGCCCGGGATGCTCATA |
| | | GAAGAATACGTTGAAGGGGAAGTGGTCGCGCTTGCGGT |
| | | CATAGGGGATGGAAATAATTTTGCTGTGGTAAAGGAAA |
| | | CCCTTGTACATATCGATGACACCTATGACTGCCATATGG |
| | | TGACCCCTCTCCCTCTAGACCCTTCCTTCAGGGAACTATC |
| | | CTACTCCCTTGCAGCAAACCTGCCCTTAAAAGGAATTAT |
| | | GAATGTGGAAGCGATTTCCGGCCCCCTGGGGTTAAAAGT |
| | | TATTGAGATAGATGCCCGTTTCCCGAGCCAGTGTCCGAC |
| | | TGCGGTCTATTATTCTTCCGGGATCAACCTCATAGAACT |
| | | CCTGTTCCGGGCTTTTAATGGAGGCATAGAAGAGATCAA |
| | | AACTCTCCCTGAAGACAGGTACTGCATTTACGAACATCT |
| | | CATGCTTGCAGAAAATGGAGTACTTATCCCTGTGGGAGA |
| | | ACAGGTCCTGTCCATGGGAAATGATTACGGCAATTATTA |
| | | TGAAGAACCTGGAATAGAGATTTTCCTGTGCAAAGGAG |
| | | AGAACCCTGTATTCACCCTGGTTTTCTGGGGCAGAGACA |
| | | GGGAAGAAGCTGAAGCTAGAAAAAACAAAGGGCTTTCG |
| | | ATTCTAAAAAGCCGTTTCGGAGCTGCTGCATAA |
| 37 | PylC Variant K75- | MRESWGASLKTICLIGGKLQGFEAAYLSKKAGMKVLVIDK |
| | 30 Polypeptide | NPQALIRNYADEFQCFNITEEPEKLVAISKNVDAILPVNENL |
| | Sequence; | ECIEFLNSIKEKFSCPVLFDFEAYRISRDKRKSKEYFASIGTP |
| | mutations are | TPQDKPSEPPYFVKPPCESSSVGARIIHDRKELKELEPGMLIE |
| | shown in bold and | EYVEGEVVALAVIGDGNNFAVVKETLVHIDDTYDCHMVT |
| | underlined letters | PLPLDPSFRELSYSLAANLPLKGIMNVEAISGPLGLKVIEIDA |
| | PylC Variant | RFPSQCPTAVYYSSGINLIELLFRAFNGGIEEIKTLPEDRYCI |
| | | YEHLMLAENGVLIPVGEQVLSMGNDYGNYYEEPGIEIFLC |
| | | KGENPVFTLVFWGRDREEAEARKNKGLSILKSRFGAAA |
| 38 | K150-1 | ATGAGAGAGTCCTGGGGTGCTAGCCTGAAAACAATATG |
| | Polynucleotide | CCTTATAGGCGGGAAGCTGCAGGGCTTCGAGGCTGCATA |
| | Sequence; | CCTATCTAAGAAAGCCGGAATGAAAGTGCTTGTAATAG |
| | mutations are | ACAAAAACCCGCAGGCGCTTATAAGGAATTATGCGGAT |
| | shown in bold and | GAGTTCCAGTGTTTTAACATAACGGAAGAGCCGGAAAA |
| | underlined letters | ACTCGTCGCGATATCAAAAAATGTTGATGCCATACTGCC |
| | | GGTAAATGAAAACCTTGAATGTATAGAATTTCTGAATTC |
| | | TATAAAAGAAAAATTCTCCTGCCCGGTACTTTTCGATTTT |
| | | GAAGCTTACAGGATCAGCAGGGATAAGAGAAAATCAAA |
| | | AGAATACTTCGCATCCATAGGAACCCCGACCCCTCAGGA |
| | | CAAACCGTCGGAACCACCTTATTTTGTAAAGCCTCCCTG |
| | | CGAAAGCAGCAGTGTGGGAGCGAGAATAATCCATGACA |
| | | GGAAAGAGCTTAAAGAGCTTGAGCCCGGGATGCTCATA |
| | | GAAGAATACGTTGAAGGGGAAGTGGTCTGTCTTCCTGT |
| | | CATAGGGGATGGAAATAATTTTGCTGTGGTAAAGGAAA |
| | | CCCTTGTACATATCGATGACACCTATGACTGCCATATGG |
| | | TGACCCCTCTCCCTCTAGACCCTTCCTTCAGGGAACTATC |
| | | CTACTCCCTTGCAGCAAACCTGCCCTTAAAAGGAATTAT |
| | | GCATGTGGAAGCGATTTCCGGCCCCCTGGGGTTAAAAGT |
| | | TATTGAGATAGATGCCCGTTTCCCGAGCCAGCTTCCGAC |
| | | TGCGGTCTATTATTCTTCCGGGATCAACCTCATAGAACT |
| | | CCTGTTCCGGGCTTTTAATGGAGGCATAGAAGAGATCAA |
| | | AACTCTCCCTGAAGACAGGTACTGCATTTACGAACATCT |
| | | CATGCTTGCAGAAAATGGAGTACTTATCCCTGTGGGAGA |
| | | ACAGGTCCTGTCCATGGGAAATGATTACGGCAATTATTA |
| | | TGAAGAACCTGGAATAGAGATTTTCCTGTGCAAAGGAG |
| | | AGAACCCTGTATTCACCCTGGTTTTCTGGGGCAGAGACA |
| | | GGGAAGAAGCTGAAGCTAGAAAAAACAAAGGGCTTTCG |
| | | ATTCTAAAAAGCCGTTTCGGAGCTGCTGCATAA |
| 39 | PylC Variant | MRESWGASLKTICLIGGKLQGFEAAYLSKKAGMKVLVIDK |
| | K150-1 | NPQALIRNYADEFQCFNITEEPEKLVAISKNVDAILPVNENL |
| | Polypeptide | ECIEFLNSIKEKFSCPVLFDFEAYRISRDKRKSKEYFASIGTP |
| | Sequence; | TPQDKPSEPPYFVKPPCESSSVGARIIHDRKELKELEPGMLIE |
| | mutations are | EYVEGEVVCLPVIGDGNNFAVVKETLVHIDDTYDCHMVTP |
| | shown in bold and | LPLDPSFRELSYSLAANLPLKGIMHVEAISGPLGLKVIEIDA |
| | underlined letters | RFPSQLPTAVYYSSGINLIELLFRAFNGGIEEIKTLPEDRYCI |
| | | YEHLMLAENGVLIPVGEQVLSMGNDYGNYYEEPGIEIFLC |
| | | KGENPVFTLVFWGRDREEAEARKNKGLSILKSRFGAAA |
| 40 | PylC Variant | ATGAGAGAGTCCTGGGGTGCTAGCCTGAAAACAATATG |
| | K150-37 | CCTTATAGGCGGGAAGCTGCAGGGCTTCGAGGCTGCATA |
| | Polynucleotide | CCTATCTAAGAAAGCCGGAATGAAAGTGCTTGTAATAG |
| | Sequence; | ACAAAAACCCGCAGGCGCTTATAAGGAATTATGCGGAT |
| | mutations are | GAGTTCCAGTGTTTTAACATAACGGAAGAGCCGGAAAA |
| | shown in bold and | ACTCGTCGCGATATCAAAAAATGTTGATGCCATACTGCC |
| | underlined letters | GGTAAATGAAAACCTTGAATGTATAGAATTTCTGAATTC |
| | | TATAAAAGAAAAATTCTCCTGCCCGGTACTTTTCGATTTT |
| | | GAAGCTTACAGGATCAGCAGGGATAAGAGAAAATCAAA |
| | | AGAATACTTCGCATCCATAGGAACCCCGACCCCTCAGGA |
| | | CAAACCGTCGGAACCACCTTATTTTGTAAAGCCTCCCTG |
| | | CGAAAGCAGCAGTGTGGGAGCGAGAATAATCCATGACA |
| | | GGAAAGAGCTTAAAGAGCTTGAGCCCGGGATGCTCATA |
| | | GAAGAATACGTTGAAGGGGAAGTGGTCGGTCTTATTGT |
| | | CATAGGGGATGGAAATAATTTTGCTGTGGTAAAGGAAA |
| | | CCCTTGTACATATCGATGACACCTATGACTGCCATATGG |
| | | TGACCCCTCTCCCTCTAGACCCTTCCTTCAGGGAACTATC |
| | | CTACTCCCTTGCAGCAAACCTGCCCTTAAAAGGAATTAT |
| | | GCATGTGGAAGCGATTTCCGGCCCCCTGGGGTTAAAAGT |
| | | TATTGAGATAGATGCCCGTTTCCCGAGCCAGACGCCGAC |
| | | TGCGGTCTATTATTCTTCCGGGATCAACCTCATAGAACT |
| | | CCTGTTCCGGGCTTTTAATGGAGGCATAGAAGAGATCAA |
| | | AACTCTCCCTGAAGACAGGTACTGCATTTACGAACATCT |
| | | CATGCTTGCAGAAAATGGAGTACTTATCCCTGTGGGAGA |
| | | ACAGGTCCTGTCCATGGGAAATGATTACGGCAATTATTA |
| | | TGAAGAACCTGGAATAGAGATTTTCCTGTGCAAAGGAG |
| | | AGAACCCTGTATTCACCCTGGTTTTCTGGGGCAGAGACA |
| | | GGGAAGAAGCTGAAGCTAGAAAAAACAAAGGGCTTTCG |
| | | ATTCTAAAAAGCCGTTTCGGAGCTGCTGCATAA |
| 41 | PylC Variant | MRESWGASLKTICLIGGKLQGFEAAYLSKKAGMKVLVIDK |
| | K150-37 | NPQALIRNYADEFQCFNITEEPEKLVAISKNVDAILPVNENL |
| | Polypeptide | ECIEFLNSIKEKFSCPVLFDFEAYRISRDKRKSKEYFASIGTP |
| | Sequence; | TPQDKPSEPPYFVKPPCESSSVGARIIHDRKELKELEPGMLIE |
| | mutations are | EYVEGEVVGLIVIGDGNNFAVVKETLVHIDDTYDCHMVTP |
| | shown in bold and | LPLDPSFRELSYSLAANLPLKGIMHVEAISGPLGLKVIEIDA |
| | underlined letters | RFPSQTPTAVYYSSGINLIELLFRAFNGGIEEIKTLPEDRYCI |
| | | YEHLMLAENGVLIPVGEQVLSMGNDYGNYYEEPGIEIFLC |
| | | KGENPVFTLVFWGRDREEAEARKNKGLSILKSRFGAAA |
| 42 | PylC Variant | ATGAGAGAGTCCTGGGGTGCTAGCCTGAAAACAATATG |
| | K200-10 | CCTTATAGGCGGGAAGCTGCAGGGCTTCGAGGCTGCATA |
| | Polynucleotide | CCTATCTAAGAAAGCCGGAATGAAAGTGCTTGTAATAG |
| | Sequence; | ACAAAAACCCGCAGGCGCTTATAAGGAATTATGCGGAT |
| | mutations are | GAGTTCCAGTGTTTTAACATAACGGAAGAGCCGGAAAA |
| | shown in bold and | ACTCGTCGCGATATCAAAAAATGTTGATGCCATACTGCC |
| | underlined letters | GGTAAATGAAAACCTTGAATGTATAGAATTTCTGAATTC |
| | | TATAAAAGAAAAATTCTCCTGCCCGGTACTTTTCGATTTT |
| | | GAAGCTTACAGGATCAGCAGGGATAAGAGAAAATCAAA |
| | | AGAATACTTCGCATCCATAGGAACCCCGACCCCTCAGGA |
| | | CAAACCGTCGGAACCACCTTATTTTGTAAAGCCTCCCTG |
| | | CGAAAGCAGCAGTGTGGGAGCGAGAATAATCCATGACA |
| | | GGAAAGAGCTTAAAGAGCTTGAGCCCGGGATGCTCATA |
| | | GAAGAATACGTTGAAGGGGAAGTGGTCATGCTTGTTGT |
| | | CATAGGGGATGGAAATAATTTTGCTGTGGTAAAGGAAA |
| | | CCCTTGTACATATCGATGACACCTATGACTGCCATATGG |
| | | TGACCCCTCTCCCTCTAGACCCTTCCTTCAGGGAACTATC |
| | | CTACTCCCTTGCAGCAAACCTGCCCTTAAAAGGAATTAT |
| | | GCATGTGGAAGCGATTTCCGGCCCCCTGGGGTTAAAAGT |
| | | TATTGAGATAGATGCCCGTTTCCCGAGCCAGGTTCCGAC |
| | | TGCGGTCTATTATTCTTCCGGGATCAACCTCATAGAACT |
| | | CCTGTTCCGGGCTTTTAATGGAGGCATAGAAGAGATCAA |
| | | AACTCTCCCTGAAGACAGGTACTGCATTTACGAACATCT |
| | | CATGCTTGCAGAAAATGGAGTACTTATCCCTGTGGGAGA |
| | | ACAGGTCCTGTCCATGGGAAATGATTACGGCAATTATTA |
| | | TGAAGAACCTGGAATAGAGATTTTCCTGTGCAAAGGAG |
| | | AGAACCCTGTATTCACCCTGGTTTTCTGGGGCAGAGACA |
| | | GGGAAGAAGCTGAAGCTAGAAAAAACAAAGGGCTTTCG |
| | | ATTCTAAAAAGCCGTTTCGGAGCTGCTGCATAA |
| 43 | PylC Variant | MRESWGASLKTICLIGGKLQGFEAAYLSKKAGMKVLVIDK |
| | K200-10 | NPQALIRNYADEFQCFNITEEPEKLVAISKNVDAILPVNENL |
| | Polypeptide | ECIEFLNSIKEKFSCPVLFDFEAYRISRDKRKSKEYFASIGTP |
| | Sequence; | TPQDKPSEPPYFVKPPCESSSVGARIIHDRKELKELEPGMLIE |
| | mutations are | EYVEGEVVMLVVIGDGNNFAVVKETLVHIDDTYDCHMVT |
| | shown in bold and | PLPLDPSFRELSYSLAANLPLKGIMHVEAISGPLGLKVIEIDA |
| | underlined letters | RFPSQVPTAVYYSSGINLIELLFRAFNGGIEEIKTLPEDRYCI |
| | | YEHLMLAENGVLIPVGEQVLSMGNDYGNYYEEPGIEIFLC |
| | | KGENPVFTLVFWGRDREEAEARKNKGLSILKSRFGAAA |
| 44 | PylC Variant | ATGAGAGAGTCCTGGGGTGCTAGCCTGAAAACAATATG |
| | K150-21 | CCTTATAGGCGGGAAGCTGCAGGGCTTCGAGGCTGCATA |
| | Polynucleotide | CCTATCTAAGAAAGCCGGAATGAAAGTGCTTGTAATAG |
| | Sequence; | ACAAAAACCCGCAGGCGCTTATAAGGAATTATGCGGAT |
| | mutations are | GAGTTCCAGTGTTTTAACATAACGGAAGAGCCGGAAAA |
| | shown in bold and | ACTCGTCGCGATATCAAAAAATGTTGATGCCATACTGCC |
| | underlined letters | GGTAAATGAAAACCTTGAATGTATAGAATTTCTGAATTC |
| | | TATAAAAGAAAAATTCTCCTGCCCGGTACTTTTCGATTTT |
| | | GAAGCTTACAGGATCAGCAGGGATAAGAGAAAATCAAA |
| | | AGAATACTTCGCATCCATAGGAACCCCGACCCCTCAGGA |
| | | CAAACCGTCGGAACCACCTTATTTTGTAAAGCCTCCCTG |
| | | CGAAAGCAGCAGTGTGGGAGCGAGAATAATCCATGACA |
| | | GGAAAGAGCTTAAAGAGCTTGAGCCCGGGATGCTCATA |
| | | GAAGAATACGTTGAAGGGGAAGTGGTCGTGCTTGTGGT |
| | | CATAGGGGATGGAAATAATTTTGCTGTGGTAAAGGAAA |
| | | CCCTTGTACATATCGATGACACCTATGACTGCCATATGG |
| | | TGACCCCTCTCCCTCTAGACCCTTCCTTCAGGGAACTATC |
| | | CTACTCCCTTGCAGCAAACCTGCCCTTAAAAGGAATTAT |
| | | GCATGTGGAAGCGATTTCCGGCCCCCTGGGGTTAAAAGT |
| | | TATTGAGATAGATGCCCGTTTCCCGAGCCAGGTGCCGAC |
| | | TGCGGTCTATTATTCTTCCGGGATCAACCTCATAGAACT |
| | | CCTGTTCCGGGCTTTTAATGGAGGCATAGAAGAGATCAA |
| | | AACTCTCCCTGAAGACAGGTACTGCATTTACGAACATCT |
| | | CATGCTTGCAGAAAATGGAGTACTTATCCCTGTGGGAGA |
| | | ACAGGTCCTGTCCATGGGAAATGATTACGGCAATTATTA |
| | | TGAAGAACCTGGAATAGAGATTTTCCTGTGCAAAGGAG |
| | | AGAACCCTGTATTCACCCTGGTTTTCTGGGGCAGAGACA |
| | | GGGAAGAAGCTGAAGCTAGAAAAAACAAAGGGCTTTCG |
| | | ATTCTAAAAAGCCGTTTCGGAGCTGCTGCATAA |
| 45 | PylC Variant | MRESWGASLKTICLIGGKLQGFEAAYLSKKAGMKVLVIDK |
| | K150-21 | NPQALIRNYADEFQCFNITEEPEKLVAISKNVDAILPVNENL |
| | Polypeptide | ECIEFLNSIKEKFSCPVLFDFEAYRISRDKRKSKEYFASIGTP |
| | Sequence; | TPQDKPSEPPYFVKPPCESSSVGARIIHDRKELKELEPGMLIE |
| | mutations are | EYVEGEVVVLVVIGDGNNFAVVKETLVHIDDTYDCHMVT |
| | shown in bold and | PLPLDPSFRELSYSLAANLPLKGIMHVEAISGPLGLKVIEIDA |
| | underlined letters | RFPSQVPTAVYYSSGINLIELLFRAFNGGIEEIKTLPEDRYCI |
| | | YEHLMLAENGVLIPVGEQVLSMGNDYGNYYEEPGIEIFLC |
| | | KGENPVFTLVFWGRDREEAEARKNKGLSILKSRFGAAA |
| 46 | PylC Variant | ATGAGAGAGTCCTGGGGTGCTAGCCTGAAAACAATATG |
| | K150-45 | CCTTATAGGCGGGAAGCTGCAGGGCTTCGAGGCTGCATA |
| | Polynucleotide | CCTATCTAAGAAAGCCGGAATGAAAGTGCTTGTAATAG |
| | Sequence; | ACAAAAACCCGCAGGCGCTTATAAGGAATTATGCGGAT |
| | mutations are | GAGTTCCAGTGTTTTAACATAACGGAAGAGCCGGAAAA |
| | shown in bold and | ACTCGTCGCGATATCAAAAAATGTTGATGCCATACTGCC |
| | underlined letters | GGTAAATGAAAACCTTGAATGTATAGAATTTCTGAATTC |
| | | TATAAAAGAAAAATTCTCCTGCCCGGTACTTTTCGATTTT |
| | | GAAGCTTACAGGATCAGCAGGGATAAGAGAAAATCAAA |
| | | AGAATACTTCGCATCCATAGGAACCCCGACCCCTCAGGA |
| | | CAAACCGTCGGAACCACCTTATTTTGTAAAGCCTCCCTG |
| | | CGAAAGCAGCAGTGTGGGAGCGAGAATAATCCATGACA |
| | | GGAAAGAGCTTAAAGAGCTTGAGCCCGGGATGCTCATA |
| | | GAAGAATACGTTGAAGGGGAAGTGGTCGCGCTTGCTGT |
| | | CATAGGGGATGGAAATAATTTTGCTGTGGTAAAGGAAA |
| | | CCCTTGTACATATCGATGACACCTATGACTGCCATATGG |
| | | TGACCCCTCTCCCTCTAGACCCTTCCTTCAGGGAACTATC |
| | | CTACTCCCTTGCAGCAAACCTGCCCTTAAAAGGAATTAT |
| | | GTCGGTGGAAGCGATTTCCGGCCCCCTGGGGTTAAAAG |
| | | TTATTGAGATAGATGCCCGTTTCCCGAGCCAGGTGCCGA |
| | | CTGCGGTCTATTATTCTTCCGGGATCAACCTCATAGAAC |
| | | TCCTGTTCCGGGCTTTTAATGGAGGCATAGAAGAGATCA |
| | | AAACTCTCCCTGAAGACAGGTACTGCATTTACGAACATC |
| | | TCATGCTTGCAGAAAATGGAGTACTTATCCCTGTGGGAG |
| | | AACAGGTCCTGTCCATGGGAAATGATTACGGCAATTATT |
| | | ATGAAGAACCTGGAATAGAGATTTTCCTGTGCAAAGGA |
| | | GAGAACCCTGTATTCACCCTGGTTTTCTGGGGCAGAGAC |
| | | AGGGAAGAAGCTGAAGCTAGAAAAAACAAAGGGCTTTC |
| | | GATTCTAAAAAGCCGTTTCGGAGCTGCTGCATAA |
| 47 | PylC Variant | MRESWGASLKTICLIGGKLQGFEAAYLSKKAGMKVLVIDK |
| | K150-45 | NPQALIRNYADEFQCFNITEEPEKLVAISKNVDAILPVNENL |
| | Polypeptide | ECIEFLNSIKEKFSCPVLFDFEAYRISRDKRKSKEYFASIGTP |
| | Sequence; | TPQDKPSEPPYFVKPPCESSSVGARIIHDRKELKELEPGMLIE |
| | mutations are | EYVEGEVVALAVIGDGNNFAVVKETLVHIDDTYDCHMVT |
| | shown in bold and | PLPLDPSFRELSYSLAANLPLKGIMSVEAISGPLGLKVIEIDA |
| | underlined letters | RFPSQVPTAVYYSSGINLIELLFRAFNGGIEEIKTLPEDRYCI |
| | | YEHLMLAENGVLIPVGEQVLSMGNDYGNYYEEPGIEIFLC |
| | | KGENPVFTLVFWGRDREEAEARKNKGLSILKSRFGAAA |
| 48 | PylC Variant | ATGAGAGAGTCCTGGGGTGCTAGCCTGAAAACAATATG |
| | K150-32 | CCTTATAGGCGGGAAGCTGCAGGGCTTCGAGGCTGCATA |
| | Polynucleotide | CCTATCTAAGAAAGCCGGAATGAAAGTGCTTGTAATAG |
| | Sequence; | ACAAAAACCCGCAGGCGCTTATAAGGAATTATGCGGAT |
| | mutations are | GAGTTCCAGTGTTTTAACATAACGGAAGAGCCGGAAAA |
| | shown in bold and | ACTCGTCGCGATATCAAAAAATGTTGATGCCATACTGCC |
| | underlined letters | GGTAAATGAAAACCTTGAATGTATAGAATTTCTGAATTC |
| | | TATAAAAGAAAAATTCTCCTGCCCGGTACTTTTCGATTTT |
| | | GAAGCTTACAGGATCAGCAGGGATAAGAGAAAATCAAA |
| | | AGAATACTTCGCATCCATAGGAACCCCGACCCCTCAGGA |
| | | CAAACCGTCGGAACCACCTTATTTTGTAAAGCCTCCCTG |
| | | CGAAAGCAGCAGTGTGGGAGCGAGAATAATCCATGACA |
| | | GGAAAGAGCTTAAAGAGCTTGAGCCCGGGATGCTCATA |
| | | GAAGAATACGTTGAAGGGGAAGTGGTCGGGCTTATGGT |
| | | CATAGGGGATGGAAATAATTTTGCTGTGGTAAAGGAAA |
| | | CCCTTGTACATATCGATGACACCTATGACTGCCATATGG |
| | | TGACCCCTCTCCCTCTAGACCCTTCCTTCAGGGAACTATC |
| | | CTACTCCCTTGCAGCAAACCTGCCCTTAAAAGGAATTAT |
| | | GAATGTGGAAGCGATTTCCGGCCCCCTGGGGTTAAAAGT |
| | | TATTGAGATAGATGCCCGTTTCCCGAGCCAGGTTCCGAC |
| | | TGCGGTCTATTATTCTTCCGGGATCAACCTCATAGAACT |
| | | CCTGTTCCGGGCTTTTAATGGAGGCATAGAAGAGATCAA |
| | | AACTCTCCCTGAAGACAGGTACTGCATTTACGAACATCT |
| | | CATGCTTGCAGAAAATGGAGTACTTATCCCTGTGGGAGA |
| | | ACAGGTCCTGTCCATGGGAAATGATTACGGCAATTATTA |
| | | TGAAGAACCTGGAATAGAGATTTTCCTGTGCAAAGGAG |
| | | AGAACCCTGTATTCACCCTGGTTTTCTGGGGCAGAGACA |
| | | GGGAAGAAGCTGAAGCTAGAAAAAACAAAGGGCTTTCG |
| | | ATTCTAAAAAGCCGTTTCGGAGCTGCTGCATAA |
| 49 | PylC Variant | MRESWGASLKTICLIGGKLQGFEAAYLSKKAGMKVLVIDK |
| | K150-32 | NPQALIRNYADEFQCFNITEEPEKLVAISKNVDAILPVNENL |
| | Polypeptide | ECIEFLNSIKEKFSCPVLFDFEAYRISRDKRKSKEYFASIGTP |
| | Sequence; | TPQDKPSEPPYFVKPPCESSSVGARIIHDRKELKELEPGMLIE |
| | mutations are | EYVEGEVVGLMVIGDGNNFAVVKETLVHIDDTYDCHMVT |
| | shown in bold and | PLPLDPSFRELSYSLAANLPLKGIMNVEAISGPLGLKVIEIDA |
| | underlined letters | RFPSQVPTAVYYSSGINLIELLFRAFNGGIEEIKTLPEDRYCI |
| | | YEHLMLAENGVLIPVGEQVLSMGNDYGNYYEEPGIEIFLC |
| | | KGENPVFTLVFWGRDREEAEARKNKGLSILKSRFGAAA |
| 50 | PylC Variant | ATGAGAGAGTCCTGGGGTGCTAGCCTGAAAACAATATG |
| | K200-46 | CCTTATAGGCGGGAAGCTGCAGGGCTTCGAGGCTGCATA |
| | Polynucleotide | CCTATCTAAGAAAGCCGGAATGAAAGTGCTTGTAATAG |
| | Sequence; | ACAAAAACCCGCAGGCGCTTATAAGGAATTATGCGGAT |
| | mutations are | GAGTTCCAGTGTTTTAACATAACGGAAGAGCCGGAAAA |
| | shown in bold and | ACTCGTCGCGATATCAAAAAATGTTGATGCCATACTGCC |
| | underlined letters | GGTAAATGAAAACCTTGAATGTATAGAATTTCTGAATTC |
| | | TATAAAAGAAAAATTCTCCTGCCCGGTACTTTTCGATTTT |
| | | GAAGCTTACAGGATCAGCAGGGATAAGAGAAAATCAAA |
| | | AGAATACTTCGCATCCATAGGAACCCCGACCCCTCAGGA |
| | | CAAACCGTCGGAACCACCTTATTTTGTAAAGCCTCCCTG |
| | | CGAAAGCAGCAGTGTGGGAGCGAGAATAATCCATGACA |
| | | GGAAAGAGCTTAAAGAGCTTGAGCCCGGGATGCTCATA |
| | | GAAGAATACGTTGAAGGGGAAGTGGTCGGGCTTATTGT |
| | | CATAGGGGATGGAAATAATTTTGCTGTGGTAAAGGAAA |
| | | CCCTTGTACATATCGATGACACCTATGACTGCCATATGG |
| | | TGACCCCTCTCCCTCTAGACCCTTCCTTCAGGGAACTATC |
| | | CTACTCCCTTGCAGCAAACCTGCCCTTAAAAGGAATTAT |
| | | GAATGTGGAAGCGATTTCCGGCCCCCTGGGGTTAAAAGT |
| | | TATTGAGATAGATGCCCGTTTCCCGAGCCAGGTGCCGAC |
| | | TGCGGTCTATTATTCTTCCGGGATCAACCTCATAGAACT |
| | | CCTGTTCCGGGCTTTTAATGGAGGCATAGAAGAGATCAA |
| | | AACTCTCCCTGAAGACAGGTACTGCATTTACGAACATCT |
| | | CATGCTTGCAGAAAATGGAGTACTTATCCCTGTGGGAGA |
| | | ACAGGTCCTGTCCATGGGAAATGATTACGGCAATTATTA |
| | | TGAAGAACCTGGAATAGAGATTTTCCTGTGCAAAGGAG |
| | | AGAACCCTGTATTCACCCTGGTTTTCTGGGGCAGAGACA |
| | | GGGAAGAAGCTGAAGCTAGAAAAAACAAAGGGCTTTCG |
| | | ATTCTAAAAAGCCGTTTCGGAGCTGCTGCATAA |
| 51 | PylC Variant | MRESWGASLKTICLIGGKLQGFEAAYLSKKAGMKVLVIDK |
| | K200-46 | NPQALIRNYADEFQCFNITEEPEKLVAISKNVDAILPVNENL |
| | Polypeptide | ECIEFLNSIKEKFSCPVLFDFEAYRISRDKRKSKEYFASIGTP |
| | Sequence; | TPQDKPSEPPYFVKPPCESSSVGARIIHDRKELKELEPGMLIE |
| | mutations are | EYVEGEVVGLIVIGDGNNFAVVKETLVHIDDTYDCHMVTP |
| | shown in bold and | LPLDPSFRELSYSLAANLPLKGIMNVEAISGPLGLKVIEIDA |
| | underlined letters | RFPSQVPTAVYYSSGINLIELLFRAFNGGIEEIKTLPEDRYCI |
| | | YEHLMLAENGVLIPVGEQVLSMGNDYGNYYEEPGIEIFLC |
| | | KGENPVFTLVFWGRDREEAEARKNKGLSILKSRFGAAA |
| 52 | PylC Variant | ATGAGAGAGTCCTGGGGTGCTAGCCTGAAAACAATATG |
| | K150-15 | CCTTATAGGCGGGAAGCTGCAGGGCTTCGAGGCTGCATA |
| | Polynucleotide | CCTATCTAAGAAAGCCGGAATGAAAGTGCTTGTAATAG |
| | Sequence; | ACAAAAACCCGCAGGCGCTTATAAGGAATTATGCGGAT |
| | mutations are | GAGTTCCAGTGTTTTAACATAACGGAAGAGCCGGAAAA |
| | shown in bold and | ACTCGTCGCGATATCAAAAAATGTTGATGCCATACTGCC |
| | underlined letters | GGTAAATGAAAACCTTGAATGTATAGAATTTCTGAATTC |
| | | TATAAAAGAAAAATTCTCCTGCCCGGTACTTTTCGATTTT |
| | | GAAGCTTACAGGATCAGCAGGGATAAGAGAAAATCAAA |
| | | AGAATACTTCGCATCCATAGGAACCCCGACCCCTCAGGA |
| | | CAAACCGTCGGAACCACCTTATTTTGTAAAGCCTCCCTG |
| | | CGAAAGCAGCAGTGTGGGAGCGAGAATAATCCATGACA |
| | | GGAAAGAGCTTAAAGAGCTTGAGCCCGGGATGCTCATA |
| | | GAAGAATACGTTGAAGGGGAAGTGGTCGGTCTTACTGT |
| | | CATAGGGGATGGAAATAATTTTGCTGTGGTAAAGGAAA |
| | | CCCTTGTACATATCGATGACACCTATGACTGCCATATGG |
| | | TGACCCCTCTCCCTCTAGACCCTTCCTTCAGGGAACTATC |
| | | CTACTCCCTTGCAGCAAACCTGCCCTTAAAAGGAATTAT |
| | | GTCGGTGGAAGCGATTTCCGGCCCCCTGGGGTTAAAAG |
| | | TTATTGAGATAGATGCCCGTTTCCCGAGCCAGCTGCCGA |
| | | CTGCGGTCTATTATTCTTCCGGGATCAACCTCATAGAAC |
| | | TCCTGTTCCGGGCTTTTAATGGAGGCATAGAAGAGATCA |
| | | AAACTCTCCCTGAAGACAGGTACTGCATTTACGAACATC |
| | | TCATGCTTGCAGAAAATGGAGTACTTATCCCTGTGGGAG |
| | | AACAGGTCCTGTCCATGGGAAATGATTACGGCAATTATT |
| | | ATGAAGAACCTGGAATAGAGATTTTCCTGTGCAAAGGA |
| | | GAGAACCCTGTATTCACCCTGGTTTTCTGGGGCAGAGAC |
| | | AGGGAAGAAGCTGAAGCTAGAAAAAACAAAGGGCTTTC |
| | | GATTCTAAAAAGCCGTTTCGGAGCTGCTGCATAA |
| 53 | PylC Variant | MRESWGASLKTICLIGGKLQGFEAAYLSKKAGMKVLVIDK |
| | K150-15 | NPQALIRNYADEFQCFNITEEPEKLVAISKNVDAILPVNENL |
| | Polypeptide | ECIEFLNSIKEKFSCPVLFDFEAYRISRDKRKSKEYFASIGTP |
| | Sequence; | TPQDKPSEPPYFVKPPCESSSVGARIIHDRKELKELEPGMLIE |
| | mutations are | EYVEGEVVGLTVIGDGNNFAVVKETLVHIDDTYDCHMVT |
| | shown in bold and | PLPLDPSFRELSYSLAANLPLKGIMSVEAISGPLGLKVIEIDA |
| | underlined letters | RFPSQLPTAVYYSSGINLIELLFRAFNGGIEEIKTLPEDRYCI |
| | | YEHLMLAENGVLIPVGEQVLSMGNDYGNYYEEPGIEIFLC |
| | | KGENPVFTLVFWGRDREEAEARKNKGLSILKSRFGAAA |
| 54 | PylC Variant | ATGAGAGAGTCCTGGGGTGCTAGCCTGAAAACAATATG |
| | K150-22 | CCTTATAGGCGGGAAGCTGCAGGGCTTCGAGGCTGCATA |
| | Polynucleotide | CCTATCTAAGAAAGCCGGAATGAAAGTGCTTGTAATAG |
| | Sequence; | ACAAAAACCCGCAGGCGCTTATAAGGAATTATGCGGAT |
| | mutations are | GAGTTCCAGTGTTTTAACATAACGGAAGAGCCGGAAAA |
| | shown in bold and | ACTCGTCGCGATATCAAAAAATGTTGATGCCATACTGCC |
| | underlined letters | GGTAAATGAAAACCTTGAATGTATAGAATTTCTGAATTC |
| | | TATAAAAGAAAAATTCTCCTGCCCGGTACTTTTCGATTTT |
| | | GAAGCTTACAGGATCAGCAGGGATAAGAGAAAATCAAA |
| | | AGAATACTTCGCATCCATAGGAACCCCGACCCCTCAGGA |
| | | CAAACCGTCGGAACCACCTTATTTTGTAAAGCCTCCCTG |
| | | CGAAAGCAGCAGTGTGGGAGCGAGAATAATCCATGACA |
| | | GGAAAGAGCTTAAAGAGCTTGAGCCCGGGATGCTCATA |
| | | GAAGAATACGTTGAAGGGGAAGTGGTCGCGCTTACGGT |
| | | CATAGGGGATGGAAATAATTTTGCTGTGGTAAAGGAAA |
| | | CCCTTGTACATATCGATGACACCTATGACTGCCATATGG |
| | | TGACCCCTCTCCCTCTAGACCCTTCCTTCAGGGAACTATC |
| | | CTACTCCCTTGCAGCAAACCTGCCCTTAAAAGGAATTAT |
| | | GTCTGTGGAAGCGATTTCCGGCCCCCTGGGGTTAAAAGT |
| | | TATTGAGATAGATGCCCGTTTCCCGAGCCAGCTGCCGAC |
| | | TGCGGTCTATTATTCTTCCGGGATCAACCTCATAGAACT |
| | | CCTGTTCCGGGCTTTTAATGGAGGCATAGAAGAGATCAA |
| | | AACTCTCCCTGAAGACAGGTACTGCATTTACGAACATCT |
| | | CATGCTTGCAGAAAATGGAGTACTTATCCCTGTGGGAGA |
| | | ACAGGTCCTGTCCATGGGAAATGATTACGGCAATTATTA |
| | | TGAAGAACCTGGAATAGAGATTTTCCTGTGCAAAGGAG |
| | | AGAACCCTGTATTCACCCTGGTTTTCTGGGGCAGAGACA |
| | | GGGAAGAAGCTGAAGCTAGAAAAAACAAAGGGCTTTCG |
| | | ATTCTAAAAAGCCGTTTCGGAGCTGCTGCATAA |
| 55 | PylC Variant | MRESWGASLKTICLIGGKLQGFEAAYLSKKAGMKVLVIDK |
| | K150-22 | NPQALIRNYADEFQCFNITEEPEKLVAISKNVDAILPVNENL |
| | Polypeptide | ECIEFLNSIKEKFSCPVLFDFEAYRISRDKRKSKEYFASIGTP |
| | Sequence; | TPQDKPSEPPYFVKPPCESSSVGARIIHDRKELKELEPGMLIE |
| | mutations are | EYVEGEVVALTVIGDGNNFAVVKETLVHIDDTYDCHMVT |
| | shown in bold and | PLPLDPSFRELSYSLAANLPLKGIMSVEAISGPLGLKVIEIDA |
| | underlined letters | RFPSQLPTAVYYSSGINLIELLFRAFNGGIEEIKTLPEDRYCI |
| | | YEHLMLAENGVLIPVGEQVLSMGNDYGNYYEEPGIEIFLC |
| | | KGENPVFTLVFWGRDREEAEARKNKGLSILKSRFGAAA |
| 56 | PylC Variant | ATGAGAGAGTCCTGGGGTGCTAGCCTGAAAACAATATG |
| | K150-35 | CCTTATAGGCGGGAAGCTGCAGGGCTTCGAGGCTGCATA |
| | Polynucleotide | CCTATCTAAGAAAGCCGGAATGAAAGTGCTTGTAATAG |
| | Sequence; | ACAAAAACCCGCAGGCGCTTATAAGGAATTATGCGGAT |
| | mutations are | GAGTTCCAGTGTTTTAACATAACGGAAGAGCCGGAAAA |
| | shown in bold and | ACTCGTCGCGATATCAAAAAATGTTGATGCCATACTGCC |
| | underlined letters | GGTAAATGAAAACCTTGAATGTATAGAATTTCTGAATTC |
| | | TATAAAAGAAAAATTCTCCTGCCCGGTACTTTTCGATTTT |
| | | GAAGCTTACAGGATCAGCAGGGATAAGAGAAAATCAAA |
| | | AGAATACTTCGCATCCATAGGAACCCCGACCCCTCAGGA |
| | | CAAACCGTCGGAACCACCTTATTTTGTAAAGCCTCCCTG |
| | | CGAAAGCAGCAGTGTGGGAGCGAGAATAATCCATGACA |
| | | GGAAAGAGCTTAAAGAGCTTGAGCCCGGGATGCTCATA |
| | | GAAGAATACGTTGAAGGGGAAGTGGTCCTTCTTACTGT |
| | | CATAGGGGATGGAAATAATTTTGCTGTGGTAAAGGAAA |
| | | CCCTTGTACATATCGATGACACCTATGACTGCCATATGG |
| | | TGACCCCTCTCCCTCTAGACCCTTCCTTCAGGGAACTATC |
| | | CTACTCCCTTGCAGCAAACCTGCCCTTAAAAGGAATTAT |
| | | GTCGGTGGAAGCGATTTCCGGCCCCCTGGGGTTAAAAG |
| | | TTATTGAGATAGATGCCCGTTTCCCGAGCCAGATTCCGA |
| | | CTGCGGTCTATTATTCTTCCGGGATCAACCTCATAGAAC |
| | | TCCTGTTCCGGGCTTTTAATGGAGGCATAGAAGAGATCA |
| | | AAACTCTCCCTGAAGACAGGTACTGCATTTACGAACATC |
| | | TCATGCTTGCAGAAAATGGAGTACTTATCCCTGTGGGAG |
| | | AACAGGTCCTGTCCATGGGAAATGATTACGGCAATTATT |
| | | ATGAAGAACCTGGAATAGAGATTTTCCTGTGCAAAGGA |
| | | GAGAACCCTGTATTCACCCTGGTTTTCTGGGGCAGAGAC |
| | | AGGGAAGAAGCTGAAGCTAGAAAAAACAAAGGGCTTTC |
| | | GATTCTAAAAAGCCGTTTCGGAGCTGCTGCATAA |
| 57 | PylC Variant | MRESWGASLKTICLIGGKLQGFEAAYLSKKAGMKVLVIDK |
| | K150-35 | NPQALIRNYADEFQCFNITEEPEKLVAISKNVDAILPVNENL |
| | Polypeptide | ECIEFLNSIKEKFSCPVLFDFEAYRISRDKRKSKEYFASIGTP |
| | Sequence; | TPQDKPSEPPYFVKPPCESSSVGARIIHDRKELKELEPGMLIE |
| | mutations are | EYVEGEVVLLTVIGDGNNFAVVKETLVHIDDTYDCHMVTP |
| | shown in bold and | LPLDPSFRELSYSLAANLPLKGIMSVEAISGPLGLKVIEIDAR |
| | underlined letters | FPSQIPTAVYYSSGINLIELLFRAFNGGIEEIKTLPEDRYCIYE |
| | | HLMLAENGVLIPVGEQVLSMGNDYGNYYEEPGIEIFLCKG |
| | | ENPVFTLVFWGRDREEAEARKNKGLSILKSRFGAAA |
| 58 | PylC Variant | ATGAGAGAGTCCTGGGGTGCTAGCCTGAAAACAATATG |
| | K200-16 | CCTTATAGGCGGGAAGCTGCAGGGCTTCGAGGCTGCATA |
| | Polynucleotide | CCTATCTAAGAAAGCCGGAATGAAAGTGCTTGTAATAG |
| | Sequence; | ACAAAAACCCGCAGGCGCTTATAAGGAATTATGCGGAT |
| | mutations are | GAGTTCCAGTGTTTTAACATAACGGAAGAGCCGGAAAA |
| | shown in bold and | ACTCGTCGCGATATCAAAAAATGTTGATGCCATACTGCC |
| | underlined letters | GGTAAATGAAAACCTTGAATGTATAGAATTTCTGAATTC |
| | | TATAAAAGAAAAATTCTCCTGCCCGGTACTTTTCGATTTT |
| | | GAAGCTTACAGGATCAGCAGGGATAAGAGAAAATCAAA |
| | | AGAATACTTCGCATCCATAGGAACCCCGACCCCTCAGGA |
| | | CAAACCGTCGGAACCACCTTATTTTGTAAAGCCTCCCTG |
| | | CGAAAGCAGCAGTGTGGGAGCGAGAATAATCCATGACA |
| | | GGAAAGAGCTTAAAGAGCTTGAGCCCGGGATGCTCATA |
| | | GAAGAATACGTTGAAGGGGAAGTGGTCACTCTTCCTGT |
| | | CATAGGGGATGGAAATAATTTTGCTGTGGTAAAGGAAA |
| | | CCCTTGTACATATCGATGACACCTATGACTGCCATATGG |
| | | TGACCCCTCTCCCTCTAGACCCTTCCTTCAGGGAACTATC |
| | | CTACTCCCTTGCAGCAAACCTGCCCTTAAAAGGAATTAT |
| | | GCATGTGGAAGCGATTTCCGGCCCCCTGGGGTTAAAAGT |
| | | TATTGAGATAGATGCCCGTTTCCCGAGCCAGATGCCGAC |
| | | TGCGGTCTATTATTCTTCCGGGATCAACCTCATAGAACT |
| | | CCTGTTCCGGGCTTTTAATGGAGGCATAGAAGAGATCAA |
| | | AACTCTCCCTGAAGACAGGTACTGCATTTACGAACATCT |
| | | CATGCTTGCAGAAAATGGAGTACTTATCCCTGTGGGAGA |
| | | ACAGGTCCTGTCCATGGGAAATGATTACGGCAATTATTA |
| | | TGAAGAACCTGGAATAGAGATTTTCCTGTGCAAAGGAG |
| | | AGAACCCTGTATTCACCCTGGTTTTCTGGGGCAGAGACA |
| | | GGGAAGAAGCTGAAGCTAGAAAAAACAAAGGGCTTTCG |
| | | ATTCTAAAAAGCCGTTTCGGAGCTGCTGCATAA |
| 59 | PylC Variant | MRESWGASLKTICLIGGKLQGFEAAYLSKKAGMKVLVIDK |
| | K200-16 | NPQALIRNYADEFQCFNITEEPEKLVAISKNVDAILPVNENL |
| | Polypeptide | ECIEFLNSIKEKFSCPVLFDFEAYRISRDKRKSKEYFASIGTP |
| | Sequence; | TPQDKPSEPPYFVKPPCESSSVGARIIHDRKELKELEPGMLIE |
| | mutations are | EYVEGEVVTLPVIGDGNNFAVVKETLVHIDDTYDCHMVTP |
| | shown in bold and | LPLDPSFRELSYSLAANLPLKGIMHVEAISGPLGLKVIEIDA |
| | underlined letters | RFPSQMPTAVYYSSGINLIELLFRAFNGGIEEIKTLPEDRYCI |
| | | YEHLMLAENGVLIPVGEQVLSMGNDYGNYYEEPGIEIFLC |
| | | KGENPVFTLVFWGRDREEAEARKNKGLSILKSRFGAAA |
| 60 | PylC Variant | ATGAGAGAGTCCTGGGGTGCTAGCCTGAAAACAATATG |
| | K200-17 | CCTTATAGGCGGGAAGCTGCAGGGCTTCGAGGCTGCATA |
| | Polynucleotide | CCTATCTAAGAAAGCCGGAATGAAAGTGCTTGTAATAG |
| | Sequence; | ACAAAAACCCGCAGGCGCTTATAAGGAATTATGCGGAT |
| | mutations are | GAGTTCCAGTGTTTTAACATAACGGAAGAGCCGGAAAA |
| | shown in bold and | ACTCGTCGCGATATCAAAAAATGTTGATGCCATACTGCC |
| | underlined letters | GGTAAATGAAAACCTTGAATGTATAGAATTTCTGAATTC |
| | | TATAAAAGAAAAATTCTCCTGCCCGGTACTTTTCGATTTT |
| | | GAAGCTTACAGGATCAGCAGGGATAAGAGAAAATCAAA |
| | | AGAATACTTCGCATCCATAGGAACCCCGACCCCTCAGGA |
| | | CAAACCGTCGGAACCACCTTATTTTGTAAAGCCTCCCTG |
| | | CGAAAGCAGCAGTGTGGGAGCGAGAATAATCCATGACA |
| | | GGAAAGAGCTTAAAGAGCTTGAGCCCGGGATGCTCATA |
| | | GAAGAATACGTTGAAGGGGAAGTGGTCAGTCTTTTGGT |
| | | CATAGGGGATGGAAATAATTTTGCTGTGGTAAAGGAAA |
| | | CCCTTGTACATATCGATGACACCTATGACTGCCATATGG |
| | | TGACCCCTCTCCCTCTAGACCCTTCCTTCAGGGAACTATC |
| | | CTACTCCCTTGCAGCAAACCTGCCCTTAAAAGGAATTAT |
| | | GCTTGTGGAAGCGATTTCCGGCCCCCTGGGGTTAAAAGT |
| | | TATTGAGATAGATGCCCGTTTCCCGAGCCAGGTGCCGAC |
| | | TGCGGTCTATTATTCTTCCGGGATCAACCTCATAGAACT |
| | | CCTGTTCCGGGCTTTTAATGGAGGCATAGAAGAGATCAA |
| | | AACTCTCCCTGAAGACAGGTACTGCATTTACGAACATCT |
| | | CATGCTTGCAGAAAATGGAGTACTTATCCCTGTGGGAGA |
| | | ACAGGTCCTGTCCATGGGAAATGATTACGGCAATTATTA |
| | | TGAAGAACCTGGAATAGAGATTTTCCTGTGCAAAGGAG |
| | | AGAACCCTGTATTCACCCTGGTTTTCTGGGGCAGAGACA |
| | | GGGAAGAAGCTGAAGCTAGAAAAAACAAAGGGCTTTCG |
| | | ATTCTAAAAAGCCGTTTCGGAGCTGCTGCATAA |
| 61 | PylC Variant | MRESWGASLKTICLIGGKLQGFEAAYLSKKAGMKVLVIDK |
| | K200-17; | NPQALIRNYADEFQCFNITEEPEKLVAISKNVDAILPVNENL |
| | mutations are | ECIEFLNSIKEKFSCPVLFDFEAYRISRDKRKSKEYFASIGTP |
| | shown in bold and | TPQDKPSEPPYFVKPPCESSSVGARIIHDRKELKELEPGMLIE |
| | underlined | EYVEGEVVSLLVIGDGNNFAVVKETLVHIDDTYDCHMVTP |
| | lettersPolypeptide | LPLDPSFRELSYSLAANLPLKGIMLVEAISGPLGLKVIEIDAR |
| | Sequence; | FPSQVPTAVYYSSGINLIELLFRAFNGGIEEIKTLPEDRYCIY |
| | mutations are | EHLMLAENGVLIPVGEQVLSMGNDYGNYYEEPGIEIFLCK |
| | shown in bold and | GENPVFTLVFWGRDREEAEARKNKGLSILKSRFGAAA |
| 62 | underlined letters | ATGAGAGAGTCCTGGGGTGCTAGCCTGAAAACAATATG |
| | PylC Variant | CCTTATAGGCGGGAAGCTGCAGGGCTTCGAGGCTGCATA |
| | K200-12 | CCTATCTAAGAAAGCCGGAATGAAAGTGCTTGTAATAG |
| | Polynucleotide | ACAAAAACCCGCAGGCGCTTATAAGGAATTATGCGGAT |
| | Sequence; | GAGTTCCAGTGTTTTAACATAACGGAAGAGCCGGAAAA |
| | mutations are | ACTCGTCGCGATATCAAAAAATGTTGATGCCATACTGCC |
| | shown in bold and | GGTAAATGAAAACCTTGAATGTATAGAATTTCTGAATTC |
| | underlined letters | TATAAAAGAAAAATTCTCCTGCCCGGTACTTTTCGATTTT |
| | | GAAGCTTACAGGATCAGCAGGGATAAGAGAAAATCAAA |
| | | AGAATACTTCGCATCCATAGGAACCCCGACCCCTCAGGA |
| | | CAAACCGTCGGAACCACCTTATTTTGTAAAGCCTCCCTG |
| | | CGAAAGCAGCAGTGTGGGAGCGAGAATAATCCATGACA |
| | | GGAAAGAGCTTAAAGAGCTTGAGCCCGGGATGCTCATA |
| | | GAAGAATACGTTGAAGGGGAAGTGGTCGCTCTTGTTGT |
| | | CATAGGGGATGGAAATAATTTTGCTGTGGTAAAGGAAA |
| | | CCCTTGTACATATCGATGACACCTATGACTGCCATATGG |
| | | TGACCCCTCTCCCTCTAGACCCTTCCTTCAGGGAACTATC |
| | | CTACTCCCTTGCAGCAAACCTGCCCTTAAAAGGAATTAT |
| | | GCAGGTGGAAGCGATTTCCGGCCCCCTGGGGTTAAAAG |
| | | TTATTGAGATAGATGCCCGTTTCCCGAGCCAGTGTCCGA |
| | | CTGCGGTCTATTATTCTTCCGGGATCAACCTCATAGAAC |
| | | TCCTGTTCCGGGCTTTTAATGGAGGCATAGAAGAGATCA |
| | | AAACTCTCCCTGAAGACAGGTACTGCATTTACGAACATC |
| | | TCATGCTTGCAGAAAATGGAGTACTTATCCCTGTGGGAG |
| | | AACAGGTCCTGTCCATGGGAAATGATTACGGCAATTATT |
| | | ATGAAGAACCTGGAATAGAGATTTTCCTGTGCAAAGGA |
| | | GAGAACCCTGTATTCACCCTGGTTTTCTGGGGCAGAGAC |
| | | AGGGAAGAAGCTGAAGCTAGAAAAAACAAAGGGCTTTC |
| | | GATTCTAAAAAGCCGTTTCGGAGCTGCTGCATAA |
| 63 | PylC Variant | MRESWGASLKTICLIGGKLQGFEAAYLSKKAGMKVLVIDK |
| | K200-12 | NPQALIRNYADEFQCFNITEEPEKLVAISKNVDAILPVNENL |
| | Polypeptide | ECIEFLNSIKEKFSCPVLFDFEAYRISRDKRKSKEYFASIGTP |
| | Sequence; | TPQDKPSEPPYFVKPPCESSSVGARIIHDRKELKELEPGMLIE |
| | mutations are | EYVEGEVVALVVIGDGNNFAVVKETLVHIDDTYDCHMVT |
| | shown in bold and | PLPLDPSFRELSYSLAANLPLKGIMQVEAISGPLGLKVIEIDA |
| | underlined letters | RFPSQCPTAVYYSSGINLIELLFRAFNGGIEEIKTLPEDRYCI |
| | | YEHLMLAENGVLIPVGEQVLSMGNDYGNYYEEPGIEIFLC |
| | | KGENPVFTLVFWGRDREEAEARKNKGLSILKSRFGAAA |
| 64 | PylC Variant | ATGAGAGAGTCCTGGGGTGCTAGCCTGAAAACAATATG |
| | K200-7 | CCTTATAGGCGGGAAGCTGCAGGGCTTCGAGGCTGCATA |
| | Polynucleotide | CCTATCTAAGAAAGCCGGAATGAAAGTGCTTGTAATAG |
| | Sequence; | ACAAAAACCCGCAGGCGCTTATAAGGAATTATGCGGAT |
| | mutations are | GAGTTCCAGTGTTTTAACATAACGGAAGAGCCGGAAAA |
| | shown in bold and | ACTCGTCGCGATATCAAAAAATGTTGATGCCATACTGCC |
| | underlined letters | GGTAAATGAAAACCTTGAATGTATAGAATTTCTGAATTC |
| | | TATAAAAGAAAAATTCTCCTGCCCGGTACTTTTCGATTTT |
| | | GAAGCTTACAGGATCAGCAGGGATAAGAGAAAATCAAA |
| | | AGAATACTTCGCATCCATAGGAACCCCGACCCCTCAGGA |
| | | CAAACCGTCGGAACCACCTTATTTTGTAAAGCCTCCCTG |
| | | CGAAAGCAGCAGTGTGGGAGCGAGAATAATCCATGACA |
| | | GGAAAGAGCTTAAAGAGCTTGAGCCCGGGATGCTCATA |
| | | GAAGAATACGTTGAAGGGGAAGTGGTCCTGCTTGTTGT |
| | | CATAGGGGATGGAAATAATTTTGCTGTGGTAAAGGAAA |
| | | CCCTTGTACATATCGATGACACCTATGACTGCCATATGG |
| | | TGACCCCTCTCCCTCTAGACCCTTCCTTCAGGGAACTATC |
| | | CTACTCCCTTGCAGCAAACCTGCCCTTAAAAGGAATTAT |
| | | GAGTGTGGAAGCGATTTCCGGCCCCCTGGGGTTAAAAG |
| | | TTATTGAGATAGATGCCCGTTTCCCGAGCCAGGCGCCGA |
| | | CTGCGGTCTATTATTCTTCCGGGATCAACCTCATAGAAC |
| | | TCCTGTTCCGGGCTTTTAATGGAGGCATAGAAGAGATCA |
| | | AAACTCTCCCTGAAGACAGGTACTGCATTTACGAACATC |
| | | TCATGCTTGCAGAAAATGGAGTACTTATCCCTGTGGGAG |
| | | AACAGGTCCTGTCCATGGGAAATGATTACGGCAATTATT |
| | | ATGAAGAACCTGGAATAGAGATTTTCCTGTGCAAAGGA |
| | | GAGAACCCTGTATTCACCCTGGTTTTCTGGGGCAGAGAC |
| | | AGGGAAGAAGCTGAAGCTAGAAAAAACAAAGGGCTTTC |
| | | GATTCTAAAAAGCCGTTTCGGAGCTGCTGCATAA |
| 65 | PylC Variant | MRESWGASLKTICLIGGKLQGFEAAYLSKKAGMKVLVIDK |
| | K200-7 | NPQALIRNYADEFQCFNITEEPEKLVAISKNVDAILPVNENL |
| | Polypeptide | ECIEFLNSIKEKFSCPVLFDFEAYRISRDKRKSKEYFASIGTP |
| | Sequence; | TPQDKPSEPPYFVKPPCESSSVGARIIHDRKELKELEPGMLIE |
| | mutations are | EYVEGEVVLLVVIGDGNNFAVVKETLVHIDDTYDCHMVT |
| | shown in bold and | PLPLDPSFRELSYSLAANLPLKGIMSVEAISGPLGLKVIEIDA |
| | underlined letters | RFPSQAPTAVYYSSGINLIELLFRAFNGGIEEIKTLPEDRYCI |
| | | YEHLMLAENGVLIPVGEQVLSMGNDYGNYYEEPGIEIFLC |
| | | KGENPVFTLVFWGRDREEAEARKNKGLSILKSRFGAAA |
| 66 | PylC Variant | ATGAGAGAGTCCTGGGGTGCTAGCCTGAAAACAATATG |
| | K200-6 | CCTTATAGGCGGGAAGCTGCAGGGCTTCGAGGCTGCATA |
| | Polynucleotide | CCTATCTAAGAAAGCCGGAATGAAAGTGCTTGTAATAG |
| | Sequence; | ACAAAAACCCGCAGGCGCTTATAAGGAATTATGCGGAT |
| | mutations are | GAGTTCCAGTGTTTTAACATAACGGAAGAGCCGGAAAA |
| | shown in bold and | ACTCGTCGCGATATCAAAAAATGTTGATGCCATACTGCC |
| | underlined letters | GGTAAATGAAAACCTTGAATGTATAGAATTTCTGAATTC |
| | | TATAAAAGAAAAATTCTCCTGCCCGGTACTTTTCGATTTT |
| | | GAAGCTTACAGGATCAGCAGGGATAAGAGAAAATCAAA |
| | | AGAATACTTCGCATCCATAGGAACCCCGACCCCTCAGGA |
| | | CAAACCGTCGGAACCACCTTATTTTGTAAAGCCTCCCTG |
| | | CGAAAGCAGCAGTGTGGGAGCGAGAATAATCCATGACA |
| | | GGAAAGAGCTTAAAGAGCTTGAGCCCGGGATGCTCATA |
| | | GAAGAATACGTTGAAGGGGAAGTGGTCGCTCTTTGTGT |
| | | CATAGGGGATGGAAATAATTTTGCTGTGGTAAAGGAAA |
| | | CCCTTGTACATATCGATGACACCTATGACTGCCATATGG |
| | | TGACCCCTCTCCCTCTAGACCCTTCCTTCAGGGAACTATC |
| | | CTACTCCCTTGCAGCAAACCTGCCCTTAAAAGGAATTAT |
| | | GCTGGTGGAAGCGATTTCCGGCCCCCTGGGGTTAAAAG |
| | | TTATTGAGATAGATGCCCGTTTCCCGAGCCAGTGTCCGA |
| | | CTGCGGTCTATTATTCTTCCGGGATCAACCTCATAGAAC |
| | | TCCTGTTCCGGGCTTTTAATGGAGGCATAGAAGAGATCA |
| | | AAACTCTCCCTGAAGACAGGTACTGCATTTACGAACATC |
| | | TCATGCTTGCAGAAAATGGAGTACTTATCCCTGTGGGAG |
| | | AACAGGTCCTGTCCATGGGAAATGATTACGGCAATTATT |
| | | ATGAAGAACCTGGAATAGAGATTTTCCTGTGCAAAGGA |
| | | GAGAACCCTGTATTCACCCTGGTTTTCTGGGGCAGAGAC |
| | | AGGGAAGAAGCTGAAGCTAGAAAAAACAAAGGGCTTTC |
| | | GATTCTAAAAAGCCGTTTCGGAGCTGCTGCATAA |
| 67 | PylC Variant | MRESWGASLKTICLIGGKLQGFEAAYLSKKAGMKVLVIDK |
| | K200-6 | NPQALIRNYADEFQCFNITEEPEKLVAISKNVDAILPVNENL |
| | Polypeptide | ECIEFLNSIKEKFSCPVLFDFEAYRISRDKRKSKEYFASIGTP |
| | Sequence; | TPQDKPSEPPYFVKPPCESSSVGARIIHDRKELKELEPGMLIE |
| | mutations are | EYVEGEVVALCVIGDGNNFAVVKETLVHIDDTYDCHMVT |
| | shown in bold and | PLPLDPSFRELSYSLAANLPLKGIMLVEAISGPLGLKVIEIDA |
| | underlined letters | RFPSQCPTAVYYSSGINLIELLFRAFNGGIEEIKTLPEDRYCI |
| | | YEHLMLAENGVLIPVGEQVLSMGNDYGNYYEEPGIEIFLC |
| | | KGENPVFTLVFWGRDREEAEARKNKGLSILKSRFGAAA |
| 68 | PylC Variant | ATGAGAGAGTCCTGGGGTGCTAGCCTGAAAACAATATG |
| | K200-3 | CCTTATAGGCGGGAAGCTGCAGGGCTTCGAGGCTGCATA |
| | Polynucleotide | CCTATCTAAGAAAGCCGGAATGAAAGTGCTTGTAATAG |
| | Sequence; | ACAAAAACCCGCAGGCGCTTATAAGGAATTATGCGGAT |
| | mutations are | GAGTTCCAGTGTTTTAACATAACGGAAGAGCCGGAAAA |
| | shown in bold and | ACTCGTCGCGATATCAAAAAATGTTGATGCCATACTGCC |
| | underlined letters | GGTAAATGAAAACCTTGAATGTATAGAATTTCTGAATTC |
| | | TATAAAAGAAAAATTCTCCTGCCCGGTACTTTTCGATTTT |
| | | GAAGCTTACAGGATCAGCAGGGATAAGAGAAAATCAAA |
| | | AGAATACTTCGCATCCATAGGAACCCCGACCCCTCAGGA |
| | | CAAACCGTCGGAACCACCTTATTTTGTAAAGCCTCCCTG |
| | | CGAAAGCAGCAGTGTGGGAGCGAGAATAATCCATGACA |
| | | GGAAAGAGCTTAAAGAGCTTGAGCCCGGGATGCTCATA |
| | | GAAGAATACGTTGAAGGGGAAGTGGTCAATCTTTGTGT |
| | | CATAGGGGATGGAAATAATTTTGCTGTGGTAAAGGAAA |
| | | CCCTTGTACATATCGATGACACCTATGACTGCCATATGG |
| | | TGACCCCTCTCCCTCTAGACCCTTCCTTCAGGGAACTATC |
| | | CTACTCCCTTGCAGCAAACCTGCCCTTAAAAGGAATTAT |
| | | GACTGTGGAAGCGATTTCCGGCCCCCTGGGGTTAAAAGT |
| | | TATTGAGATAGATGCCCGTTTCCCGAGCCAGTGTCCGAC |
| | | TGCGGTCTATTATTCTTCCGGGATCAACCTCATAGAACT |
| | | CCTGTTCCGGGCTTTTAATGGAGGCATAGAAGAGATCAA |
| | | AACTCTCCCTGAAGACAGGTACTGCATTTACGAACATCT |
| | | CATGCTTGCAGAAAATGGAGTACTTATCCCTGTGGGAGA |
| | | ACAGGTCCTGTCCATGGGAAATGATTACGGCAATTATTA |
| | | TGAAGAACCTGGAATAGAGATTTTCCTGTGCAAAGGAG |
| | | AGAACCCTGTATTCACCCTGGTTTTCTGGGGCAGAGACA |
| | | GGGAAGAAGCTGAAGCTAGAAAAAACAAAGGGCTTTCG |
| | | ATTCTAAAAAGCCGTTTCGGAGCTGCTGCATAA |
| 69 | PylC Variant | MRESWGASLKTICLIGGKLQGFEAAYLSKKAGMKVLVIDK |
| | K200-3 | NPQALIRNYADEFQCFNITEEPEKLVAISKNVDAILPVNENL |
| | Polypeptide | ECIEFLNSIKEKFSCPVLFDFEAYRISRDKRKSKEYFASIGTP |
| | Sequence; | TPQDKPSEPPYFVKPPCESSSVGARIIHDRKELKELEPGMLIE |
| | mutations are | EYVEGEVVNLCVIGDGNNFAVVKETLVHIDDTYDCHMVT |
| | shown in bold and | PLPLDPSFRELSYSLAANLPLKGIMTVEAISGPLGLKVIEIDA |
| | underlined letters | RFPSQCPTAVYYSSGINLIELLFRAFNGGIEEIKTLPEDRYCI |
| | | YEHLMLAENGVLIPVGEQVLSMGNDYGNYYEEPGIEIFLC |
| | | KGENPVFTLVFWGRDREEAEARKNKGLSILKSRFGAAA |
| 70 | PylC Variant | ATGAGAGAGTCCTGGGGTGCTAGCCTGAAAACAATATG |
| | K200-9 | CCTTATAGGCGGGAAGCTGCAGGGCTTCGAGGCTGCATA |
| | Polynucleotide | CCTATCTAAGAAAGCCGGAATGAAAGTGCTTGTAATAG |
| | Sequence; | ACAAAAACCCGCAGGCGCTTATAAGGAATTATGCGGAT |
| | mutations are | GAGTTCCAGTGTTTTAACATAACGGAAGAGCCGGAAAA |
| | shown in bold and | ACTCGTCGCGATATCAAAAAATGTTGATGCCATACTGCC |
| | underlined letters | GGTAAATGAAAACCTTGAATGTATAGAATTTCTGAATTC |
| | | TATAAAAGAAAAATTCTCCTGCCCGGTACTTTTCGATTTT |
| | | GAAGCTTACAGGATCAGCAGGGATAAGAGAAAATCAAA |
| | | AGAATACTTCGCATCCATAGGAACCCCGACCCCTCAGGA |
| | | CAAACCGTCGGAACCACCTTATTTTGTAAAGCCTCCCTG |
| | | CGAAAGCAGCAGTGTGGGAGCGAGAATAATCCATGACA |
| | | GGAAAGAGCTTAAAGAGCTTGAGCCCGGGATGCTCATA |
| | | GAAGAATACGTTGAAGGGGAAGTGGTCCATCTTCTGGT |
| | | CATAGGGGATGGAAATAATTTTGCTGTGGTAAAGGAAA |
| | | CCCTTGTACATATCGATGACACCTATGACTGCCATATGG |
| | | TGACCCCTCTCCCTCTAGACCCTTCCTTCAGGGAACTATC |
| | | CTACTCCCTTGCAGCAAACCTGCCCTTAAAAGGAATTAT |
| | | GTCTGTGGAAGCGATTTCCGGCCCCCTGGGGTTAAAAGT |
| | | TATTGAGATAGATGCCCGTTTCCCGAGCCAGAGTCCGAC |
| | | TGCGGTCTATTATTCTTCCGGGATCAACCTCATAGAACT |
| | | CCTGTTCCGGGCTTTTAATGGAGGCATAGAAGAGATCAA |
| | | AACTCTCCCTGAAGACAGGTACTGCATTTACGAACATCT |
| | | CATGCTTGCAGAAAATGGAGTACTTATCCCTGTGGGAGA |
| | | ACAGGTCCTGTCCATGGGAAATGATTACGGCAATTATTA |
| | | TGAAGAACCTGGAATAGAGATTTTCCTGTGCAAAGGAG |
| | | AGAACCCTGTATTCACCCTGGTTTTCTGGGGCAGAGACA |
| | | GGGAAGAAGCTGAAGCTAGAAAAAACAAAGGGCTTTCG |
| | | ATTCTAAAAAGCCGTTTCGGAGCTGCTGCATAA |
| 71 | PylC Variant | MRESWGASLKTICLIGGKLQGFEAAYLSKKAGMKVLVIDK |
| | K200-9 | NPQALIRNYADEFQCFNITEEPEKLVAISKNVDAILPVNENL |
| | Polypeptide | ECIEFLNSIKEKFSCPVLFDFEAYRISRDKRKSKEYFASIGTP |
| | Sequence; | TPQDKPSEPPYFVKPPCESSSVGARIIHDRKELKELEPGMLIE |
| | mutations are | EYVEGEVVHLLVIGDGNNFAVVKETLVHIDDTYDCHMVT |
| | shown in bold and | PLPLDPSFRELSYSLAANLPLKGIMSVEAISGPLGLKVIEIDA |
| | underlined letters | RFPSQSPTAVYYSSGINLIELLFRAFNGGIEEIKTLPEDRYCI |
| | | YEHLMLAENGVLIPVGEQVLSMGNDYGNYYEEPGIEIFLC |
| | | KGENPVFTLVFWGRDREEAEARKNKGLSILKSRFGAAA |
| 72 | PylC Variant | ATGAGAGAGTCCTGGGGTGCTAGCCTGAAAACAATATG |
| | K150-23 | CCTTATAGGCGGGAAGCTGCAGGGCTTCGAGGCTGCATA |
| | Polynucleotide | CCTATCTAAGAAAGCCGGAATGAAAGTGCTTGTAATAG |
| | Sequence; | ACAAAAACCCGCAGGCGCTTATAAGGAATTATGCGGAT |
| | mutations are | GAGTTCCAGTGTTTTAACATAACGGAAGAGCCGGAAAA |
| | shown in bold and | ACTCGTCGCGATATCAAAAAATGTTGATGCCATACTGCC |
| | underlined letters | GGTAAATGAAAACCTTGAATGTATAGAATTTCTGAATTC |
| | | TATAAAAGAAAAATTCTCCTGCCCGGTACTTTTCGATTTT |
| | | GAAGCTTACAGGATCAGCAGGGATAAGAGAAAATCAAA |
| | | AGAATACTTCGCATCCATAGGAACCCCGACCCCTCAGGA |
| | | CAAACCGTCGGAACCACCTTATTTTGTAAAGCCTCCCTG |
| | | CGAAAGCAGCAGTGTGGGAGCGAGAATAATCCATGACA |
| | | GGAAAGAGCTTAAAGAGCTTGAGCCCGGGATGCTCATA |
| | | GAAGAATACGTTGAAGGGGAAGTGGTCTCTCTTGGTGT |
| | | CATAGGGGATGGAAATAATTTTGCTGTGGTAAAGGAAA |
| | | CCCTTGTACATATCGATGACACCTATGACTGCCATATGG |
| | | TGACCCCTCTCCCTCTAGACCCTTCCTTCAGGGAACTATC |
| | | CTACTCCCTTGCAGCAAACCTGCCCTTAAAAGGAATTAT |
| | | GCTGGTGGAAGCGATTTCCGGCCCCCTGGGGTTAAAAG |
| | | TTATTGAGATAGATGCCCGTTTCCCGAGCCAGGTGCCGA |
| | | CTGCGGTCTATTATTCTTCCGGGATCAACCTCATAGAAC |
| | | TCCTGTTCCGGGCTTTTAATGGAGGCATAGAAGAGATCA |
| | | AAACTCTCCCTGAAGACAGGTACTGCATTTACGAACATC |
| | | TCATGCTTGCAGAAAATGGAGTACTTATCCCTGTGGGAG |
| | | AACAGGTCCTGTCCATGGGAAATGATTACGGCAATTATT |
| | | ATGAAGAACCTGGAATAGAGATTTTCCTGTGCAAAGGA |
| | | GAGAACCCTGTATTCACCCTGGTTTTCTGGGGCAGAGAC |
| | | AGGGAAGAAGCTGAAGCTAGAAAAAACAAAGGGCTTTC |
| | | GATTCTAAAAAGCCGTTTCGGAGCTGCTGCATAA |
| 73 | PylC Variant | MRESWGASLKTICLIGGKLQGFEAAYLSKKAGMKVLVIDK |
| | K150-23 | NPQALIRNYADEFQCFNITEEPEKLVAISKNVDAILPVNENL |
| | Polypeptide | ECIEFLNSIKEKFSCPVLFDFEAYRISRDKRKSKEYFASIGTP |
| | Sequence; | TPQDKPSEPPYFVKPPCESSSVGARIIHDRKELKELEPGMLIE |
| | mutations are | EYVEGEVVSLGVIGDGNNFAVVKETLVHIDDTYDCHMVTP |
| | shown in bold and | LPLDPSFRELSYSLAANLPLKGIMLVEAISGPLGLKVIEIDAR |
| | underlined letters | FPSQVPTAVYYSSGINLIELLFRAFNGGIEEIKTLPEDRYCIY |
| | | EHLMLAENGVLIPVGEQVLSMGNDYGNYYEEPGIEIFLCK |
| | | GENPVFTLVFWGRDREEAEARKNKGLSILKSRFGAAA |
| 74 | PylC Variant | ATGAGAGAGTCCTGGGGTGCTAGCCTGAAAACAATATG |
| | K150-19 | CCTTATAGGCGGGAAGCTGCAGGGCTTCGAGGCTGCATA |
| | Polynucleotide | CCTATCTAAGAAAGCCGGAATGAAAGTGCTTGTAATAG |
| | Sequence | ACAAAAACCCGCAGGCGCTTATAAGGAATTATGCGGAT |
| | (PylCNPSV); | GAGTTCCAGTGTTTTAACATAACGGAAGAGCCGGAAAA |
| | mutations are | ACTCGTCGCGATATCAAAAAATGTTGATGCCATACTGCC |
| | shown in bold and | GGTAAATGAAAACCTTGAATGTATAGAATTTCTGAATTC |
| | underlined letters | TATAAAAGAAAAATTCTCCTGCCCGGTACTTTTCGATTTT |
| | | GAAGCTTACAGGATCAGCAGGGATAAGAGAAAATCAAA |
| | | AGAATACTTCGCATCCATAGGAACCCCGACCCCTCAGGA |
| | | CAAACCGTCGGAACCACCTTATTTTGTAAAGCCTCCCTG |
| | | CGAAAGCAGCAGTGTGGGAGCGAGAATAATCCATGACA |
| | | GGAAAGAGCTTAAAGAGCTTGAGCCCGGGATGCTCATA |
| | | GAAGAATACGTTGAAGGGGAAGTGGTCAATCTTCCTGT |
| | | CATAGGGGATGGAAATAATTTTGCTGTGGTAAAGGAAA |
| | | CCCTTGTACATATCGATGACACCTATGACTGCCATATGG |
| | | TGACCCCTCTCCCTCTAGACCCTTCCTTCAGGGAACTATC |
| | | CTACTCCCTTGCAGCAAACCTGCCCTTAAAAGGAATTAT |
| | | GTCTGTGGAAGCGATTTCCGGCCCCCTGGGGTTAAAAGT |
| | | TATTGAGATAGATGCCCGTTTCCCGAGCCAGGTTCCGAC |
| | | TGCGGTCTATTATTCTTCCGGGATCAACCTCATAGAACT |
| | | CCTGTTCCGGGCTTTTAATGGAGGCATAGAAGAGATCAA |
| | | AACTCTCCCTGAAGACAGGTACTGCATTTACGAACATCT |
| | | CATGCTTGCAGAAAATGGAGTACTTATCCCTGTGGGAGA |
| | | ACAGGTCCTGTCCATGGGAAATGATTACGGCAATTATTA |
| | | TGAAGAACCTGGAATAGAGATTTTCCTGTGCAAAGGAG |
| | | AGAACCCTGTATTCACCCTGGTTTTCTGGGGCAGAGACA |
| | | GGGAAGAAGCTGAAGCTAGAAAAAACAAAGGGCTTTCG |
| | | ATTCTAAAAAGCCGTTTCGGAGCTGCTGCATAA |
| 75 | PylC Variant | MRESWGASLKTICLIGGKLQGFEAAYLSKKAGMKVLVIDK |
| | K150-19 | NPQALIRNYADEFQCFNITEEPEKLVAISKNVDAILPVNENL |
| | Polypeptide | ECIEFLNSIKEKFSCPVLFDFEAYRISRDKRKSKEYFASIGTP |
| | Sequence | TPQDKPSEPPYFVKPPCESSSVGARIIHDRKELKELEPGMLIE |
| | (PylCNPSV); | EYVEGEVVNLPVIGDGNNFAVVKETLVHIDDTYDCHMVTP |
| | mutations are | LPLDPSFRELSYSLAANLPLKGIMSVEAISGPLGLKVIEIDAR |
| | shown in bold and | FPSQVPTAVYYSSGINLIELLFRAFNGGIEEIKTLPEDRYCIY |
| | underlined letters | EHLMLAENGVLIPVGEQVLSMGNDYGNYYEEPGIEIFLCK |
| | | GENPVFTLVFWGRDREEAEARKNKGLSILKSRFGAAA |
| 76 | PylC Variant | ATGAGAGAGTCCTGGGGTGCTAGCCTGAAAACAATATG |
| | K150-60 | CCTTATAGGCGGGAAGCTGCAGGGCTTCGAGGCTGCATA |
| | Polynucleotide | CCTATCTAAGAAAGCCGGAATGAAAGTGCTTGTAATAG |
| | Sequence; | ACAAAAACCCGCAGGCGCTTATAAGGAATTATGCGGAT |
| | mutations are | GAGTTCCAGTGTTTTAACATAACGGAAGAGCCGGAAAA |
| | shown in bold and | ACTCGTCGCGATATCAAAAAATGTTGATGCCATACTGCC |
| | underlined letters | GGTAAATGAAAACCTTGAATGTATAGAATTTCTGAATTC |
| | | TATAAAAGAAAAATTCTCCTGCCCGGTACTTTTCGATTTT |
| | | GAAGCTTACAGGATCAGCAGGGATAAGAGAAAATCAAA |
| | | AGAATACTTCGCATCCATAGGAACCCCGACCCCTCAGGA |
| | | CAAACCGTCGGAACCACCTTATTTTGTAAAGCCTCCCTG |
| | | CGAAAGCAGCAGTGTGGGAGCGAGAATAATCCATGACA |
| | | GGAAAGAGCTTAAAGAGCTTGAGCCCGGGATGCTCATA |
| | | GAAGAATACGTTGAAGGGGAAGTGGTCATGCTTGGTGT |
| | | CATAGGGGATGGAAATAATTTTGCTGTGGTAAAGGAAA |
| | | CCCTTGTACATATCGATGACACCTATGACTGCCATATGG |
| | | TGACCCCTCTCCCTCTAGACCCTTCCTTCAGGGAACTATC |
| | | CTACTCCCTTGCAGCAAACCTGCCCTTAAAAGGAATTAT |
| | | GCATGTGGAAGCGATTTCCGGCCCCCTGGGGTTAAAAGT |
| | | TATTGAGATAGATGCCCGTTTCCCGAGCCAGTATCCGAC |
| | | TGCGGTCTATTATTCTTCCGGGATCAACCTCATAGAACT |
| | | CCTGTTCCGGGCTTTTAATGGAGGCATAGAAGAGATCAA |
| | | AACTCTCCCTGAAGACAGGTACTGCATTTACGAACATCT |
| | | CATGCTTGCAGAAAATGGAGTACTTATCCCTGTGGGAGA |
| | | ACAGGTCCTGTCCATGGGAAATGATTACGGCAATTATTA |
| | | TGAAGAACCTGGAATAGAGATTTTCCTGTGCAAAGGAG |
| | | AGAACCCTGTATTCACCCTGGTTTTCTGGGGCAGAGACA |
| | | GGGAAGAAGCTGAAGCTAGAAAAAACAAAGGGCTTTCG |
| | | ATTCTAAAAAGCCGTTTCGGAGCTGCTGCATAA |
| 77 | PylC Variant | MRESWGASLKTICLIGGKLQGFEAAYLSKKAGMKVLVIDK |
| | K150-60 | NPQALIRNYADEFQCFNITEEPEKLVAISKNVDAILPVNENL |
| | Polypeptide | ECIEFLNSIKEKFSCPVLFDFEAYRISRDKRKSKEYFASIGTP |
| | Sequence; | TPQDKPSEPPYFVKPPCESSSVGARIIHDRKELKELEPGMLIE |
| | mutations are | EYVEGEVVMLGVIGDGNNFAVVKETLVHIDDTYDCHMVT |
| | shown in bold and | PLPLDPSFRELSYSLAANLPLKGIMHVEAISGPLGLKVIEIDA |
| | underlined letters | RFPSQYPTAVYYSSGINLIELLFRAFNGGIEEIKTLPEDRYCI |
| | | YEHLMLAENGVLIPVGEQVLSMGNDYGNYYEEPGIEIFLC |
| | | KGENPVFTLVFWGRDREEAEARKNKGLSILKSRFGAAA |
| 78 | PylC Variant | ATGAGAGAGTCCTGGGGTGCTAGCCTGAAAACAATATG |
| | K200-5 | CCTTATAGGCGGGAAGCTGCAGGGCTTCGAGGCTGCATA |
| | Polynucleotide | CCTATCTAAGAAAGCCGGAATGAAAGTGCTTGTAATAG |
| | Sequence; | ACAAAAACCCGCAGGCGCTTATAAGGAATTATGCGGAT |
| | mutations are | GAGTTCCAGTGTTTTAACATAACGGAAGAGCCGGAAAA |
| | shown in bold and | ACTCGTCGCGATATCAAAAAATGTTGATGCCATACTGCC |
| | underlined letters | GGTAAATGAAAACCTTGAATGTATAGAATTTCTGAATTC |
| | | TATAAAAGAAAAATTCTCCTGCCCGGTACTTTTCGATTTT |
| | | GAAGCTTACAGGATCAGCAGGGATAAGAGAAAATCAAA |
| | | AGAATACTTCGCATCCATAGGAACCCCGACCCCTCAGGA |
| | | CAAACCGTCGGAACCACCTTATTTTGTAAAGCCTCCCTG |
| | | CGAAAGCAGCAGTGTGGGAGCGAGAATAATCCATGACA |
| | | GGAAAGAGCTTAAAGAGCTTGAGCCCGGGATGCTCATA |
| | | GAAGAATACGTTGAAGGGGAAGTGGTCATGCTTTTGGT |
| | | CATAGGGGATGGAAATAATTTTGCTGTGGTAAAGGAAA |
| | | CCCTTGTACATATCGATGACACCTATGACTGCCATATGG |
| | | TGACCCCTCTCCCTCTAGACCCTTCCTTCAGGGAACTATC |
| | | CTACTCCCTTGCAGCAAACCTGCCCTTAAAAGGAATTAT |
| | | GTCTGTGGAAGCGATTTCCGGCCCCCTGGGGTTAAAAGT |
| | | TATTGAGATAGATGCCCGTTTCCCGAGCCAGTGTCCGAC |
| | | TGCGGTCTATTATTCTTCCGGGATCAACCTCATAGAACT |
| | | CCTGTTCCGGGCTTTTAATGGAGGCATAGAAGAGATCAA |
| | | AACTCTCCCTGAAGACAGGTACTGCATTTACGAACATCT |
| | | CATGCTTGCAGAAAATGGAGTACTTATCCCTGTGGGAGA |
| | | ACAGGTCCTGTCCATGGGAAATGATTACGGCAATTATTA |
| | | TGAAGAACCTGGAATAGAGATTTTCCTGTGCAAAGGAG |
| | | AGAACCCTGTATTCACCCTGGTTTTCTGGGGCAGAGACA |
| | | GGGAAGAAGCTGAAGCTAGAAAAAACAAAGGGCTTTCG |
| | | ATTCTAAAAAGCCGTTTCGGAGCTGCTGCATAA |
| 79 | PylC Variant | MRESWGASLKTICLIGGKLQGFEAAYLSKKAGMKVLVIDK |
| | K200-5 | NPQALIRNYADEFQCFNITEEPEKLVAISKNVDAILPVNENL |
| | Polypeptide | ECIEFLNSIKEKFSCPVLFDFEAYRISRDKRKSKEYFASIGTP |
| | Sequence; | TPQDKPSEPPYFVKPPCESSSVGARIIHDRKELKELEPGMLIE |
| | mutations are | EYVEGEVVMLLVIGDGNNFAVVKETLVHIDDTYDCHMVT |
| | shown in bold and | PLPLDPSFRELSYSLAANLPLKGIMSVEAISGPLGLKVIEIDA |
| | underlined letters | RFPSQCPTAVYYSSGINLIELLFRAFNGGIEEIKTLPEDRYCI |
| | | YEHLMLAENGVLIPVGEQVLSMGNDYGNYYEEPGIEIFLC |
| | | KGENPVFTLVFWGRDREEAEARKNKGLSILKSRFGAAA |
| 80 | PylC Variant | ATGAGAGAGTCCTGGGGTGCTAGCCTGAAAACAATATG |
| | K150-54 | CCTTATAGGCGGGAAGCTGCAGGGCTTCGAGGCTGCATA |
| | Polynucleotide | CCTATCTAAGAAAGCCGGAATGAAAGTGCTTGTAATAG |
| | Sequence; | ACAAAAACCCGCAGGCGCTTATAAGGAATTATGCGGAT |
| | mutations are | GAGTTCCAGTGTTTTAACATAACGGAAGAGCCGGAAAA |
| | shown in bold and | ACTCGTCGCGATATCAAAAAATGTTGATGCCATACTGCC |
| | underlined letters | GGTAAATGAAAACCTTGAATGTATAGAATTTCTGAATTC |
| | | TATAAAAGAAAAATTCTCCTGCCCGGTACTTTTCGATTTT |
| | | GAAGCTTACAGGATCAGCAGGGATAAGAGAAAATCAAA |
| | | AGAATACTTCGCATCCATAGGAACCCCGACCCCTCAGGA |
| | | CAAACCGTCGGAACCACCTTATTTTGTAAAGCCTCCCTG |
| | | CGAAAGCAGCAGTGTGGGAGCGAGAATAATCCATGACA |
| | | GGAAAGAGCTTAAAGAGCTTGAGCCCGGGATGCTCATA |
| | | GAAGAATACGTTGAAGGGGAAGTGGTCGCTCTTTGTGT |
| | | CATAGGGGATGGAAATAATTTTGCTGTGGTAAAGGAAA |
| | | CCCTTGTACATATCGATGACACCTATGACTGCCATATGG |
| | | TGACCCCTCTCCCTCTAGACCCTTCCTTCAGGGAACTATC |
| | | CTACTCCCTTGCAGCAAACCTGCCCTTAAAAGGAATTAT |
| | | GCATGTGGAAGCGATTTCCGGCCCCCTGGGGTTAAAAGT |
| | | TATTGAGATAGATGCCCGTTTCCCGAGCCAGACTCCGAC |
| | | TGCGGTCTATTATTCTTCCGGGATCAACCTCATAGAACT |
| | | CCTGTTCCGGGCTTTTAATGGAGGCATAGAAGAGATCAA |
| | | AACTCTCCCTGAAGACAGGTACTGCATTTACGAACATCT |
| | | CATGCTTGCAGAAAATGGAGTACTTATCCCTGTGGGAGA |
| | | ACAGGTCCTGTCCATGGGAAATGATTACGGCAATTATTA |
| | | TGAAGAACCTGGAATAGAGATTTTCCTGTGCAAAGGAG |
| | | AGAACCCTGTATTCACCCTGGTTTTCTGGGGCAGAGACA |
| | | GGGAAGAAGCTGAAGCTAGAAAAAACAAAGGGCTTTCG |
| | | ATTCTAAAAAGCCGTTTCGGAGCTGCTGCATAA |
| 81 | PylC Variant | MRESWGASLKTICLIGGKLQGFEAAYLSKKAGMKVLVIDK |
| | K150-54 | NPQALIRNYADEFQCFNITEEPEKLVAISKNVDAILPVNENL |
| | Polypeptide | ECIEFLNSIKEKFSCPVLFDFEAYRISRDKRKSKEYFASIGTP |
| | Sequence; | TPQDKPSEPPYFVKPPCESSSVGARIIHDRKELKELEPGMLIE |
| | mutations are | EYVEGEVVALCVIGDGNNFAVVKETLVHIDDTYDCHMVT |
| | shown in bold and | PLPLDPSFRELSYSLAANLPLKGIMHVEAISGPLGLKVIEIDA |
| | underlined letters | RFPSQTPTAVYYSSGINLIELLFRAFNGGIEEIKTLPEDRYCI |
| | | YEHLMLAENGVLIPVGEQVLSMGNDYGNYYEEPGIEIFLC |
| | | KGENPVFTLVFWGRDREEAEARKNKGLSILKSRFGAAA |
| 82 | PylC Variant | ATGAGAGAGTCCTGGGGTGCTAGCCTGAAAACAATATG |
| | K200-1 | CCTTATAGGCGGGAAGCTGCAGGGCTTCGAGGCTGCATA |
| | Polynucleotide | CCTATCTAAGAAAGCCGGAATGAAAGTGCTTGTAATAG |
| | Sequence; | ACAAAAACCCGCAGGCGCTTATAAGGAATTATGCGGAT |
| | mutations are | GAGTTCCAGTGTTTTAACATAACGGAAGAGCCGGAAAA |
| | shown in bold and | ACTCGTCGCGATATCAAAAAATGTTGATGCCATACTGCC |
| | underlined letters | GGTAAATGAAAACCTTGAATGTATAGAATTTCTGAATTC |
| | | TATAAAAGAAAAATTCTCCTGCCCGGTACTTTTCGATTTT |
| | | GAAGCTTACAGGATCAGCAGGGATAAGAGAAAATCAAA |
| | | AGAATACTTCGCATCCATAGGAACCCCGACCCCTCAGGA |
| | | CAAACCGTCGGAACCACCTTATTTTGTAAAGCCTCCCTG |
| | | CGAAAGCAGCAGTGTGGGAGCGAGAATAATCCATGACA |
| | | GGAAAGAGCTTAAAGAGCTTGAGCCCGGGATGCTCATA |
| | | GAAGAATACGTTGAAGGGGAAGTGGTCTCGCTTCTTGT |
| | | CATAGGGGATGGAAATAATTTTGCTGTGGTAAAGGAAA |
| | | CCCTTGTACATATCGATGACACCTATGACTGCCATATGG |
| | | TGACCCCTCTCCCTCTAGACCCTTCCTTCAGGGAACTATC |
| | | CTACTCCCTTGCAGCAAACCTGCCCTTAAAAGGAATTAT |
| | | GCTGGTGGAAGCGATTTCCGGCCCCCTGGGGTTAAAAG |
| | | TTATTGAGATAGATGCCCGTTTCCCGAGCCAGATTCCGA |
| | | CTGCGGTCTATTATTCTTCCGGGATCAACCTCATAGAAC |
| | | TCCTGTTCCGGGCTTTTAATGGAGGCATAGAAGAGATCA |
| | | AAACTCTCCCTGAAGACAGGTACTGCATTTACGAACATC |
| | | TCATGCTTGCAGAAAATGGAGTACTTATCCCTGTGGGAG |
| | | AACAGGTCCTGTCCATGGGAAATGATTACGGCAATTATT |
| | | ATGAAGAACCTGGAATAGAGATTTTCCTGTGCAAAGGA |
| | | GAGAACCCTGTATTCACCCTGGTTTTCTGGGGCAGAGAC |
| | | AGGGAAGAAGCTGAAGCTAGAAAAAACAAAGGGCTTTC |
| | | GATTCTAAAAAGCCGTTTCGGAGCTGCTGCATAA |
| 83 | PylC Variant | MRESWGASLKTICLIGGKLQGFEAAYLSKKAGMKVLVIDK |
| | K200-1 | NPQALIRNYADEFQCFNITEEPEKLVAISKNVDAILPVNENL |
| | Polypeptide | ECIEFLNSIKEKFSCPVLFDFEAYRISRDKRKSKEYFASIGTP |
| | Sequence; | TPQDKPSEPPYFVKPPCESSSVGARIIHDRKELKELEPGMLIE |
| | mutations are | EYVEGEVVSLLVIGDGNNFAVVKETLVHIDDTYDCHMVTP |
| | shown in bold and | LPLDPSFRELSYSLAANLPLKGIMLVEAISGPLGLKVIEIDAR |
| | underlined letters | FPSQIPTAVYYSSGINLIELLFRAFNGGIEEIKTLPEDRYCIYE |
| | | HLMLAENGVLIPVGEQVLSMGNDYGNYYEEPGIEIFLCKG |
| | | ENPVFTLVFWGRDREEAEARKNKGLSILKSRFGAAA |
| 84 | PylC Variant | ATGAGAGAGTCCTGGGGTGCTAGCCTGAAAACAATATG |
| | K150-24 | CCTTATAGGCGGGAAGCTGCAGGGCTTCGAGGCTGCATA |
| | Polynucleotide | CCTATCTAAGAAAGCCGGAATGAAAGTGCTTGTAATAG |
| | Sequence; | ACAAAAACCCGCAGGCGCTTATAAGGAATTATGCGGAT |
| | mutations are | GAGTTCCAGTGTTTTAACATAACGGAAGAGCCGGAAAA |
| | shown in bold and | ACTCGTCGCGATATCAAAAAATGTTGATGCCATACTGCC |
| | underlined letters | GGTAAATGAAAACCTTGAATGTATAGAATTTCTGAATTC |
| | | TATAAAAGAAAAATTCTCCTGCCCGGTACTTTTCGATTTT |
| | | GAAGCTTACAGGATCAGCAGGGATAAGAGAAAATCAAA |
| | | AGAATACTTCGCATCCATAGGAACCCCGACCCCTCAGGA |
| | | CAAACCGTCGGAACCACCTTATTTTGTAAAGCCTCCCTG |
| | | CGAAAGCAGCAGTGTGGGAGCGAGAATAATCCATGACA |
| | | GGAAAGAGCTTAAAGAGCTTGAGCCCGGGATGCTCATA |
| | | GAAGAATACGTTGAAGGGGAAGTGGTCCTTCTTGTTGT |
| | | CATAGGGGATGGAAATAATTTTGCTGTGGTAAAGGAAA |
| | | CCCTTGTACATATCGATGACACCTATGACTGCCATATGG |
| | | TGACCCCTCTCCCTCTAGACCCTTCCTTCAGGGAACTATC |
| | | CTACTCCCTTGCAGCAAACCTGCCCTTAAAAGGAATTAT |
| | | GCATGTGGAAGCGATTTCCGGCCCCCTGGGGTTAAAAGT |
| | | TATTGAGATAGATGCCCGTTTCCCGAGCCAGTATCCGAC |
| | | TGCGGTCTATTATTCTTCCGGGATCAACCTCATAGAACT |
| | | CCTGTTCCGGGCTTTTAATGGAGGCATAGAAGAGATCAA |
| | | AACTCTCCCTGAAGACAGGTACTGCATTTACGAACATCT |
| | | CATGCTTGCAGAAAATGGAGTACTTATCCCTGTGGGAGA |
| | | ACAGGTCCTGTCCATGGGAAATGATTACGGCAATTATTA |
| | | TGAAGAACCTGGAATAGAGATTTTCCTGTGCAAAGGAG |
| | | AGAACCCTGTATTCACCCTGGTTTTCTGGGGCAGAGACA |
| | | GGGAAGAAGCTGAAGCTAGAAAAAACAAAGGGCTTTCG |
| | | ATTCTAAAAAGCCGTTTCGGAGCTGCTGCATAA |
| 85 | PylC Variant | MRESWGASLKTICLIGGKLQGFEAAYLSKKAGMKVLVIDK |
| | K150-24 | NPQALIRNYADEFQCFNITEEPEKLVAISKNVDAILPVNENL |
| | Polypeptide | ECIEFLNSIKEKFSCPVLFDFEAYRISRDKRKSKEYFASIGTP |
| | Sequence; | TPQDKPSEPPYFVKPPCESSSVGARIIHDRKELKELEPGMLIE |
| | mutations are | EYVEGEVVLLVVIGDGNNFAVVKETLVHIDDTYDCHMVT |
| | shown in bold and | PLPLDPSFRELSYSLAANLPLKGIMHVEAISGPLGLKVIEIDA |
| | underlined letters | RFPSQYPTAVYYSSGINLIELLFRAFNGGIEEIKTLPEDRYCI |
| | | YEHLMLAENGVLIPVGEQVLSMGNDYGNYYEEPGIEIFLC |
| | | KGENPVFTLVFWGRDREEAEARKNKGLSILKSRFGAAA |
| 86 | PylC Variant | ATGAGAGAGTCCTGGGGTGCTAGCCTGAAAACAATATG |
| | K200-41 | CCTTATAGGCGGGAAGCTGCAGGGCTTCGAGGCTGCATA |
| | Polynucleotide | CCTATCTAAGAAAGCCGGAATGAAAGTGCTTGTAATAG |
| | Sequence; | ACAAAAACCCGCAGGCGCTTATAAGGAATTATGCGGAT |
| | mutations are | GAGTTCCAGTGTTTTAACATAACGGAAGAGCCGGAAAA |
| | shown in bold and | ACTCGTCGCGATATCAAAAAATGTTGATGCCATACTGCC |
| | underlined letters | GGTAAATGAAAACCTTGAATGTATAGAATTTCTGAATTC |
| | | TATAAAAGAAAAATTCTCCTGCCCGGTACTTTTCGATTTT |
| | | GAAGCTTACAGGATCAGCAGGGATAAGAGAAAATCAAA |
| | | AGAATACTTCGCATCCATAGGAACCCCGACCCCTCAGGA |
| | | CAAACCGTCGGAACCACCTTATTTTGTAAAGCCTCCCTG |
| | | CGAAAGCAGCAGTGTGGGAGCGAGAATAATCCATGACA |
| | | GGAAAGAGCTTAAAGAGCTTGAGCCCGGGATGCTCATA |
| | | GAAGAATACGTTGAAGGGGAAGTGGTCGCTCTTCAGGT |
| | | CATAGGGGATGGAAATAATTTTGCTGTGGTAAAGGAAA |
| | | CCCTTGTACATATCGATGACACCTATGACTGCCATATGG |
| | | TGACCCCTCTCCCTCTAGACCCTTCCTTCAGGGAACTATC |
| | | CTACTCCCTTGCAGCAAACCTGCCCTTAAAAGGAATTAT |
| | | GAATGTGGAAGCGATTTCCGGCCCCCTGGGGTTAAAAGT |
| | | TATTGAGATAGATGCCCGTTTCCCGAGCCAGTGTCCGAC |
| | | TGCGGTCTATTATTCTTCCGGGATCAACCTCATAGAACT |
| | | CCTGTTCCGGGCTTTTAATGGAGGCATAGAAGAGATCAA |
| | | AACTCTCCCTGAAGACAGGTACTGCATTTACGAACATCT |
| | | CATGCTTGCAGAAAATGGAGTACTTATCCCTGTGGGAGA |
| | | ACAGGTCCTGTCCATGGGAAATGATTACGGCAATTATTA |
| | | TGAAGAACCTGGAATAGAGATTTTCCTGTGCAAAGGAG |
| | | AGAACCCTGTATTCACCCTGGTTTTCTGGGGCAGAGACA |
| | | GGGAAGAAGCTGAAGCTAGAAAAAACAAAGGGCTTTCG |
| | | ATTCTAAAAAGCCGTTTCGGAGCTGCTGCATAA |
| 87 | PylC Variant | MRESWGASLKTICLIGGKLQGFEAAYLSKKAGMKVLVIDK |
| | K200-41 | NPQALIRNYADEFQCFNITEEPEKLVAISKNVDAILPVNENL |
| | Polypeptide | ECIEFLNSIKEKFSCPVLFDFEAYRISRDKRKSKEYFASIGTP |
| | Sequence; | TPQDKPSEPPYFVKPPCESSSVGARIIHDRKELKELEPGMLIE |
| | mutations are | EYVEGEVVALQVIGDGNNFAVVKETLVHIDDTYDCHMVT |
| | shown in bold and | PLPLDPSFRELSYSLAANLPLKGIMNVEAISGPLGLKVIEIDA |
| | underlined letters | RFPSQCPTAVYYSSGINLIELLFRAFNGGIEEIKTLPEDRYCI |
| | | YEHLMLAENGVLIPVGEQVLSMGNDYGNYYEEPGIEIFLC |
| | | KGENPVFTLVFWGRDREEAEARKNKGLSILKSRFGAAA |
| 88 | PylC Variant | ATGAGAGAGTCCTGGGGTGCTAGCCTGAAAACAATATG |
| | K200-40 | CCTTATAGGCGGGAAGCTGCAGGGCTTCGAGGCTGCATA |
| | Polynucleotide | CCTATCTAAGAAAGCCGGAATGAAAGTGCTTGTAATAG |
| | Sequence; | ACAAAAACCCGCAGGCGCTTATAAGGAATTATGCGGAT |
| | mutations are | GAGTTCCAGTGTTTTAACATAACGGAAGAGCCGGAAAA |
| | shown in bold and | ACTCGTCGCGATATCAAAAAATGTTGATGCCATACTGCC |
| | underlined letters | GGTAAATGAAAACCTTGAATGTATAGAATTTCTGAATTC |
| | | TATAAAAGAAAAATTCTCCTGCCCGGTACTTTTCGATTTT |
| | | GAAGCTTACAGGATCAGCAGGGATAAGAGAAAATCAAA |
| | | AGAATACTTCGCATCCATAGGAACCCCGACCCCTCAGGA |
| | | CAAACCGTCGGAACCACCTTATTTTGTAAAGCCTCCCTG |
| | | CGAAAGCAGCAGTGTGGGAGCGAGAATAATCCATGACA |
| | | GGAAAGAGCTTAAAGAGCTTGAGCCCGGGATGCTCATA |
| | | GAAGAATACGTTGAAGGGGAAGTGGTCTTGCTTGCGGT |
| | | CATAGGGGATGGAAATAATTTTGCTGTGGTAAAGGAAA |
| | | CCCTTGTACATATCGATGACACCTATGACTGCCATATGG |
| | | TGACCCCTCTCCCTCTAGACCCTTCCTTCAGGGAACTATC |
| | | CTACTCCCTTGCAGCAAACCTGCCCTTAAAAGGAATTAT |
| | | GCATGTGGAAGCGATTTCCGGCCCCCTGGGGTTAAAAGT |
| | | TATTGAGATAGATGCCCGTTTCCCGAGCCAGGTTCCGAC |
| | | TGCGGTCTATTATTCTTCCGGGATCAACCTCATAGAACT |
| | | CCTGTTCCGGGCTTTTAATGGAGGCATAGAAGAGATCAA |
| | | AACTCTCCCTGAAGACAGGTACTGCATTTACGAACATCT |
| | | CATGCTTGCAGAAAATGGAGTACTTATCCCTGTGGGAGA |
| | | ACAGGTCCTGTCCATGGGAAATGATTACGGCAATTATTA |
| | | TGAAGAACCTGGAATAGAGATTTTCCTGTGCAAAGGAG |
| | | AGAACCCTGTATTCACCCTGGTTTTCTGGGGCAGAGACA |
| | | GGGAAGAAGCTGAAGCTAGAAAAAACAAAGGGCTTTCG |
| | | ATTCTAAAAAGCCGTTTCGGAGCTGCTGCATAA |
| 89 | PylC Variant | MRESWGASLKTICLIGGKLQGFEAAYLSKKAGMKVLVIDK |
| | K200-40 | NPQALIRNYADEFQCFNITEEPEKLVAISKNVDAILPVNENL |
| | Polypeptide | ECIEFLNSIKEKFSCPVLFDFEAYRISRDKRKSKEYFASIGTP |
| | Sequence; | TPQDKPSEPPYFVKPPCESSSVGARIIHDRKELKELEPGMLIE |
| | mutations are | EYVEGEVVLLAVIGDGNNFAVVKETLVHIDDTYDCHMVT |
| | shown in bold and | PLPLDPSFRELSYSLAANLPLKGIMHVEAISGPLGLKVIEIDA |
| | underlined letters | RFPSQVPTAVYYSSGINLIELLFRAFNGGIEEIKTLPEDRYCI |
| | | YEHLMLAENGVLIPVGEQVLSMGNDYGNYYEEPGIEIFLC |
| | | KGENPVFTLVFWGRDREEAEARKNKGLSILKSRFGAAA |

Sequences for PylC Variants for Synthesizing D-Pra-ε-Lys

| | | |
|---|---|---|
| 90 | K50-1 | ATGAGAGAGTCCTGGGGTGCTAGCCTGAAAACAATATG |
| | Polynucleotide | CCTTATAGGCGGGAAGCTGCAGGGCTTCGAGGCTGCATA |
| | Sequence; | CCTATCTAAGAAAGCCGGAATGAAAGTGCTTGTAATAG |
| | mutations are | ACAAAAACCCGCAGGCGCTTATAAGGAATTATGCGGAT |
| | shown in bold and | GAGTTCCAGTGTTTTAACATAACGGAAGAGCCGGAAAA |
| | underlined letters | ACTCGTCGCGATATCAAAAAATGTTGATGCCATACTGCC |
| | | GGTAAATGAAAACCTTGAATGTATAGAATTTCTGAATTC |
| | | TATAAAAGAAAAATTCTCCTGCCCGGTACTTTTCGATTTT |
| | | GAAGCTTACAGGATCAGCAGGGATAAGAGAAAATCAAA |
| | | AGAATACTTCGCATCCATAGGAACCCCGACCCCTCAGGA |
| | | CAAACCGTCGGAACCACCTTATTTTGTAAAGCCTCCCTG |
| | | CGAAAGCAGCAGTGTGGGAGCGAGAATAATCCATGACA |
| | | GGAAAGAGCTTAAAGAGCTTGAGCCCGGGATGCTCATA |
| | | GAAGAATACGTTGAAGGGGAAGTGGTCGTGCTTCTTGT |
| | | CATAGGGGATGGAAATAATTTTGCTGTGGTAAAGGAAA |
| | | CCCTTGTACATATCGATGACACCTATGACTGCCATATGG |
| | | TGACCCCTCTCCCTCTAGACCCTTCCTTCAGGGAACTATC |
| | | CTACTCCCTTGCAGCAAACCTGCCCTTAAAAGGAATTAT |
| | | GCATGTGGAAGCGATTTCCGGCCCCCTGGGGTTAAAAGT |
| | | TATTGAGATAGATGCCCGTTTCCCGAGCCAGCTGCCGAC |
| | | TGCGGTCTATTATTCTTCCGGGATCAACCTCATAGAACT |
| | | CCTGTTCCGGGCTTTTAATGGAGGCATAGAAGAGATCAA |
| | | AACTCTCCCTGAAGACAGGTACTGCATTTACGAACATCT |
| | | CATGCTTGCAGAAAATGGAGTACTTATCCCTGTGGGAGA |
| | | ACAGGTCCTGTCCATGGGAAATGATTACGGCAATTATTA |
| | | TGAAGAACCTGGAATAGAGATTTTCCTGTGCAAAGGAG |
| | | AGAACCCTGTATTCACCCTGGTTTTCTGGGGCAGAGACA |
| | | GGGAAGAAGCTGAAGCTAGAAAAAACAAAGGGCTTTCG |
| | | ATTCTAAAAAGCCGTTTCGGAGCTGCTGCATAA |
| 91 | K50-1 Polypeptide | MRESWGASLKTICLIGGKLQGFEAAYLSKKAGMKVLVIDK |
| | Sequence; | NPQALIRNYADEFQCFNITEEPEKLVAISKNVDAILPVNENL |
| | mutations are | ECIEFLNSIKEKFSCPVLFDFEAYRISRDKRKSKEYFASIGTP |
| | shown in bold and | TPQDKPSEPPYFVKPPCESSSVGARIIHDRKELKELEPGMLIE |
| | underlined letters | EYVEGEVVVLLVIGDGNNFAVVKETLVHIDDTYDCHMVT |
| | | PLPLDPSFRELSYSLAANLPLKGIMHVEAISGPLGLKVIEIDA |
| | | RFPSQLPTAVYYSSGINLIELLFRAFNGGIEEIKTLPEDRYCI |
| | | YEHLMLAENGVLIPVGEQVLSMGNDYGNYYEEPGIEIFLC |
| | | KGENPVFTLVFWGRDREEAEARKNKGLSILKSRFGAAA |
| 92 | K50-2 | ATGAGAGAGTCCTGGGGTGCTAGCCTGAAAACAATATG |
| | Polynucleotide | CCTTATAGGCGGGAAGCTGCAGGGCTTCGAGGCTGCATA |
| | Sequence; | CCTATCTAAGAAAGCCGGAATGAAAGTGCTTGTAATAG |
| | mutations are | ACAAAAACCCGCAGGCGCTTATAAGGAATTATGCGGAT |
| | shown in bold and | GAGTTCCAGTGTTTTAACATAACGGAAGAGCCGGAAAA |
| | underlined letters | ACTCGTCGCGATATCAAAAAATGTTGATGCCATACTGCC |
| | | GGTAAATGAAAACCTTGAATGTATAGAATTTCTGAATTC |
| | | TATAAAAGAAAAATTCTCCTGCCCGGTACTTTTCGATTTT |
| | | GAAGCTTACAGGATCAGCAGGGATAAGAGAAAATCAAA |
| | | AGAATACTTCGCATCCATAGGAACCCCGACCCCTCAGGA |
| | | CAAACCGTCGGAACCACCTTATTTTGTAAAGCCTCCCTG |
| | | CGAAAGCAGCAGTGTGGGAGCGAGAATAATCCATGACA |
| | | GGAAAGAGCTTAAAGAGCTTGAGCCCGGGATGCTCATA |
| | | GAAGAATACGTTGAAGGGGAAGTGGTCGGGCTTGTTGT |
| | | CATAGGGGATGGAAATAATTTTGCTGTGGTAAAGGAAA |
| | | CCCTTGTACATATCGATGACACCTATGACTGCCATATGG |
| | | TGACCCCTCTCCCTCTAGACCCTTCCTTCAGGGAACTATC |
| | | CTACTCCCTTGCAGCAAACCTGCCCTTAAAAGGAATTAT |
| | | GCATGTGGAAGCGATTTCCGGCCCCCTGGGGTTAAAAGT |
| | | TATTGAGATAGATGCCCGTTTCCCGAGCCAGGCTCCGAC |
| | | TGCGGTCTATTATTCTTCCGGGATCAACCTCATAGAACT |
| | | CCTGTTCCGGGCTTTTAATGGAGGCATAGAAGAGATCAA |
| | | AACTCTCCCTGAAGACAGGTACTGCATTTACGAACATCT |
| | | CATGCTTGCAGAAAATGGAGTACTTATCCCTGTGGGAGA |
| | | ACAGGTCCTGTCCATGGGAAATGATTACGGCAATTATTA |
| | | TGAAGAACCTGGAATAGAGATTTTCCTGTGCAAAGGAG |
| | | AGAACCCTGTATTCACCCTGGTTTTCTGGGGCAGAGACA |
| | | GGGAAGAAGCTGAAGCTAGAAAAAACAAAGGGCTTTCG |
| | | ATTCTAAAAAGCCGTTTCGGAGCTGCTGCATAA |
| 93 | K50-2 Polypeptide | MRESWGASLKTICLIGGKLQGFEAAYLSKKAGMKVLVIDK |
| | Sequence; | NPQALIRNYADEFQCFNITEEPEKLVAISKNVDAILPVNENL |
| | mutations are | ECIEFLNSIKEKFSCPVLFDFEAYRISRDKRKSKEYFASIGTP |
| | shown in bold and | TPQDKPSEPPYFVKPPCESSSVGARIIHDRKELKELEPGMLIE |
| | underlined letters | EYVEGEVVGLVVIGDGNNFAVVKETLVHIDDTYDCHMVT |
| | | PLPLDPSFRELSYSLAANLPLKGIMHVEAISGPLGLKVIEIDA |
| | | RFPSQAPTAVYYSSGINLIELLFRAFNGGIEEIKTLPEDRYCI |
| | | YEHLMLAENGVLIPVGEQVLSMGNDYGNYYEEPGIEIFLC |
| | | KGENPVFTLVFWGRDREEAEARKNKGLSILKSRFGAAA |
| 94 | K50-3 | ATGAGAGAGTCCTGGGGTGCTAGCCTGAAAACAATATG |
| | Polynucleotide | CCTTATAGGCGGGAAGCTGCAGGGCTTCGAGGCTGCATA |
| | Sequence; | CCTATCTAAGAAAGCCGGAATGAAAGTGCTTGTAATAG |
| | mutations are | ACAAAAACCCGCAGGCGCTTATAAGGAATTATGCGGAT |
| | shown in bold and | GAGTTCCAGTGTTTTAACATAACGGAAGAGCCGGAAAA |
| | underlined letters | ACTCGTCGCGATATCAAAAAATGTTGATGCCATACTGCC |
| | | GGTAAATGAAAACCTTGAATGTATAGAATTTCTGAATTC |
| | | TATAAAAGAAAAATTCTCCTGCCCGGTACTTTTCGATTTT |
| | | GAAGCTTACAGGATCAGCAGGGATAAGAGAAAATCAAA |
| | | AGAATACTTCGCATCCATAGGAACCCCGACCCCTCAGGA |
| | | CAAACCGTCGGAACCACCTTATTTTGTAAAGCCTCCCTG |
| | | CGAAAGCAGCAGTGTGGGAGCGAGAATAATCCATGACA |
| | | GGAAAGAGCTTAAAGAGCTTGAGCCCGGGATGCTCATA |
| | | GAAGAATACGTTGAAGGGGAAGTGGTCGTTCTTTGTGT |
| | | CATAGGGGATGGAAATAATTTTGCTGTGGTAAAGGAAA |
| | | CCCTTGTACATATCGATGACACCTATGACTGCCATATGG |
| | | TGACCCCTCTCCCTCTAGACCCTTCCTTCAGGGAACTATC |
| | | CTACTCCCTTGCAGCAAACCTGCCCTTAAAAGGAATTAT |
| | | GCATGTGGAAGCGATTTCCGGCCCCCTGGGGTTAAAAGT |
| | | TATTGAGATAGATGCCCGTTTCCCGAGCCAGCTGCCGAC |
| | | TGCGGTCTATTATTCTTCCGGGATCAACCTCATAGAACT |
| | | CCTGTTCCGGGCTTTTAATGGAGGCATAGAAGAGATCAA |
| | | AACTCTCCCTGAAGACAGGTACTGCATTTACGAACATCT |
| | | CATGCTTGCAGAAAATGGAGTACTTATCCCTGTGGGAGA |
| | | ACAGGTCCTGTCCATGGGAAATGATTACGGCAATTATTA |
| | | TGAAGAACCTGGAATAGAGATTTTCCTGTGCAAAGGAG |
| | | AGAACCCTGTATTCACCCTGGTTTTCTGGGGCAGAGACA |
| | | GGGAAGAAGCTGAAGCTAGAAAAAACAAAGGGCTTTCG |
| | | ATTCTAAAAAGCCGTTTCGGAGCTGCTGCATAA |
| 95 | K50-3 Polypeptide | MRESWGASLKTICLIGGKLQGFEAAYLSKKAGMKVLVIDK |
| | Sequence; | NPQALIRNYADEFQCFNITEEPEKLVAISKNVDAILPVNENL |
| | mutations are | ECIEFLNSIKEKFSCPVLFDFEAYRISRDKRKSKEYFASIGTP |
| | shown in bold and | TPQDKPSEPPYFVKPPCESSSVGARIIHDRKELKELEPGMLIE |
| | underlined letters | EYVEGEVVVLCVIGDGNNFAVVKETLVHIDDTYDCHMVT |
| | | PLPLDPSFRELSYSLAANLPLKGIMHVEAISGPLGLKVIEIDA |
| | | RFPSQLPTAVYYSSGINLIELLFRAFNGGIEEIKTLPEDRYCI |
| | | YEHLMLAENGVLIPVGEQVLSMGNDYGNYYEEPGIEIFLC |
| | | KGENPVFTLVFWGRDREEAEARKNKGLSILKSRFGAAA |
| 96 | K50-4 | ATGAGAGAGTCCTGGGGTGCTAGCCTGAAAACAATATG |
| | Polynucleotide | CCTTATAGGCGGGAAGCTGCAGGGCTTCGAGGCTGCATA |
| | Sequence; | CCTATCTAAGAAAGCCGGAATGAAAGTGCTTGTAATAG |
| | mutations are | ACAAAAACCCGCAGGCGCTTATAAGGAATTATGCGGAT |
| | shown in bold and | GAGTTCCAGTGTTTTAACATAACGGAAGAGCCGGAAAA |
| | underlined letters | ACTCGTCGCGATATCAAAAAATGTTGATGCCATACTGCC |
| | | GGTAAATGAAAACCTTGAATGTATAGAATTTCTGAATTC |
| | | TATAAAAGAAAAATTCTCCTGCCCGGTACTTTTCGATTTT |
| | | GAAGCTTACAGGATCAGCAGGGATAAGAGAAAATCAAA |
| | | AGAATACTTCGCATCCATAGGAACCCCGACCCCTCAGGA |
| | | CAAACCGTCGGAACCACCTTATTTTGTAAAGCCTCCCTG |
| | | CGAAAGCAGCAGTGTGGGAGCGAGAATAATCCATGACA |
| | | GGAAAGAGCTTAAAGAGCTTGAGCCCGGGATGCTCATA |
| | | GAAGAATACGTTGAAGGGGAAGTGGTCATGCTTGTGGT |
| | | CATAGGGGATGGAAATAATTTTGCTGTGGTAAAGGAAA |
| | | CCCTTGTACATATCGATGACACCTATGACTGCCATATGG |
| | | TGACCCCTCTCCCTCTAGACCCTTCCTTCAGGGAACTATC |
| | | CTACTCCCTTGCAGCAAACCTGCCCTTAAAAGGAATTAT |
| | | GCATGTGGAAGCGATTTCCGGCCCCCTGGGGTTAAAAGT |
| | | TATTGAGATAGATGCCCGTTTCCCGAGCCAGTATCCGAC |
| | | TGCGGTCTATTATTCTTCCGGGATCAACCTCATAGAACT |
| | | CCTGTTCCGGGCTTTTAATGGAGGCATAGAAGAGATCAA |
| | | AACTCTCCCTGAAGACAGGTACTGCATTTACGAACATCT |
| | | CATGCTTGCAGAAAATGGAGTACTTATCCCTGTGGGAGA |
| | | ACAGGTCCTGTCCATGGGAAATGATTACGGCAATTATTA |
| | | TGAAGAACCTGGAATAGAGATTTTCCTGTGCAAAGGAG |
| | | AGAACCCTGTATTCACCCTGGTTTTCTGGGGCAGAGACA |
| | | GGGAAGAAGCTGAAGCTAGAAAAAACAAAGGGCTTTCG |
| | | ATTCTAAAAAGCCGTTTCGGAGCTGCTGCATAA |
| 97 | K50-4 Polypeptide | MRESWGASLKTICLIGGKLQGFEAAYLSKKAGMKVLVIDK |
| | Sequence; | NPQALIRNYADEFQCFNITEEPEKLVAISKNVDAILPVNENL |
| | mutations are | ECIEFLNSIKEKFSCPVLFDFEAYRISRDKRKSKEYFASIGTP |
| | shown in bold and | TPQDKPSEPPYFVKPPCESSSVGARIIHDRKELKELEPGMLIE |
| | underlined letters | EYVEGEVVMLVVIGDGNNFAVVKETLVHIDDTYDCHMVT |
| | | PLPLDPSFRELSYSLAANLPLKGIMHVEAISGPLGLKVIEIDA |
| | | RFPSQYPTAVYYSSGINLIELLFRAFNGGIEEIKTLPEDRYCI |
| | | YEHLMLAENGVLIPVGEQVLSMGNDYGNYYEEPGIEIFLC |
| | | KGENPVFTLVFWGRDREEAEARKNKGLSILKSRFGAAA |
| 98 | K50-5 | ATGAGAGAGTCCTGGGGTGCTAGCCTGAAAACAATATG |
| | Polynucleotide | CCTTATAGGCGGGAAGCTGCAGGGCTTCGAGGCTGCATA |
| | Sequence; | CCTATCTAAGAAAGCCGGAATGAAAGTGCTTGTAATAG |
| | mutations are | ACAAAAACCCGCAGGCGCTTATAAGGAATTATGCGGAT |
| | shown in bold and | GAGTTCCAGTGTTTTAACATAACGGAAGAGCCGGAAAA |
| | underlined letters | ACTCGTCGCGATATCAAAAAATGTTGATGCCATACTGCC |
| | | GGTAAATGAAAACCTTGAATGTATAGAATTTCTGAATTC |
| | | TATAAAAGAAAAATTCTCCTGCCCGGTACTTTTCGATTTT |
| | | GAAGCTTACAGGATCAGCAGGGATAAGAGAAAATCAAA |
| | | AGAATACTTCGCATCCATAGGAACCCCGACCCCTCAGGA |
| | | CAAACCGTCGGAACCACCTTATTTTGTAAAGCCTCCCTG |
| | | CGAAAGCAGCAGTGTGGGAGCGAGAATAATCCATGACA |
| | | GGAAAGAGCTTAAAGAGCTTGAGCCCGGGATGCTCATA |
| | | GAAGAATACGTTGAAGGGGAAGTGGTCTGTCTTCCGGT |
| | | CATAGGGGATGGAAATAATTTTGCTGTGGTAAAGGAAA |
| | | CCCTTGTACATATCGATGACACCTATGACTGCCATATGG |
| | | TGACCCCTCTCCCTCTAGACCCTTCCTTCAGGGAACTATC |
| | | CTACTCCCTTGCAGCAAACCTGCCCTTAAAAGGAATTAT |
| | | GCATGTGGAAGCGATTTCCGGCCCCCTGGGGTTAAAAGT |
| | | TATTGAGATAGATGCCCGTTTCCCGAGCCAGCTGCCGAC |
| | | TGCGGTCTATTATTCTTCCGGGATCAACCTCATAGAACT |
| | | CCTGTTCCGGGCTTTTAATGGAGGCATAGAAGAGATCAA |
| | | AACTCTCCCTGAAGACAGGTACTGCATTTACGAACATCT |
| | | CATGCTTGCAGAAAATGGAGTACTTATCCCTGTGGGAGA |
| | | ACAGGTCCTGTCCATGGGAAATGATTACGGCAATTATTA |
| | | TGAAGAACCTGGAATAGAGATTTTCCTGTGCAAAGGAG |
| | | AGAACCCTGTATTCACCCTGGTTTTCTGGGGCAGAGACA |
| | | GGGAAGAAGCTGAAGCTAGAAAAAACAAAGGGCTTTCG |
| | | ATTCTAAAAAGCCGTTTCGGAGCTGCTGCATAA |
| 99 | K50-5 Polypeptide | MRESWGASLKTICLIGGKLQGFEAAYLSKKAGMKVLVIDK |
| | Sequence; | NPQALIRNYADEFQCFNITEEPEKLVAISKNVDAILPVNENL |
| | mutations are | ECIEFLNSIKEKFSCPVLFDFEAYRISRDKRKSKEYFASIGTP |
| | shown in bold and | TPQDKPSEPPYFVKPPCESSSVGARIIHDRKELKELEPGMLIE |
| | underlined letters | EYVEGEVVCLPVIGDGNNFAVVKETLVHIDDTYDCHMVTP |
| | | LPLDPSFRELSYSLAANLPLKGIMHVEAISGPLGLKVIEIDA |
| | | RFPSQLPTAVYYSSGINLIELLFRAFNGGIEEIKTLPEDRYCI |
| | | YEHLMLAENGVLIPVGEQVLSMGNDYGNYYEEPGIEIFLC |
| | | KGENPVFTLVFWGRDREEAEARKNKGLSILKSRFGAAA |
| 100 | K50-6 | ATGAGAGAGTCCTGGGGTGCTAGCCTGAAAACAATATG |
| | Polynucleotide | CCTTATAGGCGGGAAGCTGCAGGGCTTCGAGGCTGCATA |
| | Sequence; | CCTATCTAAGAAAGCCGGAATGAAAGTGCTTGTAATAG |
| | mutations are | ACAAAAACCCGCAGGCGCTTATAAGGAATTATGCGGAT |
| | shown in bold and | GAGTTCCAGTGTTTTAACATAACGGAAGAGCCGGAAAA |
| | underlined letters | ACTCGTCGCGATATCAAAAAATGTTGATGCCATACTGCC |
| | | GGTAAATGAAAACCTTGAATGTATAGAATTTCTGAATTC |
| | | TATAAAAGAAAAATTCTCCTGCCCGGTACTTTTCGATTTT |
| | | GAAGCTTACAGGATCAGCAGGGATAAGAGAAAATCAAA |
| | | AGAATACTTCGCATCCATAGGAACCCCGACCCCTCAGGA |
| | | CAAACCGTCGGAACCACCTTATTTTGTAAAGCCTCCCTG |
| | | CGAAAGCAGCAGTGTGGGAGCGAGAATAATCCATGACA |
| | | GGAAAGAGCTTAAAGAGCTTGAGCCCGGGATGCTCATA |
| | | GAAGAATACGTTGAAGGGGAAGTGGTCGGGCTTGCTGT |
| | | CATAGGGGATGGAAATAATTTTGCTGTGGTAAAGGAAA |
| | | CCCTTGTACATATCGATGACACCTATGACTGCCATATGG |
| | | TGACCCCTCTCCCTCTAGACCCTTCCTTCAGGGAACTATC |
| | | CTACTCCCTTGCAGCAAACCTGCCCTTAAAAGGAATTAT |
| | | GTCGGTGGAAGCGATTTCCGGCCCCCTGGGGTTAAAAG |
| | | TTATTGAGATAGATGCCCGTTTCCCGAGCCAGCTTCCGA |
| | | CTGCGGTCTATTATTCTTCCGGGATCAACCTCATAGAAC |
| | | TCCTGTTCCGGGCTTTTAATGGAGGCATAGAAGAGATCA |
| | | AAACTCTCCCTGAAGACAGGTACTGCATTTACGAACATC |
| | | TCATGCTTGCAGAAAATGGAGTACTTATCCCTGTGGGAG |
| | | AACAGGTCCTGTCCATGGGAAATGATTACGGCAATTATT |
| | | ATGAAGAACCTGGAATAGAGATTTTCCTGTGCAAAGGA |
| | | GAGAACCCTGTATTCACCCTGGTTTTCTGGGGCAGAGAC |
| | | AGGGAAGAAGCTGAAGCTAGAAAAAACAAAGGGCTTTC |
| | | GATTCTAAAAAGCCGTTTCGGAGCTGCTGCATAA |
| 101 | K50-6 Polypeptide | MRESWGASLKTICLIGGKLQGFEAAYLSKKAGMKVLVIDK |
| | Sequence; | NPQALIRNYADEFQCFNITEEPEKLVAISKNVDAILPVNENL |
| | mutations are | ECIEFLNSIKEKFSCPVLFDFEAYRISRDKRKSKEYFASIGTP |
| | shown in bold and | TPQDKPSEPPYFVKPPCESSSVGARIIHDRKELKELEPGMLIE |
| | underlined letters | EYVEGEVVGLAVIGDGNNFAVVKETLVHIDDTYDCHMVT |
| | | PLPLDPSFRELSYSLAANLPLKGIMSVEAISGPLGLKVIEIDA |
| | | RFPSQLPTAVYYSSGINLIELLFRAFNGGIEEIKTLPEDRYCI |
| | | YEHLMLAENGVLIPVGEQVLSMGNDYGNYYEEPGIEIFLC |
| | | KGENPVFTLVFWGRDREEAEARKNKGLSILKSRFGAAA |
| 102 | K50-7 | ATGAGAGAGTCCTGGGGTGCTAGCCTGAAAACAATATG |
| | Polynucleotide | CCTTATAGGCGGGAAGCTGCAGGGCTTCGAGGCTGCATA |
| | Sequence; | CCTATCTAAGAAAGCCGGAATGAAAGTGCTTGTAATAG |
| | mutations are | ACAAAAACCCGCAGGCGCTTATAAGGAATTATGCGGAT |
| | shown in bold and | GAGTTCCAGTGTTTTAACATAACGGAAGAGCCGGAAAA |
| | underlined letters | ACTCGTCGCGATATCAAAAAATGTTGATGCCATACTGCC |
| | | GGTAAATGAAAACCTTGAATGTATAGAATTTCTGAATTC |
| | | TATAAAAGAAAAATTCTCCTGCCCGGTACTTTTCGATTTT |
| | | GAAGCTTACAGGATCAGCAGGGATAAGAGAAAATCAAA |
| | | AGAATACTTCGCATCCATAGGAACCCCGACCCCTCAGGA |
| | | CAAACCGTCGGAACCACCTTATTTTGTAAAGCCTCCCTG |
| | | CGAAAGCAGCAGTGTGGGAGCGAGAATAATCCATGACA |
| | | GGAAAGAGCTTAAAGAGCTTGAGCCCGGGATGCTCATA |
| | | GAAGAATACGTTGAAGGGGAAGTGGTCGTTCTTCTTGT |
| | | CATAGGGGATGGAAATAATTTTGCTGTGGTAAAGGAAA |
| | | CCCTTGTACATATCGATGACACCTATGACTGCCATATGG |
| | | TGACCCCTCTCCCTCTAGACCCTTCCTTCAGGGAACTATC |
| | | CTACTCCCTTGCAGCAAACCTGCCCTTAAAAGGAATTAT |
| | | GCATGTGGAAGCGATTTCCGGCCCCCTGGGGTTAAAAGT |
| | | TATTGAGATAGATGCCCGTTTCCCGAGCCAGGTGCCGAC |
| | | TGCGGTCTATTATTCTTCCGGGATCAACCTCATAGAACT |
| | | CCTGTTCCGGGCTTTTAATGGAGGCATAGAAGAGATCAA |
| | | AACTCTCCCTGAAGACAGGTACTGCATTTACGAACATCT |
| | | CATGCTTGCAGAAAATGGAGTACTTATCCCTGTGGGAGA |
| | | ACAGGTCCTGTCCATGGGAAATGATTACGGCAATTATTA |
| | | TGAAGAACCTGGAATAGAGATTTTCCTGTGCAAAGGAG |
| | | AGAACCCTGTATTCACCCTGGTTTTCTGGGGCAGAGACA |
| | | GGGAAGAAGCTGAAGCTAGAAAAAACAAAGGGCTTTCG |
| | | ATTCTAAAAAGCCGTTTCGGAGCTGCTGCATAA |
| 103 | K50-7 Polypeptide | MRESWGASLKTICLIGGKLQGFEAAYLSKKAGMKVLVIDK |
| | Sequence; | NPQALIRNYADEFQCFNITEEPEKLVAISKNVDAILPVNENL |
| | mutations are | ECIEFLNSIKEKFSCPVLFDFEAYRISRDKRKSKEYFASIGTP |
| | shown in bold and | TPQDKPSEPPYFVKPPCESSSVGARIIHDRKELKELEPGMLIE |
| | underlined letters | EYVEGEVVVLLVIGDGNNFAVVKETLVHIDDTYDCHMVT |
| | | PLPLDPSFRELSYSLAANLPLKGIMHVEAISGPLGLKVIEIDA |
| | | RFPSQVPTAVYYSSGINLIELLFRAFNGGIEEIKTLPEDRYCI |
| | | YEHLMLAENGVLIPVGEQVLSMGNDYGNYYEEPGIEIFLC |
| | | KGENPVFTLVFWGRDREEAEARKNKGLSILKSRFGAAA |
| 104 | K50-8 | ATGAGAGAGTCCTGGGGTGCTAGCCTGAAAACAATATG |
| | Polynucleotide | CCTTATAGGCGGGAAGCTGCAGGGCTTCGAGGCTGCATA |
| | Sequence; | CCTATCTAAGAAAGCCGGAATGAAAGTGCTTGTAATAG |
| | mutations are | ACAAAAACCCGCAGGCGCTTATAAGGAATTATGCGGAT |
| | shown in bold and | GAGTTCCAGTGTTTTAACATAACGGAAGAGCCGGAAAA |
| | underlined letters | ACTCGTCGCGATATCAAAAAATGTTGATGCCATACTGCC |
| | | GGTAAATGAAAACCTTGAATGTATAGAATTTCTGAATTC |
| | | TATAAAAGAAAAATTCTCCTGCCCGGTACTTTTCGATTTT |
| | | GAAGCTTACAGGATCAGCAGGGATAAGAGAAAATCAAA |
| | | AGAATACTTCGCATCCATAGGAACCCCGACCCCTCAGGA |
| | | CAAACCGTCGGAACCACCTTATTTTGTAAAGCCTCCCTG |
| | | CGAAAGCAGCAGTGTGGGAGCGAGAATAATCCATGACA |
| | | GGAAAGAGCTTAAAGAGCTTGAGCCCGGGATGCTCATA |
| | | GAAGAATACGTTGAAGGGGAAGTGGTCGGGCTTCTTGT |
| | | CATAGGGGATGGAAATAATTTTGCTGTGGTAAAGGAAA |
| | | CCCTTGTACATATCGATGACACCTATGACTGCCATATGG |
| | | TGACCCCTCTCCCTCTAGACCCTTCCTTCAGGGAACTATC |
| | | CTACTCCCTTGCAGCAAACCTGCCCTTAAAAGGAATTAT |
| | | GCATGTGGAAGCGATTTCCGGCCCCCTGGGGTTAAAAGT |
| | | TATTGAGATAGATGCCCGTTTCCCGAGCCAGCTTCCGAC |
| | | TGCGGTCTATTATTCTTCCGGGATCAACCTCATAGAACT |
| | | CCTGTTCCGGGCTTTTAATGGAGGCATAGAAGAGATCAA |
| | | AACTCTCCCTGAAGACAGGTACTGCATTTACGAACATCT |
| | | CATGCTTGCAGAAAATGGAGTACTTATCCCTGTGGGAGA |
| | | ACAGGTCCTGTCCATGGGAAATGATTACGGCAATTATTA |
| | | TGAAGAACCTGGAATAGAGATTTTCCTGTGCAAAGGAG |
| | | AGAACCCTGTATTCACCCTGGTTTTCTGGGGCAGAGACA |
| | | GGGAAGAAGCTGAAGCTAGAAAAAACAAAGGGCTTTCG |
| | | ATTCTAAAAAGCCGTTTCGGAGCTGCTGCATAA |
| 105 | K50-8 Polypeptide | MRESWGASLKTICLIGGKLQGFEAAYLSKKAGMKVLVIDK |
| | Sequence; | NPQALIRNYADEFQCFNITEEPEKLVAISKNVDAILPVNENL |
| | mutations are | ECIEFLNSIKEKFSCPVLFDFEAYRISRDKRKSKEYFASIGTP |
| | shown in bold and | TPQDKPSEPPYFVKPPCESSSVGARIIHDRKELKELEPGMLIE |
| | underlined letters | EYVEGEVVGLLVIGDGNNFAVVKETLVHIDDTYDCHMVT |
| | | PLPLDPSFRELSYSLAANLPLKGIMHVEAISGPLGLKVIEIDA |
| | | RFPSQLPTAVYYSSGINLIELLFRAFNGGIEEIKTLPEDRYCI |
| | | YEHLMLAENGVLIPVGEQVLSMGNDYGNYYEEPGIEIFLC |
| | | KGENPVFTLVFWGRDREEAEARKNKGLSILKSRFGAAA |
| 106 | K100-1 | ATGAGAGAGTCCTGGGGTGCTAGCCTGAAAACAATATG |
| | Polynucleotide | CCTTATAGGCGGGAAGCTGCAGGGCTTCGAGGCTGCATA |
| | Sequence; | CCTATCTAAGAAAGCCGGAATGAAAGTGCTTGTAATAG |
| | mutations are | ACAAAAACCCGCAGGCGCTTATAAGGAATTATGCGGAT |
| | shown in bold and | GAGTTCCAGTGTTTTAACATAACGGAAGAGCCGGAAAA |
| | underlined letters | ACTCGTCGCGATATCAAAAAATGTTGATGCCATACTGCC |
| | | GGTAAATGAAAACCTTGAATGTATAGAATTTCTGAATTC |
| | | TATAAAAGAAAAATTCTCCTGCCCGGTACTTTTCGATTTT |
| | | GAAGCTTACAGGATCAGCAGGGATAAGAGAAAATCAAA |
| | | AGAATACTTCGCATCCATAGGAACCCCGACCCCTCAGGA |
| | | CAAACCGTCGGAACCACCTTATTTTGTAAAGCCTCCCTG |
| | | CGAAAGCAGCAGTGTGGGAGCGAGAATAATCCATGACA |
| | | GGAAAGAGCTTAAAGAGCTTGAGCCCGGGATGCTCATA |
| | | GAAGAATACGTTGAAGGGGAAGTGGTCATTCTTGAGGT |
| | | CATAGGGGATGGAAATAATTTTGCTGTGGTAAAGGAAA |
| | | CCCTTGTACATATCGATGACACCTATGACTGCCATATGG |
| | | TGACCCCTCTCCCTCTAGACCCTTCCTTCAGGGAACTATC |
| | | CTACTCCCTTGCAGCAAACCTGCCCTTAAAAGGAATTAT |
| | | GCATGTGGAAGCGATTTCCGGCCCCCTGGGGTTAAAAGT |
| | | TATTGAGATAGATGCCCGTTTCCCGAGCCAGGTTCCGAC |
| | | TGCGGTCTATTATTCTTCCGGGATCAACCTCATAGAACT |
| | | CCTGTTCCGGGCTTTTAATGGAGGCATAGAAGAGATCAA |
| | | AACTCTCCCTGAAGACAGGTACTGCATTTACGAACATCT |
| | | CATGCTTGCAGAAAATGGAGTACTTATCCCTGTGGGAGA |
| | | ACAGGTCCTGTCCATGGGAAATGATTACGGCAATTATTA |
| | | TGAAGAACCTGGAATAGAGATTTTCCTGTGCAAAGGAG |
| | | AGAACCCTGTATTCACCCTGGTTTTCTGGGGCAGAGACA |
| | | GGGAAGAAGCTGAAGCTAGAAAAAACAAAGGGCTTTCG |
| | | ATTCTAAAAAGCCGTTTCGGAGCTGCTGCATAA |
| 107 | K100-1 | MRESWGASLKTICLIGGKLQGFEAAYLSKKAGMKVLVIDK |
| | Polypeptide | NPQALIRNYADEFQCFNITEEPEKLVAISKNVDAILPVNENL |
| | Sequence; | ECIEFLNSIKEKFSCPVLFDFEAYRISRDKRKSKEYFASIGTP |
| | mutations are | TPQDKPSEPPYFVKPPCESSSVGARIIHDRKELKELEPGMLIE |
| | shown in bold and | EYVEGEVVILEVIGDGNNFAVVKETLVHIDDTYDCHMVTP |
| | underlined letters | LPLDPSFRELSYSLAANLPLKGIMHVEAISGPLGLKVIEIDA |
| | | RFPSQVPTAVYYSSGINLIELLFRAFNGGIEEIKTLPEDRYCI |
| | | YEHLMLAENGVLIPVGEQVLSMGNDYGNYYEEPGIEIFLC |
| | | KGENPVFTLVFWGRDREEAEARKNKGLSILKSRFGAAA |
| 108 | K100-2 | ATGAGAGAGTCCTGGGGTGCTAGCCTGAAAACAATATG |
| | Polynucleotide | CCTTATAGGCGGGAAGCTGCAGGGCTTCGAGGCTGCATA |
| | Sequence; | CCTATCTAAGAAAGCCGGAATGAAAGTGCTTGTAATAG |
| | mutations are | ACAAAAACCCGCAGGCGCTTATAAGGAATTATGCGGAT |
| | shown in bold and | GAGTTCCAGTGTTTTAACATAACGGAAGAGCCGGAAAA |
| | underlined letters | ACTCGTCGCGATATCAAAAAATGTTGATGCCATACTGCC |
| | | GGTAAATGAAAACCTTGAATGTATAGAATTTCTGAATTC |
| | | TATAAAAGAAAAATTCTCCTGCCCGGTACTTTTCGATTTT |
| | | GAAGCTTACAGGATCAGCAGGGATAAGAGAAAATCAAA |
| | | AGAATACTTCGCATCCATAGGAACCCCGACCCCTCAGGA |
| | | CAAACCGTCGGAACCACCTTATTTTGTAAAGCCTCCCTG |
| | | CGAAAGCAGCAGTGTGGGAGCGAGAATAATCCATGACA |
| | | GGAAAGAGCTTAAAGAGCTTGAGCCCGGGATGCTCATA |
| | | GAAGAATACGTTGAAGGGGAAGTGGTCTTGCTTGTGGT |
| | | CATAGGGGATGGAAATAATTTTGCTGTGGTAAAGGAAA |
| | | CCCTTGTACATATCGATGACACCTATGACTGCCATATGG |
| | | TGACCCCTCTCCCTCTAGACCCTTCCTTCAGGGAACTATC |
| | | CTACTCCCTTGCAGCAAACCTGCCCTTAAAAGGAATTAT |
| | | GACGGTGGAAGCGATTTCCGGCCCCCTGGGGTTAAAAG |
| | | TTATTGAGATAGATGCCCGTTTCCCGAGCCAGATTCCGA |
| | | CTGCGGTCTATTATTCTTCCGGGATCAACCTCATAGAAC |
| | | TCCTGTTCCGGGCTTTTAATGGAGGCATAGAAGAGATCA |
| | | AAACTCTCCCTGAAGACAGGTACTGCATTTACGAACATC |
| | | TCATGCTTGCAGAAAATGGAGTACTTATCCCTGTGGGAG |
| | | AACAGGTCCTGTCCATGGGAAATGATTACGGCAATTATT |
| | | ATGAAGAACCTGGAATAGAGATTTTCCTGTGCAAAGGA |
| | | GAGAACCCTGTATTCACCCTGGTTTTCTGGGGCAGAGAC |
| | | AGGGAAGAAGCTGAAGCTAGAAAAAACAAAGGGCTTTC |
| | | GATTCTAAAAAGCCGTTTCGGAGCTGCTGCATAA |
| 109 | K100-2 | MRESWGASLKTICLIGGKLQGFEAAYLSKKAGMKVLVIDK |
| | Polypeptide | NPQALIRNYADEFQCFNITEEPEKLVAISKNVDAILPVNENL |
| | Sequence; | ECIEFLNSIKEKFSCPVLFDFEAYRISRDKRKSKEYFASIGTP |
| | mutations are | TPQDKPSEPPYFVKPPCESSSVGARIIHDRKELKELEPGMLIE |
| | shown in bold and | EYVEGEVVLLVVIGDGNNFAVVKETLVHIDDTYDCHMVT |
| | underlined letters | PLPLDPSFRELSYSLAANLPLKGIMTVEAISGPLGLKVIEIDA |
| | | RFPSQIPTAVYYSSGINLIELLFRAFNGGIEEIKTLPEDRYCIY |
| | | EHLMLAENGVLIPVGEQVLSMGNDYGNYYEEPGIEIFLCK |
| | | GENPVFTLVFWGRDREEAEARKNKGLSILKSRFGAAA |
| 110 | K100-3 | ATGAGAGAGTCCTGGGGTGCTAGCCTGAAAACAATATG |
| | Polynucleotide | CCTTATAGGCGGGAAGCTGCAGGGCTTCGAGGCTGCATA |
| | Sequence; | CCTATCTAAGAAAGCCGGAATGAAAGTGCTTGTAATAG |
| | mutations are | ACAAAAACCCGCAGGCGCTTATAAGGAATTATGCGGAT |
| | shown in bold and | GAGTTCCAGTGTTTTAACATAACGGAAGAGCCGGAAAA |
| | underlined letters | ACTCGTCGCGATATCAAAAAATGTTGATGCCATACTGCC |
| | | GGTAAATGAAAACCTTGAATGTATAGAATTTCTGAATTC |
| | | TATAAAAGAAAAATTCTCCTGCCCGGTACTTTTCGATTTT |
| | | GAAGCTTACAGGATCAGCAGGGATAAGAGAAAATCAAA |
| | | AGAATACTTCGCATCCATAGGAACCCCGACCCCTCAGGA |
| | | CAAACCGTCGGAACCACCTTATTTTGTAAAGCCTCCCTG |
| | | CGAAAGCAGCAGTGTGGGAGCGAGAATAATCCATGACA |
| | | GGAAAGAGCTTAAAGAGCTTGAGCCCGGGATGCTCATA |
| | | GAAGAATACGTTGAAGGGGAAGTGGTCCTGCTTGCTGT |
| | | CATAGGGGATGGAAATAATTTTGCTGTGGTAAAGGAAA |
| | | CCCTTGTACATATCGATGACACCTATGACTGCCATATGG |
| | | TGACCCCTCTCCCTCTAGACCCTTCCTTCAGGGAACTATC |
| | | CTACTCCCTTGCAGCAAACCTGCCCTTAAAAGGAATTAT |
| | | GCATGTGGAAGCGATTTCCGGCCCCCTGGGGTTAAAAGT |
| | | TATTGAGATAGATGCCCGTTTCCCGAGCCAGATGCCGAC |
| | | TGCGGTCTATTATTCTTCCGGGATCAACCTCATAGAACT |
| | | CCTGTTCCGGGCTTTTAATGGAGGCATAGAAGAGATCAA |
| | | AACTCTCCCTGAAGACAGGTACTGCATTTACGAACATCT |
| | | CATGCTTGCAGAAAATGGAGTACTTATCCCTGTGGGAGA |
| | | ACAGGTCCTGTCCATGGGAAATGATTACGGCAATTATTA |
| | | TGAAGAACCTGGAATAGAGATTTTCCTGTGCAAAGGAG |
| | | AGAACCCTGTATTCACCCTGGTTTTCTGGGGCAGAGACA |
| | | GGGAAGAAGCTGAAGCTAGAAAAAACAAAGGGCTTTCG |
| | | ATTCTAAAAAGCCGTTTCGGAGCTGCTGCATAA |
| 111 | K100-3 | MRESWGASLKTICLIGGKLQGFEAAYLSKKAGMKVLVIDK |
| | Polypeptide | NPQALIRNYADEFQCFNITEEPEKLVAISKNVDAILPVNENL |
| | Sequence; | ECIEFLNSIKEKFSCPVLFDFEAYRISRDKRKSKEYFASIGTP |
| | mutations are | TPQDKPSEPPYFVKPPCESSSVGARIIHDRKELKELEPGMLIE |
| | shown in bold and | EYVEGEVVLLAVIGDGNNFAVVKETLVHIDDTYDCHMVT |
| | underlined letters | PLPLDPSFRELSYSLAANLPLKGIMHVEAISGPLGLKVIEIDA |
| | | RFPSQMPTAVYYSSGINLIELLFRAFNGGIEEIKTLPEDRYCI |
| | | YEHLMLAENGVLIPVGEQVLSMGNDYGNYYEEPGIEIFLC |
| | | KGENPVFTLVFWGRDREEAEARKNKGLSILKSRFGAAA |
| 112 | K100-4 | ATGAGAGAGTCCTGGGGTGCTAGCCTGAAAACAATATG |
| | Polynucleotide | CCTTATAGGCGGGAAGCTGCAGGGCTTCGAGGCTGCATA |
| | Sequence; | CCTATCTAAGAAAGCCGGAATGAAAGTGCTTGTAATAG |
| | mutations are | ACAAAAACCCGCAGGCGCTTATAAGGAATTATGCGGAT |
| | shown in bold and | GAGTTCCAGTGTTTTAACATAACGGAAGAGCCGGAAAA |
| | underlined letters | ACTCGTCGCGATATCAAAAAATGTTGATGCCATACTGCC |
| | | GGTAAATGAAAACCTTGAATGTATAGAATTTCTGAATTC |
| | | TATAAAAGAAAAATTCTCCTGCCCGGTACTTTTCGATTTT |
| | | GAAGCTTACAGGATCAGCAGGGATAAGAGAAAATCAAA |
| | | AGAATACTTCGCATCCATAGGAACCCCGACCCCTCAGGA |
| | | CAAACCGTCGGAACCACCTTATTTTGTAAAGCCTCCCTG |
| | | CGAAAGCAGCAGTGTGGGAGCGAGAATAATCCATGACA |
| | | GGAAAGAGCTTAAAGAGCTTGAGCCCGGGATGCTCATA |
| | | GAAGAATACGTTGAAGGGGAAGTGGTCCTGCTTGTTGT |
| | | CATAGGGGATGGAAATAATTTTGCTGTGGTAAAGGAAA |
| | | CCCTTGTACATATCGATGACACCTATGACTGCCATATGG |
| | | TGACCCCTCTCCCTCTAGACCCTTCCTTCAGGGAACTATC |
| | | CTACTCCCTTGCAGCAAACCTGCCCTTAAAAGGAATTAT |
| | | GCATGTGGAAGCGATTTCCGGCCCCCTGGGGTTAAAAGT |
| | | TATTGAGATAGATGCCCGTTTCCCGAGCCAGGCGCCGAC |
| | | TGCGGTCTATTATTCTTCCGGGATCAACCTCATAGAACT |
| | | CCTGTTCCGGGCTTTTAATGGAGGCATAGAAGAGATCAA |
| | | AACTCTCCCTGAAGACAGGTACTGCATTTACGAACATCT |
| | | CATGCTTGCAGAAAATGGAGTACTTATCCCTGTGGGAGA |
| | | ACAGGTCCTGTCCATGGGAAATGATTACGGCAATTATTA |
| | | TGAAGAACCTGGAATAGAGATTTTCCTGTGCAAAGGAG |
| | | AGAACCCTGTATTCACCCTGGTTTTCTGGGGCAGAGACA |
| | | GGGAAGAAGCTGAAGCTAGAAAAAACAAAGGGCTTTCG |
| | | ATTCTAAAAAGCCGTTTCGGAGCTGCTGCATAA |
| 113 | K100-4 | MRESWGASLKTICLIGGKLQGFEAAYLSKKAGMKVLVIDK |
| | Polypeptide | NPQALIRNYADEFQCFNITEEPEKLVAISKNVDAILPVNENL |
| | Sequence; | ECIEFLNSIKEKFSCPVLFDFEAYRISRDKRKSKEYFASIGTP |
| | mutations are | TPQDKPSEPPYFVKPPCESSSVGARIIHDRKELKELEPGMLIE |
| | shown in bold and | EYVEGEVVLLVVIGDGNNFAVVKETLVHIDDTYDCHMVT |
| | underlined letters | PLPLDPSFRELSYSLAANLPLKGIMHVEAISGPLGLKVIEIDA |
| | | RFPSQAPTAVYYSSGINLIELLFRAFNGGIEEIKTLPEDRYCI |
| | | YEHLMLAENGVLIPVGEQVLSMGNDYGNYYEEPGIEIFLC |
| | | KGENPVFTLVFWGRDREEAEARKNKGLSILKSRFGAAA |
| 114 | K100-5 | ATGAGAGAGTCCTGGGGTGCTAGCCTGAAAACAATATG |
| | Polynucleotide | CCTTATAGGCGGGAAGCTGCAGGGCTTCGAGGCTGCATA |
| | Sequence; | CCTATCTAAGAAAGCCGGAATGAAAGTGCTTGTAATAG |
| | mutations are | ACAAAAACCCGCAGGCGCTTATAAGGAATTATGCGGAT |
| | shown in bold and | GAGTTCCAGTGTTTTAACATAACGGAAGAGCCGGAAAA |
| | underlined letters | ACTCGTCGCGATATCAAAAAATGTTGATGCCATACTGCC |
| | | GGTAAATGAAAACCTTGAATGTATAGAATTTCTGAATTC |
| | | TATAAAAGAAAAATTCTCCTGCCCGGTACTTTTCGATTTT |
| | | GAAGCTTACAGGATCAGCAGGGATAAGAGAAAATCAAA |
| | | AGAATACTTCGCATCCATAGGAACCCCGACCCCTCAGGA |
| | | CAAACCGTCGGAACCACCTTATTTTGTAAAGCCTCCCTG |
| | | CGAAAGCAGCAGTGTGGGAGCGAGAATAATCCATGACA |
| | | GGAAAGAGCTTAAAGAGCTTGAGCCCGGGATGCTCATA |
| | | GAAGAATACGTTGAAGGGGAAGTGGTCATTCTTATGGT |
| | | CATAGGGGATGGAAATAATTTTGCTGTGGTAAAGGAAA |
| | | CCCTTGTACATATCGATGACACCTATGACTGCCATATGG |
| | | TGACCCCTCTCCCTCTAGACCCTTCCTTCAGGGAACTATC |
| | | CTACTCCCTTGCAGCAAACCTGCCCTTAAAAGGAATTAT |
| | | GCATGTGGAAGCGATTTCCGGCCCCCTGGGGTTAAAAGT |
| | | TATTGAGATAGATGCCCGTTTCCCGAGCCAGCTTCCGAC |
| | | TGCGGTCTATTATTCTTCCGGGATCAACCTCATAGAACT |
| | | CCTGTTCCGGGCTTTTAATGGAGGCATAGAAGAGATCAA |
| | | AACTCTCCCTGAAGACAGGTACTGCATTTACGAACATCT |
| | | CATGCTTGCAGAAAATGGAGTACTTATCCCTGTGGGAGA |
| | | ACAGGTCCTGTCCATGGGAAATGATTACGGCAATTATTA |
| | | TGAAGAACCTGGAATAGAGATTTTCCTGTGCAAAGGAG |
| | | AGAACCCTGTATTCACCCTGGTTTTCTGGGGCAGAGACA |
| | | GGGAAGAAGCTGAAGCTAGAAAAAACAAAGGGCTTTCG |
| | | ATTCTAAAAAGCCGTTTCGGAGCTGCTGCATAA |
| 115 | K100-5 | MRESWGASLKTICLIGGKLQGFEAAYLSKKAGMKVLVIDK |
| | Polypeptide | NPQALIRNYADEFQCFNITEEPEKLVAISKNVDAILPVNENL |
| | Sequence; | ECIEFLNSIKEKFSCPVLFDFEAYRISRDKRKSKEYFASIGTP |
| | mutations are | TPQDKPSEPPYFVKPPCESSSVGARIIHDRKELKELEPGMLIE |
| | shown in bold and | EYVEGEVVILMVIGDGNNFAVVKETLVHIDDTYDCHMVTP |
| | underlined letters | LPLDPSFRELSYSLAANLPLKGIMHVEAISGPLGLKVIEIDA |
| | | RFPSQLPTAVYYSSGINLIELLFRAFNGGIEEIKTLPEDRYCI |
| | | YEHLMLAENGVLIPVGEQVLSMGNDYGNYYEEPGIEIFLC |
| | | KGENPVFTLVFWGRDREEAEARKNKGLSILKSRFGAAA |
| 116 | K100-6 | ATGAGAGAGTCCTGGGGTGCTAGCCTGAAAACAATATG |
| | Polynucleotide | CCTTATAGGCGGGAAGCTGCAGGGCTTCGAGGCTGCATA |
| | Sequence; | CCTATCTAAGAAAGCCGGAATGAAAGTGCTTGTAATAG |
| | mutations are | ACAAAAACCCGCAGGCGCTTATAAGGAATTATGCGGAT |
| | shown in bold and | GAGTTCCAGTGTTTTAACATAACGGAAGAGCCGGAAAA |
| | underlined letters | ACTCGTCGCGATATCAAAAAATGTTGATGCCATACTGCC |
| | | GGTAAATGAAAACCTTGAATGTATAGAATTTCTGAATTC |
| | | TATAAAAGAAAAATTCTCCTGCCCGGTACTTTTCGATTTT |
| | | GAAGCTTACAGGATCAGCAGGGATAAGAGAAAATCAAA |
| | | AGAATACTTCGCATCCATAGGAACCCCGACCCCTCAGGA |
| | | CAAACCGTCGGAACCACCTTATTTTGTAAAGCCTCCCTG |
| | | CGAAAGCAGCAGTGTGGGAGCGAGAATAATCCATGACA |
| | | GGAAAGAGCTTAAAGAGCTTGAGCCCGGGATGCTCATA |
| | | GAAGAATACGTTGAAGGGGAAGTGGTCGCGCTTATTGT |
| | | CATAGGGGATGGAAATAATTTTGCTGTGGTAAAGGAAA |
| | | CCCTTGTACATATCGATGACACCTATGACTGCCATATGG |
| | | TGACCCCTCTCCCTCTAGACCCTTCCTTCAGGGAACTATC |
| | | CTACTCCCTTGCAGCAAACCTGCCCTTAAAAGGAATTAT |
| | | GAATGTGGAAGCGATTTCCGGCCCCCTGGGGTTAAAAGT |
| | | TATTGAGATAGATGCCCGTTTCCCGAGCCAGATTCCGAC |
| | | TGCGGTCTATTATTCTTCCGGGATCAACCTCATAGAACT |
| | | CCTGTTCCGGGCTTTTAATGGAGGCATAGAAGAGATCAA |
| | | AACTCTCCCTGAAGACAGGTACTGCATTTACGAACATCT |
| | | CATGCTTGCAGAAAATGGAGTACTTATCCCTGTGGGAGA |
| | | ACAGGTCCTGTCCATGGGAAATGATTACGGCAATTATTA |
| | | TGAAGAACCTGGAATAGAGATTTTCCTGTGCAAAGGAG |
| | | AGAACCCTGTATTCACCCTGGTTTTCTGGGGCAGAGACA |
| | | GGGAAGAAGCTGAAGCTAGAAAAAACAAAGGGCTTTCG |
| | | ATTCTAAAAAGCCGTTTCGGAGCTGCTGCATAA |
| 117 | K100-6 | MRESWGASLKTICLIGGKLQGFEAAYLSKKAGMKVLVIDK |
| | Polypeptide | NPQALIRNYADEFQCFNITEEPEKLVAISKNVDAILPVNENL |
| | Sequence; | ECIEFLNSIKEKFSCPVLFDFEAYRISRDKRKSKEYFASIGTP |
| | mutations are | TPQDKPSEPPYFVKPPCESSSVGARIIHDRKELKELEPGMLIE |
| | shown in bold and | EYVEGEVVALIVIGDGNNFAVVKETLVHIDDTYDCHMVTP |
| | underlined letters | LPLDPSFRELSYSLAANLPLKGIMNVEAISGPLGLKVIEIDA |
| | | RFPSQIPTAVYYSSGINLIELLFRAFNGGIEEIKTLPEDRYCIY |
| | | EHLMLAENGVLIPVGEQVLSMGNDYGNYYEEPGIEIFLCK |
| | | GENPVFTLVFWGRDREEAEARKNKGLSILKSRFGAAA |
| 118 | K100-7 | ATGAGAGAGTCCTGGGGTGCTAGCCTGAAAACAATATG |
| | Polynucleotide | CCTTATAGGCGGGAAGCTGCAGGGCTTCGAGGCTGCATA |
| | Sequence; | CCTATCTAAGAAAGCCGGAATGAAAGTGCTTGTAATAG |
| | mutations are | ACAAAAACCCGCAGGCGCTTATAAGGAATTATGCGGAT |
| | shown in bold and | GAGTTCCAGTGTTTTAACATAACGGAAGAGCCGGAAAA |
| | underlined letters | ACTCGTCGCGATATCAAAAAATGTTGATGCCATACTGCC |
| | | GGTAAATGAAAACCTTGAATGTATAGAATTTCTGAATTC |
| | | TATAAAAGAAAAATTCTCCTGCCCGGTACTTTTCGATTTT |
| | | GAAGCTTACAGGATCAGCAGGGATAAGAGAAAATCAAA |
| | | AGAATACTTCGCATCCATAGGAACCCCGACCCCTCAGGA |
| | | CAAACCGTCGGAACCACCTTATTTTGTAAAGCCTCCCTG |
| | | CGAAAGCAGCAGTGTGGGAGCGAGAATAATCCATGACA |
| | | GGAAAGAGCTTAAAGAGCTTGAGCCCGGGATGCTCATA |
| | | GAAGAATACGTTGAAGGGGAAGTGGTCATTCTTTGTGT |
| | | CATAGGGGATGGAAATAATTTTGCTGTGGTAAAGGAAA |
| | | CCCTTGTACATATCGATGACACCTATGACTGCCATATGG |
| | | TGACCCCTCTCCCTCTAGACCCTTCCTTCAGGGAACTATC |
| | | CTACTCCCTTGCAGCAAACCTGCCCTTAAAAGGAATTAT |
| | | GCATGTGGAAGCGATTTCCGGCCCCCTGGGGTTAAAAGT |
| | | TATTGAGATAGATGCCCGTTTCCCGAGCCAGCTGCCGAC |
| | | TGCGGTCTATTATTCTTCCGGGATCAACCTCATAGAACT |
| | | CCTGTTCCGGGCTTTTAATGGAGGCATAGAAGAGATCAA |
| | | AACTCTCCCTGAAGACAGGTACTGCATTTACGAACATCT |
| | | CATGCTTGCAGAAAATGGAGTACTTATCCCTGTGGGAGA |
| | | ACAGGTCCTGTCCATGGGAAATGATTACGGCAATTATTA |
| | | TGAAGAACCTGGAATAGAGATTTTCCTGTGCAAAGGAG |
| | | AGAACCCTGTATTCACCCTGGTTTTCTGGGGCAGAGACA |
| | | GGGAAGAAGCTGAAGCTAGAAAAAACAAAGGGCTTTCG |
| | | ATTCTAAAAAGCCGTTTCGGAGCTGCTGCATAA |
| 119 | K100-7 | MRESWGASLKTICLIGGKLQGFEAAYLSKKAGMKVLVIDK |
| | Polypeptide | NPQALIRNYADEFQCFNITEEPEKLVAISKNVDAILPVNENL |
| | Sequence; | ECIEFLNSIKEKFSCPVLFDFEAYRISRDKRKSKEYFASIGTP |
| | mutations are | TPQDKPSEPPYFVKPPCESSSVGARIIHDRKELKELEPGMLIE |
| | shown in bold and | EYVEGEVVILCVIGDGNNFAVVKETLVHIDDTYDCHMVTP |
| | underlined letters | LPLDPSFRELSYSLAANLPLKGIMHVEAISGPLGLKVIEIDA |
| | | RFPSQLPTAVYYSSGINLIELLFRAFNGGIEEIKTLPEDRYCI |
| | | YEHLMLAENGVLIPVGEQVLSMGNDYGNYYEEPGIEIFLC |
| | | KGENPVFTLVFWGRDREEAEARKNKGLSILKSRFGAAA |
| 120 | K100-8 | ATGAGAGAGTCCTGGGGTGCTAGCCTGAAAACAATATG |
| | Polynucleotide | CCTTATAGGCGGGAAGCTGCAGGGCTTCGAGGCTGCATA |
| | Sequence; | CCTATCTAAGAAAGCCGGAATGAAAGTGCTTGTAATAG |
| | mutations are | ACAAAAACCCGCAGGCGCTTATAAGGAATTATGCGGAT |
| | shown in bold and | GAGTTCCAGTGTTTTAACATAACGGAAGAGCCGGAAAA |
| | underlined letters | ACTCGTCGCGATATCAAAAAATGTTGATGCCATACTGCC |
| | | GGTAAATGAAAACCTTGAATGTATAGAATTTCTGAATTC |
| | | TATAAAAGAAAAATTCTCCTGCCCGGTACTTTTCGATTTT |
| | | GAAGCTTACAGGATCAGCAGGGATAAGAGAAAATCAAA |
| | | AGAATACTTCGCATCCATAGGAACCCCGACCCCTCAGGA |
| | | CAAACCGTCGGAACCACCTTATTTTGTAAAGCCTCCCTG |
| | | CGAAAGCAGCAGTGTGGGAGCGAGAATAATCCATGACA |
| | | GGAAAGAGCTTAAAGAGCTTGAGCCCGGGATGCTCATA |
| | | GAAGAATACGTTGAAGGGGAAGTGGTCAGTCTTTCTGT |
| | | CATAGGGGATGGAAATAATTTTGCTGTGGTAAAGGAAA |
| | | CCCTTGTACATATCGATGACACCTATGACTGCCATATGG |
| | | TGACCCCTCTCCCTCTAGACCCTTCCTTCAGGGAACTATC |
| | | CTACTCCCTTGCAGCAAACCTGCCCTTAAAAGGAATTAT |
| | | GCATGTGGAAGCGATTTCCGGCCCCCTGGGGTTAAAAGT |
| | | TATTGAGATAGATGCCCGTTTCCCGAGCCAGACGCCGAC |
| | | TGCGGTCTATTATTCTTCCGGGATCAACCTCATAGAACT |
| | | CCTGTTCCGGGCTTTTAATGGAGGCATAGAAGAGATCAA |
| | | AACTCTCCCTGAAGACAGGTACTGCATTTACGAACATCT |
| | | CATGCTTGCAGAAAATGGAGTACTTATCCCTGTGGGAGA |
| | | ACAGGTCCTGTCCATGGGAAATGATTACGGCAATTATTA |
| | | TGAAGAACCTGGAATAGAGATTTTCCTGTGCAAAGGAG |
| | | AGAACCCTGTATTCACCCTGGTTTTCTGGGGCAGAGACA |
| | | GGGAAGAAGCTGAAGCTAGAAAAAACAAAGGGCTTTCG |
| | | ATTCTAAAAAGCCGTTTCGGAGCTGCTGCATAA |
| 121 | K100-8 | MRESWGASLKTICLIGGKLQGFEAAYLSKKAGMKVLVIDK |
| | Polypeptide | NPQALIRNYADEFQCFNITEEPEKLVAISKNVDAILPVNENL |
| | Sequence; | ECIEFLNSIKEKFSCPVLFDFEAYRISRDKRKSKEYFASIGTP |
| | mutations are | TPQDKPSEPPYFVKPPCESSSVGARIIHDRKELKELEPGMLIE |
| | shown in bold and | EYVEGEVVSLSVIGDGNNFAVVKETLVHIDDTYDCHMVTP |
| | underlined letters | LPLDPSFRELSYSLAANLPLKGIMHVEAISGPLGLKVIEIDA |
| | | RFPSQTPTAVYYSSGINLIELLFRAFNGGIEEIKTLPEDRYCI |
| | | YEHLMLAENGVLIPVGEQVLSMGNDYGNYYEEPGIEIFLC |
| | | KGENPVFTLVFWGRDREEAEARKNKGLSILKSRFGAAA |
| 122 | K150-1 | ATGAGAGAGTCCTGGGGTGCTAGCCTGAAAACAATATG |
| | Polynucleotide | CCTTATAGGCGGGAAGCTGCAGGGCTTCGAGGCTGCATA |
| | Sequence; | CCTATCTAAGAAAGCCGGAATGAAAGTGCTTGTAATAG |
| | mutations are | ACAAAAACCCGCAGGCGCTTATAAGGAATTATGCGGAT |
| | shown in bold and | GAGTTCCAGTGTTTTAACATAACGGAAGAGCCGGAAAA |
| | underlined letters | ACTCGTCGCGATATCAAAAAATGTTGATGCCATACTGCC |
| | | GGTAAATGAAAACCTTGAATGTATAGAATTTCTGAATTC |
| | | TATAAAAGAAAAATTCTCCTGCCCGGTACTTTTCGATTTT |
| | | GAAGCTTACAGGATCAGCAGGGATAAGAGAAAATCAAA |
| | | AGAATACTTCGCATCCATAGGAACCCCGACCCCTCAGGA |
| | | CAAACCGTCGGAACCACCTTATTTTGTAAAGCCTCCCTG |
| | | CGAAAGCAGCAGTGTGGGAGCGAGAATAATCCATGACA |
| | | GGAAAGAGCTTAAAGAGCTTGAGCCCGGGATGCTCATA |
| | | GAAGAATACGTTGAAGGGGAAGTGGTCGTGCTTGTTGT |
| | | CATAGGGGATGGAAATAATTTTGCTGTGGTAAAGGAAA |
| | | CCCTTGTACATATCGATGACACCTATGACTGCCATATGG |
| | | TGACCCCTCTCCCTCTAGACCCTTCCTTCAGGGAACTATC |
| | | CTACTCCCTTGCAGCAAACCTGCCCTTAAAAGGAATTAT |
| | | GCATGTGGAAGCGATTTCCGGCCCCCTGGGGTTAAAAGT |
| | | TATTGAGATAGATGCCCGTTTCCCGAGCCAGGCTCCGAC |
| | | TGCGGTCTATTATTCTTCCGGGATCAACCTCATAGAACT |
| | | CCTGTTCCGGGCTTTTAATGGAGGCATAGAAGAGATCAA |
| | | AACTCTCCCTGAAGACAGGTACTGCATTTACGAACATCT |
| | | CATGCTTGCAGAAAATGGAGTACTTATCCCTGTGGGAGA |
| | | ACAGGTCCTGTCCATGGGAAATGATTACGGCAATTATTA |
| | | TGAAGAACCTGGAATAGAGATTTTCCTGTGCAAAGGAG |
| | | AGAACCCTGTATTCACCCTGGTTTTCTGGGGCAGAGACA |
| | | GGGAAGAAGCTGAAGCTAGAAAAAACAAAGGGCTTTCG |
| | | ATTCTAAAAAGCCGTTTCGGAGCTGCTGCATAA |
| 123 | K150-1 | MRESWGASLKTICLIGGKLQGFEAAYLSKKAGMKVLVIDK |
| | Polypeptide | NPQALIRNYADEFQCFNITEEPEKLVAISKNVDAILPVNENL |
| | Sequence; | ECIEFLNSIKEKFSCPVLFDFEAYRISRDKRKSKEYFASIGTP |
| | mutations are | TPQDKPSEPPYFVKPPCESSSVGARIIHDRKELKELEPGMLIE |
| | shown in bold and | EYVEGEVVVLVVIGDGNNFAVVKETLVHIDDTYDCHMVT |
| | underlined letters | PLPLDPSFRELSYSLAANLPLKGIMHVEAISGPLGLKVIEIDA |
| | | RFPSQAPTAVYYSSGINLIELLFRAFNGGIEEIKTLPEDRYCI |
| | | YEHLMLAENGVLIPVGEQVLSMGNDYGNYYEEPGIEIFLC |
| | | KGENPVFTLVFWGRDREEAEARKNKGLSILKSRFGAAA |
| 124 | K150-2 | ATGAGAGAGTCCTGGGGTGCTAGCCTGAAAACAATATG |
| | Polynucleotide | CCTTATAGGCGGGAAGCTGCAGGGCTTCGAGGCTGCATA |
| | Sequence; | CCTATCTAAGAAAGCCGGAATGAAAGTGCTTGTAATAG |
| | mutations are | ACAAAAACCCGCAGGCGCTTATAAGGAATTATGCGGAT |
| | shown in bold and | GAGTTCCAGTGTTTTAACATAACGGAAGAGCCGGAAAA |
| | underlined letters | ACTCGTCGCGATATCAAAAAATGTTGATGCCATACTGCC |
| | | GGTAAATGAAAACCTTGAATGTATAGAATTTCTGAATTC |
| | | TATAAAAGAAAAATTCTCCTGCCCGGTACTTTTCGATTTT |
| | | GAAGCTTACAGGATCAGCAGGGATAAGAGAAAATCAAA |
| | | AGAATACTTCGCATCCATAGGAACCCCGACCCCTCAGGA |
| | | CAAACCGTCGGAACCACCTTATTTTGTAAAGCCTCCCTG |
| | | CGAAAGCAGCAGTGTGGGAGCGAGAATAATCCATGACA |
| | | GGAAAGAGCTTAAAGAGCTTGAGCCCGGGATGCTCATA |
| | | GAAGAATACGTTGAAGGGGAAGTGGTCTGTCTTGTTGT |
| | | CATAGGGGATGGAAATAATTTTGCTGTGGTAAAGGAAA |
| | | CCCTTGTACATATCGATGACACCTATGACTGCCATATGG |
| | | TGACCCCTCTCCCTCTAGACCCTTCCTTCAGGGAACTATC |
| | | CTACTCCCTTGCAGCAAACCTGCCCTTAAAAGGAATTAT |
| | | GCATGTGGAAGCGATTTCCGGCCCCCTGGGGTTAAAAGT |
| | | TATTGAGATAGATGCCCGTTTCCCGAGCCAGTATCCGAC |
| | | TGCGGTCTATTATTCTTCCGGGATCAACCTCATAGAACT |
| | | CCTGTTCCGGGCTTTTAATGGAGGCATAGAAGAGATCAA |
| | | AACTCTCCCTGAAGACAGGTACTGCATTTACGAACATCT |
| | | CATGCTTGCAGAAAATGGAGTACTTATCCCTGTGGGAGA |
| | | ACAGGTCCTGTCCATGGGAAATGATTACGGCAATTATTA |
| | | TGAAGAACCTGGAATAGAGATTTTCCTGTGCAAAGGAG |
| | | AGAACCCTGTATTCACCCTGGTTTTCTGGGGCAGAGACA |
| | | GGGAAGAAGCTGAAGCTAGAAAAAACAAAGGGCTTTCG |
| | | ATTCTAAAAAGCCGTTTCGGAGCTGCTGCATAA |
| 125 | K150-2 | MRESWGASLKTICLIGGKLQGFEAAYLSKKAGMKVLVIDK |
| | Polypeptide | NPQALIRNYADEFQCFNITEEPEKLVAISKNVDAILPVNENL |
| | Sequence; | ECIEFLNSIKEKFSCPVLFDFEAYRISRDKRKSKEYFASIGTP |
| | mutations are | TPQDKPSEPPYFVKPPCESSSVGARIIHDRKELKELEPGMLIE |
| | shown in bold and | EYVEGEVVCLVVIGDGNNFAVVKETLVHIDDTYDCHMVT |
| | underlined letters | PLPLDPSFRELSYSLAANLPLKGIMHVEAISGPLGLKVIEIDA |
| | | RFPSQYPTAVYYSSGINLIELLFRAFNGGIEEIKTLPEDRYCI |
| | | YEHLMLAENGVLIPVGEQVLSMGNDYGNYYEEPGIEIFLC |
| | | KGENPVFTLVFWGRDREEAEARKNKGLSILKSRFGAAA |
| 126 | K150-3 | ATGAGAGAGTCCTGGGGTGCTAGCCTGAAAACAATATG |
| | Polynucleotide | CCTTATAGGCGGGAAGCTGCAGGGCTTCGAGGCTGCATA |
| | Sequence; | CCTATCTAAGAAAGCCGGAATGAAAGTGCTTGTAATAG |
| | mutations are | ACAAAAACCCGCAGGCGCTTATAAGGAATTATGCGGAT |
| | shown in bold and | GAGTTCCAGTGTTTTAACATAACGGAAGAGCCGGAAAA |
| | underlined letters | ACTCGTCGCGATATCAAAAAATGTTGATGCCATACTGCC |
| | | GGTAAATGAAAACCTTGAATGTATAGAATTTCTGAATTC |
| | | TATAAAAGAAAAATTCTCCTGCCCGGTACTTTTCGATTTT |
| | | GAAGCTTACAGGATCAGCAGGGATAAGAGAAAATCAAA |
| | | AGAATACTTCGCATCCATAGGAACCCCGACCCCTCAGGA |
| | | CAAACCGTCGGAACCACCTTATTTTGTAAAGCCTCCCTG |
| | | CGAAAGCAGCAGTGTGGGAGCGAGAATAATCCATGACA |
| | | GGAAAGAGCTTAAAGAGCTTGAGCCCGGGATGCTCATA |
| | | GAAGAATACGTTGAAGGGGAAGTGGTCATTCTTGCGGT |
| | | CATAGGGGATGGAAATAATTTTGCTGTGGTAAAGGAAA |
| | | CCCTTGTACATATCGATGACACCTATGACTGCCATATGG |
| | | TGACCCCTCTCCCTCTAGACCCTTCCTTCAGGGAACTATC |
| | | CTACTCCCTTGCAGCAAACCTGCCCTTAAAAGGAATTAT |
| | | GCATGTGGAAGCGATTTCCGGCCCCCTGGGGTTAAAAGT |
| | | TATTGAGATAGATGCCCGTTTCCCGAGCCAGCATCCGAC |
| | | TGCGGTCTATTATTCTTCCGGGATCAACCTCATAGAACT |
| | | CCTGTTCCGGGCTTTTAATGGAGGCATAGAAGAGATCAA |
| | | AACTCTCCCTGAAGACAGGTACTGCATTTACGAACATCT |
| | | CATGCTTGCAGAAAATGGAGTACTTATCCCTGTGGGAGA |
| | | ACAGGTCCTGTCCATGGGAAATGATTACGGCAATTATTA |
| | | TGAAGAACCTGGAATAGAGATTTTCCTGTGCAAAGGAG |
| | | AGAACCCTGTATTCACCCTGGTTTTCTGGGGCAGAGACA |
| | | GGGAAGAAGCTGAAGCTAGAAAAAACAAAGGGCTTTCG |
| | | ATTCTAAAAAGCCGTTTCGGAGCTGCTGCATAA |
| 127 | K150-3 | MRESWGASLKTICLIGGKLQGFEAAYLSKKAGMKVLVIDK |
| | Polypeptide | NPQALIRNYADEFQCFNITEEPEKLVAISKNVDAILPVNENL |
| | Sequence; | ECIEFLNSIKEKFSCPVLFDFEAYRISRDKRKSKEYFASIGTP |
| | mutations are | TPQDKPSEPPYFVKPPCESSSVGARIIHDRKELKELEPGMLIE |
| | shown in bold and | EYVEGEVVILAVIGDGNNFAVVKETLVHIDDTYDCHMVTP |
| | underlined letters | LPLDPSFRELSYSLAANLPLKGIMHVEAISGPLGLKVIEIDA |
| | | RFPSQHPTAVYYSSGINLIELLFRAFNGGIEEIKTLPEDRYCI |
| | | YEHLMLAENGVLIPVGEQVLSMGNDYGNYYEEPGIEIFLC |
| | | KGENPVFTLVFWGRDREEAEARKNKGLSILKSRFGAAA |
| 128 | K150-4 | ATGAGAGAGTCCTGGGGTGCTAGCCTGAAAACAATATG |
| | Polynucleotide | CCTTATAGGCGGGAAGCTGCAGGGCTTCGAGGCTGCATA |
| | Sequence; | CCTATCTAAGAAAGCCGGAATGAAAGTGCTTGTAATAG |
| | mutations are | ACAAAAACCCGCAGGCGCTTATAAGGAATTATGCGGAT |
| | shown in bold and | GAGTTCCAGTGTTTTAACATAACGGAAGAGCCGGAAAA |
| | underlined letters | ACTCGTCGCGATATCAAAAAATGTTGATGCCATACTGCC |
| | | GGTAAATGAAAACCTTGAATGTATAGAATTTCTGAATTC |
| | | TATAAAAGAAAAATTCTCCTGCCCGGTACTTTTCGATTTT |
| | | GAAGCTTACAGGATCAGCAGGGATAAGAGAAAATCAAA |
| | | AGAATACTTCGCATCCATAGGAACCCCGACCCCTCAGGA |
| | | CAAACCGTCGGAACCACCTTATTTTGTAAAGCCTCCCTG |
| | | CGAAAGCAGCAGTGTGGGAGCGAGAATAATCCATGACA |
| | | GGAAAGAGCTTAAAGAGCTTGAGCCCGGGATGCTCATA |
| | | GAAGAATACGTTGAAGGGGAAGTGGTCCTGCTTTGTGT |
| | | CATAGGGGATGGAAATAATTTTGCTGTGGTAAAGGAAA |
| | | CCCTTGTACATATCGATGACACCTATGACTGCCATATGG |
| | | TGACCCCTCTCCCTCTAGACCCTTCCTTCAGGGAACTATC |
| | | CTACTCCCTTGCAGCAAACCTGCCCTTAAAAGGAATTAT |
| | | GCATGTGGAAGCGATTTCCGGCCCCCTGGGGTTAAAAGT |
| | | TATTGAGATAGATGCCCGTTTCCCGAGCCAGGTGCCGAC |
| | | TGCGGTCTATTATTCTTCCGGGATCAACCTCATAGAACT |
| | | CCTGTTCCGGGCTTTTAATGGAGGCATAGAAGAGATCAA |
| | | AACTCTCCCTGAAGACAGGTACTGCATTTACGAACATCT |
| | | CATGCTTGCAGAAAATGGAGTACTTATCCCTGTGGGAGA |
| | | ACAGGTCCTGTCCATGGGAAATGATTACGGCAATTATTA |
| | | TGAAGAACCTGGAATAGAGATTTTCCTGTGCAAAGGAG |
| | | AGAACCCTGTATTCACCCTGGTTTTCTGGGGCAGAGACA |
| | | GGGAAGAAGCTGAAGCTAGAAAAAACAAAGGGCTTTCG |
| | | ATTCTAAAAAGCCGTTTCGGAGCTGCTGCATAA |
| 129 | K150-4 | MRESWGASLKTICLIGGKLQGFEAAYLSKKAGMKVLVIDK |
| | Polypeptide | NPQALIRNYADEFQCFNITEEPEKLVAISKNVDAILPVNENL |
| | Sequence; | ECIEFLNSIKEKFSCPVLFDFEAYRISRDKRKSKEYFASIGTP |
| | mutations are | TPQDKPSEPPYFVKPPCESSSVGARIIHDRKELKELEPGMLIE |
| | shown in bold and | EYVEGEVVLLCVIGDGNNFAVVKETLVHIDDTYDCHMVT |
| | underlined letters | PLPLDPSFRELSYSLAANLPLKGIMHVEAISGPLGLKVIEIDA |
| | | RFPSQVPTAVYYSSGINLIELLFRAFNGGIEEIKTLPEDRYCI |
| | | YEHLMLAENGVLIPVGEQVLSMGNDYGNYYEEPGIEIFLC |
| | | KGENPVFTLVFWGRDREEAEARKNKGLSILKSRFGAAA |
| 130 | K150-5 | ATGAGAGAGTCCTGGGGTGCTAGCCTGAAAACAATATG |
| | Polynucleotide | CCTTATAGGCGGGAAGCTGCAGGGCTTCGAGGCTGCATA |
| | Sequence; | CCTATCTAAGAAAGCCGGAATGAAAGTGCTTGTAATAG |
| | mutations are | ACAAAAACCCGCAGGCGCTTATAAGGAATTATGCGGAT |
| | shown in bold and | GAGTTCCAGTGTTTTAACATAACGGAAGAGCCGGAAAA |
| | underlined letters | ACTCGTCGCGATATCAAAAAATGTTGATGCCATACTGCC |
| | | GGTAAATGAAAACCTTGAATGTATAGAATTTCTGAATTC |
| | | TATAAAAGAAAAATTCTCCTGCCCGGTACTTTTCGATTTT |
| | | GAAGCTTACAGGATCAGCAGGGATAAGAGAAAATCAAA |
| | | AGAATACTTCGCATCCATAGGAACCCCGACCCCTCAGGA |
| | | CAAACCGTCGGAACCACCTTATTTTGTAAAGCCTCCCTG |
| | | CGAAAGCAGCAGTGTGGGAGCGAGAATAATCCATGACA |
| | | GGAAAGAGCTTAAAGAGCTTGAGCCCGGGATGCTCATA |
| | | GAAGAATACGTTGAAGGGGAAGTGGTCTCTCTTGTCGT |
| | | CATAGGGGATGGAAATAATTTTGCTGTGGTAAAGGAAA |
| | | CCCTTGTACATATCGATGACACCTATGACTGCCATATGG |
| | | TGACCCCTCTCCCTCTAGACCCTTCCTTCAGGGAACTATC |
| | | CTACTCCCTTGCAGCAAACCTGCCCTTAAAAGGAATTAT |
| | | GCATGTGGAAGCGATTTCCGGCCCCCTGGGGTTAAAAGT |
| | | TATTGAGATAGATGCCCGTTTCCCGAGCCAGACTCCGAC |
| | | TGCGGTCTATTATTCTTCCGGGATCAACCTCATAGAACT |
| | | CCTGTTCCGGGCTTTTAATGGAGGCATAGAAGAGATCAA |
| | | AACTCTCCCTGAAGACAGGTACTGCATTTACGAACATCT |
| | | CATGCTTGCAGAAAATGGAGTACTTATCCCTGTGGGAGA |
| | | ACAGGTCCTGTCCATGGGAAATGATTACGGCAATTATTA |
| | | TGAAGAACCTGGAATAGAGATTTTCCTGTGCAAAGGAG |
| | | AGAACCCTGTATTCACCCTGGTTTTCTGGGGCAGAGACA |
| | | GGGAAGAAGCTGAAGCTAGAAAAAACAAAGGGCTTTCG |
| | | ATTCTAAAAAGCCGTTTCGGAGCTGCTGCATAA |
| 131 | K150-5 | MRESWGASLKTICLIGGKLQGFEAAYLSKKAGMKVLVIDK |
| | Polypeptide | NPQALIRNYADEFQCFNITEEPEKLVAISKNVDAILPVNENL |
| | Sequence; | ECIEFLNSIKEKFSCPVLFDFEAYRISRDKRKSKEYFASIGTP |
| | mutations are | TPQDKPSEPPYFVKPPCESSSVGARIIHDRKELKELEPGMLIE |
| | shown in bold and | EYVEGEVVSLVVIGDGNNFAVVKETLVHIDDTYDCHMVTP |
| | underlined letters | LPLDPSFRELSYSLAANLPLKGIMHVEAISGPLGLKVIEIDA |
| | | RFPSQTPTAVYYSSGINLIELLFRAFNGGIEEIKTLPEDRYCI |
| | | YEHLMLAENGVLIPVGEQVLSMGNDYGNYYEEPGIEIFLC |
| | | KGENPVFTLVFWGRDREEAEARKNKGLSILKSRFGAAA |
| 132 | K150-11 | ATGAGAGAGTCCTGGGGTGCTAGCCTGAAAACAATATG |
| | Polynucleotide | CCTTATAGGCGGGAAGCTGCAGGGCTTCGAGGCTGCATA |
| | Sequence; | CCTATCTAAGAAAGCCGGAATGAAAGTGCTTGTAATAG |
| | mutations are | ACAAAAACCCGCAGGCGCTTATAAGGAATTATGCGGAT |
| | shown in bold and | GAGTTCCAGTGTTTTAACATAACGGAAGAGCCGGAAAA |
| | underlined letters | ACTCGTCGCGATATCAAAAAATGTTGATGCCATACTGCC |
| | | GGTAAATGAAAACCTTGAATGTATAGAATTTCTGAATTC |
| | | TATAAAAGAAAAATTCTCCTGCCCGGTACTTTTCGATTTT |
| | | GAAGCTTACAGGATCAGCAGGGATAAGAGAAAATCAAA |
| | | AGAATACTTCGCATCCATAGGAACCCCGACCCCTCAGGA |
| | | CAAACCGTCGGAACCACCTTATTTTGTAAAGCCTCCCTG |
| | | CGAAAGCAGCAGTGTGGGAGCGAGAATAATCCATGACA |
| | | GGAAAGAGCTTAAAGAGCTTGAGCCCGGGATGCTCATA |
| | | GAAGAATACGTTGAAGGGGAAGTGGTCGTTCTTGTGGT |
| | | CATAGGGGATGGAAATAATTTTGCTGTGGTAAAGGAAA |
| | | CCCTTGTACATATCGATGACACCTATGACTGCCATATGG |
| | | TGACCCCTCTCCCTCTAGACCCTTCCTTCAGGGAACTATC |
| | | CTACTCCCTTGCAGCAAACCTGCCCTTAAAAGGAATTAT |
| | | GCATGTGGAAGCGATTTCCGGCCCCCTGGGGTTAAAAGT |
| | | TATTGAGATAGATGCCCGTTTCCCGAGCCAGCTTCCGAC |
| | | TGCGGTCTATTATTCTTCCGGGATCAACCTCATAGAACT |
| | | CCTGTTCCGGGCTTTTAATGGAGGCATAGAAGAGATCAA |
| | | AACTCTCCCTGAAGACAGGTACTGCATTTACGAACATCT |
| | | CATGCTTGCAGAAAATGGAGTACTTATCCCTGTGGGAGA |
| | | ACAGGTCCTGTCCATGGGAAATGATTACGGCAATTATTA |
| | | TGAAGAACCTGGAATAGAGATTTTCCTGTGCAAAGGAG |
| | | AGAACCCTGTATTCACCCTGGTTTTCTGGGGCAGAGACA |
| | | GGGAAGAAGCTGAAGCTAGAAAAAACAAAGGGCTTTCG |
| | | ATTCTAAAAAGCCGTTTCGGAGCTGCTGCATAA |
| 133 | K150-11 | MRESWGASLKTICLIGGKLQGFEAAYLSKKAGMKVLVIDK |
| | Polypeptide | NPQALIRNYADEFQCFNITEEPEKLVAISKNVDAILPVNENL |
| | Sequence; | ECIEFLNSIKEKFSCPVLFDFEAYRISRDKRKSKEYFASIGTP |
| | mutations are | TPQDKPSEPPYFVKPPCESSSVGARIIHDRKELKELEPGMLIE |
| | shown in bold and | EYVEGEVVVLVVIGDGNNFAVVKETLVHIDDTYDCHMVT |
| | underlined letters | PLPLDPSFRELSYSLAANLPLKGIMHVEAISGPLGLKVIEIDA |
| | | RFPSQLPTAVYYSSGINLIELLFRAFNGGIEEIKTLPEDRYCI |
| | | YEHLMLAENGVLIPVGEQVLSMGNDYGNYYEEPGIEIFLC |
| | | KGENPVFTLVFWGRDREEAEARKNKGLSILKSRFGAAA |
| 134 | K150-15 | ATGAGAGAGTCCTGGGGTGCTAGCCTGAAAACAATATG |
| | Polynucleotide | CCTTATAGGCGGGAAGCTGCAGGGCTTCGAGGCTGCATA |
| | Sequence; | CCTATCTAAGAAAGCCGGAATGAAAGTGCTTGTAATAG |
| | mutations are | ACAAAAACCCGCAGGCGCTTATAAGGAATTATGCGGAT |
| | shown in bold and | GAGTTCCAGTGTTTTAACATAACGGAAGAGCCGGAAAA |
| | underlined letters | ACTCGTCGCGATATCAAAAAATGTTGATGCCATACTGCC |
| | | GGTAAATGAAAACCTTGAATGTATAGAATTTCTGAATTC |
| | | TATAAAAGAAAAATTCTCCTGCCCGGTACTTTTCGATTTT |
| | | GAAGCTTACAGGATCAGCAGGGATAAGAGAAAATCAAA |
| | | AGAATACTTCGCATCCATAGGAACCCCGACCCCTCAGGA |
| | | CAAACCGTCGGAACCACCTTATTTTGTAAAGCCTCCCTG |
| | | CGAAAGCAGCAGTGTGGGAGCGAGAATAATCCATGACA |
| | | GGAAAGAGCTTAAAGAGCTTGAGCCCGGGATGCTCATA |
| | | GAAGAATACGTTGAAGGGGAAGTGGTCATTCTTCTTGT |
| | | CATAGGGGATGGAAATAATTTTGCTGTGGTAAAGGAAA |
| | | CCCTTGTACATATCGATGACACCTATGACTGCCATATGG |
| | | TGACCCCTCTCCCTCTAGACCCTTCCTTCAGGGAACTATC |
| | | CTACTCCCTTGCAGCAAACCTGCCCTTAAAAGGAATTAT |
| | | GCATGTGGAAGCGATTTCCGGCCCCCTGGGGTTAAAAGT |
| | | TATTGAGATAGATGCCCGTTTCCCGAGCCAGGCGCCGAC |
| | | TGCGGTCTATTATTCTTCCGGGATCAACCTCATAGAACT |
| | | CCTGTTCCGGGCTTTTAATGGAGGCATAGAAGAGATCAA |
| | | AACTCTCCCTGAAGACAGGTACTGCATTTACGAACATCT |
| | | CATGCTTGCAGAAAATGGAGTACTTATCCCTGTGGGAGA |
| | | ACAGGTCCTGTCCATGGGAAATGATTACGGCAATTATTA |
| | | TGAAGAACCTGGAATAGAGATTTTCCTGTGCAAAGGAG |
| | | AGAACCCTGTATTCACCCTGGTTTTCTGGGGCAGAGACA |
| | | GGGAAGAAGCTGAAGCTAGAAAAAACAAAGGGCTTTCG |
| | | ATTCTAAAAAGCCGTTTCGGAGCTGCTGCATAA |
| 135 | K150-15 | MRESWGASLKTICLIGGKLQGFEAAYLSKKAGMKVLVIDK |
| | Polypeptide | NPQALIRNYADEFQCFNITEEPEKLVAISKNVDAILPVNENL |
| | Sequence; | ECIEFLNSIKEKFSCPVLFDFEAYRISRDKRKSKEYFASIGTP |
| | mutations are | TPQDKPSEPPYFVKPPCESSSVGARIIHDRKELKELEPGMLIE |
| | shown in bold and | EYVEGEVVILLVIGDGNNFAVVKETLVHIDDTYDCHMVTP |
| | underlined letters | LPLDPSFRELSYSLAANLPLKGIMHVEAISGPLGLKVIEIDA |
| | | RFPSQAPTAVYYSSGINLIELLFRAFNGGIEEIKTLPEDRYCI |
| | | YEHLMLAENGVLIPVGEQVLSMGNDYGNYYEEPGIEIFLC |
| | | KGENPVFTLVFWGRDREEAEARKNKGLSILKSRFGAAA |
| 136 | K200-6 | ATGAGAGAGTCCTGGGGTGCTAGCCTGAAAACAATATG |
| | Polynucleotide | CCTTATAGGCGGGAAGCTGCAGGGCTTCGAGGCTGCATA |
| | Sequence; | CCTATCTAAGAAAGCCGGAATGAAAGTGCTTGTAATAG |
| | mutations are | ACAAAAACCCGCAGGCGCTTATAAGGAATTATGCGGAT |
| | shown in bold and | GAGTTCCAGTGTTTTAACATAACGGAAGAGCCGGAAAA |
| | underlined letters | ACTCGTCGCGATATCAAAAAATGTTGATGCCATACTGCC |
| | | GGTAAATGAAAACCTTGAATGTATAGAATTTCTGAATTC |
| | | TATAAAAGAAAAATTCTCCTGCCCGGTACTTTTCGATTTT |
| | | GAAGCTTACAGGATCAGCAGGGATAAGAGAAAATCAAA |
| | | AGAATACTTCGCATCCATAGGAACCCCGACCCCTCAGGA |
| | | CAAACCGTCGGAACCACCTTATTTTGTAAAGCCTCCCTG |
| | | CGAAAGCAGCAGTGTGGGAGCGAGAATAATCCATGACA |
| | | GGAAAGAGCTTAAAGAGCTTGAGCCCGGGATGCTCATA |
| | | GAAGAATACGTTGAAGGGGAAGTGGTCCTGCTTGCGGT |
| | | CATAGGGGATGGAAATAATTTTGCTGTGGTAAAGGAAA |
| | | CCCTTGTACATATCGATGACACCTATGACTGCCATATGG |
| | | TGACCCCTCTCCCTCTAGACCCTTCCTTCAGGGAACTATC |
| | | CTACTCCCTTGCAGCAAACCTGCCCTTAAAAGGAATTAT |
| | | GCATGTGGAAGCGATTTCCGGCCCCCTGGGGTTAAAAGT |
| | | TATTGAGATAGATGCCCGTTTCCCGAGCCAGTATCCGAC |
| | | TGCGGTCTATTATTCTTCCGGGATCAACCTCATAGAACT |
| | | CCTGTTCCGGGCTTTTAATGGAGGCATAGAAGAGATCAA |
| | | AACTCTCCCTGAAGACAGGTACTGCATTTACGAACATCT |
| | | CATGCTTGCAGAAAATGGAGTACTTATCCCTGTGGGAGA |
| | | ACAGGTCCTGTCCATGGGAAATGATTACGGCAATTATTA |
| | | TGAAGAACCTGGAATAGAGATTTTCCTGTGCAAAGGAG |
| | | AGAACCCTGTATTCACCCTGGTTTTCTGGGGCAGAGACA |
| | | GGGAAGAAGCTGAAGCTAGAAAAAACAAAGGGCTTTCG |
| | | ATTCTAAAAAGCCGTTTCGGAGCTGCTGCATAA |
| 137 | K200-6 | MRESWGASLKTICLIGGKLQGFEAAYLSKKAGMKVLVIDK |
| | Polypeptide | NPQALIRNYADEFQCFNITEEPEKLVAISKNVDAILPVNENL |
| | Sequence; | ECIEFLNSIKEKFSCPVLFDFEAYRISRDKRKSKEYFASIGTP |
| | mutations are | TPQDKPSEPPYFVKPPCESSSVGARIIHDRKELKELEPGMLIE |
| | shown in bold and | EYVEGEVVLLAVIGDGNNFAVVKETLVHIDDTYDCHMVT |
| | underlined letters | PLPLDPSFRELSYSLAANLPLKGIMHVEAISGPLGLKVIEIDA |
| | | RFPSQYPTAVYYSSGINLIELLFRAFNGGIEEIKTLPEDRYCI |
| | | YEHLMLAENGVLIPVGEQVLSMGNDYGNYYEEPGIEIFLC |
| | | KGENPVFTLVFWGRDREEAEARKNKGLSILKSRFGAAA |
| 138 | K200-10 | ATGAGAGAGTCCTGGGGTGCTAGCCTGAAAACAATATG |
| | Polynucleotide | CCTTATAGGCGGGAAGCTGCAGGGCTTCGAGGCTGCATA |
| | Sequence; | CCTATCTAAGAAAGCCGGAATGAAAGTGCTTGTAATAG |
| | mutations are | ACAAAAACCCGCAGGCGCTTATAAGGAATTATGCGGAT |
| | shown in bold and | GAGTTCCAGTGTTTTAACATAACGGAAGAGCCGGAAAA |
| | underlined letters | ACTCGTCGCGATATCAAAAAATGTTGATGCCATACTGCC |
| | | GGTAAATGAAAACCTTGAATGTATAGAATTTCTGAATTC |
| | | TATAAAAGAAAAATTCTCCTGCCCGGTACTTTTCGATTTT |
| | | GAAGCTTACAGGATCAGCAGGGATAAGAGAAAATCAAA |
| | | AGAATACTTCGCATCCATAGGAACCCCGACCCCTCAGGA |
| | | CAAACCGTCGGAACCACCTTATTTTGTAAAGCCTCCCTG |
| | | CGAAAGCAGCAGTGTGGGAGCGAGAATAATCCATGACA |
| | | GGAAAGAGCTTAAAGAGCTTGAGCCCGGGATGCTCATA |
| | | GAAGAATACGTTGAAGGGGAAGTGGTCGGTCTTGTTGT |
| | | CATAGGGGATGGAAATAATTTTGCTGTGGTAAAGGAAA |
| | | CCCTTGTACATATCGATGACACCTATGACTGCCATATGG |
| | | TGACCCCTCTCCCTCTAGACCCTTCCTTCAGGGAACTATC |
| | | CTACTCCCTTGCAGCAAACCTGCCCTTAAAAGGAATTAT |
| | | GTTGGTGGAAGCGATTTCCGGCCCCCTGGGGTTAAAAGT |
| | | TATTGAGATAGATGCCCGTTTCCCGAGCCAGCTGCCGAC |
| | | TGCGGTCTATTATTCTTCCGGGATCAACCTCATAGAACT |
| | | CCTGTTCCGGGCTTTTAATGGAGGCATAGAAGAGATCAA |
| | | AACTCTCCCTGAAGACAGGTACTGCATTTACGAACATCT |
| | | CATGCTTGCAGAAAATGGAGTACTTATCCCTGTGGGAGA |
| | | ACAGGTCCTGTCCATGGGAAATGATTACGGCAATTATTA |
| | | TGAAGAACCTGGAATAGAGATTTTCCTGTGCAAAGGAG |
| | | AGAACCCTGTATTCACCCTGGTTTTCTGGGGCAGAGACA |
| | | GGGAAGAAGCTGAAGCTAGAAAAAACAAAGGGCTTTCG |
| | | ATTCTAAAAAGCCGTTTCGGAGCTGCTGCATAA |
| 139 | K200-10 | MRESWGASLKTICLIGGKLQGFEAAYLSKKAGMKVLVIDK |
| | Polypeptide | NPQALIRNYADEFQCFNITEEPEKLVAISKNVDAILPVNENL |
| | Sequence; | ECIEFLNSIKEKFSCPVLFDFEAYRISRDKRKSKEYFASIGTP |
| | mutations are | TPQDKPSEPPYFVKPPCESSSVGARIIHDRKELKELEPGMLIE |
| | shown in bold and | EYVEGEVVGLVVIGDGNNFAVVKETLVHIDDTYDCHMVT |
| | underlined letters | PLPLDPSFRELSYSLAANLPLKGIMLVEAISGPLGLKVIEIDA |
| | | RFPSQLPTAVYYSSGINLIELLFRAFNGGIEEIKTLPEDRYCI |
| | | YEHLMLAENGVLIPVGEQVLSMGNDYGNYYEEPGIEIFLC |
| | | KGENPVFTLVFWGRDREEAEARKNKGLSILKSRFGAAA |
| 140 | K200-11 | ATGAGAGAGTCCTGGGGTGCTAGCCTGAAAACAATATG |
| | Polynucleotide | CCTTATAGGCGGGAAGCTGCAGGGCTTCGAGGCTGCATA |
| | Sequence; | CCTATCTAAGAAAGCCGGAATGAAAGTGCTTGTAATAG |
| | mutations are | ACAAAAACCCGCAGGCGCTTATAAGGAATTATGCGGAT |
| | shown in bold and | GAGTTCCAGTGTTTTAACATAACGGAAGAGCCGGAAAA |
| | underlined letters | ACTCGTCGCGATATCAAAAAATGTTGATGCCATACTGCC |
| | | GGTAAATGAAAACCTTGAATGTATAGAATTTCTGAATTC |
| | | TATAAAAGAAAAATTCTCCTGCCCGGTACTTTTCGATTTT |
| | | GAAGCTTACAGGATCAGCAGGGATAAGAGAAAATCAAA |
| | | AGAATACTTCGCATCCATAGGAACCCCGACCCCTCAGGA |
| | | CAAACCGTCGGAACCACCTTATTTTGTAAAGCCTCCCTG |
| | | CGAAAGCAGCAGTGTGGGAGCGAGAATAATCCATGACA |
| | | GGAAAGAGCTTAAAGAGCTTGAGCCCGGGATGCTCATA |
| | | GAAGAATACGTTGAAGGGGAAGTGGTCAGTCTTCCGGT |
| | | CATAGGGGATGGAAATAATTTTGCTGTGGTAAAGGAAA |
| | | CCCTTGTACATATCGATGACACCTATGACTGCCATATGG |
| | | TGACCCCTCTCCCTCTAGACCCTTCCTTCAGGGAACTATC |
| | | CTACTCCCTTGCAGCAAACCTGCCCTTAAAAGGAATTAT |
| | | GAATGTGGAAGCGATTTCCGGCCCCCTGGGGTTAAAAGT |
| | | TATTGAGATAGATGCCCGTTTCCCGAGCCAGGTGCCGAC |
| | | TGCGGTCTATTATTCTTCCGGGATCAACCTCATAGAACT |
| | | CCTGTTCCGGGCTTTTAATGGAGGCATAGAAGAGATCAA |
| | | AACTCTCCCTGAAGACAGGTACTGCATTTACGAACATCT |
| | | CATGCTTGCAGAAAATGGAGTACTTATCCCTGTGGGAGA |
| | | ACAGGTCCTGTCCATGGGAAATGATTACGGCAATTATTA |
| | | TGAAGAACCTGGAATAGAGATTTTCCTGTGCAAAGGAG |
| | | AGAACCCTGTATTCACCCTGGTTTTCTGGGGCAGAGACA |
| | | GGGAAGAAGCTGAAGCTAGAAAAAACAAAGGGCTTTCG |
| | | ATTCTAAAAAGCCGTTTCGGAGCTGCTGCATAA |

PylC Variants for Synthesizing D-Pra-ε-Lys

| | | |
|---|---|---|
| 141 | K200-11 | MRESWGASLKTICLIGGKLQGFEAAYLSKKAGMKVLVIDK |
| | Polypeptide | NPQALIRNYADEFQCFNITEEPEKLVAISKNVDAILPVNENL |
| | Sequence; | ECIEFLNSIKEKFSCPVLFDFEAYRISRDKRKSKEYFASIGTP |
| | mutations are | TPQDKPSEPPYFVKPPCESSSVGARIIHDRKELKELEPGMLIE |
| | shown in bold and | EYVEGEVVSLPVIGDGNNFAVVKETLVHIDDTYDCHMVTP |
| | underlined letters | LPLDPSFRELSYSLAANLPLKGIMNVEAISGPLGLKVIEIDA |
| | | RFPSQVPTAVYYSSGINLIELLFRAFNGGIEEIKTLPEDRYCI |
| | | YEHLMLAENGVLIPVGEQVLSMGNDYGNYYEEPGIEIFLC |
| | | KGENPVFTLVFWGRDREEAEARKNKGLSILKSRFGAAA |
| 142 | K200-12 | ATGAGAGAGTCCTGGGGTGCTAGCCTGAAAACAATATG |
| | Polynucleotide | CCTTATAGGCGGGAAGCTGCAGGGCTTCGAGGCTGCATA |
| | Sequence | CCTATCTAAGAAAGCCGGAATGAAAGTGCTTGTAATAG |
| | (PylC^{GVNV}); | ACAAAAACCCGCAGGCGCTTATAAGGAATTATGCGGAT |
| | mutations are | GAGTTCCAGTGTTTTAACATAACGGAAGAGCCGGAAAA |
| | shown in bold and | ACTCGTCGCGATATCAAAAAATGTTGATGCCATACTGCC |
| | underlined letters | GGTAAATGAAAACCTTGAATGTATAGAATTTCTGAATTC |
| | | TATAAAAGAAAAATTCTCCTGCCCGGTACTTTTCGATTTT |
| | | GAAGCTTACAGGATCAGCAGGGATAAGAGAAAATCAAA |
| | | AGAATACTTCGCATCCATAGGAACCCCGACCCCTCAGGA |
| | | CAAACCGTCGGAACCACCTTATTTTGTAAAGCCTCCCTG |
| | | CGAAAGCAGCAGTGTGGGAGCGAGAATAATCCATGACA |
| | | GGAAAGAGCTTAAAGAGCTTGAGCCCGGGATGCTCATA |
| | | GAAGAATACGTTGAAGGGGAAGTGGTCGGGCTTGTTGT |
| | | CATAGGGGATGGAAATAATTTTGCTGTGGTAAAGGAAA |
| | | CCCTTGTACATATCGATGACACCTATGACTGCCATATGG |
| | | TGACCCCTCTCCCTCTAGACCCTTCCTTCAGGGAACTATC |
| | | CTACTCCCTTGCAGCAAACCTGCCCTTAAAAGGAATTAT |
| | | GAATGTGGAAGCGATTTCCGGCCCCCTGGGGTTAAAAGT |
| | | TATTGAGATAGATGCCCGTTTCCCGAGCCAGGTTCCGAC |
| | | TGCGGTCTATTATTCTTCCGGGATCAACCTCATAGAACT |
| | | CCTGTTCCGGGCTTTTAATGGAGGCATAGAAGAGATCAA |
| | | AACTCTCCCTGAAGACAGGTACTGCATTTACGAACATCT |
| | | CATGCTTGCAGAAAATGGAGTACTTATCCCTGTGGGAGA |
| | | ACAGGTCCTGTCCATGGGAAATGATTACGGCAATTATTA |
| | | TGAAGAACCTGGAATAGAGATTTTCCTGTGCAAAGGAG |
| | | AGAACCCTGTATTCACCCTGGTTTTCTGGGGCAGAGACA |
| | | GGGAAGAAGCTGAAGCTAGAAAAAACAAAGGGCTTTCG |
| | | ATTCTAAAAAGCCGTTTCGGAGCTGCTGCATAA |
| 143 | K200-12 | MRESWGASLKTICLIGGKLQGFEAAYLSKKAGMKVLVIDK |
| | Polypeptide | NPQALIRNYADEFQCFNITEEPEKLVAISKNVDAILPVNENL |
| | Sequence | ECIEFLNSIKEKFSCPVLFDFEAYRISRDKRKSKEYFASIGTP |
| | (PylC^{GVNV}); | TPQDKPSEPPYFVKPPCESSSVGARIIHDRKELKELEPGMLIE |
| | mutations are | EYVEGEVVGLVVIGDGNNFAVVKETLVHIDDTYDCHMVT |
| | shown in bold and | PLPLDPSFRELSYSLAANLPLKGIMNVEAISGPLGLKVIEIDA |
| | underlined letters | RFPSQVPTAVYYSSGINLIELLFRAFNGGIEEIKTLPEDRYCI |
| | | YEHLMLAENGVLIPVGEQVLSMGNDYGNYYEEPGIEIFLC |
| | | KGENPVFTLVFWGRDREEAEARKNKGLSILKSRFGAAA |

Sequences for PylC Variants for Synthesizing D-Allyl-ε-Lys

| | | |
|---|---|---|
| 142 | K200-12 | ATGAGAGAGTCCTGGGGTGCTAGCCTGAAAACAATATG |
| | Polynucleotide | CCTTATAGGCGGGAAGCTGCAGGGCTTCGAGGCTGCATA |
| | Sequence | CCTATCTAAGAAAGCCGGAATGAAAGTGCTTGTAATAG |
| | (PylC^{GVNV}); | ACAAAAACCCGCAGGCGCTTATAAGGAATTATGCGGAT |
| | mutations are | GAGTTCCAGTGTTTTAACATAACGGAAGAGCCGGAAAA |
| | shown in bold and | ACTCGTCGCGATATCAAAAAATGTTGATGCCATACTGCC |
| | underlined letters | GGTAAATGAAAACCTTGAATGTATAGAATTTCTGAATTC |
| | | TATAAAAGAAAAATTCTCCTGCCCGGTACTTTTCGATTTT |
| | | GAAGCTTACAGGATCAGCAGGGATAAGAGAAAATCAAA |
| | | AGAATACTTCGCATCCATAGGAACCCCGACCCCTCAGGA |
| | | CAAACCGTCGGAACCACCTTATTTTGTAAAGCCTCCCTG |
| | | CGAAAGCAGCAGTGTGGGAGCGAGAATAATCCATGACA |
| | | GGAAAGAGCTTAAAGAGCTTGAGCCCGGGATGCTCATA |
| | | GAAGAATACGTTGAAGGGGAAGTGGTCGGGCTTGTTGT |
| | | CATAGGGGATGGAAATAATTTTGCTGTGGTAAAGGAAA |
| | | CCCTTGTACATATCGATGACACCTATGACTGCCATATGG |
| | | TGACCCCTCTCCCTCTAGACCCTTCCTTCAGGGAACTATC |
| | | CTACTCCCTTGCAGCAAACCTGCCCTTAAAAGGAATTAT |
| | | GAATGTGGAAGCGATTTCCGGCCCCCTGGGGTTAAAAGT |
| | | TATTGAGATAGATGCCCGTTTCCCGAGCCAGGTTCCGAC |
| | | TGCGGTCTATTATTCTTCCGGGATCAACCTCATAGAACT |
| | | CCTGTTCCGGGCTTTTAATGGAGGCATAGAAGAGATCAA |
| | | AACTCTCCCTGAAGACAGGTACTGCATTTACGAACATCT |
| | | CATGCTTGCAGAAAATGGAGTACTTATCCCTGTGGGAGA |
| | | ACAGGTCCTGTCCATGGGAAATGATTACGGCAATTATTA |
| | | TGAAGAACCTGGAATAGAGATTTTCCTGTGCAAAGGAG |
| | | AGAACCCTGTATTCACCCTGGTTTTCTGGGGCAGAGACA |
| | | GGGAAGAAGCTGAAGCTAGAAAAAACAAAGGGCTTTCG |
| | | ATTCTAAAAAGCCGTTTCGGAGCTGCTGCATAA |
| 143 | K200-12 | MRESWGASLKTICLIGGKLQGFEAAYLSKKAGMKVLVIDK |
| | Polypeptide | NPQALIRNYADEFQCFNITEEPEKLVAISKNVDAILPVNENL |
| | Sequence | ECIEFLNSIKEKFSCPVLFDFEAYRISRDKRKSKEYFASIGTP |
| | (PylC^{GVNV}); | TPQDKPSEPPYFVKPPCESSSVGARIIHDRKELKELEPGMLIE |
| | mutations are | EYVEGEVVGLVVIGDGNNFAVVKETLVHIDDTYDCHMVT |
| | shown in bold and | PLPLDPSFRELSYSLAANLPLKGIMNVEAISGPLGLKVIEIDA |
| | underlined letters | RFPSQVPTAVYYSSGINLIELLFRAFNGGIEEIKTLPEDRYCI |
| | | YEHLMLAENGVLIPVGEQVLSMGNDYGNYYEEPGIEIFLC |
| | | KGENPVFTLVFWGRDREEAEARKNKGLSILKSRFGAAA |

PylRS and tRNA Sequences

| | | |
|---|---|---|
| 144 | Wild-type M. | MDKKPLNTLISATGLWMSRTGTIHKIKHHEVSRSKIYIEMA |
| | mazei PylRS | CGDHLVVNNSRSSRTARALRHHKYRKTCKRCRVSDEDLN |
| | (UniProt ID: | KFLTKANEDQTSVKVKVVSAPTRTKKAMPKSVARAPKPLE |
| | Q8PWY1; amino | NTEAAQAQPSGSKFSPAIPVSTQESVSVPASVSTSISSISTGA |
| | acids that | TASALVKGNTNPITSMSAPVQASAPALTKSQTDRLEVLLNP |
| | correspond to G14, | KDEISLNSGKPFRELESELLSRRKKDLQQIYAEERENYLGKL |
| | S451, and C348 | EREITRFFVDRGFLEIKSPILIPLEYIERMGIDNDTELSKQIFR |
| | that may be | VDKNFCLRPMLAPNLYNYLRKLDRALPDPIKIFEIGPCYRK |
| | mutated are shown | ESDGKEHLEEFTMLNFCQMGSGCTRENLESIITDFLNHLGI |
| | in bold and | DFKIVGDSCMVYGDTLDVMHGDLELSSAVVGPIPLDREWG |
| | underlined letters | IDKPWIGAGFGLERLLKVKHDFKNIKRAARSESYYNGISTN |
| | | L |
| 145 | PylRS^{EVF} | ATGGATAAAAAACCACTAAACACTCTGATATCTGCAACC |
| | Polynucleotide | GAGCTCTGGATGTCCAGGACCGGAACAATTCATAAAAT |
| | Sequence; | AAAACACCACGAAGTCTCTCGAAGCAAAATCTATATTGA |
| | mutations are | AATGGCATGCGGAGACCACCTTGTTGTAAACAACTCCAG |
| | shown in bold and | GAGCAGCAGGACTGCAAGAGCGCTCAGGCACCACAAAT |
| | underlined letters | ACAGGAAGACCTGCAAACGCTGCAGGGTTTCGGATGAG |
| | | GATCTCAATAAGTTCCTCACAAAGGCAAACGAAGACCA |
| | | GACAAGCGTAAAAGTCAAGGTCGTTTCTGCCCCTACCAG |
| | | AACGAAAAAGGCAATGCCAAAATCCGTTGCGAGAGCCC |
| | | CGAAACCTCTTGAGAATACAGAAGCGGCACAGGCTCAA |
| | | CCTTCTGGATCTAAATTTTCACCTGCGATACCGGTTTCCA |
| | | CCCAAGAGTCAGTTTCTGTCCCGGCATCTGTTTCAACAT |
| | | CAATATCAAGCATTTCTACAGGAGCAACTGCATCCGCAC |
| | | TGGTAAAAGGGAATACGAACCCCATTACATCCATGTCTG |
| | | CCCCTGTTCAGGCAAGTGCCCCCGCACTTACGAAGAGCC |
| | | AGACTGACAGGCTTGAAGTCCTGTTAAACCCAAAAGAT |
| | | GAGATTTCCCTGAATTCCGGCAAGCCTTTCAGGGAGCTT |
| | | GAGTCCGAATTGCTCTCTCGCAGAAAAAAAGACCTGCA |
| | | GCAGATCTACGCGGAAGAAAGGGAGAATTATCTGGGGA |
| | | AACTCGAGCGTGAAATTACCAGGTTCTTTGTGGACAGGG |
| | | GTTTTCTGGAAATAAAATCCCCGATCCTGATCCCTCTTG |
| | | AGTATATCGAAAGGATGGGCATTGATAATGATACCGAA |
| | | CTTTCAAAACAGATCTTCAGGGTTGACAAGAACTTCTGC |
| | | CTGAGACCCATGCTTGCTCCAAACCTTTACAACTACCTG |
| | | CGCAAGCTTGACAGGGCCCTGCCTGATCCAATAAAAATT |
| | | TTTGAAATAGGCCCATGCTACAGAAAAGAGTCCGACGG |
| | | CAAAGAACACCTCGAAGAGTTTACCATGCTGAACTTCGT |
| | | GCAGATGGGATCGGGATGCACACGGGAAAATCTTGAAA |
| | | GCATAATTACGGACTTCCTGAACCACCTGGGAATTGATT |
| | | TCAAGATCGTAGGCGATTCCTGCATGGTCTATGGGGATA |
| | | CCCTTGATGTAATGCACGGAGACCTGGAACTTTCCTCTG |
| | | CAGTAGTCGGACCCATACCGCTTGACCGGGAATGGGGT |
| | | ATTGATAAACCCTGGATAGGGGCAGGTTTCGGGCTCGAA |
| | | CGCCTTCTAAAGGTTAAACACGACTTTAAAAATATCAAG |
| | | AGAGCTGCAAGGTCCGAGTCTTACTATAACGGGATTTTT |
| | | ACCAACCTGTAA |
| 146 | PyIRS^{EVF} | MDKKPLNTLISATELWMSRTGTIHKIKHHEVSRSKIYIEMA |
| | Polypeptide | CGDHLVVNNSRSSRTARALRHHKYRKTCKRCRVSDEDLN |
| | Sequence; | KFLTKANEDQTSVKVKVVSAPTRTKKAMPKSVARAPKPLE |
| | mutations are | NTEAAQAQPSGSKFSPAIPVSTQESVSVPASVSTSISSISTGA |
| | shown in bold and | TASALVKGNTNPITSMSAPVQASAPALTKSQTDRLEVLLNP |
| | underlined letters | KDEISLNSGKPFRELESELLSRRKKDLQQIYAEERENYLGKL |
| | | EREITRFFVDRGFLEIKSPILIPLEYIERMGIDNDTELSKQIFR |
| | | VDKNFCLRPMLAPNLYNYLRKLDRALPDPIKIFEIGPCYRK |
| | | ESDGKEHLEEFTMLNFVQMGSGCTRENLESIITDFLNHLGI |
| | | DFKIVGDSCMVYGDTLDVMHGDLELSSAVVGPIPLDREWG |
| | | IDKPWIGAGFGLERLLKVKHDFKNIKRAARSESYYNGIFTN |
| | | L |
| 147 | tRNA^{Pyl} | GGAAACCUGAUCAUGUAGAUCGAAUGGACUCUAAAUC |
| | | CGUUCAGCCGGGUUAGAUUCCCGGGGUUUCCGCCA |

Primer and Promoter Sequences

| | | |
|---|---|---|
| 148 | PylTm15_Fwd | CAAGGAGGAAACCTGGTCAGGGAGACCGAACGGACTCT |
| | | AAATC |
| 149 | PylTm15_Rev | GATTTAGAGTCCGTTCGGTCTCCCTGACCAGGTTTCCTCC |
| | | TTG |
| 150 | PylTstarSecondC_ | CCGTTCAGCCGGGTTCGATTCCCGGGGTTTCCGCCACGT |
| | Fwd | TTC |
| 151 | PylTstarSecondC_ | GAAACGTGGCGGAAACCCCGGGAATCGAACCCGGCTGA |
| | Rev | ACGG |
| 152 | PT_{7lac} | TAATACGACTCACTATAGGGGAATTGTGAGCGGATAAC |
| | | AATTCC |
| 153 | PL_{lacO1} | AATTGTGAGCGGATAACAATTGACATTGTGAGCGGATA |
| | | ACAAGATACTGAGCAC |
| 154 | P_{tac} | TTGACAATTAATCATCGGCTCGTATAATGTGTGGAATTG |
| | | TGAGCGGATAACAA |
| 155 | Pₗₚₚ | CCCATCAAAAAAATATTCTCAACATAAAAAACTTTGTGT |
| | | AATACTTGTAACGCT |
| 156 | PylS Fwd primer | CAGAGTCTATATCTAGAAATAATTTTGTTTAACTTTAAG |
| | for EpPCR of PylS | AAGGAGATATACCATGG |
| 157 | R4 primer for | GCGCGCCGAGCTCGAATTCGGATCC |
| | EpPCR of PylS | |
| 158 | F2-PylS-Cys348 | CATGCTGAACTTCNNNCAGATGGGATC |
| | primer for Site- | |
| | saturation | |
| | mutagenesis of | |
| | PylS C348 | |
| 159 | PylTm15_Fwd | CAAGGAGGAAACCTGGTCAGGGAGACCGAACGGACTCT |
| | primer for | AAATC |
| | Mutagenesis of | |
| | tRNA^{M15} | |
| 160 | PylTm15_Rev | GATTTAGAGTCCGTTCGGTCTCCCTGACCAGGTTTCCTCC |
| | primer for | TTG |
| | Mutagenesis of | |
| | tRNA^{M15} | |
| 161 | PylTStarSecondC_ | CCGTTCAGCCGGGTTCGATTCCCGGGGTTTCCGCCACGT |
| | Fwd primer for | TTC |
| | Mutagenesis of | |
| | tRNA^{M15} | |
| 162 | PylTStarSecondC_ | GAAACGTGGCGGAAACCCCGGGAATCGAACCCGGCTGA |
| | Rev primer for | ACGG |
| | Mutagenesis of | |
| | tRNA^{M15} | |
| 163 | PAmpRrev_Fwd | GGGTTATTGTCTCATGAGCGGATAC |
| | primer for | |
| | Amplification of | |
| | the pPylST.tL | |
| | vector | |
| 164 | DuetUP2- | CGCTCACAATGTCAATTGTTATCCGCTCACAATTATTTCG |
| | MCS2PLlacO1_ | ATTATGCGGCCGTGTA |
| | Rev primer for | |
| | Amplification of | |
| | the pPylST.tL | |
| | vector | |
| 165 | pDuet- | CCATCTTAGTATATTAGTTAAGTA |
| | MCS2Pdown_Fwd | |
| | primer for | |
| | Amplification of | |
| | mCh(TAG) | |
| 166 | pDuetupAmpR_ | CTGTCAGACCAAGTTTACTCATATATACTTTAG |
| | Rev primer for | |
| | Amplification of | |
| | mCh(TAG) | |
| 167 | pDuet- | TAAACTTGGTCTGACAGTTATTTGCCGACTACCTTGGTG |
| | SmRrev_Fwd | |
| | primer for | |
| | Amplification of | |
| | smR gene | |
| 168 | PAmpRrev_Rev | GTATCCGCTCATGAGACAATAACCC |
| | primer for | |
| | Amplification of | |
| | smR gene | |
| 169 | MCSIPtac- | CGGCTCGTATAATGTGTGGAATTGTGAGCGGATAACAAC |
| | PylT_Fwd primer | C |
| | for Replacement of | |
| | T7lac | |
| | promoter with P_{tac} | |
| 170 | rbs-NcoI- | CAGAGTGTTTAGTGGTTTTTTATCCATGGTATATCTCCTT |
| | 5PylS_Rev primer | CTTAAAGTTAAACA |
| | for Replacement of | |
| | T7lac promoter | |
| | with P_{tac} | |
| 171 | pDuet- | ATCTCGATCCCGCGAAATTTGACAATTAATCATCGGCTC |
| | MCS1Ptac_Fwd | GTATAATGTGTGGA |
| | primer for | |
| | Replacement of | |
| | T7lac promoter | |
| | with P_{tac} | |
| 172 | PylCS177E179mut_ | GAATACGTTGAAGGGGAAGTGGTCNNKCTTNNKGTCAT |
| | Fwd primer for | AGGGGATGGAAATAATTTTGC |
| | Generation of 4 | |
| | PylC mutations | |
| 173 | PylCD233mut_ | GCCGGAAATCGCTTCCACMNNCATAATTCCTTTTAAGGG |
| | Rev primer for | CAGGTTTGC |
| | Generation of 4 | |
| | PylC mutations | |
| 174 | PylCD233mut_ | GCAAACCTGCCCTTAAAAGGAATTATGNNKGTGGAAGC |
| | Fwd primer for | GATTTCCGG |
| | Generation of 4 | |
| | PylC mutations | |
| 175 | PylCT256mut_ | GGAAGAATAATAGACCGCAGTCGGMNNCTGGCTCGGGA |
| | Rev primer for | AACGG |
| | Generation of 4 | |
| | PylC mutations | |
| 176 | rbs- | AAGTATAAGAAGGAGATATACATATGTCTGACCAGGAG |
| | NdeISUMO_Fwd | GCAAAAC |
| | primer for | |
| | Extension of | |
| | SUMO-PylC | |
| | fragment | |
| 177 | PylCS177up_Rev | GACCACTTCCCCTTCAACGTATTC |
| | primer for | |
| | Extension of | |
| | SUMO-PylC | |
| | fragment | |
| 178 | PylCT256down_ | CCGACTGCGGTCTATTATTCTTCC |
| | Fwd primer for | |
| | Extension of | |
| | SUMO-PylC | |
| | fragment | |
| 179 | PylC- | GGTTTCTTTACCAGACTCGAGGGTACCTTATGCAGCAGC |
| | KpnIpDuet_Rev | TCCGAAAC |
| | primer for | |
| | Extension of | |
| | SUMO-PylC | |
| | fragment | |
| 180 | Plpp-rbs_Fwd | CATAAAAAACTTTGTGTAATACTTGTAACGCTCCATCTT |
| | primer for | AGTATATTAGTTAAGTATAAGAAGGAGAT |
| | Replacement of | |
| | promoter to Pₗₚₚ | |
| 181 | O-to- | ATACAAAGGAGGAAGTGGAGCGGGACACGACGCTGC |
| | Alamutant_Fwd | |
| | primer for | |
| | Mutagenesis of | |
| | TAG to GCG | |
| 182 | O-to- | GCAGCGTCGTGTCCCGCTCCACTTCCTCCTTTGTAT |
| | Alamutant_Rev | |
| | primer for | |
| | Mutagenesis of | |
| | TAG to GCG | |

1. A recombinant host cell comprising a polynucleotide sequence encoding a mutant PylC capable of synthesizing a noncanonical amino acid (ncAA), wherein the mutant PylC comprises a polypeptide sequence with at least 95% sequence identity with SEQ ID NO: 1 (wild-type M. mazei PylC).

2. The recombinant host cell of claim 1, further comprising
(a) a polynucleotide sequence encoding a wild-type PylRS or a mutant PylRS capable of using the ncAA as a substrate, and
(b) a polynucleotide sequence encoding a tRNA that incorporates the ncAA at an amber codon.

3. The recombinant host cell of claim 1, wherein the mutant PylC comprises a combination of mutations corresponding to residues 177, 179, 233, and 256 of wild-type archaeal PylC.

4. The recombinant host cell of claim 3, wherein the mutant PylC combination of mutations is chosen from:
1) S177N, E179P, D233S, and T256V;
2) S177C, E179T, D233S, and T256V;
3) E179C and D233N;
4) S177A, E179A, D233H, and T256M;
5) S177A, E179S, D233H, and T256L;
6) E179C, D233H, and T256V;
7) E179V, D233N, and T256V;
8) E179V, D233H, and T256N;
9) S177T, E179P, D233H, and T256M;
10) S177G, E179V, D233S, and T256L;
11) E179N, D233H, and T256L;
12) S177A, E179V, D233H, and T256I;
13) S177L, E179I, D233H, and T256C;
14) S177A, E179I, D233T, and T256L;
15) S177H, E179L, D233S, and T256V;
16) S177A, E179M, D233S, and T256L;
17) E179I, D233H, and T256I;
18) S177A, E179T, D233H, and T256V;
19) S177A, E179A, D233N, and T256C;
20) S177C, E179P, D233H, and T256L;
21) S177G, E179I, and D233H;
22) S177M, E179V, D233H, and T256V;
23) S177V, E179V, D233H, and T256V;
24) S177A, E179A, D233S, and T256V;
25) S177G, E179M, D233N, and T256V;
26) S177G, E179I, D233N, and T256V;
27) S177G, E179T, D233S, and T256L;
28) S177A, E179T, D233S, and T256L;
29) S177L, E179T, D233S, and T256I;
30) S177T, E179P, D233H, and T256M;
31) E179L, D233L, and T256V;
32) S177A, E179V, D233Q, and T256C;
33) S177L, E179V, D233S, and T256A;
34) S177A, E179C, D233L, and T256C;
35) S177N, E179C, D233T, and T256C;
36) S177H, E17L9, D233S, and T256S;
37) E179G, D233L, and T256V;
38) S177N, E179P, D233S, and T256V;
39) S177M, E179G, D233H, and T256Y;
40) S177M, E179L, D233S, and T256C;
41) S177A, E179C, AND D233H;
42) E179L, D233L, and T256I;
43) S177L, E179V, D233H, and T256Y;
44) S177A, E179Q, D233N, and T256C; and
45) S177L, E179A, D233H, and T256V.

5. (canceled)

6. The recombinant host cell of claim 3, wherein the mutant PylC combination of mutations is chosen from:
1) S177G, E179V, D233N, and T256V;
2) S177V, E179L, D233H, and T256L;
3) S177G, E179V, D233H, and T256A;
4) S177V, E179C, D233H, and T256L;
5) S177M, E179V, D233H, and T256Y;
6) S177C, E179P, D233H, and T256L;
7) S177G, E179A, D233S, and T256L;
8) S177V, E179L, D233H, and T256V;
9) S177G E179L, D233H, and T256L;
10) S177I, D233H, and T256V;
11) S177L, E179V, D233T, and T256I;
12) S177L, E179A, D233H, and T256M;
13) S177L, E179V, D233H, and T256A;
14) S177I, E179M, D233H, and T256L;
15) S177A, E179I, D233N, and T256I;
16) S177I, E179C, D233H, and T256L;
17) E179S and D233H;
18) S177V, E179V, D233H, and T256A;
19) S177C, E179V, D233H, and T256Y;
20) S177I, E179A, D233H, and T256H;
21) S177L, E179C, D233H, and T256V;
22) E179V and D233H;
23) S177V, E179V, D233H, and T256L;
24) S177I, E179L, D233H, and T256A;
25) S177L, E179A, D233H, and T256Y;
26) S177G, E179V, D233L, and T256L;
27) E179P, D233N, and T256V;
28) S177G, E179V, D233N, and T256N;
29) S177L, E179C, D233G, and T256H;
30) S177H, E179G, D233S, and T256A;
31) S177A, E179C, D233S, and T256L;
32) S177G, E179V, D233L, and T256Y;
33) S177L, E179T, D233T, and T256M;
34) S177G, E179L, D233W, and T256S;
35) S177V, E179L, D233S, and T256A;
36) E179V, D233L, and T256L;
37) S177L, E179P, D233S, and T256N; and
38) S177V, E179V, D233T, and T256L.

7. (canceled)

8. The recombinant host cell of claim 1, wherein the mutant PylRS comprises mutations that correspond to (a) G114E, C348V, and S451F of wild-type archaeal PylRS, or (b) L301M, L305I, Y306L, L309A, and C348F of wild-type archaeal PylRS.

9. The recombinant host cell of claim 1, wherein its genomic sequence encoding Release Factor 1 (RF1) is at least partially deleted.

10. The recombinant host cell of claim 1, wherein the cell is a prokaryotic cell.

11.-12. (canceled)

12. A composition comprising the recombinant host cell of claim 1.

13. A lysate of the recombinant host cell of claim 1.

14. A method for recombinant synthesis of a protein comprising:
(a) introducing a nucleotide sequence encoding the protein into the recombinant host cell of claim 1, wherein the nucleotide sequence comprises (i) at least one TAG codon within the nucleotide sequence's coding sequence, and (ii) a TAA codon or a TGA codon at the end of the nucleotide sequence's coding sequence; and
(b) incubating the recombinant host cell under conditions permissible for transcription from the nucleotide sequence and for protein synthesis, thereby producing the protein; and wherein
the protein comprises one or more ncAA, optionally wherein the one or more ncAA is chosen from D-Cys-ε-Lys, D-Pra-ε-Lys, D-Allyl-ε-Lys, and 2-Chloro-Acetyl-ε-Lys.

15. The method of claim 14, further comprising isolating the protein produced in step (b).

16. The method of claim 15, further comprising placing the isolated protein under conditions permissive for the protein to cyclize by forming a covalent bond between a (i) first moiety on a first ncAA and (ii) a second moiety on a cysteine, C-terminal thioester, or a second moiety on a second ncAA.

17. (canceled)

18. A method for recombinant synthesis of a protein comprising contacting a nucleotide sequence encoding the protein with a cell lysate of the recombinant host cell of claim 1.

19. (canceled)

20. A nucleic acid comprising a polynucleotide sequence encoding a mutant PylC capable of synthesizing a ncAA, wherein the mutant PylC comprises a combination of mutations corresponding to residues 177, 179, 233, and 256 of wild-type archaeal PylC.

21. (canceled)

22. The nucleic acid of claim 21, wherein the mutant PylC combination of mutations is chosen from:
1) S177N, E179P, D233S, and T256V;
2) S177C, E179T, D233S, and T256V;
3) E179C and D233N;
4) S177A, E179A, D233H, and T256M;
5) S177A, E179S, D233H, and T256L;
6) E179C, D233H, and T256V;
7) E179V, D233N, and T256V;
8) E179V, D233H, and T256N;
9) S177T, E179P, D233H, and T256M;
10) S177G, E179V, D233S, and T256L;
11) E179N, D233H, and T256L;
12) S177A, E179V, D233H, and T256I;
13) S177L, E179I, D233H, and T256C;
14) S177A, E179I, D233T, and T256L;
15) S177H, E179L, D233S, and T256V;
16) S177A, E179M, D233S, and T256L;
17) E179I, D233H, and T256I;
18) S177A, E179T, D233H, and T256V;
19) S177A, E179A, D233N, and T256C;
20) S177C, E179P, D233H, and T256L;
21) S177G, E179I, and D233H;
22) S177M, E179V, D233H, and T256V;
23) S177V, E179V, D233H, and T256V;
24) S177A, E179A, D233S, and T256V;
25) S177G, E179M, D233N, and T256V;
26) S177G, E179I, D233N, and T256V;
27) S177G, E179T, D233S, and T256L;
28) S177A, E179T, D233S, and T256L;
29) S177L, E179T, D233S, and T256I;
30) S177T, E179P, D233H, and T256M;
31) E179L, D233L, and T256V;
32) S177A, E179V, D233Q, and T256C;
33) S177L, E179V, D233S, and T256A;
34) S177A, E179C, D233L, and T256C;
35) S177N, E179C, D233T, and T256C;
36) S177H, E17L9, D233S, and T256S;
37) E179G, D233L, and T256V;
38) S177N, E179P, D233S, and T256V;
39) S177M, E179G, D233H, and T256Y;
40) S177M, E179L, D233S, and T256C;
41) S177A, E179C, AND D233H;
42) E179L, D233L, and T256I;
43) S177L, E179V, D233H, and T256Y;
44) S177A, E179Q, D233N, and T256C; and
45) S177L, E179A, D233H, and T256V.

23. (canceled)

24. The nucleic acid of claim 21, wherein the mutant PylC combination of mutations is chosen from:
1. S177G, E179V, D233N, and T256V;
2. S177V, E179L, D233H, and T256L;
3. S177G, E179V, D233H, and T256A;
4. S177V, E179C, D233H, and T256L;
5. S177M, E179V, D233H, and T256Y;
6. S177C, E179P, D233H, and T256L;
7. S177G, E179A, D233S, and T256L;
8. S177V, E179L, D233H, and T256V;
9. S177G E179L, D233H, and T256L;
10. S177I, D233H, and T256V;
11. S177L, E179V, D233T, and T256I;
12. S177L, E179A, D233H, and T256M;
13. S177L, E179V, D233H, and T256A;
14. S177I, E179M, D233H, and T256L;
15. S177A, E179I, D233N, and T256I;
16. S177I, E179C, D233H, and T256L;
17. E179S and D233H;
18. S177V, E179V, D233H, and T256A;
19. S177C, E179V, D233H, and T256Y;
20. S177I, E179A, D233H, and T256H;
21. S177L, E179C, D233H, and T256V;
22. E179V and D233H;
23. S177V, E179V, D233H, and T256L;
24. S177I, E179L, D233H, and T256A;
25. S177L, E179A, D233H, and T256Y;
26. S177G, E179V, D233L, and T256L;
27. E179P, D233N, and T256V; and
28. S177G, E179V, D233N, and T256N.

25. (canceled)

26. A nucleic acid comprising a polynucleotide sequence encoding a mutant PylRS capable of using an ncAA as a substrate, wherein the mutant PylRS comprises mutations that correspond to (a) G14E, C348V and S451F of wild-type archaeal PylRS, or (b) L301M, L305I, Y306L, L309A, and C348F of wild-type archaeal PylRS.

27. (canceled)

28. A composition comprising the nucleic acid of claim 21.

29. An expression cassette or a vector comprising a polynucleotide sequence of the nucleic acid of claim 21.
